(19) United States
(12) Patent Application Publication
(10) Pub. No.: US 2025/0073271 A1
(43) Pub. Date: Mar. 6, 2025

(54) METHODS OF ADMINISTERING PLATELET DERIVATIVE COMPOSITIONS TO REDUCE BLEEDING IN SUBJECTS REFRACTORY TO PLATELET TRANSFUSION

(71) Applicant: Cellphire, Inc., Rockville, MD (US)

(72) Inventors: Keith Andrew MOSKOWITZ, Westfield, IN (US); Glen Michael FITZPATRICK, North Potomac, MD (US)

(73) Assignee: Cellphire, Inc., Rockville, MD (US)

(21) Appl. No.: 18/949,648

(22) Filed: Nov. 15, 2024

Related U.S. Application Data
(63) Continuation-in-part of application No. 18/052,709, filed on Nov. 4, 2022.
(60) Provisional application No. 63/709,341, filed on Oct. 18, 2024.
(60) Provisional application No. 63/625,196, filed on Jan. 25, 2024.
(60) Provisional application No. 63/599,917, filed on Nov. 16, 2023.
(60) Provisional application No. 63/599,987, filed on Nov. 16, 2023.
(60) Provisional application No. 63/376,986, filed on Sep. 23, 2022.
(60) Provisional application No. 63/371,849, filed on Aug. 18, 2022.
(60) Provisional application No. 63/364,620, filed on May 12, 2022.
(60) Provisional application No. 63/264,227, filed on Nov. 17, 2021.
(60) Provisional application No. 63/264,226, filed on Nov. 17, 2021.
(60) Provisional application No. 63/276,420, filed on Nov. 5, 2021.
(60) Provisional application No. 63/275,937, filed on Nov. 4, 2021.

(51) Int. Cl. A61K 35/19; A61P 7/04

(52) U.S. Cl. CPC A61K 35/19; A61P 7/04

(57) ABSTRACT

Provided herein are compositions, and methods for administering a platelet derivative composition for reducing bleeding in a subject who is refractory to platelet transfusion. Methods herein can further include administering a platelet derivative composition to a subject having low platelet counts (e.g., having thrombocytopenia) and/or to maintain the number of platelets over a threshold amount, or to increase the number of platelets in the subject. Methods herein can include administering a therapeutic agent before and optionally after administering the platelet derivative composition to the subject. Methods herein can reduce or eliminate any dosing and/or scheduling adjustments of a therapeutic regimen due to a low platelet count.

### CROSS-REFERENCE TO RELATED APPLICATIONS

This application claims priority to U.S. Provisional Application Ser. No. 63/599,917, filed on Nov. 16, 2023, U.S. Provisional Application Ser. No. 63/599,987, filed on Nov. 16, 2023, U.S. Provisional Application Ser. No. 63/625,196, filed on Jan. 25, 2024, U.S. Provisional Application Ser. No. 63/709,341, filed on Oct. 18, 2024, and is a continuation in part of U.S. application Ser. No. 18/052,709, filed on Nov. 4, 2022. U.S. application Ser. No. 18/052,709 claims priority to U.S. Provisional Application Ser. No. 63/275,937, filed on Nov. 4, 2021, U.S. Provisional Application Ser. No. 63/276,420, filed on Nov. 5, 2021, U.S. Provisional Application Ser. No. 63/264,226, filed on Nov. 17, 2021, U.S. Provisional Application Ser. No. 63/264,227, filed on Nov. 17, 2021, U.S. Provisional Application Ser. No. 63/364,620, filed on May 12, 2022, U.S. Provisional Application Ser. No. 63/371,849, filed on Aug. 18, 2022, and U.S. Provisional Application Ser. No. 63/376,986, filed on Sep. 23, 2022. Each of the applications listed in this paragraph is incorporated herein by reference in its entirety.

### STATEMENT OF GOVERNMENT INTEREST

This invention was made with government support under Contract No. HHS0100201300021 awarded by the Biomedical Advanced Research and Development Authority (BARDA) of the U.S. Department of Health and Human Services. The government has certain rights in the invention.

### TECHNICAL FIELD

The present disclosure generally relates to blood products, including those containing platelet derivatives, methods of producing such blood products, and methods of treating a subject using such blood products.

### BACKGROUND

Blood is a complex mixture of numerous components. In general, blood can be described as comprising four main parts: red blood cells, white blood cells, platelets, and plasma. The first three are cellular or cell-like components, whereas the fourth (plasma) is a liquid component comprising a wide and variable mixture of salts, proteins, and other factors necessary for numerous bodily functions. The components of blood can be separated from each other by various methods. In general, differential centrifugation is most commonly used currently to separate the different components of blood based on size and, in some applications, density.

Platelets generally function by adhering to the lining of broken blood vessels, in the process becoming activated, changing to an amorphous shape, and interacting with components of the clotting system that are present in plasma or are released by the platelets themselves or other components of the blood. Although, fresh platelets have found use in treating subjects with low platelet count (thrombocytopenia) in many cases they are ineffective. For example, patients can become refractory to platelet transfusions. And the current standards of care for treating many diseases, such as many types of cancer, cause thrombocytopenia, which can be drug-induced thrombocytopenia (DIT) and more specifically chemotherapy-induced thrombocytopenia (CIT), where the drug is a chemotherapeutic agent. DIT occurs when certain therapeutic agents destroy platelets or interfere with the body's ability to make enough of them. While chemotherapy drugs are the most common cancer therapeutic agents that can cause DIT, there are other therapeutic agents that can lead to DIT including¹: furosemide, gold, used to treat arthritis; nonsteroidal anti-inflammatory drugs (NSAIDs); penicillin; quinidine; quinine; ranitidine; sulfonamides; linezolid and other antibiotics and statins. CIT is a common side effect of cancer therapies and aside from the risk of bleeding, thrombocytopenia limits chemotherapy dose and frequency. There are many chemotherapy agents and cancer regimes (including those that involve radiation) that cause thrombocytopenia and there are no FDA or EU approved treatments for CIT. Platelet transfusion is still the standard of care for DIT and drugs approved to treat idiopathic thrombocytopenic purpura (ITP) are currently being investigated for treating CIT. Although there are approved therapies for treating such thrombocytopenia, such as recombinant thrombopoietin (TPO), such therapies are often ineffective, and thus, for example, do not provide uninterrupted chemotherapy to the patients without the risk of bleeding. Thus, there is a need for improved methods of treating thrombocytopenia.

Since thrombocytopenia increases the risk of bleeding in a patient, it can have multiple negative effects on a patient. Not only can it result in bleeding episode(s), it can also have indirect effects, such as eliminating certain treatment options for patients not only for their cancer, but for unrelated health issues they may be facing, for example surgery. Furthermore, the fear of bleeding can have negative psychological implications for patients. Thus, DIT and CIT can affect a patient's quality of life in many ways, thus increasing the importance of supportive care for these patients.

Supportive care includes various types of intervention within oncology treatment. For example, supportive care can describe intensive medical treatment, “palliative” anticancer treatment, pain and symptom control (i.e., cancer-related, cancer treatment-related), and psychosocial support. Principles of supportive care as laid out by the Multinational Association of Supportive Care in Cancer (MASCC) includes: (a) maintaining or improving quality of life, and to ensure that people with cancer can achieve the maximum benefit from their anticancer treatment; (b) ensuring support to the patients throughout the continuum of the cancer experience, from diagnosis through treatment to post-treatment (encompassing cancer survivorship and palliative and end of life care); (c) involving a coordinated, person-centric, and holistic (whole-person) approach; and (d) ensuring basic rights for all people with cancer, irrespective of their personal circumstances, their type of cancer, their stage of cancer, or their anticancer treatment. Current support care strategies for cancer patients are not effective enough for many patients, for example to relieve the negative effects of thrombocytopenia. Accordingly, there is a need for improved supportive care for cancer patients who develop thrombocytopenia as a side-effect of their cancer therapeutic regimen.

The risk of bleeding from thrombocytopenia presents a serious health risk because there remains a need for effective therapies for treating bleeding not only in thrombocytopenic patients and severe thrombocytopenic patients, but in any subject who is experiencing bleeding. For example, there remains a need for improved treatments for decreasing bleeding in a subject who is refractory to platelet transfusions.

### SUMMARY

In order to overcome the current problems and long-standing needs in the art, for example those discussed above, the following non-limiting exemplary aspects and embodiments are provided.

Provided herein in one aspect, is a method of treating a subject, for example, reducing or decreasing bleeding in the subject, comprising:
rehydrating a platelet derivative composition in the form of a powder to form a rehydrated platelet derivative composition comprising rehydrated platelet derivatives, and
administering a dose of the rehydrated platelet derivative composition to the subject, wherein the dose comprises a first dose of the rehydrated platelet derivatives,
wherein the subject is refractory to platelet transfusion,
wherein the first dose of the rehydrated platelet derivatives is an amount of between 5×10⁸ particles/kg and 1.0×10¹² particles/kg of the subject,
wherein the administering leads to the subject having decreased bleeding without a bleeding score of WHO Grade 2A or greater at 15 minutes, 30 minutes, 45 minutes, 1 hour, 2, 4, 6, 8, 10, or 12 hours after the administering the first dose of the platelet derivatives.

Provided herein in one aspect is a method for administering a platelet derivative composition to a subject, comprising:
administering a cancer therapeutic agent to the subject, according to a pre-platelet derivative cancer therapeutic dosing regimen,
determining that the subject has a low platelet count, wherein the low platelet count is a blood platelet count of less than 150,000 platelets/μl of circulating blood,
administering an effective dose of platelet derivatives in the platelet derivative composition to the subject, and
administering the cancer therapeutic agent to the subject according to a post-platelet derivative cancer therapeutic dosing regimen,
wherein the first dose administered in the post-platelet derivative cancer therapeutic dosing regimen is not delayed and/or the first dose is not reduced due to the low platelet count.

Provided herein in one aspect is a method for administering a platelet derivative composition to a subject, comprising:
administering a cancer therapeutic agent to the subject according to a pre-platelet derivative cancer therapeutic dosing regimen,
administering an effective dose of platelet derivatives in the platelet derivative composition to the subject, and
administering the cancer therapeutic agent to the subject according to a post-platelet derivative chemotherapeutic dosing regimen,
wherein the first dose administered in the post-platelet derivative cancer therapeutic dosing regimen is not delayed and/or the first dose is not reduced due to a low platelet count in the subject.

Provided herein in one aspect is a method for administering a platelet derivative composition to a subject, comprising:
administering an effective dose of platelet derivatives in the platelet derivative composition to the subject before and/or after an n^{th} round of administering a cancer therapeutic agent according to a cancer therapeutic dosing regimen, and
continuing the cancer therapeutic dosing regimen by administering the cancer therapeutic agent in an additional round to the subject, wherein the first dose of the cancer therapeutic agent in the additional round is not reduced and/or the first dose is not delayed due to a low platelet count in the subject.

Provided herein in one aspect is a method for administering a platelet derivative composition to a subject having thrombocytopenia, comprising:
administering a first round of platelets at an initial timepoint to the subject having thrombocytopenia;
after the administering, determining that a platelet count in the subject did not increase by an expected amount;
administering an effective dose of the platelet derivatives in a platelet derivative composition to the subject;
administering a subsequent round(s) of platelets to the subject at a subsequent timepoint(s); and
after the administering of the subsequent round of the platelets, determining that the platelet count in the subject is increased to an amount closer to the expected amount, than after the first round of administering, or fall within the expected amount.

In another aspect, provided herein is a method for administering a platelet derivative composition to a subject having thrombocytopenia, comprising:
administering platelets at an initial timepoint to the subject having thrombocytopenia, in an amount expected to increase a platelet count by an expected amount within a target platelet increase timeframe after the initial timepoint;
after the administering and during the target platelet increase timeframe, determining that the platelet count did not increase by the expected amount; and
within 24, 12, 8, 6, 4, 2, or 1 hour after the initial timepoint, administering an effective dose of the platelet derivatives in the platelet derivative composition to the subject.

In another aspect, provided herein is a method for increasing platelet counts in a subject having thrombocytopenia, comprising:
administering platelets at an initial timepoint to the subject having thrombocytopenia;
after the administering and during a target platelet increase timeframe after the initial timepoint, determining that the platelet count in the subject did not increase by an expected amount based on the number of platelets that were administered;
within 24, 12, 8, 6, 4, 2, or 1 hour after the initial timepoint, administering an effective dose of the platelet derivatives in the platelet derivative composition to the subject; and
after the administering the effective dose of the platelet derivatives, administering platelets at a subsequent timepoint to the subject, wherein the platelet count in the subject is increased closer to the expected amount, than the administering platelets at the initial timepoint or is increased by the expected amount.

In another aspect, provided herein is a method of treating a subject having refractory thrombocytopenia, comprising:
administering an effective dose of platelet derivatives in a platelet derivative composition to the subject having refractory thrombocytopenia.

In another aspect, provided herein is a method for administering a platelet derivative composition to a subject having an initial low platelet count, comprising:
administering platelets to the subject having the initial low platelet count at an initial timepoint in an amount predicted to increase a platelet count by an expected amount;
after the administering the platelets, determining that the platelet count in the subject remains below the expected amount;
administering an effective dose of the platelet derivatives in the platelet derivative composition to the subject; and
after the administering the effective dose of the platelet derivatives, administering platelets at a subsequent timepoint to the subject, wherein the administering the platelets increases the platelet count closer to or by the expected amount.

In another aspect, provided herein is a method of treating a subject having thrombocytopenia, comprising:
a) administering an effective dose of platelet derivatives in a platelet derivative composition at an initial platelet derivative timepoint to the subject having thrombocytopenia; and
b) within less than 1 hour after the administering the effective dose of the platelet derivatives, administering platelets to the subject, wherein after the administering platelets, the number of platelets in the subject increases.

In another aspect, provided herein is a method of treating a subject having thrombocytopenia, comprising:
a) administering platelets at an initial timepoint to the subject having thrombocytopenia,
b) administering an effective dose of platelet derivatives in a platelet derivative composition at an initial platelet derivative timepoint to the subject, wherein the initial timepoint is more than 6 hours before the initial platelet derivative timepoint; and
c) administering platelets to the subject at a subsequent timepoint, wherein after the administering platelets, the number of platelets in the subject increases.

Further details regarding aspects and embodiments of the present disclosure are provided throughout this patent application. The preceding paragraphs in this Summary section is not an exhaustive list of aspects and embodiments disclosed herein. Sections and section headers are for ease of reading and are not intended to limit combinations of disclosure, such as methods, compositions, and kits or functional elements therein across sections.

### BRIEF DESCRIPTION OF THE DRAWINGS

FIG. 1 illustrates the primary endpoint data depicting percentage of subjects with WHO bleeding score of Grade 1 or less, at 12 hours and 24 hours after the administration of a first dose of platelet derivatives.

FIG. 2A, FIG. 2B, and FIG. 2C illustrates the WHO bleeding score at the primary site after 12 hours, 24 hours, and 72 hours, respectively of the administration of a first dose of platelet derivatives.

FIG. 3 illustrates the cross-reactivity of platelet derivatives in the presence of samples from two thrombocytopenic (TCP) subjects, 01-001, and 01-012.

FIG. 4 illustrates thrombin generation (peak thrombin) ability of the platelet derivatives in the presence of samples from two thrombocytopenic (TCP) subjects, 01-001, and 01-012 as compared to a control.

FIG. 5 illustrates thrombin generation (peak thrombin) ability of the platelet derivatives, and its comparison to apheresis platelet units (APU) in the presence or absence of anti-platelet agent abciximab.

FIG. 6 illustrates the average Mean Fluorescence Intensity (MFI) corresponding to the presence of IgG on the surface of FPH, from the three FPH lots, with error bars representing standard deviation.

FIG. 7A illustrates the peak thrombin for the different HPS serum samples when incubated with FPH.

FIG. 7B illustrates the lag time, time until thrombin first generated for the different HPS samples when incubated with FPH.

FIG. 7C illustrates the endogenous thrombin potential (ETP) or the net amount of thrombin that HPS plasma samples can generate when incubated with FPH.

FIG. 7D illustrates the time to peak or the time for maximum concentration of thrombin generated for the different HPS samples when incubated with FPH.

FIG. 7E illustrates the velocity index or the slope of the thrombin potential between the lag time and the time to peak for the different HPS samples when incubated with FPH.

FIG. 8A illustrates the occlusion start time in the presence of FPH and HPS serum samples, in T-TAS assay.

FIG. 8B illustrates the occlusion time in the presence of FPH and HPS serum samples, in T-TAS assay.

FIG. 8C illustrates the area under the curve (AUC) in the presence of FPH and HPS serum samples, in T-TAS assay.

FIG. 9 illustrates the platelet count in the subjects at time periods starting from baseline to day 7 after administering a first dose of the platelet derivatives.

### DEFINITIONS

Unless otherwise noted, technical terms are used according to conventional usage. Unless otherwise explained, all technical and scientific terms used herein have the same meaning as commonly understood by one of ordinary skill in the art to which this disclosure belongs. The singular terms “a,” “an,” and “the” include plural referents unless the context clearly indicates otherwise. “Comprising A or B” means including A, or B, or A and B. It is further to be understood that all base sizes or amino acid sizes, and all molecular weight or molecular mass values, given for nucleic acids or polypeptides are approximate, and are provided for description.

Further, ranges provided herein are understood to be shorthand for all of the values within the range. For example, a range of 1 to 50 is understood to include any number, combination of numbers, or sub-range from the group consisting, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 1 to 49, 1 to 25, 1.7 to 31.9, and so forth (as well as fractions thereof unless the context clearly dictates otherwise). Any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. When multiple low and multiple high values for ranges are given that overlap, a skilled artisan will recognize that a selected range will include a low value that is less than the high value.

As used herein, “about” or “consisting essentially of” mean±10% of the indicated range, value, or structure, unless otherwise indicated. As used herein, the terms “include” and “comprise” are open ended and are used synonymously. As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

Although methods and materials similar or equivalent to those described herein can be used in the practice or testing of the present disclosure, suitable methods and materials are described below. All publications, patent applications, patents, and other references mentioned herein are incorporated by reference in their entireties. In case of conflict, the present specification, including explanations of terms, will control. In addition, the materials, methods, and examples are illustrative only and not intended to be limiting.

It is appreciated that certain features of aspects and embodiments herein, which are, for clarity, discussed in the context of separate embodiments, may also be provided in combination in a single embodiment. Conversely, various aspects and embodiments, which are, for brevity, discussed in the context of a single aspect or embodiment, may also be provided separately or in any suitable sub-combination. All combinations of aspects and embodiments are specifically embraced herein and are disclosed herein just as if each and every combination was individually and explicitly disclosed. In addition, all sub-combinations of the various aspects and embodiments and elements thereof are also specifically disclosed herein even if each and every such sub-combination is not individually and explicitly disclosed herein.

As used herein, “hemostatic properties” include the following properties: (a) the ability to generate thrombin in a thrombin formation assay, for example in the presence of tissue factor and phospholipids; (b) the ability to occlude a collagen-coated microchannel in vitro, for example under conditions in which fresh platelets can occlude a collagen-coated microchannel in vitro; (c) the capability of thrombin-induced trapping in the presence of thrombin. As demonstrated in Examples herein, platelet derivatives, such as a freeze-dried platelet derivatives (e.g., thrombosomes) herein, in illustrative embodiments are hemostats, and thus have one, two, or all of the aforementioned hemostatic properties. [00043]“Platelets” may include, for example, platelets in whole blood, platelets in plasma, platelets in buffer optionally supplemented with select plasma proteins, cold stored platelets. Platelets may be from a mammal(s), such as of humans, or such as non-human mammals.

As used herein, “thrombosomes” (sometimes also called Tsomes) or “thrombosomes platelet derivatives” are platelet derivatives that have been treated with a preparation agent (e.g., any of the preparation agents described herein) and lyopreserved (such as freeze-dried). In some cases, thrombosomes platelet derivatives can be prepared from pooled platelets. Thrombosomes platelet derivatives can have a shelf life of 2-3 years in dry form at ambient temperature and can be rehydrated with sterile water within minutes (e.g. 1, 2, 3, 4, 5, 10 15, 20, 25, or 30 minutes) for immediate infusion. One example of thrombosomes freeze-dried platelet derivatives are THROMBOSOMES® freeze-dried platelet derivatives (Cellphire Inc., Rockville, MD), which are in clinical trials. In non-limiting illustrative embodiments, FDPD compositions (e.g., thrombosome) are prepared according to Example 1 of U.S. Pat. No. 11,529,587 B2, incorporated by reference herein in its entirety, or Example 1 of PCT app no. PCT/US2022/079280, incorporated by reference herein in its entirety. In non-limiting illustrative embodiments, thrombosome compositions, or illustrative freeze-dried platelet-derivative compositions herein, are compositions that include platelet derivatives, wherein at least 50% of the platelet derivatives are CD 41-positive platelet derivatives, wherein less than 15%, 10%, or in further, non-limiting illustrative embodiments less than 5% of the CD 41-positive platelet derivatives are microparticles having a diameter of less than 0.5 μm, and wherein the platelet derivatives have a potency of at least 0.5, 1.0 and in further, non-limiting illustrative embodiments 1.5 thrombin generation potency units (TGPU) per 10⁶ platelet derivatives. In certain illustrative embodiments, including non-limiting examples of the illustrative embodiment in the preceding sentence, the platelet derivatives are at least 0.5 μm in diameter, and in some embodiments 0.5 to 2.5 μm in diameter. Thus, thrombosomes are typically freeze-dried platelet derivatives (FDPDs). Freeze-dried platelet derivatives herein (e.g. thrombosomes) typically have at least 1 hemostatic property, and thus can function as hemostatic agents. Therefore, FDPDs and compositions herein comprising the same, can also be referred to as hemostat(s), hemostatic product(s), freeze-dried platelet derived hemostat(s) (FDPDH or FPDH), freeze-dried platelet hemostat(s) (FDPH or FPH), dry platelet derivative hemostat(s) (PDH), freeze-dried platelet derivative hemostatic(s) (i.e., freeze-dried platelet derivative hemostatic agents), or freeze-dried platelet hemostatic(s) (i.e. freeze-dried platelet hemostatic agent(s)).

### DETAILED DESCRIPTION

Provided herein are methods and compositions for administering platelet derivatives to a subject, such as a human subject/patient, for treating bleeding of the subject and/or for treating an indication for which the subject is afflicted.

### Platelet Derivatives for Treating Bleeding and for Treating Disorders

In some embodiments, a platelet derivative composition, in illustrative embodiment a freeze-dried platelet derivative, including, but not limited to, those of any of the aspects or embodiments herein, or the platelet derivative composition prepared by any of the process described in the aspects or embodiments herein, can be administered, or delivered to a subject that is bleeding and/or having an indication and thus afflicted with a disorder or disease that could benefit from delivery of such platelet derivative compositions. Such disorder or disease in illustrative embodiments is a disorder or disease that causes bleeding in a subject. The bleeding in the subject for example in illustrative embodiments, is a bleeding that is scored as a WHO Grade 2 but not solely the result of cutaneous bleeding, which can be referred to herein as WHO Grade 2A. After administration of platelet derivative compositions using methods herein, in some embodiments bleeding is reduced in the subject such that bleeding of the subject, for example at a primary site, and in illustrative embodiments in secondary sites too, is below WHO Grade 2A (i.e., no sites of bleeding are WHO Grade 2A or greater).

In some embodiments, indications of subjects treated with methods herein can be any one or a combination of Von Willebrand disease, immune thrombocytopenia (ITP), intracranial hemorrhage (ICH), traumatic brain injury (TBI), Hermansky Pudlak Syndrome (HPS), chemotherapy induced thrombocytopenia (CIT), Scott syndrome, Evans syndrome, hematopoietic stem cell transplantation, fetal and neonatal alloimmune thrombocytopenia, Bernard Soulier syndrome, acute myeloid leukemia, Glanzmann thrombasthenia, myelodysplastic syndrome, hemorrhagic shock, coronary thrombosis (myocardial infarction), ischemic stroke, arterial thromboembolism, Wiskott Aldrich syndrome, venous thromboembolism, MYH9 related disease, acute lymphoblastic lymphoma (ALL), acute coronary syndrome, chronic lymphocytic leukemia (CLL), acute promyelocytic leukemia, cerebral venous sinus thrombosis (CVST), liver cirrhosis, factor v deficiency (Owren Parahemophilia), thrombocytopenia absent radius syndrome, Kasabach Merritt syndrome, Gray platelet syndrome, aplastic anemia, chronic liver disease, acute radiation syndrome, Dengue hemorrhagic fever, pre-eclampsia, snakebite envenomation, HELLP syndrome, haemorrhagic cystitis, multiple myeloma, disseminated intravascular coagulation, heparin induced thrombocytopenia, pre-eclampsia, labor and delivery, hemophilia, cerebral (fatal) malaria, Alexander's disease (Factor VII Deficiency), hemophilia C (Factor XI Deficiency), familial hemophagocytic lymphohistiocytosis, acute lung injury, hemolytic uremic syndrome, menorrhagia, chronic myeloid leukemia. In illustrative embodiments, a platelet derivative composition of any of the aspects or embodiments herein, or the platelet derivative composition prepared by any of the process described in the aspects or embodiments herein can be administered, or delivered to a subject afflicted by Immune thrombocytopenia. In certain illustrative embodiments, a platelet derivative composition of any of the aspects or embodiments herein, or the platelet derivative composition prepared by any of the process described in the aspects or embodiments herein can be administered, or delivered to a subject afflicted by Von Willebrand disease. In some embodiments, a method of treating of any of the aspects or embodiments herein, can include a method of treating a subject afflicted with any of the indications as described herein. In any of the methods herein wherein platelet derivatives are administered to a subject having any of the listed indications/disorders, the subject can have an anti-coagulant or antiplatelet agent in their body, such as in their blood, and can be, or have been within 1 month, 1 week, 5 days, 4 days, 3, days, 2 days, 1 day, 12 hours, 8 hours, or 4 hours, taking or administered an anti-coagulant and/or an anti-platelet agent.

In any of the aspects and embodiments herein, a subject herein does not have one or more of the following indications: thrombotic or ischemic conditions, does not consume any pro- or anti-thrombotic medications, and the blood sample from the subject shows liver enzyme or blood creatinine levels>3× upper limit of normal (ULN).

In some embodiments, a platelet derivative composition of any of the aspects or embodiments herein, or the platelet derivative composition prepared by any of the process described in the aspects or embodiments can be used to treat a subject having any one or a combination of any of the indications as described herein. In certain embodiments, a platelet derivative composition as described herein can be used, for example as a medicament or in the manufacture of a kit, for treating a subject having any one or a combination of indication as disclosed herein.

In some embodiments, an indication or indications can include those type of indications which require a much higher dose of the platelet derivatives herein, or would require an unsafe dose of, or cannot be treated with, unmodified, cold stored, naturally-occurring, endogenous, autologous, allogeneic, or normal platelets, platelets having the characteristics of in-vivo platelets, or conventional platelets (e.g. platelets collected by a conventional method for collecting platelets such as, for example, a platelet-rich plasma-method, a buff coat-method, or by apheresis), but are treatable using a platelet derivative composition of any of the aspects or embodiments herein, or the platelet derivative composition prepared by any of the processes disclosed herein, in illustrative embodiments freeze-dried platelet derivatives. In some embodiments, such an indication can be an indication that is associated with defective platelet production in a subject. In some embodiments, such an indication can be an indication that is associated with a defective platelet activity in a subject. In some embodiments, such an indication can be any of the indication as described herein. In some embodiments, indications that are typically cannot be treated with conventional platelets, but are treatable with a platelet derivative composition as disclosed herein are Von Willebrand disease, immune thrombocytopenia (ITP), intracranial hemorrhage (ICH), traumatic brain injury (TBI), Hermansky Pudlak Syndrome (HPS), Chemotherapy induced thrombocytopenia (CIT), Scott syndrome, Evans syndrome, Hematopoietic Stem Cell Transplantation, Fetal and neonatal alloimmune thrombocytopenia, Bernard Soulier syndrome, Acute myeloid leukemia, or combinations thereof. In some embodiments, such an indication can be Von Willebrand disease. In some embodiments, such an indication can be Immune thrombocytopenia. In some embodiments, such an indication can be Chemotherapy induced thrombocytopenia (CIT). In some embodiments, such an indication can be fetal and neonatal alloimmune thrombocytopenia.

In some embodiments, an indication or indications can include those type of indications which typically, can be well-suited for treatment using a platelet derivative composition as described herein. In some embodiments, such an indication or indications can include Von Willebrand disease, immune thrombocytopenia, intracranial hemorrhage (ICH), traumatic brain injury (TBI), Hermansky Pudlak Syndrome (HPS), Scott syndrome, Evans syndrome, Bernard Soulier syndrome, Glanzmann thrombasthenia, coronary thrombosis (myocardial infarction), arterial thromboembolism, Wiskott Aldrich syndrome, venous thromboembolism, and acute coronary syndrome.

In some embodiments, platelet derivatives as described herein can have several applications in terms of treating a subject suffering with a disorder selected from the group consisting of alopecia areata, Von Willebrand Disease, hemophilia, thrombasthenia, thrombocytopenia, thrombocytopenic purpura, trauma, or a combination thereof. In some embodiments, the platelet derivatives can be used to treat clotting-related disorders. The platelet derivatives as disclosed herein can be used both as a topical application and systemic administration. In some embodiments, there is provided a method for treating a clotting-related disorder in a subject, said method comprising administering to the subject a therapeutically effective amount of the platelet derivative composition of any of the aspects or embodiments herein, or the platelet derivative composition prepared by any of the process described in the aspects or embodiments herein. In some embodiments, the clotting-related disorder is selected from the group consisting of Von Willebrand Disease, hemophilia, thrombasthenia, thrombocytopenia, thrombocytopenic purpura, trauma, or a combination thereof. In some embodiments, a platelet derivative composition is passed through a filter of 18 μm before administering to the subject. A skilled artisan would be able to appreciate that the platelet derivative composition in the form of a powder which would be commercialized in vials would be rehydrated with an appropriate amount of a solution before administering to a subject. In some embodiments, such a rehydrated platelet derivative composition is passed through a filter of 18 μm before administering to the subject. In some embodiments, the platelet derivative composition as disclosed herein can be used in treating a coagulopathy in a subject that has been administered or is being administered an antiplatelet agent. In some embodiments, the platelet derivative composition of any of the aspects or embodiments herein is provided for use an anti-platelet reversal agent. In some embodiments, the platelet derivative composition of any of the aspects or embodiments herein can be used in treating a coagulopathy in a subject that has been administered or is being administered an anticoagulant agent.

In some embodiments, the platelet derivatives disclosed herein can be used for healing wounds in a subject. In some embodiments, there is provided a method for healing a wound in a subject, comprising administering a therapeutically effective amount of a platelet derivative composition of any of the aspects or embodiments herein, or the platelet derivative composition prepared by any of the process described in the aspects or embodiments herein, to the subject and/or a wound of the subject.

In some embodiments, the administering can include administering topically. Administering can include administering parenterally. Administering can include administering intravenously. Administering can include administering intramuscularly. Administering can include administering intrathecally. Administering can include administering subcutaneously. Administering can include administering intraperitoneally.

Platelet derivative compositions comprising platelet derivatives as described herein can be used as a medicament for treating a subject. Further, there is also provided herein, methods of treating a subject by administering to a subject a therapeutically effective amount or dose of a platelet derivative composition comprising platelet derivatives as described herein. In some embodiments, the subject is suffering from a condition, or a disease selected from the group consisting of thrombocytopenia, hematologic malignancy, bone marrow aplasia, myeloproliferative disorders, myelodysplastic syndromes, and platelet refractoriness. In some embodiments, the subject is suffering from one or more diseases or condition as described herein. In some embodiments, a therapeutically effective dose of platelet derivatives is based on units of thrombin generation activity administered per kilogram of body weight of the subject and/or the number of platelet derivatives delivered to the subject. In some embodiments of any aspect or embodiment herein the effective dose is based on the weight of the subject. It can be contemplated by a person of skill in the art that the effective dose can be based on any criteria that suits the requirement of the medical intervention of the subject.

A person of skill in the art can contemplate the effective dose of platelet derivatives that can be required to treat a subject in need thereof. The need may differ based on the condition of the subject. The effective dosage can be categorized into a) low dosage; b) medium dosage; and c) high dosage. In some embodiments, a medicament or a method of treating a subject can have the effective dose as low, medium, or high dosage of platelet derivatives that can broadly range from 1.0×10⁷ on the low end of the range to 1.0×10¹⁰/kg, 1.0×10¹¹/kg or 1.0×10¹²/kg of the subject on the high end of the range.

In some embodiments, a platelet derivative composition as described herein can be administered or delivered to a subject, such as a subject afflicted with any one or combination of indications as described herein, and the dose of a platelet derivative composition can be in the range of 1.0×10⁷ to 1.0×10¹² particles/kg of the subject. For example, in some embodiments, a dose of a composition comprising platelets or platelet derivatives (e.g., FDPDs) can include between about or exactly 1.0×10⁷ on the low end of the range to 1.0×10¹² particles (e.g. FDPDs)/kg of a subject on the high end of the range, 1.0×10⁷ to 1.0×10¹¹ particles (e.g. FDPDs)/kg of a subject, 1.0×10⁷ to 1.0×10¹⁰ particles (e.g. FDPDs)/kg of a subject, 1.6×10⁷ to 1.0×10¹¹ particles (e.g. FDPDs)/kg of a subject, 1.6×10⁷ to 1.0×10¹⁰ particles (e.g. FDPDs/kg of subject, 1.6×10⁷ to 5.1×10⁹ particles (e.g. FDPDs/kg of a subject, 1.6×10⁷ to 3.0×10⁹ particles (e.g. FDPDs)/kg of a subject, 1.6×10⁷ to 1.0×10⁹ particles (e.g. FDPDs)/kg of a subject, 1.6×10⁷ to 5.0×10⁸ particles (e.g. FDPDs)/kg of a subject, 1.6×10⁷ to 1.0×10⁸ particles (e.g. FDPDs)/kg of a subject, 1.6×10⁷ to 5.0×10⁷ particles (e.g. FDPDs)/kg of a subject, 5.0×10⁷ to 1.0×10⁸ particles (e.g. FDPDs)/kg of a subject, 1.0×10⁸ to 5.0×10⁸ particles (e.g. FDPDs)/kg of a subject, 5.0×10⁸ to 1.0×10⁹ particles (e.g. FDPDs)/kg of a subject, 1.0×10⁹ to 5.0×10⁹ particles (e.g. FDPDs)/kg of a subject, 5.0×10⁷ to 1.0×10¹¹ particles (e.g. FDPDs)/kg of a subject, 5.0×10⁹ to 1.0×10¹⁰ particles (e.g. FDPDs)/kg of a subject), or 1.0×10¹⁰ to 1.0×10¹¹ or 1.0×10¹² particles (e.g. FDPDs)/kg of a subject on the high end of the range. In some embodiments, the dose can be in the range of 250 and 5000 TGPU per kg of the subject.

In some embodiments, a platelet derivative composition, such as that provided in any aspect or embodiment herein, can be administered or delivered to a subject afflicted with any one or combination of indications/diseases as disclosed herein, and the dose of a platelet derivative composition can be in the range of 1.0×10⁷ on the low end of the range to 1.0×10¹² particles/kg of the subject. For example, in some embodiments, a dose of a composition comprising platelets or platelet derivatives (e.g., FDPDs) can include between about or exactly 1.0×10⁷ on the low end of the range to 1.0×10¹² particles (e.g. FDPDs)/kg of a subject, 1.0×10⁷ to 1.0×10¹¹ particles (e.g. FDPDs)/kg of a subject, 1.0×10⁷ to 1.0×10¹⁰ particles (e.g. FDPDs)/kg of a subject, 1.6×10⁷ to 1.0×10¹⁰ particles (e.g. FDPDs/kg of subject, 1.6×10⁷ to 5.1×10⁹ particles (e.g. FDPDs/kg of a subject, 1.6×10⁷ to 3.0×10⁹ particles (e.g. FDPDs)/kg of a subject, 1.6×10⁷ to 1.0×10⁹ particles (e.g. FDPDs)/kg of a subject, 1.6×10⁷ to 5.0×10⁸ particles (e.g. FDPDs)/kg of a subject, 1.6×10⁷ to 1.0×10⁸ particles (e.g. FDPDs)/kg of a subject, 1.6×10⁷ to 5.0×10⁷ particles (e.g. FDPDs)/kg of a subject, 5.0×10⁷ to 1.0×10⁸ particles (e.g. FDPDs)/kg of a subject, 1.0×10⁸ to 5.0×10⁸ particles (e.g. FDPDs)/kg of a subject, 5.0×10⁸ to 1.0×10⁹ particles (e.g. FDPDs)/kg of a subject, 1.0×10⁹ to 5.0×10⁹ particles (e.g. FDPDs)/kg of a subject, or 5.0×10⁹ to 1.0×10¹⁰ particles (e.g. FDPDs)/kg of a subject). In some embodiments, a dose of a composition comprising platelets or platelet derivatives (e.g., FDPDs) can be a range of between about or exactly 1.0×10⁸, 5.0×10⁸, 1.0×10⁹, 3.0×10⁹, 4.0×10⁹, 5.0×10⁹, 1.0×10¹⁰, 2.5×10¹⁰, or 5.0×10¹⁰ on the low end of the range to 1.0×10¹² particles (e.g. FDPDs)/kg of a subject on the high end of the range. In some embodiments, and in illustrative embodiments wherein a subject has indications as described herein, a dose of a composition comprising platelets or platelet derivatives (e.g., FDPDs) can be a range of between about or exactly 3.0×10⁹, 4.0×10⁹, 5.0×10⁹, 1.0×10¹⁰, 2.5×10¹⁰, or 5.0×10¹⁰ on the low end of the range to 1.0×10¹² particles (e.g. FDPDs)/kg of a subject on the high end of the range. In some embodiments, and in illustrative embodiments wherein a subject has indications as described herein, a dose of a composition comprising platelets or platelet derivatives (e.g., FDPDs) can be a range of between about or exactly 3.0×10⁹, 4.0×10⁹, 5.0×10⁹, 1.0×10¹⁰, 2.5×10¹⁰, or 5.0×10¹⁰ on the low end of the range to 5.0×10¹¹ particles or 1.0×10¹² (e.g. FDPDs)/kg of a subject on the high end of the range. In some embodiments, and in illustrative embodiments wherein a subject has indications as described herein, a dose of a composition comprising platelets or platelet derivatives (e.g., FDPDs) can be a range of between about or exactly 3.0×10⁹, 4.0×10⁹, 5.0×10⁹, 1.0×10¹⁰, 2.5×10¹⁰, or 5.0×10¹⁰ on the low end of the range to 1.0×10¹¹ particles or 1.0×10¹² (e.g. FDPDs)/kg of a subject on the high end of the range. In some embodiments, and in illustrative embodiments wherein a subject has indications as described herein, a dose of a composition comprising platelets or platelet derivatives (e.g., FDPDs) can be a range of between about or exactly 3.0×10⁹, 4.0×10⁹, or 5.0×10⁹ on the low end of the range to 1.0×10¹⁰ particles (e.g. FDPDs)/kg of a subject on the high end of the range. In some embodiments, and in illustrative embodiments wherein a subject has indications as described herein, a dose of a composition comprising platelets or platelet derivatives (e.g., FDPDs) can be in a range of greater than 1.5×10⁹ FDPDs/kg of the subject on the low end of the range and 1.5×10¹⁰, 1.4×10¹⁰, 1.3×10¹⁰, 1.2×10¹⁰, or 1.1×10¹⁰ FDPDs/kg of the subject on the high end; or greater than 1.0×10¹⁰ FDPDs/kg of the subject on the low end of the range and 1.5×10¹⁰, 1.4×10¹⁰, 1.3×10¹⁰, 1.2×10¹⁰, or 1.1×10¹⁰ FDPDs/kg of the subject on the high end; or 1.1×10¹⁰ FDPDs/kg of the subject on the low end of the range and 1.5×10¹⁰, 1.4×10¹⁰, 1.3×10¹⁰, or 1.2×10¹⁰ FDPDs/kg of the subject on the high end; or 1.1×10¹⁰ FDPDs/kg of the subject on the low end and less than 1.5×10¹⁰, 1.4×10¹⁰, 1.3×10¹⁰, or 1.2×10¹⁰ FDPDs/kg of the subject on the high end of the range.

In some embodiments of any aspect or embodiment herein a therapeutically effective dose or effective dose or amount of the platelet derivatives in a platelet derivative composition is in the range of 1.5×10⁷ to 5.0×10¹⁰/kg, 2.0×10⁷ to 1.0×10¹⁰/kg, 2.5×10⁷ to 5.0×10⁹/kg, 2.75×10⁷ to 3.0×10⁹/kg, 2.8×10⁷ to 4.0×10⁹/kg, 3.0×10⁷ to 4.0×10⁹/kg, 5×10⁷ to 4.0×10⁹/kg, 6×10⁷ to 3.0×10⁹/kg, 9×10⁷ to 3.0×10⁹/kg, 1.0×10⁸ to 2.0×10⁹/kg, or 1.3×10⁸ to 1.8×10⁹/kg of the subject. In some embodiments, a therapeutically effective dose or effective dose or amount of the platelet derivatives in a platelet derivative composition is in the range of 5.0×10⁷ to 1.0×10⁹/kg, 1.0×10⁸ to 5.0×10⁸/kg, 1.2×10⁸ to 2.5×10⁸/kg, 1.6×10⁸ to 2.2×10⁸/kg, or 1.7×10⁸ to 2.0×10⁸/kg of the subject. In some embodiments, the platelet derivatives in a platelet derivative composition is 1.1×10⁸/kg, 1.2×10⁸/kg, 1.3×10⁸/kg, 1.4×10⁸/kg, 1.5×10⁸/kg, 1.6×10⁸/kg, 1.7×10⁸/kg, 1.8×10⁸/kg, 1.9×10⁸/kg, 2.0×10⁸/kg, 2.1×10⁸/kg, 2.2×10⁸/kg. 2.3×10⁸/kg, 2.4×10⁸/kg, or 2.5×10⁸/kg of the subject.

In some embodiments of any aspect or embodiment herein a therapeutically effective dose or effective dose or amount of the platelet derivatives in a platelet derivative composition is in the range of 5.1×10⁸ to 9.9×10⁸/kg, 5.5×10⁸ to 9.5×10⁸/kg, 5.8×10⁸ to 9.3×10⁸/kg, 6.1×10⁸ to 9.0×10⁸/kg, 6.5×10⁸ to 8.8×10⁸/kg, 6.8×10⁸ to 8.5×10⁸/kg, 7.0×10⁸ to 8.4×10⁸/kg, 7.5×10⁸ to 8.3×10⁸/kg, 7.8×10⁸ to 8.2×10⁸/kg, or 7.9×10⁸ to 8.1×10⁸/kg of the subject. In some embodiments, a therapeutically effective dose or effective dose or amount of the platelet derivatives in a platelet derivative composition is 7.5×10⁸/kg, 7.6×10⁸/kg, 7.7×10⁸/kg, 7.8×10⁸/kg, 7.9×10⁸/kg, 8.0×10⁸/kg, 8.1×10⁸/kg, 8.2×10⁸/kg, 8.3×10⁸/kg, 8.4×10⁸/kg, or 8.5×10⁸/kg of the subject.

In some embodiments of any aspect or embodiment herein a therapeutically effective dose or effective dose or amount of the platelet derivatives in a platelet derivative composition is in the range of 1.0×10⁹ to 1.0×10¹⁰/kg, 1.1×10⁹ to 8.0×10⁹/kg, 1.2×10⁹ to 7.0×10⁹/kg, 1.2×10⁹ to 6.0×10⁹/kg, 1.2×10⁹ to 5.0×10⁹/kg, 1.3×10⁹ to 4.0×10⁹/kg, 1.3×10⁹ to 3.0×10⁹/kg, 1.3×10⁹ to 2.5×10⁹/kg, 1.4×10⁹ to 1.9×10⁹/kg, 1.50×10⁹ to 1.75×10⁹/kg, or 1.55×10⁹ to 1.70×10⁹/kg of the subject. In some embodiments, a therapeutically effective dose or effective dose or amount of the platelet derivatives in a platelet derivative composition is 1.1×10⁹/kg, 1.2×10⁹/kg, 1.3×10⁹/kg, 1.4×10⁹/kg 1.5×10⁹/kg, 1.55×10⁹/kg, 1.56×10⁹/kg, 1.57×10⁹/kg, 1.58×10⁹/kg, 1.59×10⁹/kg, 1.6×10⁹/kg, 1.61×10⁹/kg, 1.62×10⁹/kg, 1.63×10⁹/kg, 1.64×10⁹/kg, 1.65×10⁹/kg, 1.66×10⁹/kg, 1.7×10⁹/kg, 1.8×10⁹/kg, 1.9×10⁹/kg, or 2.0×10⁹/kg of the subject.

In some embodiments, a medicament or a method of treating a subject can have a low, medium, or high dosage of platelet derivatives that has a potency in the range of 250 to 5000 TGPU per kg of the subject.

In some embodiments of any aspect or embodiment herein a therapeutically effective dose or an effective dose or amount of the platelet derivatives is an amount that has a potency in the range of 250 to 5000 TGPU per kg, 270 to 4500 TGPU per kg, 280 to 4300 TGPU per kg, 290 to 4100 TGPU per kg, 300 to 3800 TGPU per kg, 310 to 3500 TGPU per kg, or 320 to 3000 TGPU per kg of the subject. In some embodiments, a therapeutically effective dose or effective dose or amount of the platelet derivatives is an amount that has a potency in the range of 275 to 500 TGPU per kg, 280 to 450 TGPU per kg, 290 to 400 TGPU per kg, 300 to 375 TGPU per kg, 310 to 350 TGPU per kg, or 320 to 340 TGPU per kg of the subject. In some embodiments of any aspect or embodiment herein a therapeutically effective dose or effective dose or amount of the platelet derivatives is an amount that has a potency of 270 TGPU per kg, 280 TGPU per kg, 290 TGPU per kg, 300 TGPU per kg, 310 TGPU per kg, 320 TGPU per kg, 330 TGPU per kg, 340 TGPU per kg, or 350 TGPU per kg of the subject.

In some embodiments of any aspect or embodiment herein a therapeutically effective dose or effective dose or amount of the platelet derivatives is an amount that has a potency in the range of 1001 to 2000 TGPU per kg, 1200 to 2000 TGPU per kg, 1300 to 1950 TGPU per kg, 1400 to 1900 TGPU per kg, 1500 to 1900 TGPU per kg, 1600 to 1900 TGPU per kg, 1700 to 1900 TGPU per kg, 1750 to 1875 TGPU per kg, or 1800 to 1850 TGPU per kg of the subject. In some embodiments, a therapeutically effective dose or effective dose or amount of the platelet derivatives is an amount that has a potency of 1780 TGPU per kg, 1790 TGPU per kg, 1800 TGPU per kg, 1810 TGPU per kg, 1820 TGPU per kg, 1830 TGPU per kg, 1840 TGPU per kg, or 1850 TGPU per kg of the subject.

In some embodiments of any aspect or embodiment herein a therapeutically effective dose or effective dose or amount of the platelet derivatives is an amount that has a potency in the range of 2001 to 3500 TGPU per kg, 2300 to 3300 TGPU per kg, 2500 to 3100 TGPU per kg, 2600 to 3100 TGPU per kg, 2700 to 3100 TGPU per kg, 2800 to 3100 TGPU per kg, 2850 to 3050 TGPU per kg, 2900 to 3000 TGPU per kg, or 2940 to 2990 TGPU per kg of the subject. In some embodiments, a therapeutically effective dose or effective dose or amount of the platelet derivatives is an amount that has a potency of 2910 TGPU per kg, 2920 TGPU per kg, 2930 TGPU per kg, 2940 TGPU per kg, 2950 TGPU per kg, 2960 TGPU per kg, 2970 TGPU per kg, 2980 TGPU per kg, 2990 TGPU per kg, 3000 TGPU per kg, or 3100 TGPU per kg of the subject.

In certain embodiments, any of the dose ranges provided above, and in illustrative embodiments those that include less than 1×10¹¹ particles/kg, or any of the ranges provided herein, for example those provided in the paragraph immediately above, can be administered more than 1 time to a subject. For example, a dose range of between 1.0×10⁷ particles to about 1.0×10¹⁰ particles, can be administered between 2 and 10 times, or between 2 and 8 times, or between 2 and 6 times, or between 3 and 8 times, or between 3 and 6 times, or between 4 and 6 times in a timeframe between within 1, 2, 3, 4, 5, or 7 days from the first dose. In some embodiments, the methods herein can include only the first dose of the platelet derivatives or the rehydrated platelet derivatives.

In a method of treating a subject with platelet derivatives as described herein, there can be several endpoints that determine if the subject is treated. A method of treating can have one or more primary endpoints. A method can additionally have one or more secondary endpoints. In some embodiments, in a method of treatment or a composition for use as a medicament as described herein, a method or a medicament leads to cessation or decrease in bleeding at a primary bleeding site at 12 hours, 24 hours, 48 hours, 72 hours, 4 days, 5 days, 6 days, and/or 7 days after administering the platelet derivative composition. In illustrative embodiments, administering platelet derivatives herein leads to a cessation in bleeding, typically, at a primary bleeding site. In illustrative embodiments, a method or a medicament as described herein leads to cessation or decrease in bleeding at bleeding sites other than primary bleeding site at 24 hours after administering the platelet derivative composition. In some embodiments, the primary bleeding site is based upon the most severe bleeding location of the subject within 12 hours prior to administering the platelet derivative composition. In some embodiments, the administering involves infusing a platelet derivative composition. In some embodiments, a platelet derivative composition is administered on Day 1 of the treatment. In some embodiments, the cessation or decrease is evidenced by an ordinal change in WHO bleeding score of the subject evaluated at 24 hours after administering the platelet derivative composition to the subject. In some embodiments, a method or a medicament as described herein leads to cessation or decrease in bleeding at bleeding sites other than primary bleeding site at 12 hours, 24 hours, 48 hours, 72 hours, 4 days, 5 days, 6 days, and 7 days after administering the platelet derivative composition. In some embodiments, the bleeding in a subject is a non-compressible bleeding or a non-compressible hemorrhage. A non-compressible hemorrhage is a type of hemorrhage that is inaccessible to a tourniquet or pressure dressing. In some embodiments, a method or a medicament leads to an increase in platelet count in the subject at 12 hours, 24 hours, 48 hours, 72 hours, 4 days, 5 days, 6 days, and 7 days after administering the platelet derivative composition. In some embodiments, the increase is at least 500 platelets/μl, 1000 platelets/μl, 2000 platelets/μl, 3000 platelets/μl, 4000 platelets/μl, 5000 platelets/μl, 6000 platelets/μl, 7000 platelets/μl, 8000 platelets/μl, 9000 platelets/μl, or 10000 platelets/μl in the subject. In some embodiments, the increase is in the range of 500 to 10000 platelets/μl, 1000 to 10000 platelets/μl, 2000 to 8000 platelets/μl, or 3000 to 7000 platelets/μl in the subject. In some illustrative embodiments, the increase can be at least 5000 platelets/μl.

In some embodiments, a method or a medicament leads to changes, or in other embodiments, does not lead to changes, in one or more markers of endothelial cell injury in the subject from a pre-administration time through 12 hours to 35 days, 24 hours to 32 days, 24 hours to 30 days, or 48 hours to 28 days after administering the platelet derivative composition. In illustrative embodiments, a method or a medicament leads to changes, or in other embodiments, does not lead to changes, in one or more markers of endothelial cell injury in the subject from a pre-administration time through baseline to 72 hours after administering the platelet derivative composition. In some embodiments, the method or the medicament leads to changes in two or more markers, three or markers, four or more markers, five or more markers, or all of the markers selected from the group consisting of Syndecan-1, hyaluronan, thrombomodulin, vascular endothelial growth factor (VEGF), interleukin 6, and sVE cadherin. In some embodiments, the changes can be an increase or a decrease in the markers of endothelial cell injury in the subject as compared to a control.

In some embodiments, a method or a medicament leads to acceptable measures of coagulation in the subject at between 12 hours to 35 days, 24 hours to 32 days, 24 hours to 30 days, or 24 hours to 28 days after administering the platelet derivative composition. In illustrative embodiments, a method or a medicament leads to acceptable measures of coagulation in the subject at 72 hours after administering the platelet derivative composition. In some embodiments, the acceptable measure of coagulation includes one or more, two or more, three or more, four or more, five or more, or all of prothrombin time (PT), international normalized ratio (INR), fibrinogen, D-dimer, activated partial thromboplastin time (aPTT), and thromboelastography (TEG) or rotational thromboelastometry (ROTEM). In some embodiments, a method or a medicament leads to an increase or a decrease in the acceptable measure of coagulation in the subject as compared to a control.

In some embodiments, a method or a medicament leads to acceptable measures of hematology in the subject from a pre-administration time through 12 hours to 35 days, 24 hours to 32 days, 24 hours to 30 days, or 48 hours to 28 days after administering the platelet derivative composition. In some embodiments, the acceptable measures of hematology are one or more, two or more, three or more, four or more, five or more, or all selected from the group consisting of Prothrombin Fragment 1+2, thrombin generation assay (TGA), Thrombopoietin, activated Protein C, tissue plasminogen activator (TPA), and/or plasminogen activator inhibitor (PAI). In some embodiments, the acceptable measures of hematology can be an increase or a decrease in the subject as compared to a control.

In some embodiments, a method of treatment or a composition for use as a medicament as described herein, a method or a medicament leads to survival of the subject without WHO Grade 2A or greater bleeding during the first 3, 4, 5, 6, 7, 8, 9, or 10 days after administering of a platelet derivative composition.

In some embodiments, administering confers an improved survival at 10, 15, 20, 25, 30, 35, 40, 45, or 50 days after administering the platelet derivatives. In some embodiments, administering leads to a decrease in administration of secondary blood products, platelets, or platelet derivatives to the subject for the first 5, 6, 7, 8, 9, or 10 days after the administering of the platelet derivatives.

A person of skill in the art can contemplate treating a subject or using platelet derivatives as described herein as a medicament in several doses in a span of time for treating the subject. In some embodiments, administering of platelet derivatives as described herein is performed in a maximum of 2, 3, 4, 5, 6, 7, 8, 9, or 10 doses in a 72-hour period of treatment. Further, in some embodiments, the subject involved in the treatment or a medication process satisfies certain criteria involving one or more of: minimum age; minimum weight; total circulating platelets (TCP); confirmed diagnosis of hematologic malignancy, myeloproliferative disorder, myelodysplastic syndrome or aplasia; undergoing chemotherapy, immunotherapy, radiation therapy or hematopoietic stem cell transplantation; refractory to platelet transfusion defined as two 1-hour CCI of <5000 on consecutive transfusions of liquid stored platelets; and WHO Bleeding Score of 2 excluding cutaneous bleeding. In some embodiments, the subject has a count of total circulating platelets (TCP) between 5,000 to 100,000 platelets/μl, 10,000 to 90,000 platelets/μl, 10,000 to 80,000 platelets/μl, or 10,000 to 70,000 platelets/μl of blood at the time of administering. In some embodiments, the subject is undergoing one or more, two or more, three or more, or all of chemotherapy, immunotherapy, radiation therapy or hematopoietic stem cell transplantation at the time of administering. In some embodiments, the subject is refractory to platelet transfusion, wherein refractory is a two 1-hour CCI [corrected count increment] of <5000 on consecutive transfusions of liquid stored platelets. In some embodiments, the subject has a WHO bleeding score of 2 excluding cutaneous bleeding. In some embodiments, the subject at the time of administering has one, two or more, or all of: confirmed diagnosis of hematologic malignancy, myeloproliferative disorder, myelodysplastic syndrome, or aplasia; undergoing chemotherapy, immunotherapy, radiation therapy or hematopoietic stem cell transplantation; or refractory to platelet transfusion wherein refractory is a two 1-hour CCI of <5000 on consecutive transfusions of liquid stored platelets.

Methods for Administering Platelet Derivatives to a Subject with Low Platelet Counts

In some aspects, methods provided herein are for treating thrombocytopenia, in illustrative embodiments DIT, CIT, and radiation-induced thrombocytopenia (RIT). In overcoming this long-felt need, some of the methods and compositions disclosed herein, provide an additional benefit of allowing a therapeutic agent, for example a chemotherapeutic agent that caused the DIT, to be administered according to a recommended protocol for such agent, without delay or dose reduction. Furthermore, in treating DIT, CIT and/or RIT, methods and compositions herein, can improve the quality of life of a patient, and be integrated as part of improved supportive care strategies.

Methods herein include providing less interrupted, and in illustrative examples, uninterrupted, cancer treatment to a subject in need thereof. For example, certain cancer treatments using cancer therapeutic agents cause a reduction in platelet numbers in a subject, which can result in thrombocytopenia, causing the subject to become prone to excessive bleeding. This can be life threatening and can affect the cancer therapeutic regimen administered to the subject. In such cases, there can be little option left apart from reducing the dose, slowing down, or stopping the cancer treatment, which is not desirable, and may even result in the cancer becoming even more aggressive than before. Administering platelet derivatives before, and/or during the course of such cancer treatments using methods and compositions herein, can lead to uninterrupted cancer treatment, such as including administering cancer therapeutic agents as disclosed herein, typically according to an approved and/or recommended cancer therapeutic dosing regimen. Such a dosing regimen can be recommended and/or approved by a government regulatory agency, such as the US FDA or the EMA or by any one or more of various cancer societies disclosed elsewhere herein.

Accordingly, in some aspects, methods herein comprise:
administering a cancer therapeutic agent to a subject according to a pre-platelet derivative cancer therapeutic dosing regimen, wherein in illustrative embodiments as a result of this administration of the cancer therapeutic agent, the subject has a reduced number of platelets, for example to a level that would give rise to a low platelet count,
optionally determining that the subject has a low platelet count,
administering an effective dose of platelet derivatives in the platelet derivative composition to the subject, and
administering the cancer therapeutic agent to the subject according to a post-platelet derivative cancer therapeutic dosing regimen.

In some embodiments, the first dose administered in the post-platelet derivative cancer therapeutic dosing regimen is not delayed. In some embodiments, the first dose administered in the post-platelet derivative cancer therapeutic dosing regimen is not reduced, and thus is at a recommended amount. In illustrative embodiments, the first dose administered in the post-platelet derivative cancer therapeutic dosing regimen is not delayed and is not reduced, for example due to a low platelet count. In illustrative embodiments, the low platelet count is a blood platelet count of less than 150,000 platelets/μl of circulating blood.

In some aspects, methods herein comprise, administering an effective dose of platelet derivatives in the platelet derivative composition to the subject before and/or after an n^{th} round of administering a cancer therapeutic agent according to a cancer therapeutic dosing regimen, and continuing the cancer therapeutic dosing regimen by administering the cancer therapeutic agent in an additional round to the subject, wherein a first dose of the cancer therapeutic agent administered in the additional round is not reduced or delayed, in illustrative embodiments due to a low platelet count in the subject. In some embodiments, the first dose of the cancer therapeutic agent in the additional round is not reduced, in illustrative embodiments due to a low platelet count in the subject. In some embodiments, the first dose of the cancer therapeutic agent in the additional round is not delayed, in illustrative embodiments due to a low platelet count in the subject. In illustrative embodiments, the first dose of the cancer therapeutic agent in the additional round is not reduced due to a low platelet count, and the first dose of the cancer therapeutic agent in the additional round is not delayed due to a low platelet count.

“n” is a round of administering a cancer therapeutic agent according to a cancer therapeutic dosing regimen after which the subject is diagnosed to have low platelet counts. In some cases, “n” is a round of administering the cancer therapeutic agent according to a cancer therapeutic dosing regimen after which the subject is suspected to have low platelet counts. In some cases, “n” is a 1^{st}, 2^{nd}, 3^{rd}, 4^{th}, 5^{th}, 6^{th}, 7^{th}, or 8^{th} round of administering the cancer therapeutic agent according to the cancer therapeutic dosing regimen. In some cases, the administering the platelet derivatives is repeated such that “n” is two or more of a 1^{st}, 2^{nd}, 3^{rd}, 4^{th}, 5^{th}, 6^{th}, 7^{th}, or 8^{th} round of administering the cancer therapeutic agent according to the cancer therapeutic dosing regimen. In some cases, the platelet derivatives can be administered before each round of administering the chemotherapeutic agent according to the chemotherapeutic dosing regimen, and in some cases, the platelet derivatives can be administered after each round of administering the chemotherapeutic agent according to the chemotherapeutic dosing regimen. In some embodiments, a low platelet count is a platelet count less than the normal number of platelets in a healthy subject. In some embodiments, a low platelet count is a platelet count less than the number of platelets that are sufficient to start or continue the cancer therapeutic regimen to a subject. In some embodiments, a low platelet count is less than 450,000, 425,000, 400,000, 375,000, 350,000, 325,000, 300,000, 275,000, 250,000, 225,000, 200,000, 175,000, 150,000, 140,000, 130,000, 120,000, 110,000, 100,000, 90,000, 80,000, 70,000, 60,000, 50,000 platelets per microliter of blood in a subject. In illustrative embodiments, a low platelet count is less than 150,000, 140,000, 130,000, 120,000, 110,000, 100,000, 90,000, 80,000, 70,000, 60,000, 50,000 platelets per microliter of blood in a subject.

Methods herein include administering a cancer therapeutic agent to a subject in accordance with a cancer therapeutic regimen. Cancer therapeutic regimens herein can include, but are not limited to chemotherapy, radiation therapy, a targeted cancer therapy, or any other cancer treatment that can cause platelet counts to reduce or can cause thrombocytopenia in a subject. Chemotherapy includes administering a chemotherapeutic agent to a subject, radiation therapy includes administering radiation to a subject, and a targeted cancer therapy includes administering a targeted cancer therapeutic agent to a subject. A cancer therapeutic regimen, such as a chemotherapeutic dosing regimen, a radiation therapy dosing regimen, or any targeted cancer therapy regimen, can be as per a cancer therapeutic dosing regimen accepted by a national or international cancer society, or network. It can be understood that a cancer therapeutic dosing regimen can be a dosing regimen provided in a regulatory agency approved dosing amount and schedule, a dosing regimen provided in a dosing and/or clinical studies section of the regulatory agency approved full prescribing information for the chemotherapeutic agent, a dosing regimen used in a clinical trial for the chemotherapeutic agent to establish efficacy based on tumor burden response rates, progression free survival and/or overall survival, or a dosing regimen recommended by a regional, national or international oncology society or network. Non-limiting examples of such cancer society or network include National Institutes of Health (NIH), National Cancer Institute (NCI), National Comprehensive Cancer Network (NCCN), American Society of Clinical Oncology (ASCO), American Society of Hematology (ASH), American Cancer Society (ACS), The American Association for Cancer Research (AACR), American Society for Radiation Oncology (ASTRO), Society for Neuro-Oncology (SNO), Society for Immunotherapy of Cancer (SITC), American Society for Transplantation and Cellular Therapy (ASTCT), Society of Surgical Oncology (SSO), American Urological Association (AUA), Children's Oncology Group (COG), Oncology Nursing Society (ONS), Leukemia and Lymphoma Society (LLS), UPMC Hillman Cancer Center, The National Surgical Adjuvant Breast and Bowel Project (NSABP), The Pancreatic Cancer Action Network (PanCAN), The Breast Cancer Foundation (BCRF), Susan G Koman Foundation, The Multiple Myeloma Research Foundation (MMRF), European Cancer Organisation, European Organisation for Research and Treatment of Cancer (EORTC), European Society for Medical Oncology (ESMO), European Hematology Association (EHA), European association for Cancer Research (EACR), European Association of Neuro-Oncology (EANO), European Society for Blood and Marrow Transplantation (EBMT), and European Association of Urology (EAU). Methods herein, include all the cancer types including carcinoma, sarcoma, lymphoma, leukemia, myeloma, and mixed types for which the cancer therapeutic dosing regimen corresponds to. Carcinomas refer to a malignant neoplasm of epithelial origin of the internal or external lining of the body, and are divided into two major categories, adenocarcinoma, and squamous cell carcinoma. Sarcomas refer to cancer that originates in supportive and connective tissues such as bones, tendons, cartilage, muscle, and fat. Some of the examples of sarcomas are Osteosarcoma or osteogenic sarcoma (bone), chondrosarcoma (cartilage), leiomyosarcoma (smooth muscle), rhabdomyosarcoma (skeletal muscle), mesothelial sarcoma or mesothelioma (membranous lining of body cavities), fibrosarcoma (fibrous tissue), angiosarcoma or hemangioendothelioma (blood vessels), liposarcoma (adipose tissue), glioma or astrocytoma (neurogenic connective tissue found in the brain), myxosarcoma (primitive embryonic connective tissue), and mesenchymous or mixed mesodermal tumor (mixed connective tissue types). Myeloma is cancer that originates in the plasma cells of bone marrow. Leukemia is a cancer of the bone marrow, and some of the examples of leukemia include myelogenous or granulocytic leukemia (malignancy of the myeloid and granulocytic white blood cell series), lymphatic, lymphocytic, or lymphoblastic leukemia (malignancy of the lymphoid and lymphocytic blood cell series), and polycythemia vera or erythremia (malignancy of various blood cell products, but with red cells predominating). Lymphomas develop in the glands or nodes of the lymphatic system.

In accordance with methods herein, a cancer therapeutic agent administered to a subject before administering an effective dose of platelet derivatives can be in accordance with a pre-platelet derivative cancer therapeutic dosing regimen, and a cancer therapeutic agent administered to a subject after administering an effective dose of platelet derivatives can be in accordance with a post-platelet derivative cancer therapeutic dosing regimen, such that the cancer therapeutic dosing regimen comprising a pre- and a post-platelet derivative regimen is not interrupted and/or is not reduced, for example due to a low platelet count in the subject. In illustrative embodiments, such a post-platelet derivative cancer therapeutic dosing regimen is not delayed and is not reduced as compared to a pre-platelet derivative cancer therapeutic dosing regimen due to a low platelet count, in illustrative embodiments, due to thrombocytopenia in a subject. Typically, administering platelet derivatives to a subject scheduled for a cancer therapeutic dosing regimen before, and/or during the regimen can provide for a regimen that is not modified, such as, in dose, or time-period because of low platelet counts of a subject undergoing the regimen. For example, methods herein include in some cases, a pre-platelet derivative cancer therapeutic dosing regimen that is the same as that of a post-platelet derivative cancer therapeutic dosing regimen. In some cases, a pre-platelet derivative cancer therapeutic dosing regimen comprises administering more than 1 dose of the cancer therapeutic agent, and a post-platelet derivative cancer therapeutic dosing regimen comprises administering 1 dose of the cancer therapeutic agent. In some cases, a pre-platelet derivative cancer therapeutic dosing regimen comprises administering 1 dose of the cancer therapeutic agent, and a post-platelet derivative cancer therapeutic dosing regimen comprises administering more than 1 dose of the cancer therapeutic agent. In some cases, administering an effective dose of platelet derivatives is repeated during the cancer therapeutic dosing regimen. In some embodiments, a pre-platelet derivative cancer therapeutic dosing regimen can be referred to as a first dosing regimen or an initial dosing regimen, comprising administering a cancer therapeutic agent to a subject. In such embodiments, the subject can be exposed to prior rounds of the cancer therapeutic agent and the terms “first” and “initial” can be used for convenience, although in some embodiments the subject is not exposed to any rounds of the cancer therapeutic agent before the “first” or “initial” dose. Furthermore, in such embodiments a post-platelet derivative cancer therapeutic dosing regimen can be referred to as a second, continuing, or a subsequent dosing regimen comprising administering a cancer therapeutic agent to the subject.

In some aspects of methods herein, platelet derivatives herein can be administered to a subject in a prophylactic or a preventive setting in which a subject set to undergo a cancer therapeutic dosing regimen can be administered an effective dose of platelet derivatives to prevent a decrease in platelet counts such that the decrease can lead to an interruption, or any form of changes including a delay in the cancer therapeutic dosing regimen. In such a prophylactic setting, the subject is set to undergo a cancer therapeutic dosing regimen, and due to the subject's medical condition, beginning the cancer therapeutic dosing regimen would lead to lowering of platelet counts such that the cancer therapeutic dosing regimen cannot be continued beyond the 1^{st}, 2^{nd}, 3^{rd}, 4^{th} or 5^{th} round until the platelet counts are restored in the subject. In a prophylactic setting, the platelet derivatives can be administered to the subject before administering a cancer therapeutic agent according to a cancer therapeutic dosing regimen. Once the cancer therapeutic dosing regimen is started, the platelet counts in the subject can be monitored over a regular period of time, typically after each round of the cancer therapeutic regimen, and whenever it is suspected that the platelet counts in the subject can fall below a threshold level, or to a level that can lead to an interruption in the cancer therapeutic dosing regimen, platelet derivatives can be administered to the subject in between the rounds of administering the cancer therapeutic agent according to the cancer therapeutic dosing regimen. As per one of the embodiments of such a prophylactic setting or a preventive setting, the subject undergoing the cancer therapeutic dosing regimen is administered platelet derivatives before the start, and during the cancer therapeutic dosing regimen, such that the platelet counts in the subject does not fall below a threshold level, the subject does not become thrombocytopenic, or the platelet counts does not fall below a level that could lead to an interruption in the cancer therapeutic dosing regimen. The prophylactic setting includes administering platelet derivatives to the subject once or more than once, for example, 1, 2, 3, 4, 5, or more doses of the platelet derivatives that is required to maintain the platelet levels in the subject in order to sustain an un-interrupted cancer therapeutic dosing regimen. Administering platelet derivatives as per the prophylactic setting herein can lead to administering a cancer therapeutic agent to a subject as per a cancer therapeutic dosing regimen that is not interrupted due to low platelet counts in the subject.

Methods herein can include administering platelet derivatives herein to a subject administered or being administered with a chemotherapeutic agent(s). In illustrative embodiments, chemotherapeutic agents used in methods herein are known to reduce platelet numbers, or cause thrombocytopenia in a subject. Typically, administering a chemotherapeutic agent to a subject in need thereof, is done according to a chemotherapeutic dosing regimen as recognized by any of the cancer society or network as disclosed herein. The administering of a chemotherapeutic agent can be administering a single chemotherapeutic agent, more than 1 chemotherapeutic agent, or a combination of at least 2, 3, 4, or more chemotherapeutic agents. Such chemotherapeutic agents can be administered in certain combinations to a subject in need thereof. In illustrative embodiments, chemotherapeutic agent can be any such agent approved for use in treating any type of cancer approved by a regulatory agency, such as, but not limiting to the Food and Drug Administration (FDA), the European Medicines Agency (EMA), the National Medicine Products Administration (NMPA) in China, and/or the Canadian Agency for Drugs and Technologies in Health (CADTH). Typically, chemotherapeutic agents can be majorly classified into alkylating agents, antimetabolites, plant alkaloids, antitumor antibiotics, and topoisomerase inhibitors. The chemotherapeutic agents herein can be selected from any of the known classes of chemotherapeutic agents. A non-limiting list of chemotherapeutic agents from alkylating class include altretamine, bendamustine, busulfan, carboplatin, chlorambucil, cisplatin, cyclophosphamide, dacarbazine, ifosfamide, mechlorethamine, melphalan, oxaliplatin, procarbazine, temozolomide, thiotepa, trabectedin, carmustine, lomustine, and streptozocin, and any combinations thereof. A non-limiting list of chemotherapeutic agents from antimetabolites class include 5-fluorouracil, 6-mercaptopurine, azacitidine, capecitabine, cladribine, clofarabine, cytarabine, decitabine, floxuridine, fludarabine, gemcitabine, hydroxyurea, methotrexate, nelarabine, pemetrexed, pentostatin, pralatrexate, thioguanine, trifluridine/tipiracil combination, and any combinations thereof. A non-limiting list of plant alkaloids can include cabazitaxel, docetaxel, nab-paclitaxel, paclitaxel, vinblastine, vincristine, vinorelbine, and any combinations thereof. A non-limiting list of antitumor antibiotics can include daunorubicin, doxorubicin, epirubicin, idarubicin, mitoxantrone, valrubicin, bleomycin, dactinomycin, mitomycin-C, and combinations thereof. A non-limiting list of topoisomerase inhibitors can include etoposide, irinotecan, teniposide, topotecan, and combinations thereof. A list of other chemotherapeutic agents that cannot be categorized in the above classification can include all-trans-retinoic acid, arsenic trioxide, asparaginase, eribulin, ixabepilone, mitotane, omacetaxine, pegaspargase, procarbazine, romidepsin, vorinostat, and combinations thereof. A non-limiting list of such chemotherapeutic agents administered as a combination according to a chemotherapeutic dosing regimen can include ACVBP—doxorubicin, cyclophosphamide, vindesine, bleomycin, and prednisone; CHOP—doxorubicin, cyclophosphamide, vincristine, and prednisone; DHAP dexamethasone, high dose cytarabine, and cisplatin; EPOCH—etoposide phosphate, vincristine sulfate (Oncovin), cyclophosphamide, and doxorubicin hydrochloride (hydroxydaunomycin) and the steroid hormone prednisone; GDP—gemcitabine, dexamethasone, and cisplatin; GemOx—gemcitabine, and oxaliplatin; and ICE—ifosfamide, carboplatin, and etoposide phosphate. A non-limiting list of such chemotherapeutic agents administered alone or in combination can include Gemcitabine, Platinum, Anthracycline, Taxane, doxorubicin, cyclophosphamide, vindesine, bleomycin, prednisone, vincristine, dexamethasone, cytarabine, cisplatin, etoposide phosphate, gemcitabine, oxaliplatin, ifosfamide, and carboplatin.

Platelet derivatives herein can be administered to a subject undergoing, or set to undergo a chemotherapeutic dosing regimen such that the platelet levels do not drop below a threshold level, the subject does not become thrombocytopenic, and/or the platelet levels do not drop to a level that would otherwise lead to an interruption in the chemotherapeutic dosing regimen. One of the known ill-effects of some chemotherapeutic dosing regimens is a decrease in platelet counts in the subject, which if platelet counts drop low enough can result in chemotherapy-induced thrombocytopenia (CIT). This can lead to potential uncontrolled bleeding problems in the subject. A skilled artisan understands that CIT can lead to a reduction in the dose of a chemotherapy drug to reduce the risk of bleeding or due to the need of platelet transfusion in the subject, which may weaken the therapeutic effect and relative dose intensity (RDI), and as a result have a negative impact on the treatment effectiveness. Typically, in methods herein including administering platelet derivatives herein to a subject undergoing or set to undergo chemotherapy, a post-platelet derivative chemotherapeutic dosing regimen is not delayed and/or the dose of the chemotherapeutic agent is not reduced due to a low platelet count in the subject, or due to thrombocytopenia. In such cases, the pre-platelet derivative chemotherapeutic dosing regimen is typically the same as the post-platelet derivative dosing regimen, unless a modification is required due to conditions other than the platelet counts of the subject. Administering an effective dose of platelet derivatives herein to a subject set to undergo, or undergoing chemotherapy, in illustrative embodiments leads to an uninterrupted chemotherapeutic dosing regimen. For example, methods herein include administering platelet derivatives herein, which can prevent CIT in a subject undergoing or set to undergo chemotherapy, typically by providing an uninterrupted chemotherapeutic dosing regimen. The uninterrupted dosing regimen includes maintaining a dose, timing, interval, and any other schedule related to the administration of a chemotherapy agent according to the chemotherapeutic dosing regimen that is not changed due to a decrease in platelet counts in the subject, or due to any other symptoms that can result from a decrease in the platelet counts. In illustrative embodiments, the method provides an uninterrupted chemotherapeutic dosing regimen, such that the method provides no interruption in the chemotherapeutic dosing regimen of the chemotherapeutic agent to the subject due to low platelet count in the subject. Typically, administering the effective dose of the platelet derivatives to the subject during the duration of the chemotherapeutic dosing regimen maintains the platelet levels in the subject above a threshold level. In some embodiments, the threshold level is a blood platelet count of 100,000, 125,000, 150,000, 175,000, 200,000, 225,000, or 250,000 platelets/μl of circulating blood. In some embodiments, the low platelet count or a decrease in platelet count is a platelet count below 150×10⁹/L, 100×10⁹/L, 75×10⁹/L, 50×10⁹/L, or 25×10⁹/L of the blood.

In some embodiments, platelet derivatives herein can be administered to a subject having chemotherapy-induced thrombocytopenia (CIT), or as such a decrease in platelet counts in the subject after being administering a chemotherapy agent according to a chemotherapeutic dosing regimen. In illustrative embodiments, administering platelet derivatives to the subject having CIT can prevent any delay or modification to the chemotherapeutic dosing regimen due to the decrease in platelet count. Typically, administering platelet derivatives to a subject having CIT can be beneficial because it is not required to modify the chemotherapeutic dosing regimen of the subject which otherwise could compromise the therapeutic efficacy of the cancer treatment. In some embodiments, provided herein, is a method for administering a platelet derivative composition to a subject diagnosed with cancer, comprising administering a chemotherapeutic agent to a subject in need thereof, according to a pre-platelet derivative chemotherapeutic dosing regimen, determining that the subject has a low platelet count, wherein the low platelet count is a blood platelet count of less than 100,000, 125,000, 150,000, 175,000, or 200,000 platelets/μl of blood, typically circulating blood, administering an effective dose of platelet derivatives in the platelet derivative composition to the subject, and administering the chemotherapeutic agent to the subject according to a post-platelet derivative chemotherapeutic dosing regimen. In other embodiments, provided herein is a method for administering a platelet derivative composition to a subject diagnosed with cancer, comprising administering a chemotherapeutic agent to a subject in need thereof, according to a pre-platelet derivative chemotherapeutic dosing regimen, administering an effective dose of platelet derivatives in the platelet derivative composition to the subject, and administering the chemotherapeutic agent to the subject according to a post-platelet derivative chemotherapeutic dosing regimen. Typically, the first dose administered in the post-platelet derivative chemotherapeutic dosing regimen is not delayed due to a low platelet count in the subject, wherein the low platelet count is a blood platelet count of less than 150,000 platelets/μl of circulating blood. In illustrative embodiments, the first dose administered in the post-platelet derivative chemotherapeutic dosing regimen is not delayed or in any way modified due to the low platelet count. In some embodiments, the pre-platelet derivative chemotherapeutic dosing regimen and the post-platelet derivative chemotherapeutic dosing regimen are the same. For example, in some embodiments of methods herein, the regimens before and after the platelet counts in the subject decreases remain the same, and the cancer treatment is not affected due to the decrease in the platelet counts in the subject. In some cases, the administering the effective dose of the platelet derivatives to the subject is repeated. For example, in some embodiments, the administering of the effective dose of the platelet derivatives is repeated between the pre-platelet derivative chemotherapeutic dosing regimen and the post-platelet derivative chemotherapeutic regimen. For example, methods herein include administering of the effective dose of the platelet derivatives that is repeated after the administering of the chemotherapeutic agent to the subject according to the post-platelet derivative chemotherapeutic dosing regimen, such that the subject does not become thrombocytopenic during the administering with the chemotherapeutic agent.

In some embodiments, a subject can undergo chemotherapy as a part of preparative or conditioning regimen before receiving a hematopoietic stem cell transplant (HSCT), or bone marrow transplant (BMT). Typically, the objectives behind providing a preparative or a conditioning regimen include: to eradicate hematologic malignancy in case of malignant indication for HSCT; to increases the chances of engraftment and to prevent rejection and Graft versus Host Diseases (GVHD); and to provide stem cell niches in the subject (host) bone marrow for the new stem cells. Typically, conditioning includes myelo-depletion, which targets the stem cells, and lymphodepletion, which targets the lymphoid system. Non-limiting examples of chemotherapeutic agents used for conditioning include alkylating agents. Some of the non-limiting examples of chemotherapeutic agents sued for conditioning or preparative regimens are busulfan (BU), cyclophosphamide (CY), melphalan, thiotepa, etoposide, carmustine, AraC, treosulfan, fludarabine, and any combinations of such chemotherapeutic agents. In some embodiments, the combinations are busulfan and cyclophosphamide (BU/CY); melphalan and busulfan; busulfan, thiotepa, and cyclophosphamide; busulfan, cyclophosphamide, and etoposide; melphalan, and carmustine; etoposide, and AraC; cyclophosphamide, carmustine, and VP16 (CBV); carmustine, etoposide, AraC, and cyclophosphamide; treosulfan, and fludarabine. In some cases, chemotherapy can be combined with radiation therapy to achieve the objectives of conditioning or a preparative regimen. For example, as per some known regimens total body irradiation (TBI), consisting of 12 to 16 Gy radiation, typically fractionated, can be combined with any of the known chemotherapeutic agents. In some embodiments, TBI can be combined individually with each of cyclophosphamide, AraC (cytarabine), etoposide, melphalan, fludarabine, busulfan, or with any combinations of the chemotherapeutic agents. In some embodiments, TBI can be combined with BU, and CY. In some cases, the chemotherapeutic agents, and the radiation therapy can be combined with other agents, such as, but not limiting to antibodies, anti-thymocyte globulin (ATG), anti-T Lymphocytes Globulin (ATLG), alemtuzumab, and co-stimulation blockade. Typically, the agents used for conditioning or preparative therapy are based on the type of cancer, type of transplant (for examples, auto or allo), age of the subject, and other criteria as per known and accepted regimens.

In some embodiments, a subject can undergo chemotherapy and/or radiation therapy as a part of a bridging therapy, and/or immune-depletion, such as lymphodepletion, and/or neoadjuvant/adjuvant treatment, for example during Chimeric antigen receptor (CAR) T cell therapy or CAR NK cell therapy. In some cases, a bridging therapy during CART or NK-T cell therapy can also include administrating radiation therapy and/or immunotherapy to a subject. Bridging therapies can vary as per the type of cancer treated with CAR T cell therapy or CAR NK cell therapy. Typically, bridging therapy in the case of B-cell non-Hodgkin lymphoma (B-NHL) can include chemotherapy, radiation therapy, and immunotherapy including monoclonal antibodies rituximab, and obinutuzumab, and lymphodepletion therapy in the case of B-NHL include administering chemotherapeutic agents such as cyclophosphamide and fludarabine. Typically, bridging therapy in the case of acute lymphoblastic leukemia (B-ALL), can include chemotherapy, radiation therapy, and immunotherapy including monoclonal antibodies blinatumomab, and inotuzumab, and lymphodepletion therapy in the case of B-ALL include administering chemotherapeutic agents such as cyclophosphamide and fludarabine. A non-limiting list of chemotherapeutic agents that can be administered to a subject for immune-depletion includes cyclophosphamide (CY), fludarabine (FLU), a combination of CY and FLU. Chemotherapeutic agents can be administered to a subject undergoing CART therapy as a part of adjuvant or neoadjuvant treatment. In some cases, a non-limiting list of chemotherapeutic agents that can be used for adjuvant or neoadjuvant treatment can be cyclophosphamide, dacarbazine, temozolomide, gemcitabine, doxorubicin, carboplatin, cisplatin, oxaliplatin, and any combinations thereof. In some cases, chemotherapeutic agents that can be used for adjuvant or neoadjuvant treatment along with CART therapy can be any of the known chemotherapeutic agents known to have anti-cancer effect.

Platelet derivatives herein can be administered to a subject undergoing or that underwent radiation therapy. For example, methods herein can include radiation therapy administered to a subject before administering platelet derivatives, which can be in accordance with a pre-platelet derivative radiation therapy regimen, and radiation therapy administered to a subject after administering platelet derivatives can be in accordance with a post-platelet derivative radiation therapy regimen. In illustrative embodiments, such a post-platelet derivative radiation therapy regimen is not delayed and/or the dose is not reduced as compared to a pre-platelet derivative radiation therapy regime due to a low platelet count, typically, due to thrombocytopenia in a subject. Administering platelet derivatives to a subject scheduled for a radiation therapy according to some embodiments herein, can provide for a radiation therapy that is not modified, such as, in dose, or time-period because of low platelet counts of a subject undergoing the radiation therapy. In illustrative embodiments here, administering platelet derivatives to a subject scheduled for a radiation therapy provides for an un-interrupted radiation therapy. A pre-platelet derivative radiation therapy regimen, and/or a post-platelet derivative radiation therapy regimen can be in accordance with cancer society or network disclosed elsewhere in the specification. In some embodiments, platelet derivatives herein can be administered to a subject undergoing or that underwent a combination of chemotherapy and radiation therapy. In some cases, radiation therapy can be administered as a part of conditioning or preparative regimen before HSCT or BM transplantation, and in some other cases, radiation therapy can be administered as a part of bridging therapy during CART therapy in a subject. Typically, the radiations used in the radiation therapy are ionizing radiations, which deposit energy in the cancer cells and the deposited energy can kill the cancer cells, or can cause genetic changes which in turn can kill the cancer cells. Typically, a radiation therapy can include external beam radiation, in which radiation is delivered to a subject from outside the body to the location of the tumor, or internal radiation, known as, brachytherapy, in which the radiation is delivered from inside the body by radioactive sources, sealed in catheters or seeds directly into the tumor site. There can be different types of radiation used in a radiation therapy, for example, photons radiation, such as x-rays and gamma rays, and particle radiations, such as, electron, proton, and neutron beams. There can be different techniques for administering radiation therapy to a subject. Fractionation is one such technique in which radiation therapy is delivered in a fractionated regime is based on the differing radiobiological properties of cancer and various normal tissues. One other technique is 3D conformal radiotherapy (3DCRT), in which 2D radiation therapy using rectangular fields based on plain X-ray imaging is replaced by 3D radiation therapy based on CT imaging which allows accurate localization of the tumor and critical normal organ structures for optimal beam placement and shielding. Other techniques include intensity modulated radiation therapy (IMRT), image-guided radiotherapy (IGRT), and stereotactic body radiation therapy (SBRT). Doses of radiation therapy can depend upon the types of cancer being treated. For example, treating breast, head, or neck cancers includes administering a dose of 40 to 50 Gy, and in some cases, a dose of 40 to 50 Gy can be administered over a period of 6 weeks for treating breast cancer. Typically, these doses are divided into multiple smaller doses that are given over a period of one to two months. The specific dose for each patient depends on the location and severity of the tumor.

Methods herein can include administering platelet derivatives herein to a subject undergoing or that underwent a targeted cancer therapeutic regimen. Typically, a targeted cancer therapeutic regimen includes administering to a subject a targeted cancer therapeutic agent, and in such cases, a pre-platelet derivative cancer therapeutic dosing regimen is a pre-platelet derivative targeted cancer therapeutic dosing regimen comprising administering a targeted cancer therapeutic agent before administering platelet derivatives to a subject, and a post-platelet derivative cancer therapeutic dosing regimen is a pre-platelet derivative targeted cancer therapeutic dosing regimen comprising administering a targeted cancer therapeutic agent after administering platelet derivatives to a subject. For example, methods herein include a non-limiting list of such targeted cancer therapeutic agent including poly ADP ribose polymerase (PARP) inhibitors, immune checkpoint inhibitors (ICIs), kinase inhibitors, receptor tyrosine kinase inhibitors, non-receptor tyrosine kinase inhibitors, serine/threonine kinase inhibitors, epigenetic inhibitors, BCL-2 inhibitors, hedgehog pathway inhibitors, proteasome inhibitors, and combinations thereof. In such cases, the inhibitors disclosed herein as targeted cancer therapeutic agents can be a small molecule or a macromolecule. Additionally, a targeted cancer therapeutic agent can be a small molecule, a macromolecule, or a biologic involving any other type of mechanism apart from the inhibitors disclosed herein. A non-limiting list of a biologic cancer therapeutic agent can include monoclonal antibodies, stem cell therapy, T cell therapy, NK cell therapy, gene therapy, vaccines, peptides, and blood products (red blood cells or platelets). For example, a biologic herein can be therapeutic antibodies, typically, approved by a regulatory agency for treating cancer. In some cases where a subject is receiving cell therapies, platelet derivatives herein can be administered before, during, or after the steps involved in the respective therapy. In the case of T cell therapy, typically, there are two types of T cell therapy, tumor infiltrating lymphocytes (TIL) therapy, and CAR T cell therapy. In the case of TIL therapy, or CAR T cell therapy, a subject undergoing the therapy can be administered a chemotherapeutic agent, and/or a radiation therapy as a part of bridging therapy and/or immune-depletion step, such as lymphodepletion step.

Methods herein can include administering platelet derivatives herein to a subject administered or being administered a poly ADP ribose polymerase (PARP) inhibitor, typically a PARP inhibitor known to reduce platelet numbers in a subject or to cause thrombocytopenia in a subject. Some examples of PARP inhibitors include olaparib, niraparib, rucaparib, talazoparib, and veliparib. In some cases, depending upon the cancer treatment regimen, PARP inhibitors can be used in combination with one other PARP inhibitors, with other chemotherapeutic agents, or with other agents such as steroids as required.

Methods herein can include administering platelet derivatives herein to a subject administered or being administered with immune checkpoint inhibitors (ICIs), typically with the ICIs known to reduce platelet numbers in a subject, or cause thrombocytopenia in a subject. Some of the ICIs administered to a subject in need thereof, typically, as a part of the cancer treatment includes inhibitors that block cytotoxic T lymphocyte associated protein 4 (CTLA-4), programmed cell death protein 1 (PD-1), or programmed cell death ligand 1 (PD-L1). Non-limiting examples of ICIs that block PD-1 include nivolumab, and pembrolizumab. Non-limiting examples of ICIs that block CTLA-4 include ipilimumab, and tremelimumab. Non-limiting examples of ICIs that block PD-L1 include atezolizumab, avelumab, and durvalumab.

Methods herein can include administering platelet derivatives herein to a subject administered or being administered with kinase inhibitors, in some embodiments, tyrosine kinase inhibitors. Typically, methods include administering platelet derivatives herein to a subject administered or being administered with kinase inhibitors, in some embodiments, tyrosine kinase inhibitors that we known to reduce platelet numbers in a subject, or cause thrombocytopenia in a subject. In some cases, the kinase inhibitors, and tyrosine kinase inhibitors can be a small molecule used for treating cancer in a subject. For example, methods herein can include small molecules that have been approved by a regulatory agency, such as, but not limiting to Food and Drug Administration (FDA), European Medicines Agency (EMA), The National Medicine Products Administration (NMPA), Canadian Agency for Drugs and Technologies in Health (CADTH). A non-limiting list of tyrosine kinase inhibitors include imatinib, sunitinib, sorafenib, dasatinib, ibrutinib, acalabrutinib, zanubrutinib, tirabrutinib, remibrutinib, evobrutinib, tolebrutinib, fenebrutinib, BMS-935177, BMS-986195, CG-806, rilzabrutinib, vecabrutinib, pirtobrutinib, nemtabrutinib, fostamatinib (R406), entospletinib, lanraplenib, PRT-060318, cevidoplenib, MK-8457, BI1002494, PP2, ruxolitinib, tofacitinib, upadacitinib, baricitinib, oclacitinib, bosutinib, nilotinib, ponatinib, PF-573,228, GSK2256098, CEP-37440, PF-00562271, VS-6062, VS-4718, BI-853520, VS-6063, pazopanib, axitinib, cabozantinib, and vatalanib, and a combination thereof. Methods herein can include small molecules that are not kinase inhibitors, for example, hypoxia-inducible factor 1α (HIF) inhibitors.

Platelet derivatives herein, according to methods herein, can be administered to a subject to increase platelet counts in a subject undergoing or that underwent a cancer therapeutic regimen, for example to maintain platelet levels above a threshold. In some embodiments, after administering the platelet derivatives but before administering the chemotherapeutic agent to the subject according to the post-platelet derivative chemotherapeutic dosing regimen, platelet counts can increase and methods herein include determining that the platelet count in the subject is increased. Such increase can be more than without administering the platelet derivatives, or can be an increase of an expected amount for a subject that is not thrombocytopenic, and even in some examples, for a subject that is a healthy subject. In some cases, after determining that the subject has a low platelet count, methods herein include administering platelets to the subject in an amount expected to increase a platelet count by an expected amount within a target platelet increase timeframe; and optionally after the administering and during the target platelet increase timeframe, determining that the platelet count did not increase by the expected amount. For example, in some methods herein, the pre-platelet derivative chemotherapeutic dosing regimen comprises administering more than 1 dose of the chemotherapeutic agent, and the post-platelet derivative chemotherapeutic dosing regimen comprises administering 1 dose of the chemotherapeutic agent. For example, the pre-platelet derivative chemotherapeutic dosing regimen in some embodiments comprises administering 1 dose of the chemotherapeutic agent, and the post-platelet derivative chemotherapeutic dosing regimen comprises administering more than 1 dose of the chemotherapeutic agent. A skilled artisan will understand that the pre-platelet derivative chemotherapeutic dosing regimen, and the post-platelet derivative chemotherapeutic dosing regimen can be of the same or different dosing depending on the standard and approved chemotherapeutic dosing regimen irrespective of the decrease in platelet counts in the subject.

In some embodiments, a subject with thrombocytopenia, or otherwise having low platelet counts after being administering a cancer therapeutic agent, according to a cancer therapeutic dosing regimen, can be bleeding or not bleeding at the time of administering platelet derivatives, or platelets. For example, in some embodiments, a subject having CIT or otherwise having low platelets, is bleeding at the time of administering platelet derivatives, or platelets, and the bleeding is decreased or stopped (ceased) after administering of the platelet derivatives, and a chemotherapeutic agent can be administered to a subject as per the chemotherapeutic dosing regimen, for example a post-platelet derivative chemotherapeutic dosing regimen, with less modification of dosing schedule and/or amount, or without any modification and/or interruption. In such cases, in some subjects undergoing such cancer therapy, it is believed that the bleeding would not be decreased or ceased after administering only platelets without administering platelet derivatives before or after administering the platelets. However, it is believed that, surprisingly, in some such subjects taking such cancer therapeutic agent, the bleeding would be decreased or ceased by administering platelet derivatives alone, or before or after administering platelets to the subjects taking such a cancer therapeutic agent. Typically, in such embodiments, the bleeding is at a primary site, and the bleeding at the primary site is decreased or stopped after between 10 minutes on the low end and 15, 30, 45, 60 minutes, or 1.5, 2, 2.5, 3, 3.5, 4, 4.5, 5, 5.5, 6, 6.5, 7, 7.5, 8, 8.5, 9, 9.5, 10, 10.5, 11, 11.5, or 12 hours on the high end, of administering the platelet derivatives to the subject. For example, in some embodiments, the bleeding is decreased or stopped within 15 minutes to 6 hours, 15 minutes to 5 hours, 15 minutes to 4 hours, 15 minutes to 3 hours, 15 minutes to 2 hours, 15 minutes to 1 hour, 30 minutes to 6 hours, 45 minutes to 6 hours, 1 to 6 hours, 2 to 6 hours, 3 to 6 hours, or 4 to 6 hours. In illustrative embodiments, the bleeding is stopped, for example, at a primary site after administering the platelet derivatives to the subject. In some cases, the bleeding is decreased, for example, at a primary site after administering the platelet derivatives to the subject. Administering platelet derivatives herein to subjects diagnosed with cancer, undergoing chemotherapy, with CIT, or otherwise with a decreased platelet count can help in managing bleeds leading to a better quality of life as compared to the subjects which have not been administered platelet derivatives. A skilled artisan will understand that managing bleeds can lead to a better management of infections in such subjects. Therefore, administering platelet derivatives herein can not only help in decreasing or stopping bleeding, but also provide better management of bleeding, thereby managing infections leading from bleeding in such subjects. In some cases, administering platelet derivatives can lead to a better management of bleeding, and infections in subjects even after they are declared as cancer-free after the chemotherapeutic dosing regimen. For example, administering platelet derivatives can be considered in such subjects whenever the platelet counts are decreased, or as such to provide better management of infections in such subjects.

In some embodiments, administering platelet derivatives herein to a subject diagnosed with cancer can be considered as a part of supportive care provided to the subjects. In some embodiments, administering platelet derivatives herein to a subject diagnosed with cancer is part of the supportive care of that subject, and can lead to an improvement in the quality of life. Such supportive care, for example, can be part of supportive care guidelines as proposed by organizations such as, MASCC, National Cancer Institute (NCI), and European Society for Medical Oncology (ESMO). In some embodiments, the quality of life of subjects diagnosed with cancer is improved for all the age groups, typically, subjects aged over 50, 55, 60, 65, 70, 75, or 80.

Methods herein, in some embodiments, include administering platelet derivatives herein to a subject not afflicted with cancer. In some embodiments, such a subject can be a subject undergoing hematopoietic stem cell transplantation, or bone marrow transplantation. For example, in some cases, such a subject can be a subject undergoing cell therapy, or a gene therapy for treating a condition other than cancer. In some cases, such a subject is being treated for an autoimmune disorder, or a genetic disorder.

### Illustrative Methods for Treating Thrombocytopenia

It has been observed that platelet derivatives having properties disclosed herein, in illustrative embodiments rehydrated, freeze-dried platelet derivatives, when administered to a subject having thrombocytopenia, have the surprising ability to increase the effectiveness of platelet transfusions. This appears to be the case even when the platelet transfusion is performed after the vast majority of platelet derivatives have been cleared from the circulation of the thrombocytopenic subject. Thus, provided herein are methods for administering a platelet derivative composition to a subject having thrombocytopenia.

In healthy humans, the normal range of platelets are platelet counts between 150,000 per microliter to 450,000 per microliter. A subject afflicted with thrombocytopenia has fewer than 150,000 platelets per microliter of circulating blood. Thrombocytopenia can be an undesirable side-effect of a number of therapeutic agents (i.e. medications) or conditions, or it can be inherited. Not to be limited by theory, the number of circulating platelets can be reduced by trapping platelets in the spleen, decreased platelet production, or increased destruction of platelets. Some of the factors that can lead to decreased production of platelets, and that can afflict subjects used in methods provided herein, can include leukemia and other types of cancers, some types of anemia, viral infections such as hepatitis C or HIV, chemotherapy drugs and radiation therapy, and heavy alcohol consumption. Some conditions can lead the body to use up or destroy platelets faster than they are produced, leading to a shortage of platelets in the blood stream. Some examples of such conditions include pregnancy, immune thrombocytopenia, bacterial infections in blood, thrombotic thrombocytopenic purpura, hemolytic uremic syndrome, and certain medications. Thrombocytopenia, for example afflicting subjects treated using methods provided herein, can include mild, moderate, or severe thrombocytopenia. Accordingly, platelet counts for a thrombocytopenic subject can be between 101,000 and 140,000 per microliter of blood (mild thrombocytopenia), between 51,000 and 100,000 per microliter of blood (moderate thrombocytopenia), and between 21,000 and 51,000 per microliter of blood (severe thrombocytopenia) during a time period of 15 minutes, 30 minutes, 45 minutes, 1 hour, 2, 4, 6, 8, 10, 12, or 24 hours from administering a first dose of the platelet derivatives. A skilled artisan will understand that subjects with severe thrombocytopenia, in some cases can have platelet counts even below 21,000 per microliter of blood. In some embodiments, methods herein include administering a dose of the platelet derivatives herein to a subject having a platelet count below 21,000/μl, 20,000/μl, 18,000/μl, 15,000/μl, 12,000/μl, 10,000/μl, or 8,000/μl of blood. In some embodiments, the platelet count of the subject is in the range of 5,000/μl to 20,000/μl, 5,000/μl to 18,000/μl, 5,000/μl to 15,000/μl, 5,000/μl to 12,000/μl, 5,000/μl to 10,000/μl, 7,000/μl to 20,000/μl, 10,000/μl to 20,000/μl, 12,000/μl to 20,000/μl, or 15,000/μl to 20,000/μl of blood. In some embodiments, the subject has a platelet count below 50,000/μl, 45,000/μl, 40,000/μl, 35,000/μl, 30,000/μl, 25,000/μl, 20,000/μl, 18,000/μl, 16,000/μl, or 15,000/μl during a time period of 15 minutes, 30 minutes, 45 minutes, 1 hour, 2, 4, 6, 8, 10, 12, or 24 hours from administering a first dose of the platelet derivatives. In some embodiments, the subject has a platelet count below 50,000/μl, 45,000/μl, 40,000/μl, 35,000/μl, 30,000/μl, 25,000/μl, 20,000/μl, 18,000/μl, 16,000/μl, or 15,000/μl during the entire period of the administering the platelet derivatives. In some embodiments, the subject has a platelet count in the range of 5,000/μl to 40,000/μl, 10,000/μl to 40,000/μl, 10,000/μl to 35,000/μl, 10,000/μl to 30,000/μl, 5,000/μl to 20,000/μl, 5,000/μl to 18,000/μl, 5,000/μl to 15,000/μl, 5,000/μl to 12,000/μl, 5,000/μl to 10,000/μl, 7,000/μl to 20,000/μl, 10,000/μl to 20,000/μl, 12,000/μl to 20,000/μl, or 15,000/μl to 20,000/μl of blood during a time period of 15 minutes, 30 minutes, 45 minutes, 1 hour, 2, 4, 6, 8, 10, 12, or 24 hours from administering a first dose of the platelet derivatives, or during the entire time period of the administering the platelet derivatives. Methods herein can include administering platelet derivatives to a subject having low initial platelet counts. For example, in such subjects with low initial platelet counts, the initial platelet counts can be less than 250,000, 225,000, 200,000, 180,000, 170,000, or 160,000 platelets per microliter. In some cases, the subjects with low initial platelet counts can be due to reasons or conditions associated with thrombocytopenia, whereas in other cases, the subjects with low initial platelet counts can have low platelet counts due to reasons other than known to be associated with thrombocytopenia. In some embodiments, a subject with thrombocytopenia or with low initial platelet counts is not bleeding at the start of administering platelet derivatives or platelets at an initial timepoint. In some embodiments, a subject with thrombocytopenia or with low initial platelet counts is bleeding at the start of administering platelet derivatives or platelets at an initial timepoint.

Typically, in methods provided herein, a platelet derivative composition, in illustrative embodiment a freeze-dried platelet derivative, including, but not limited to, those of any of the aspects or embodiments herein, or the platelet derivative composition prepared by any of the processes described in the aspects or embodiments herein, can be administered, or delivered to a subject afflicted with a low platelet count, for example which can be low enough to be classified as thrombocytopenia. Methods herein can include treating a subject having thrombocytopenia, comprising: a) administering an effective dose of platelet derivatives in a platelet derivative composition at an initial platelet derivative timepoint to the subject having thrombocytopenia; and b) within less than 10, 15, 20, 30, 45 minutes, or 1 hour after the administering the effective dose of the platelet derivatives, administering platelets to the subject, wherein after the administering platelets, the number of platelets in the subject increases. For example, the administering platelets can be within 5 minutes to 55 minutes, 10 minutes to 55 minutes, 15 minutes to 55 minutes, 20 minutes to 55 minutes, 30 minutes to 55 minutes, 35 minutes to 55 minutes, or 40 minutes to 55 minutes after the administering the effective dose of the platelet derivatives. Accordingly, as per one of the embodiments, methods herein include a) administering an effective dose of platelet derivatives to a subject with thrombocytopenia or with an initial low platelet count, typically, in subjects that need increase in platelet counts because of an underlying condition or due to a medication (drug-induced thrombocytopenia), for example due to an undergoing cancer therapy regimen (chemotherapy-induced thrombocytopenia), and b) less than within 10, 15, 20, 30, 45 minutes, or 1 hour after the administering the effective dose of the platelet derivatives, administering platelets, typically by standard platelet transfusion method of transfusing blood-compatible, typically, ABO-compatible platelets to the subject, wherein after the administering platelets, the number of platelets counted by standard methods known in the art in the subject increases, typically within an expected amount within a target platelet increase timeframe.

In some embodiments, methods can include a) administering platelets at an initial timepoint to the subject having thrombocytopenia, b) administering an effective dose of platelet derivatives in a platelet derivative composition at an initial platelet derivative timepoint to the subject, wherein the initial timepoint is more than 6, 7, 8, 9, or 10 hours, or in a range of 6-48, 6-36, 6-24, 6-12 hours before the initial platelet derivative timepoint; and c) administering platelets to the subject at a subsequent timepoint, wherein after the administering platelets, the number of platelets in the subject increases. For example, the initial timepoint is within 6.5-48 hours, 6.5-36 hours, 6.5-24 hours, 6.5-12 hours, or 6.5-10 hours before the initial platelet derivative timepoint. Accordingly, as per one of the embodiments, methods herein include a) administering platelets, typically by standard platelet transfusion method of transfusing blood-compatible, typically, ABO-compatible platelets at an initial timepoint to a subject with thrombocytopenia or with an initial low platelet count, typically, in subjects that need an increase in platelet counts because of an underlying condition or due to being administered a therapeutic agent (e.g., drug-induced thrombocytopenia), for example due to an undergoing treatment according to a chemotherapy regimen (chemotherapy-induced thrombocytopenia), b) administering an effective dose of platelet derivatives in a platelet derivative composition at an initial platelet derivative timepoint to the subject, wherein the initial timepoint is more than 6 hours before the initial platelet derivative timepoint; and c) administering platelets, typically by standard platelet transfusion method of transfusing blood-compatible, typically, ABO-compatible platelets to the subject at a subsequent timepoint, wherein after the administering platelets at the subsequent timepoint, the number of platelets counted by standard methods known in the art in the subject increases. In illustrative embodiments, after the administering platelets at the subsequent timepoint, the number of platelets in the subject increases to fall within the expected amount.

In some embodiments, the initial timepoint can be at a timepoint after the subject experiences any symptom related to thrombocytopenia. For example, the initial timepoint can be a timepoint after the subject is diagnosed with thrombocytopenia for the first time. In some embodiments, the initial timepoint can be a timepoint during the treatment for thrombocytopenia in a subject. The initial timepoint can be after or during any therapy causing the decrease in the number of circulating platelets in the subject. In some embodiments, the subsequent timepoint is within less than 1 hour after the administering the effective dose of the platelet derivatives. For example, the subsequent timepoint can be in the range of 5 to 55 minutes, 5 to 50 minutes, 5 to 45 minutes, 5 to 30 minutes, or 5 to 20 minutes after the administering the effective dose of the platelet derivatives. In some embodiments, the subsequent timepoint can be within 10, 9, 8, 7, 6, 5, 4, 3, or 2 hours after administering the effective dose of the platelet derivatives. For example, the subsequent timepoint can be in the range of 2 to 12 hours, 2 to 10 hours, 2 to 8 hours, 2 to 6 hours, or 2 to 4 hours after administering the effective dose of the platelet derivatives.

In illustrative embodiments, the thrombocytopenia is drug-induced thrombocytopenia, chemotherapy-induced thrombocytopenia, or radiation-induced thrombocytopenia. In illustrative embodiments, the subject has taken and/or is taking a therapeutic agent for treating thrombocytopenia. For example, the subject has a prescribed, active, and/or detectable amount of a therapeutic agent for treating thrombocytopenia in their blood. In some embodiments, the therapeutic agent is capable of binding the thrombopoietin (TPO) receptor. A non-limiting list of therapeutic agents that can bind to TPO receptor can include TPO, romiplostim, recombinant human TPO, eltrombopag, avatrombopag, lusutrombopag, and hetrombopag. A non-limiting list of drugs that can be used for treating thrombocytopenia can include prednisone, promacta, Doptelet, Dexamethasone, Cerezyme, Dexamethasone Intensol, Dxevo, imiglucerase, and Mulpleta. A skilled artisan will understand that the dosage of a therapeutic agent for treating thrombocytopenia can vary as per the subject's platelet count and other factors as assessed by a medical practitioner. For example, a subject can be administered with romiplostim at a dosage of 1, 2, 3, 4, 5, 6, 7, 8, 9, or 10 μg/kg of the subject. The dosage of romiplostim can be in the range of 1-10, 1-8, 1-6, 1-4, 2-10, 2-8, 2-6, 2-4, 4-10, or 4-8 μg/kg of the subject. In some embodiments, a subject can be administered with recombinant human TPO, at a dosage of at least 300 U, 400 U, 500 U, 700 U, 1,000 U, 2,000 U, 3,000 U, 5,000 U, 7,500 U, or 10,000 U/kg/day. For example, the dosage of recombinant human TPO can be in the range of 300-40,000 U, 300-30,000 U, 300-20,000, 300-15,000 U, 300-10,000 U, 300-8,000 U, 300-6,000 U, 300-5,000 U, 300-4,000 U, 300-3,000 U, 300-2,000 U, or 300-1,000 U/kg/day. In some embodiments, a subject can be administered with eltrombopag at 12.5 mg, 25 mg, 50 mg, or 75 mg per day. For example, the dosage of eltrombopag can be in the range of 12.5-150 mg, 12.5-125 mg, 12.5-100 mg, 12.5-75 mg, or 12.5-50 mg per day.

In some embodiments herein, at least 50, 60, 70, 75, 80, 90, 95, or 99% of the platelet derivatives are cleared from the subject's blood within 2 hours, 1 hour, 45 minutes, 30 minutes, 15 minutes, 10 minutes, or 5 minutes after the administering the platelet derivatives. For example, at least 90, 95, or 99% of the platelet derivatives are cleared from the subject's blood within 2 hours, 1 hour, 45 minutes, 30 minutes, 15 minutes, 10 minutes, or 5 minutes after the administering the derivatives. For example, at least 90, 95, or 99% of the derivatives are cleared from the subject's blood within 30 minutes, 15 minutes, 10 minutes, or 5 minutes after the administering the derivatives. In some embodiments, at least 99% of the derivatives are cleared from the subject's blood within 30 minutes, 15 minutes, 10 minutes, or 5 minutes after the administering the derivatives. In some embodiments, at least 99.9% of detectable derivatives are cleared from the subject's blood within 30 minutes, 15 minutes, 10 minutes, or 5 minutes after the administering the platelet derivatives. Surprisingly, the platelet derivatives, or the freeze-dried platelet derivatives herein have the property of increasing the platelet counts in a subject when the platelets are administered after a significant percentage of the platelet derivatives are cleared from the subject's blood. For example, the administering of platelets after at least 75%, 80%, 85%, 90%, 92%, 95%, 97%, 98%, 99%, or 99.5% are cleared from the subject's blood can exhibit increased platelet counts in the subject's blood, such that the increased platelet count is closer to or within the expected amount. For example, administering the platelets after at least 5, 10, 15, 20, 25, 30, 45 minutes, 1 hour, 2 hours, 2.5 hours, 3 hours, 4 hours after the administering of the platelet derivatives, can exhibit increased platelet counts in the subject's blood. For example, administering the platelets in a range of 1-30 minutes, 1-25 minutes, 1-20 minutes, 1-15 minutes, 1-10 minutes, 1-5 minutes, 5-30 minutes, 10-30 minutes, or 15-30 minutes after the administering the platelet derivatives, can lead to the clearance of at least 90% of detectable platelet derivatives from the subject's blood, and the subject's blood can still exhibit increased platelet counts. In illustrative embodiments, administering the platelets at a timepoint that leads to the clearance of at least 92%, 94%, 95%, 96%, 98%, or 99% detectable platelet derivatives can still exhibit increased platelet counts in the subject's blood.

Administering platelets in methods herein can be carried out by administering platelets according to a standard platelet transfusion method. For example, two types of platelet products can be used for transfusion. Whole blood platelets are derived from four to five whole blood donations, pooled, leukoreduced, bacterially tested, and irradiated. Apheresis platelets are derived from donors who spend a couple of hours on a cell separator. In the apheresis process, blood is drawn from the donor into an apheresis instrument which separates the blood into its components, retains some of the platelets, and returns the remainder of the blood to the donor. Apheresis platelets are equivalent to approximately 5-6 pooled units and have a small number of red blood cells and leukocytes. Preparation for platelet transfusion can start from the production of quality-approved platelet concentrates (PC) in the blood banks. PC can be prepared from whole blood or by apheresis. Six whole blood unit-derived platelets equal one apheresis platelet, which contains 5×10¹⁰ platelets per unit. The shelf life of a PC is five days. Thus, in illustrative embodiment a PC is used within 5 days for a platelet transfusion. The normal dose of platelet transfused is calculated as 10 to 15 ml/kg of the patient. This dose will usually increase the platelet count by approximately 25K-35K/microliter. Platelet transfusion can include a single transfusion or multiple transfusions of platelets. Multiple transfusion can include 2 or more than 2, 3, 4, or 5 transfusions. For example, as per one of the methods, 2 sequential platelet transfusions can be done for assessing the increase in platelets after the transfusions. The usual platelet doses can be in the range of 3×10¹¹ to 5×10¹¹ platelets per transfusion. Typically, the platelets are transfused through intravenous tubing with an in-line filter (screen filter of 170-260 micrometer pore size) to remove fibrin clots and large debris.

+Methods herein can include methods of treating cancer in a subject, for example, in a subject that has low initial platelet counts, or thrombocytopenic as a side-effect of a cancer treatment regimen. Accordingly, methods herein can include a) administering a chemotherapy drug to a subject diagnosed with cancer at an initial chemotherapy timepoint as recommended by a clinician or a medical practitioner as per a cancer treatment regimen, b) determining that the platelet counts in the subject are decreased such that the subjects have low initial platelet counts or are thrombocytopenic, c) administering an effective dose of platelet derivatives herein to the subject, and d) administering the chemotherapy drug to the subject and continuing the cancer treatment regimen as recommended to the subject. Typically, administering platelet derivatives can maintain the number of circulating platelets in the subject, such that the decrease in platelet counts due to the chemotherapy drug does not affect the cancer treatment regimen. For example, administering platelet derivatives during or as a part of the cancer treatment regimen can maintain the platelet counts in the subject to a level that does not warrant any kinds of modification to the cancer treatment regimen due to the platelet counts of the subject. For example, administering the platelet derivatives can maintain the number of platelets in the subject above 150,000, 175,000, or 200,000 platelets per microliter of the blood throughout the cancer treatment regimen. The platelet derivatives can be administered single or multiple times such as 2, 3, 4, 5, 6, 7, 8, 9, 10 or more during the cancer treatment regimen. In some embodiments, the administering of the platelet derivatives can be succeeded with a step of administering platelets, typically, ABO-compatible platelets to the subject by standard platelet transfusion method. In some embodiments, the administering of the platelet derivatives can be preceded with a step of administering platelets, typically, ABO-compatible platelets to the subject by standard platelet transfusion method. A skilled artisan can understand that selection of platelets for transfusion can depend on many factors not limiting to, the availability of types of platelets and post-transfusion platelet count of a subject. In some embodiments herein, platelet transfusion can be performed with HLA-matched platelets, cross-matched platelets, or antigen-restricted platelets (also called as antigen-avoidance platelets). In illustrative embodiments, platelet transfusion can be performed with HLA-matched platelets. In some embodiments, the platelet transfusion can be performed with random donor platelets.

### Methods for Treating Refractory Thrombocytopenia

It has been observed that thrombocytopenic subjects who do not exhibit an expected increase in platelet counts after platelet transfusion, and who can be classified as refractory to platelet transfusions, exhibit an increase, or a larger increase in platelet counts after a platelet transfusion if they are first administered platelet derivatives disclosed herein. Refractoriness to platelet transfusions can be multifactorial. The cause can be divided into immune and non-immune causes. Accordingly, in some embodiments, a platelet derivative composition, in illustrative embodiment a freeze-dried platelet derivative, including, but not limited to, those of any of the aspects or embodiments herein, or the platelet derivative composition prepared by any of the process described in the aspects or embodiments herein, can be administered, or delivered to a subject exhibiting refractoriness to platelet administration, or transfusion. For example, a non-limiting method to administer the platelet derivatives to a subject can include: administering a first round of platelets at an initial timepoint to a subject, in illustrative embodiments, a subject having thrombocytopenia; after the administering, determining that a platelet count in the subject did not increase by an expected amount; administering an effective dose of the platelet derivatives in a platelet derivative composition to the subject; and administering a subsequent round(s) of platelets to the subject at a subsequent timepoint(s). In illustrative embodiments, methods can further comprise after the administering of the subsequent round(s) of the platelets, determining that the platelet count in the subject is increased to an amount closer to the expected amount, than after the first round of administering, or fall within the expected amount. In some embodiments, methods can include administering the platelet derivatives within 72, 48, 24, 12, 8, 6, 4, 2, or 1 hour after the initial timepoint. For example, administering the platelet derivatives can be within 1-72 hours, 1-48 hours, 1-24 hours, 1-12 hours, 1-6 hours, 1-4 hours, or 1-2 hours after the initial timepoint. In some embodiments, administering platelets at the initial timepoint can be done at an amount expected to increase the platelet count by an expected amount within a target platelet increase timeframe after the initial timepoint. For example, the target platelet increase timeframe can include between 5 minutes on the low end and 36, 24, 12, 8, 6, 4, 2, or 1 hour, or 30, 15, or 10 minutes on the high end of the range, or between 10 minutes on the low end and 36, 24, 12, 8, 6, 4, 2, or 1 hour, or 30, or 15 minutes on the high end of the range, or between 10 minutes and 1 hour. Methods herein can include target platelet increase timeframe between 5 minutes on the low end and 36, 24, 12, 8, 6, 4, 2, or 1 hour, or 30, 15, or 10 minutes on the high end of the range, or between 10 minutes on the low end and 36, 24, 12, 8, 6, 4, 2, or 1 hour, or 30, or 15 minutes on the high end of the range, or between 10 minutes and 1 hour after the administration of the platelets at the subsequent timepoint. For example, a target platelet increase timeframe can be as per the standard timeframe based on the method followed for checking the increase in platelet counts. The methods for counting platelets to assess increase in platelet counts or refractoriness in a subject can be according to standard platelet counting methods.

The expected amount herein can be any standard values known to assess the refractoriness of a human subject. A skilled artisan would understand that for assessing platelet refractoriness, post-transfusion platelet increments can be studied in a systematic fashion. The corrected count increment (CCI) and the percent platelet response (PPR) are known formulas that can be used to study the post-transfusion increment adjusted for the size of the patient and the dosage administered (Bishop, J. F., et al. “The definition of refractoriness to platelet transfusions.” Transfusion Medicine 2.1 (1992): 35-41). The CCI formula uses the body surface area (BSA) of a subject to normalize the calculation, whereas the PPR uses the total blood volume (TBV) of a subject to normalize the calculation. For the purposes of the calculation, in case the exact number of platelets transfused is unavailable, a platelet amount of 3×10¹¹ can be used in the formula shown below.

$$C C I = Post transfusion platelet count - Pre transfusion platelet count ( / L ) \times BSA / Platelets transfused ( 10 \hat{} 11 )$$

$$P P R = Post transfusion platelet count - Pre transfusion platelet count ( / L ) \times TBV \times 100 \% / platelets transfused ( 10 \hat{} 11 )$$

For example, the expected amount can include Corrected Count Increment (CCI), such as, at a target platelet increase timeframe of 1-hour, the CCI of more than 5,000, 5000/μl, 7,500, or 10,000 is the expected amount after a single platelet transfusion, non-limiting to ABO-compatible platelets. In some cases, the expected amount at a target platelet increase timeframe of 2 hours can be a CCI of more than 5000/μl, 7,500, or 10,000 after a single or multiple platelet transfusion. Typically, the expected amount can be a CCI of more than 5,000 after 2 platelet transfusions. The target platelet increase timeframe can vary as per the method used for assessing the post-transfusion platelet count. For example, in case of the expected amount calculated in terms of CCI, the target platelet increase timeframe can be 10 minutes, 20 minutes, 30 minutes, 45 minutes, 1 hour, 2, 3, 4, 5, 6, 7, 8, 9, 10, 11, 12, 18, 24, 36, 48, or 72 hours after the platelet transfusion. In some cases, the target platelet increase timeframe can be one or more than one for a single platelet transfusion. For example, a CCI count can be calculated at different timepoints in the timeframe, such as at 10 minutes, and 1 hour after a single platelet transfusion. CCI count can be calculated at one more than one timepoints within the timeframe of 10 minutes to 1 hour, 10 minutes to 2 hours, 10 minutes to 6 hours, 10 minutes to 12 hours, 10 minutes to 18 hours, 10 minutes to 24 hours, or 10 minutes to 36 hours. For example, in some cases, a CCI of more than 5000/μl, 7,500, or 10,000 can be the expected amount in any of the timeframe herein after a single platelet transfusion. In other cases, a CCI of more than 5000/μl, 7,500, or 10,000 can be the expected amount in any of the timeframe herein after 2 platelet transfusions. In some cases, a CCI of more than 5000/μl, 7,500, or 10,000 can be the expected amount in any of the timeframe herein after more than 2 or multiple platelet transfusions. The CCI calculation can also reveal an underlying cause of refractoriness, for example, a 1-hour CCI of less than 5,000 for at least two consecutive platelet transfusions, can indicate an immune cause of platelet refractoriness. Some other examples to assess the expected amount to conclude refractoriness can include percent platelet recovery (PPR), a PPR of more than 15%, 18%, 20%, 25%, or 30% can be the expected amount after a single platelet transfusion. For example, an expected amount can be a PPR of more than 15%, 18%, 20%, 25%, or 30% after 2 platelet transfusions. In some cases, an expected amount can be a PPR of more than 15%, 18%, 20%, 25%, or 30% after 3, 4, 5, 6, 7, 8, 9, or 10 platelet transfusions. The increment in platelet counts, when measured in terms of platelets/L can be a measure of expected amount. In some embodiments, an expected amount in terms of PPR can be calculated in a platelet increase timeframe of 10 minutes, 20 minutes, 30 minutes, 45 minutes, 1 hour, 2, 3, 4, 5, 6, 7, 8, 9, 10, 11, 12, 18, 24, 36, 48, or 72 hours after the platelet transfusion. For example, an expected amount in terms of PPR can be calculated in a platelet increase timeframe of 10 minutes to 1 hour, 10 minutes to 2 hours, 10 minutes to 6 hours, 10 minutes to 12 hours, 10 minutes to 18 hours, 10 minutes to 24 hours, or 10 minutes to 36 hours. In some embodiments, an expected amount for assessing the platelet counts post-transfusion can be monitored by measuring platelets/L after administering platelets, typically, by platelet transfusion, in illustrative embodiments, ABO-compatible platelet transfusion. For example, the platelet count increments above 5×10⁹ platelets/L at a target platelet increase timeframe of 18-24 hours timeframe can be the expected amount after the transfusion of the ABO-compatible platelets. In some embodiments, an expected amount in terms of platelets/L can be calculated in a platelet increase timeframe of 10 minutes, 20 minutes, 30 minutes, 45 minutes, 1 hour, 2, 3, 4, 5, 6, 7, 8, 9, 10, 11, 12, 18, 24, 36, 48, or 72 hours after the platelet transfusion. For example, an expected amount in terms of platelets/L can be calculated in a platelet increase timeframe of 10 minutes to 1 hour, 10 minutes to 2 hours, 10 minutes to 6 hours, 10 minutes to 12 hours, 10 minutes to 18 hours, 10 minutes to 24 hours, or 10 minutes to 36 hours. For assessing platelet refractoriness, the platelet counts post-transfusion can be monitored by measuring platelet counts/microliter (μl). For example, an expected amount herein, can be in terms of platelet counts or platelets/μl after administering platelets, typically, platelet transfusion of blood-compatible, or ABO-compatible platelets. For example, an expected amount can be more than 5,000, 7,500, 10,000, 11,000, 12,000, 13,000, 14,000, or 15,000 platelets/μl after a single platelet transfusion. In some cases, an expected amount can be more than 5,000, 7,500, 10,000, 11,000, 12,000, 13,000, 14,000, or 15,000 platelets/μl after 2 platelet transfusions. In some cases, an expected amount can be more than 5,000, 7,500, 10,000, 11,000, 12,000, 13,000, 14,000, or 15,000 platelets/μl after 3, 4, 5, 6, 7, 8, 9, or 10 platelet transfusions.

In illustrative embodiments, the subject has taken and/or is taking a therapeutic agent for treating thrombocytopenia. For example, the subject has a prescribed, active, and/or detectable amount of a therapeutic agent for treating thrombocytopenia in their blood. In some embodiments, the therapeutic agent is capable of binding the thrombopoietin (TPO) receptor. A non-limiting list of therapeutic agents that can bind to TPO receptor can include TPO, romiplostim, recombinant human TPO, eltrombopag, avatrombopag, lusutrombopag, and hetrombopag.

Accordingly, as per one of the embodiments, methods include a) administering a first round of platelets by standard platelet transfusion method including transfusion of blood-compatible, typically ABO-compatible platelets to a thrombocytopenic subject, typically drug- or chemotherapy-induced thrombocytopenia, b) after the administering, determining that the platelet count in the subject by standard known methods within a target platelet increase timeframe after the administering did not increase by an expected amount, for example, a measure of CCI, or PPR with a target platelet increase timeframe, c) administering an effective dose of the platelet derivatives to the subject, and d) administering a subsequent round of platelets by standard platelet transfusion method including transfusion of blood-compatible, typically ABO-compatible platelets to the subject, wherein the administering the subsequent round of platelets can lead to an increase in the platelet count in the subject within a target platelet increase timeframe that is closer to the expected amount or fall within the expected amount. For example, after administering the first round of platelets, expected amount in terms of CCI can be less than 5,000, 5000/μl, 7,500, or 10,000 at 1 hour target platelet increase timeframe after a single platelet transfusion of ABO-compatible platelets, and after administering of the subsequent round of platelets, the expected amount in terms of CCI can be more than 5,000, 5000/μl, 7,500, or 10,000 at 1 hour target platelet increase timeframe after a single platelet transfusion of ABO-compatible platelets.

Methods herein including subjects that are refractory to platelet administration or transfusion, the refractoriness can be because of immune-related refractoriness, non-immune-related refractoriness, or idiopathic refractoriness. Non-immune-related refractoriness (non-limiting list) can include refractoriness due to sepsis, infection, fever, splenomegaly, disseminated intravascular coagulation (DIC), bleeding, any medications, and hepatic sinusoidal obstruction syndrome. Immune-related refractoriness can include alloimmunization that may or may not be because of prior transplantation procedures. The alloimmunization can include the presence of anti-HLA and/or anti-HPA antibodies in the blood of the subject. The course of treatment of a subject can be based on the observation of an expected amount in a target platelet increase timeframe after administering platelets, typically, platelet transfusion. The expected amount obtained after a platelet transfusion can further be correlated with the refractoriness of the subject, for example, immune-related refractoriness, non-immune-related refractoriness, or idiopathic refractoriness, and can further decide the treatment course of the subject. For example, as per one of the treatment methods, after administering platelets, typically, 2 platelet transfusions to a subject, a platelet count increment of less than 10,000/μl after one or both platelet transfusions can be indicative of immune-related refractoriness. In case of immune-related refractoriness, the subject can be checked for panel reactive antibodies (PRA), and the subject can be typed for the HLA type. In case the PRA of the subject is elevated, immune-related refractoriness can be confirmed, and the subject can be transfused with cross-matched platelets, or antigen-restricted platelets (also known as antigen-avoidance platelets), in illustrative embodiments with HLA-matched platelets. In case the PRA of the subject is not elevated, then the possibility of HPA-mediated refractoriness or non-immune-related refractoriness can be investigated. In a different scenario, for example, after administering platelets, typically, 2 platelet transfusions to a subject, in case a platelet count increment of more than 10,000/μl after both platelet transfusions are observed, then the platelet count after 24 hours post-transfusion can be checked. If the platelet count increment after 24 hours is more than 10,000/μl, then the observation can be investigated as being inconsistent with platelet refractoriness. Alternatively, if the platelet count increment after 24 hours is less than 10,000/μl, then the observation can be consistent with non-immune-related refractoriness, and the subject can be treated accordingly. In case of using CCI as the expected amount of increase in platelet counts after platelet transfusion, one of the treatment schemes can be as follows: after administering platelets, typically, 2 platelet transfusions to a subject, if CCI is less than 5,000, then HLA/HPA antibody screening can be done of the subject, and in case the presence of antibodies against HLA and/or HPA is observed in the subject's blood, then cross-matched platelets can be administered to the subject and CCI can be then monitored. If CCI remains below 5,000 then the subject can be typed for HLA, and then HLA-matched platelets, or antigen-restricted platelets (antibody avoidance platelets) can be administered to the subject. In case the CCI still remains below 5,000 then the subject can be administered random platelets. Alternatively, during the treatment an improvement in CCI post administering cross-matched platelets, HLA-matched platelets, or antigen-restricted platelets (antibody avoidance platelets) is an indication of improved platelet count and the subject can be supported by transfusing the respective platelet types.

Accordingly, in some embodiments, herein, platelet transfusion using cross-matched platelets or antigen-restricted platelets can be performed in case the subject is diagnosed with immune-related platelet refractoriness. In illustrative embodiments, herein, platelet transfusion using HLA-matched platelets can be performed in case the subject is diagnosed with immune-related platelet refractoriness.

A person of skill in the art can contemplate treating a subject, reducing or decreasing bleeding in a subject or using platelet derivatives as described herein as a medicament in several doses in a span of time for treating or reducing bleeding in the subject. In some embodiments, administering of platelet derivatives or rehydrated platelet derivatives, as disclosed herein, is performed for at least, or at a maximum of 2, 3, 4, 5, 6, 7, 8, 9, or 10 doses in a 72-hour period of treatment. In some embodiments, administering of platelet derivatives as disclosed herein, can be performed as a continuous infusion procedure. For example, a specific dose of platelet derivatives can be decided as per the requirement of a subject and the specific dose can be provided to the subject as a continuous infusion procedure with or without an interval. The dose can be any of the doses as disclosed herein. For example, from 1.0×10⁷ on the low end of the range to 1.0×10¹⁰/kg, 1.0×10¹¹/kg or 1.0×10¹²/kg of the subject on the high end of the range. A particular dose can be any dose as disclosed herein, and the dose can vary during the time interval for which the platelet derivatives are administered to a subject or a recipient in need thereof. In some embodiments, administering can be performed as a continuous infusion procedure until the bleeding or the bleeding potential of the subject is reduced/decreased as compared to the bleeding or the bleeding potential before the administering. In some embodiments, administering can be performed as a continuous infusion until the bleeding in the subject is stopped. In some embodiments, administering of platelet derivatives as described herein can be performed at regular intervals. For example, a single, double, triple or more doses of platelet derivatives as described herein and as per the requirement of a subject, for example to reduce or stop bleeding of the subject, can be administered to the recipient subject every 10 minutes, 20 minutes, 30 minutes, 1 hour, 2 hours, 3 hours, 4 hours, 5 hours, 6 hours, 7 hours, 8 hours, 9 hours, 10 hours, 11 hours, or 12 hours depending on whether bleeding is reduced to a satisfactory level, for example such that it is no longer considered life-threatening, or no longer considered severe or serious, or continued until the bleeding is mild, or stops. In some embodiments, the doses can be administered at a regular interval for 36 hours, 48 hours, or 72 hours from the start of the first dose. In some embodiments, administering of platelet derivatives as described herein can be performed as a mixed procedure in which the continuous infusion can be interrupted with a specific dose of platelet derivatives followed by a specific interval as per the requirement. In some embodiments, administering of platelet derivatives as described herein can be performed in a maximum of 2, 3, 4, 5, 6, 7, 8, 9, or 10 doses in a 24-hour period. In some embodiments, administering of platelet derivatives as described herein is performed in a maximum of 2, 3, 4, 5, 6, 7, 8, 9, or 10 doses in a 72-hour period of treatment. In some embodiments, administering of platelet derivatives can be performed at a frequency of at least one dose every 15 minutes or more frequently. For example, administering can be performed at a frequency of at least one dose every 15 minutes or more frequently starting from the first dose until the bleeding of the subject is reduced or stopped as compared to the bleeding before the administering. In some embodiments, the administering can be performed until the bleeding stops. In some embodiments, the administering can be performed for at least 1, 10, 15, 30, 45, or 60 minutes. In some embodiments, the administering can be performed at a frequency of at least one dose in every 20 minutes, 30 minutes, 45 minutes, 60 minutes, 2 hours, 3 hours, 4 hours, 5 hours, 6 hours, 7 hours, 8 hours, 9 hours, 10 hours, 12 hours, 15 hours, 18 hours, 24 hours, 30 hours, or 36 hours or more frequently. Further, in some embodiments, the subject, patient, or recipient administered FDPDs herein, or involved in the treatment or a medication process satisfies certain criteria involving one or more of: minimum age; minimum weight; total circulating platelets (TCP); confirmed diagnosis of hematologic malignancy, myeloproliferative disorder, myelodysplastic syndrome or aplasia; undergoing chemotherapy, immunotherapy, radiation therapy or hematopoietic stem cell transplantation; refractory to platelet transfusion, for example having two 1-hour CCI of <5000 on consecutive transfusions of liquid stored platelets; and WHO Bleeding Score of 2 excluding cutaneous bleeding. In some embodiments, the subject has a count of total circulating platelets (TCP) between 5,000 to 100,000 platelets/μl, 10,000 to 90,000 platelets/μl, 10,000 to 80,000 platelets/μl, or 10,000 to 70,000 platelets/μl of blood at the time of administering. In some embodiments, the subject is undergoing one or more, two or more, three or more, or all of chemotherapy, immunotherapy, radiation therapy or hematopoietic stem cell transplantation at the time of administering. In some embodiments, the subject is refractory to platelet transfusion, wherein refractory is a two 1-hour CCI [corrected count increment] of <5000 on consecutive transfusions of liquid stored platelets. In some embodiments, the subject has a WHO bleeding score of 2 excluding cutaneous bleeding. In some embodiments, the subject at the time of administering has one, two or more, or all of: confirmed diagnosis of hematologic malignancy, myeloproliferative disorder, myelodysplastic syndrome, or aplasia; undergoing chemotherapy, immunotherapy, radiation therapy or hematopoietic stem cell transplantation; or refractory to platelet transfusion wherein refractory is a two 1-hour CCI of <5000 on consecutive transfusions of liquid stored platelets. In some embodiments, the administering is performed during surgery.

Platelet Derivatives Maintain their Functions in the Presence of Anti-HLA and/or Anti-HPA Antibodies

Methods herein, or compositions for use in methods herein, include administering an effective amount of platelet derivatives, or freeze-dried platelet derivatives in a platelet derivative composition to a subject, in illustrative embodiments, to a subject (i.e., recipient) that has anti-Human Leukocyte Antigen (HLA) and/or anti-Human Platelet Antigen (HPA) antibodies in the blood. For example, the subject can have anti-HLA antibodies in the blood. In some embodiments, the subject can have anti-HPA antibodies in the blood. The subject can have both anti-HLA and anti-HPA antibodies in the blood. The anti-HLA antibodies can be against HLA Class-1 antigens, and/or HLA Class-2 antigens. For example, in case of anti-HLA antibodies against Class-1 antigens, the antibodies can be against one or more of HLA-A, HLA-B, and HLA-C, including various sub-types. In case of anti-HLA antibodies against Class-2 antigens, the antibodies can be against one or more of DR, DQ, DPA, and DPB, including various sub-types. The anti-HPA antibodies can be against one or more antigens on the platelets, for example, glycoproteins, including different types or sub-types thereof. In illustrative embodiments, the anti-HPA antibodies can be against different glycoproteins, including GPIIb/IIIa, GPIa/IIa, GPIIIa, GPIba, GPIIb, GPIa, GPIb3, CD109, and GPIX. Typically, HPA can be categorized as different HPA systems, namely, HPA-1, HPA-2, HPA-3, HPA-4, HPA-5, and HPA-15. In some embodiments, the subject can have cross-reactive antibodies against the platelet derivatives, or the freeze-dried platelet derivatives. The cross-reactive antibodies can be present against one or more antigen that is present on the platelet derivatives, typically, including GPIIb/IIIa, GPIa/IIa, GPIIIa, GPIba, GPIIb, GPIa, GPIb3, CD109, and GPIX.

Methods herein, or compositions for use in methods herein for administering platelet derivatives to a subject, for example, a recipient can include a step of determining the presence of anti-HLA and/or anti-HPA antibodies, including cross-reactive antibodies against the platelet derivatives in a sample obtained from a subject. For example, the sample from the subject can include blood sample, typically, plasma or serum sample. The information regarding the type of antibodies (for example, alloantibodies) produced in a subject can be obtained by known techniques including, but not limited to Luminex-based phenotypic beads such as, beads coated with specific Class 1 or Class 2 HLA antigens, or HPA antigens. In some embodiments, the Luminex-based beads having only single type of antigen, such as, single-antigen bead assay (SAB) can also be used. In SAB, Luminex beads are coated with a single HLA/HPA on their surface and incubated with the serum of the recipient. If the recipient has antibodies against the specific HLA/HPA that is coated on the beads, they will bind to the bead that is coated with the respective antigen. The beads are then washed and incubated with phycoerythrin (PE)-labeled anti-human IgG antibodies. This antibody will bind to the Fc region of antibodies bound to beads. The mixture is then washed and analyzed using a Luminex instrument. Each bead is identified through a unique combination of two fluorescent dyes (red and infrared) impregnated into the bead. The anti-human IgG complex will emit fluorescence upon exposure to laser light. This fluorescence is detected on a Luminex platform and antibody reactivity is recorded as mean fluorescence intensity.

Another non-limiting example of a method for analyzing a recipient's alloantibodies includes generating a calculated panel-reactive antibody (cPRA or PRA) percentage. A cPRA can be generated by a commonly known test that employs Luminex-based phenotypic beads to screen for HLA antibodies, typically, HLA Class-1 antibodies. A cPRA provides a percentage of antibody in a test serum, typically from the recipient that are reactive against a panel of known HLA antigens. Therefore, higher cPRA percentage denotes higher number of antibodies in the serum of the recipient that are reactive against a known panel of HLA Class-1 antigens. Accordingly, in some embodiments, cPRA can be used to study the presence of antibodies against known HLA. Accordingly, subjects, patients, or recipients in methods herein can have a PRA score of greater than 10, 15, 20, 25, 30, 40, 50, 60, 70, 75 80, 85, or 90%, or between 15% on the low end and 50, 60, 70, 75 80, 85, or 90% on the high end. In some embodiments, the subject, patient or recipient is refractory to platelet transfusions. In one illustrative embodiment, the subject, patient, or recipient has a PRA score of greater than 15% and less than 70%.

The sample obtained from the subject can be positive for anti-HLA and/or anti-HPA antibodies, and further for the presence of antibodies against platelet antigen on the platelet derivatives based upon the detection of antibodies against one or more HLA, HPA, or platelet antigen on the platelet derivatives. The amount of the antibodies can vary as per the subject, such that, based on the method used to quantify the amount of antibodies against HLA, HPA, or platelet antigen on the platelet derivatives, the subject, or recipient can be categorized as high risk, moderate risk, or low risk. As per one of the methods for quantifying the anti-HLA, and/or anti-HPA antibodies, involving flow cytometry, the mean fluorescence intensity (MFI) of the antibodies over 5,000 were categorized as high risk, between 3,000 and 5,000 were categorized as medium risk, and between 1,000 and 3,000 were categorized as low risk. A non-limiting method of determining the presence of anti-HLA, and/or anti-HPA antibodies in a subject can include performing antibody screening for the presence of anti-HLA antibodies against HLA Class-1 and Class-2 antigens. For samples that are positive for the presence of anti-HLA antibodies, the category of the patients can be established based on the titer of antibodies, for example, high risk, moderate risk, or low risk. Once the anti-HLA antibodies screening is completed, the samples can further be screened for the presence of anti-HPA antibodies. For samples that are positive for the presence of anti-HPA antibodies, the category of the patients can be established based on the titer of antibodies, for example, high risk, moderate risk, or low risk.

In some embodiments, platelet derivatives herein can have a comparable hemostatic activity and/or thrombin generation activity in the presence versus absence of anti-HLA, and/or anti-HPA antibodies. The platelet derivatives herein can have a comparable hemostatic activity and/or thrombin generation activity in the presence versus absence of cross-reactive antibodies against the freeze-dried platelet derivatives. For example, in ex vivo, or in vitro experiments, the hemostatic activity and/or thrombin generation activity of platelet derivatives remain unaltered, or significantly unchanged in the presence versus absence of anti-HLA, and/or anti-HPA antibodies. In some embodiments, one or more of thrombin generation activity of the platelet derivatives, for example, in in vitro thrombin generation assays, including peak thrombin, lag time until thrombin generation, endogenous thrombin potential, and time to peak thrombin generation remains comparable, unaltered, or significantly unchanged in the presence versus absence of anti-HLA, and/or anti-HPA antibodies, including the presence or absence of cross-reactive antibodies against the platelet derivatives. For example, peak thrombin, lag time until thrombin generation, endogenous thrombin potential, and time to peak thrombin generation remains comparable, unaltered, or significantly unchanged such that, platelet derivatives have the property of yielding+/−50%, +/−40%, +/−30%, +/−25%, +/−20%, +/−15%, +/−10%, +/−5%, or less in one or more of the peak thrombin, lag time until thrombin generation, endogenous thrombin potential, and time to peak thrombin generation in the presence versus absence of anti-HLA, and/or anti-HPA antibodies, including the presence or absence of cross-reactive antibodies against the platelet derivatives. For example, the variation in the yield of platelet derivatives in the presence versus absence of anti-HLA, and/or anti-HPA antibodies, including the presence versus absence of cross-reactive antibodies can be in the range of 1-50%, 1-45%, 1-40%, 1-35%, 1-30%, 1-25%, 1-20%, 1-15%, 1-10%, 1-5%, 5-50%, 10-50%, 15-50%, or 20-50% in one or more of the peak thrombin, lag time until thrombin generation, endogenous thrombin potential, and time to peak thrombin generation. In some embodiments, one or more of hemostatic activity of the platelet derivatives, for example, in an in vitro T-TAS assay including occlusion start time, occlusion time, and area under the curve (AUC) remains comparable, unaltered, or significantly unchanged in the presence versus absence of anti-HLA, and/or anti-HPA antibodies, including the presence or absence of cross-reactive antibodies against the platelet derivatives. For example, occlusion start time, occlusion time, and area under the curve (AUC) remains comparable, unaltered, or significantly unchanged such that, platelet derivatives have the property of yielding+/−50%, +/−40%, +/−30%, +/−25%, +/−20%, +/−15%, +/−10%, +/−5%, or less in one or more of the occlusion start time, occlusion time, and the AUC in the presence or absence of anti-HLA, and/or anti-HPA antibodies, including the presence versus absence of cross-reactive antibodies against the platelet derivatives. For example, the variation in the yield of platelet derivatives in the presence versus absence of anti-HLA, and/or anti-HPA antibodies, including the presence versus absence of cross-reactive antibodies can be in the range of 1-50%, 1-45%, 1-40%, 1-35%, 1-30%, 1-25%, 1-20%, 1-15%, 1-10%, 1-5%, 5-50%, 10-50%, 15-50%, or 20-50% in one or more of occlusion start time, occlusion time, and the AUC.

### Platelet Derivatives Affect Platelet Biomarkers of Endogenous Platelets

Methods herein, or compositions for use in methods herein, include administering an effective amount of platelet derivatives, or freeze-dried platelet derivatives in a platelet derivative composition to a subject in need thereof. In illustrative embodiments, the subject in need thereof, has Hermansky Pudlak Syndrome (HPS) or has Bernard Soulier Syndrome (BSS). In illustrative embodiments, the platelet derivative composition comprises a population having a reduced propensity to aggregate such that no more than 10%, 8%, 7%, or 5% of the platelet derivatives in the population aggregate under aggregation conditions comprising an agonist but no platelets. In some embodiments, administering can include administering the platelet derivatives, freeze-dried platelet-derived hemostats (FPHs), or freeze-dried platelet derivatives for treating a subject. The treating, for example, can include a partial, or a complete restoration of platelet functions in the subject.

For example, administering platelet derivatives, freeze-dried platelet-derived hemostats (FPHs) or freeze-dried platelet derivatives (FDPDs) herein to a subject, patient, and/or recipient can have certain effects on the levels of at least one platelet biomarker of the endogenous platelets in the subject, patient, and/or recipient of the FPHs or FDPDs. A skilled artisan will understand that the endogenous platelets are the platelets of a subject, and does not include platelets, or platelet derivatives that are administered to the subject. The platelet biomarkers that can be affected upon administering, in some embodiments can include any platelet biomarker that is associated with the activation of the platelets, or endogenous platelets in a subject.

In some embodiments, the platelet biomarkers whose levels are affected by administration of FPHs or FDPDs can be any one of PAC-1, CD62P, CD63, or combinations thereof. In some embodiments, the platelet biomarkers can be at least two, or all the three of the platelet biomarkers PAC-1, CD62P, CD63. Such embodiments include those in which the subject has BSS, and in illustrative embodiments, include those in which the subject has HPS. In illustrative embodiments, the platelet biomarker can be PAC-1, CD62P, or both. In some embodiments, the administering can lead to restoring the levels of at least one platelet biomarkers of the endogenous platelets in the subject to levels similar to the corresponding platelet biomarkers of platelets in a healthy subject. Similar levels as used herein, can cover a range within lower values and higher values of the level of a corresponding platelet biomarker from a healthy subject. In other words, similar levels can include levels higher or lower than a control level or a normal level of the corresponding platelet biomarker in a healthy subject. For example, the administering herein, can restore the levels to between 30% higher to 30% lower, between 25% higher to 25% lower, between 20% higher to 20% lower, between 15% higher to 15% lower, between 10% higher to 10% lower, or between 5% higher to 5% lower levels of the endogenous platelets in the subject as compared to normal levels for these biomarkers. For example, the administering herein, can restore the levels within at least 5%, 10%, 15%, 20%, 25%, or 30% levels of the endogenous platelets in the subject as compared to the normal levels. In some embodiments, the administering herein can lead to improving, increasing, increasing platelets testing positive for the at least one platelet biomarker, affecting activation of at least one platelet biomarker, or restoring the at least one platelet biomarker as discussed herein. For example, the administering can lead to an increase or an improvement in the levels of at least one platelet biomarker of platelets, in illustrative embodiments, endogenous platelets, such that the levels are increased by at least 5%, 10%, 15%, 20%, 25%, 30%, 35%, 40%, 45%, 50%, 55%, 60%, or more as compared to the levels in the subject before administering the platelet derivatives, or freeze-dried platelet derivatives. In some embodiments, the administering can lead to an increase or an improvement in the levels of at least one platelet biomarker in the range of 2 to 70%, 2 to 65%, 2 to 60%, 2 to 55%, 2 to 50%, 2 to 45%, 2 to 40%, 2 to 35%, 2 to 30%, 5 to 70%, 10 to 70%, 15 to 70%, 20 to 70%, 25 to 70%, 30 to 70%, or 35 to 75%, as compared to the levels in the subject before administering the platelet derivatives, or freeze-dried platelet derivatives.

A subject having HPS can have HPS-related biomarker abnormalities. Thus, the subject can be treated by administering the platelet derivatives, FPHs or freeze-dried platelet derivatives to the subject, such that at least one HPS-related biomarker abnormality or BSS-related biomarker abnormality observed in the subject is improved in the subject after the administering as compared to before the administering step. In some embodiments, the HPS-related biomarker abnormalities include a change in the biomarker levels, for example in endogenous platelets of a subject as compared to a subject who does not have HPS. For example, a non-limiting list of such biomarker abnormalities can include an abnormal decrease in the levels of CD62P, PAC-1, and CD63 or in some embodiments, CD52P and PC0-1. In some embodiments, an HPS-related hemostatic abnormality can include any hemostatic abnormality that can result from HPS in a subject. For example, the HPS-related hemostatic abnormality can include a decreased clotting ability in a subject that can lead to increased bleeding time in case of an injury. In some embodiments, administering can lead to restoring the levels of at least one HPS-related biomarker to normal levels after the administering as compared to before the administering step. In some embodiments, the FDPD administration can improve, control, and/or restore the levels of biomarkers and/or hemostasis in the recipient subject, such that the subject stops taking another therapeutic for treating HPS and/or an HPS-related biomarker abnormality.

In some embodiments, administering platelet derivatives, or freeze-dried platelet derivatives can lead to an increase in the levels of at least one platelet biomarker of endogenous platelets in a recipient subject/patient compared to the levels of a corresponding platelet biomarker in a subject having HPS, but not administered with the platelet derivatives, or freeze-dried platelet derivatives. In some embodiments, administering herein, can lead to an increase in, or restore, the levels of a platelet biomarker of endogenous platelets in a recipient subject/patient as compared to a subject having HPS but administered normal platelets, in illustrative embodiments, normal apheresis platelets, and not administered, or before administering the platelet derivatives, or the freeze-dried platelet derivatives as disclosed herein.

### Platelet Derivatives Affect Thrombin Generation and Clotting Ability

Administering platelet derivatives, freeze-dried platelet derivatives (FDPDs), or freeze-dried platelet-derived hemostats (FPHs) herein to a subject (i.e. recipient), in illustrative embodiments, to a subject (i.e. recipient) having HPS or BSS can affect at least one HPS-related, or BSS, respectively, hemostatic abnormality. In some embodiments, the administering can be used to treat abnormalities caused by HPS or BSS in the subject. The treatment herein can be a partial treatment or a complete treatment. For example, administering herein can lead to an improvement in one, two, three, at least one, all but one, or all HPS-related or BSS-related hemostatic abnormality. Furthermore, administration of the FDPDs can maintain the normal levels of hemostasis in the subject. For example, after administration of the FDPDs to the subject, the levels of hemostasis can be maintained such that the subject stops taking another therapeutic that the subject is taking for treating the HPS-related hemostatic irregularity(s) or BS-related hemostatic irregularity. In some embodiments, administering of FDPDs herein can lead to an improvement in thrombin generation in the subject as compared to the subject before the administering. For example, administering herein can increase or improve thrombin generation in the subject in vivo, and/or in an ex vivo assay using the subject's blood, by at least 5%, 10%, 15%, 20%, 25%, 30%, 35%, 40%, 45%, 50%, 55%, 60%, or more. In other embodiments, administering the FDPDs herein, can increase thrombin generation in the subject in vivo, and/or in an ex vivo assay using the subject's blood, in the range of 2 to 75%, 5 to 75%, 10 to 75%, 15 to 75%, 20 to 75%, 25 to 75%, 30 to 75%, 35 to 75%, 40 to 75%, 45 to 75%, 50 to 75%, 5 to 70%, 5 to 65%, 5 to 60%, 5 to 55%, 5 to 50%, or 5 to 45%.

A skilled artisan can use any known test(s) to assess thrombin generation in a subject. For example, thrombin generation can be assessed by a thrombin generation assay, and the assay can be performed by semi-automated methods for example using a calibrated automated thrombogram, or using fully automated systems. Thrombin generation assay is a type of coagulation test and is based on the potential of a plasma to generate thrombin over time, following addition of activators like phospholipids, tissue factor, and calcium. The results of the assay can typically be calculated as a thrombogram, or thrombin generation curve using computer software after calculation of thrombogram parameters.

In some embodiments, administering FDPDs herein can lead to an improvement in clot formation in the subject as compared to the subject before the administration of FDPDs. In some embodiments, the subject has HPS. In some embodiments, the subject has BSS. For example, administering FDPDs herein can increase or improve clot formation in the subject by at least 5%, 10%, 15%, 20%, 25%, 30%, 35%, 40%, 45%, 50%, 55%, 60%, or more. In other embodiments, administering herein, can increase clot formation in the subject in the range of 2 to 75%, 5 to 75%, 10 to 75%, 15 to 75%, 20 to 75%, 25 to 75%, 30 to 75%, 35 to 75%, 40 to 75%, 45 to 75%, 50 to 75%, 5 to 70%, 5 to 65%, 5 to 60%, 5 to 55%, 5 to 50%, or 5 to 45%. Clot formation can be monitored by known techniques, for example, a thromboelastography method (TEG). TEG can also be used to analyze platelet function, and fibrinolysis along with clot formation. In some embodiments, administering can lead to an improvement in the clot amplitude by at least 2 fold, 3 fold, 4 fold, 5 fold, 6 fold or more, for example when analyzed using TEG. In some embodiments, 2-8 fold, 3-8 fold, or 3-7 fold improvement in clot amplitude. In some embodiments, administering FDPDs can lead to an improvement in the clot amplitude by at least 2 fold, 3 fold, 4 fold, 5 fold, 6 fold or more, as compared to the administering with platelets, such as apheresis platelets but not FDPDs, for example when analyzed using TEG. In some embodiments, administering FDPDs can show 2-8 fold, 3-8 fold, or 3-7 fold improvement in clot amplitude, as compared to the administering with platelets, such as apheresis platelets but not FDPDs, for example when analyzed using TEG. In some embodiments, administering can lead to an improvement or increase in both thrombin generation and clot formation. In some embodiments, the administering leads to an improvement or increase in thrombin generation and/or clot formation in the subject as compared to the subject after being administered apheresis platelets, but before the administering of the platelet derivatives, or the freeze-dried platelet derivatives.

In some embodiments, administering platelet derivatives herein can exhibit a reduction in the occlusion time for occluding a collagen-coated channel, for example, in a T-TAS assay as compared to the platelets obtained from a patient having BSS. For example, administering platelet derivatives herein can exhibit a reduction in the occlusion time to restore the occlusion time similar to that exhibited by platelets from a healthy subject. In some embodiments, administering platelet derivatives herein can exhibit a 1.5 fold, 2 fold, 3 fold, or higher reduction in the occlusion time, for example, in a T-TAS assay. As shown in Example 10 it was surprisingly observed that providing platelet derivatives in surrogate BSS model platelets decreased the occlusion time and restored it similar to that of the control. Further, it was also observed that the area under curve (AUC) was increased after providing the platelet derivatives herein. In some embodiments herein, the subject is a mammal, in illustrative embodiments, the mammal is a human.

### Reduced Plasma Protein Content and Antibodies

Transfusion-related acute lung injury (TRALI) is a condition believed to be caused by the presence of antibodies (e.g., Human Leukocyte Antigen (HLA), Human Neutrophil Antigen (HNA), or granulocyte antibodies) in a transfused blood product, which can react with antigens in a transfusion recipient.

The use of plasma-based blood products from donors considered to be high-risk or who test positive for Human Leukocyte Antigen (HLA) Class I, Class II, and neutrophil-specific antibodies are banned from use in transfusion or production of human-derived platelet products (e.g., compositions comprising platelets and/or platelet derivatives (e.g., thrombosomes)) and are therefore omitted from the donor pool.

The use of tangential flow filtration (TFF) or multi-pass centrifugation can reduce the amount of antibody in a blood product, for example, to limits not detectable by current, FDA-approved, testing methods. In some cases, reduction of certain plasma components (e.g., HLA antibodies) can allow for this donor population to be accepted for production of blood products (e.g., compositions comprising platelets and/or platelet derivatives (e.g., thrombosomes)). In some embodiments described herein, a blood product can be a composition that includes platelets and an aqueous medium. In some aspects, provided herein are platelet derivative compositions comprising platelet derivatives in the form of a solid, a composition with less than 1% water, and/or a powder. The composition in solid form, in illustrative embodiments dried form, for example with less than 1% water, can be one amongst different kinds in which the composition would be packed and commercialized. Thus, it is contemplated that the composition in the dried form would preserve the characteristics with respect to the low content, or even absence of detectable HLA Class I, HLA Class II, and HNA antibodies as described with respect to the aqueous medium in the embodiments described herein. In some embodiments, the platelet derivative composition in the form of a powder is negative for HLA Class I antibodies based on a regulatory agency approved test for HLA Class I antibodies. In some embodiments, the platelet derivative composition in the form of a powder is negative for HLA Class II antibodies based on a regulatory agency approved test for HLA Class II antibodies. In some embodiments, the platelet derivative composition in the form of a powder is negative for HNA antibodies based on a regulatory agency approved test for HNA antibodies. In some embodiments, the platelet derivative composition in the form of a powder is negative for HLA Class I antibodies, HLA Class II antibodies, and HNA antibodies based on a regulatory agency approved test for HLA Class I antibodies, HLA Class II antibodies, and HNA antibodies, respectively.

Delivery to a Subject being Treated with an Anti-Platelet or Anti-Coagulant Agent

In some aspects and embodiments, platelet derivatives provided herein can be used to treat a coagulopathy in a subject that has been administered or is being administered an antiplatelet agent or an anticoagulant agent. Accordingly, in related aspects and embodiments platelet derivatives as provided herein can be used as an anti-platelet reversal agent, or an anti-coagulant reversal agent. In some embodiments, platelet derivatives provided herein can be used to treat a coagulopathy in a subject that has been administered or is being administered an antiplatelet agent and an anticoagulant agent. In some embodiments, platelet derivatives provided herein can be used to treat a coagulopathy in a subject that has been administered or is being administered aspirin, an antiplatelet agent and an anticoagulant agent. The antiplatelet class of drugs, which an illustrative class of antiplatelet agents, is widely used to prevent unwanted clotting episodes that lead to heart failure, stroke, and the like. In many cases, an antiplatelet drug may need to be reversed or stopped. In the case of advanced notice, as in a pre-planned surgery situation, the antiplatelet drug dose can sometimes be stopped before the surgery, preventing unwanted bleeding during surgery. In the case where an antiplatelet agent needs reversing quickly, reversal agents are typically not readily available, are expensive, or carry significant risk to the patient. In the case of need for rapid antiplatelet reversal, a platelet transfusion is typically administered, though the response to this is often only partial reversal. The caveat of this course of reversal is that the newly-infused platelets themselves are susceptible to circulating drug antiplatelet activity whereas, in some embodiments, compositions as described herein (e.g., including thrombosomes) are not. In some embodiments, compositions as described herein (e.g., including thrombosomes) are an active reversal agent. In some embodiments, the hemostatic activity of compositions as described herein (e.g., including thrombosomes) does not succumb to antiplatelet drugs. In some embodiments, an antiplatelet agent can be selected from the group consisting of aspirin, cangrelor, ticagrelor, clopidogrel, prasugrel, eptifibatide, tirofiban, abciximab, and combinations thereof.

In some embodiments of any of the methods for treating or methods of administering aspects herein, the subject is being treated or was treated with an anti-coagulant. In certain embodiments, the anticoagulant is dabigatran, argatroban, hirudin, rivaroxaban, apixaban, edoxaban, fondaparinux, warfarin, heparin, a low molecular weight heparin, a supplement, or a combination thereof. In some embodiments of any of the methods for administering or for treating aspects herein, wherein the subject is being treated with an anticoagulant, the anticoagulant is dabigatran, argatroban, hirudin, rivaroxaban, apixaban, edoxaban, fondaparinux, warfarin, heparin, low molecular weight heparins, tifacogin, Factor VIIai, SB249417, pegnivacogin (with or without anivamersen), TTP889, idraparinux, idrabiotaparinux, SR23781A, apixaban, betrixaban, lepirudin, bivalirudin, ximelagatran, phenprocoumon, acenocoumarol, indandiones, fluindione, a health and wellness supplement with anti-coagulant properties, or a combination thereof.

In some embodiments, there is provided a method of administering to a subject, or a method of treating coagulopathy in a subject, wherein the subject has been treated or is being treated with an antiplatelet agent or an anti-coagulant, the method comprising administering to the subject in need thereof an effective amount of a composition comprising platelet derivatives as described herein and an incubating agent comprising one or more salts, a buffer, optionally a cryoprotectant, and optionally an organic solvent. In some embodiments, there is provided a method of restoring normal hemostasis in a subject, the method comprising administering to the subject in need thereof an effective amount of a composition comprising platelet derivatives as described herein and an incubating agent comprising one or more salts, a buffer, optionally a cryoprotectant, and optionally an organic solvent. In some embodiments, there is provided a method of preparing a subject for surgery, the method comprising administering to the subject in need thereof an effective amount of a composition comprising platelet derivative composition as described herein and an incubating agent comprising one or more salts, a buffer, optionally a cryoprotectant, and optionally an organic solvent.

In some embodiments, the subject that has been administered or is being administered an antiplatelet agent or an anticoagulant agent in a subject having an indication and thus afflicted with a disorder or disease, that is any one or a combination of Von Willebrand disease, immune thrombocytopenia, intracranial hemorrhage (ICH), traumatic brain injury (TBI), Hermansky Pudlak Syndrome (HPS), chemotherapy induced thrombocytopenia (CIT), Scott syndrome, Evans syndrome, Hematopoietic Stem Cell Transplantation, fetal and neonatal alloimmune thrombocytopenia, Bernard Soulier syndrome, acute myeloid leukemia, Glanzmann thrombasthenia, myelodysplastic syndrome, hemorrhagic shock, coronary thrombosis (myocardial infarction), ischemic stroke, arterial thromboembolism, Wiskott Aldrich Syndrome, venous thromboembolism, MYH9 related disease, Acute Lymphoblastic Lymphoma (ALL), Acute Coronary Syndrome, Chronic Lymphocytic Leukemia (CLL), Acute Promyelocytic Leukemia, Cerebral Venous Sinus Thrombosis (CVST), Liver Cirrhosis, Factor V Deficiency (Owren Parahemophilia), Thrombocytopenia absent radius syndrome, Kasabach Merritt syndrome, Gray platelet syndrome, aplastic anemia, chronic liver disease, acute radiation syndrome, Dengue hemorrhagic fever, pre-eclampsia, snakebite envenomation, HELLP syndrome, haemorrhagic cystitis, multiple myeloma, disseminated intravascular coagulation, heparin induced thrombocytopenia, pre-eclampsia, labor and delivery, hemophilia, cerebral (fatal) malaria, Alexander's Disease (Factor VII deficiency), hemophilia c (factor xi deficiency), familial hemophagocytic lymphohistiocytosis, acute lung injury, hemolytic uremic syndrome, menorrhagia, chronic myeloid leukemia.

Provided herein in one aspect is a method of treating a coagulopathy in a subject, the method including administering to the subject in need thereof an effective amount of a composition including platelets, or in illustrative embodiments platelet derivatives, and in further illustrative embodiments FDPDs, thereby treating the coagulopathy. In illustrative embodiments, the composition comprising the platelet derivatives is administered such that the bleeding potential of the subject is reduced, and in illustrative embodiments such that normal hemostasis is restored in the subject.

In one aspect, provided herein is a method of treating a coagulopathy in a subject, the method including administering to the subject in need thereof an effective amount of a composition prepared by a process including incubating platelets with an incubating agent including one or more salts, a buffer, optionally a cryoprotectant, and optionally an organic solvent, and in illustrative embodiments freeze-drying the incubated platelets, to form the composition, wherein the composition includes platelet derivatives, and in illustrative embodiments FDPDs, thereby treating the coagulopathy. In illustrative embodiments, the composition comprising the platelet derivatives is administered such that the bleeding potential of the subject is reduced, and in illustrative embodiments such that normal hemostasis is restored in the subject.

In one aspect, provided herein is a method of restoring normal hemostasis in a subject, the method including administering to the subject in need thereof an effective amount of a composition including platelets, or in illustrative embodiments platelet derivatives, and in further illustrative embodiments FDPDs, thereby treating the coagulopathy. In illustrative embodiments, the composition comprising the platelet derivatives is administered such that the bleeding potential of the subject is reduced, and in illustrative embodiments such that normal hemostasis is restored in the subject.

In one aspect, provided herein is a method of restoring normal hemostasis in a subject, the method including administering to the subject in need thereof an effective amount of a composition prepared by a process including incubating platelets with an incubating agent including one or more salts, a buffer, optionally a cryoprotectant, and optionally an organic solvent, and in illustrative embodiments freeze-drying the incubated platelets, to form the composition, wherein the composition comprises platelet derivatives, and in further illustrative embodiments FDPDs, thereby treating the coagulopathy. In illustrative embodiments, the composition comprising the platelet derivatives is administered such that the bleeding potential of the subject is reduced, and in illustrative embodiments such that normal hemostasis is restored in the subject.

In one aspect, provided herein is a method of preparing a subject for surgery, the method including administering to the subject in need thereof an effective amount of a composition including platelets, or in illustrative embodiments platelet derivatives, and in further illustrative embodiments FDPDs. Various properties of exemplary embodiments of such FDPDs are provided herein, thereby treating the coagulopathy. In illustrative embodiments, the composition comprising the platelet derivatives is administered such that the bleeding potential of the subject is reduced, and in illustrative embodiments such that normal hemostasis is restored in the subject. Implementations can include one or more of the following features. The surgery can be an emergency surgery. The surgery can be a scheduled surgery.

In one aspect, provided herein is a method of preparing a subject for surgery, the method including administering to the subject in need thereof an effective amount of a composition prepared by a process including incubating platelets with an incubating agent including one or more salts, a buffer, optionally a cryoprotectant, and optionally an organic solvent, and in illustrative embodiments freeze-drying the incubated platelets, to form the composition, wherein the composition includes platelet derivatives, and in further illustrative embodiments FDPDs, thereby treating the coagulopathy. In illustrative embodiments, the composition comprising the platelet derivatives is administered such that the bleeding potential of the subject is reduced, and in illustrative embodiments such that normal hemostasis is restored in the subject. Various properties of exemplary embodiments of such FDPDs are provided herein. Implementations can include one or more of the following features. The surgery can be an emergency surgery. The surgery can be a scheduled surgery.

In one aspect, provided herein is a method of ameliorating the effects of an antiplatelet agent in a subject, the method including administering to the subject in need thereof an effective amount of a composition platelets, or in illustrative embodiments platelet derivatives, and in further illustrative embodiments FDPDs, thereby treating the coagulopathy. In illustrative embodiments, the composition comprising the platelet derivatives is administered such that the bleeding potential of the subject is reduced, and in illustrative embodiments such that normal hemostasis is restored in the subject.

In one aspect, provided herein is a method of ameliorating the effects of an antiplatelet agent in a subject, the method including administering to the subject in need thereof an effective amount of a composition prepared by a process including incubating platelets with an incubating agent including one or more salts, a buffer, optionally a cryoprotectant, and optionally an organic solvent, to form the composition, wherein the composition includes platelet derivatives, and in further illustrative embodiments FDPDs, thereby treating the coagulopathy. In illustrative embodiments, the composition comprising the platelet derivatives is administered such that the bleeding potential of the subject is reduced, and in illustrative embodiments such that normal hemostasis is restored in the subject.

In one aspect, provided herein is a method of treating a coagulopathy in a subject, or of restoring hemostasis in a subject, or of reducing bleeding potential of a subject that is being administered, or has been administered, an antiplatelet agent, the method comprising: administering to the subject in need thereof an effective amount of a composition comprising platelet derivatives, thereby treating the coagulopathy. In illustrative embodiments, the platelet derivatives are freeze-dried platelet derivatives (FDPDs). In further illustrative embodiments, the composition comprising the platelet derivatives is administered such that the bleeding potential of the subject is reduced, and in illustrative embodiments such that normal hemostasis is restored in the subject.

In another aspect, provided herein is a method of treating a coagulopathy in a subject, or of restoring hemostasis in a subject, or of reducing bleeding potential of a subject, wherein the subject is being administered, or has been administered, an antiplatelet agent, the method comprising administering to the subject in need thereof an effective amount of the composition comprising FDPDs, wherein the composition comprising FDPDs comprises a population of FDPDs having a reduced propensity to aggregate such that no more than 10% of the FDPDs in the population aggregate under aggregation conditions comprising an agonist but no platelets, thereby treating the coagulopathy. In further illustrative embodiments, the composition comprising the FDPDs is administered such that the bleeding potential of the subject is reduced, and in illustrative embodiments such that normal hemostasis is restored in the subject.

In another aspect, provided herein is a method of preventing or mitigating the potential for a coagulopathy in a subject, the method comprises: (a) determining that information regarding whether the subject was administered an antiplatelet agent is unavailable; and (b) administering to the subject an effective amount of a composition comprising freeze-dried platelet derivatives (FDPDs). In some embodiments of such a method, information regarding whether the subject was administered an antiplatelet agent is unavailable for a reason comprising that the subject cannot be identified. In some embodiments of the method, information regarding whether the subject was administered an antiplatelet agent is unavailable for a reason comprising that the medical history of the subject is unavailable. In further embodiments information regarding whether the subject was administered an antiplatelet agent is unavailable for a reason comprising that the subject is in need of emergency treatment.

In another aspect, provided herein is a method of treating a coagulopathy in a subject or of reducing the bleeding potential of a subject, or of restoring hemostasis in a subject, wherein the method comprises: administering to the subject in need thereof an effective amount of a composition comprising platelet derivatives, in illustrative embodiments, FDPDs, wherein the subject before the administering the composition comprising platelet derivatives, was administered an antiplatelet agent and a second agent that decreases platelet function, thereby treating the coagulopathy. In further illustrative embodiments, the composition comprising the platelet derivatives is administered such that the bleeding potential of the subject is reduced, and in illustrative embodiments such that normal hemostasis is restored in the subject. In illustrative embodiments, before the administering of the composition comprising FDPDs the subject was in need thereof because of an increased risk of bleeding due to, or as a result of being administered the anti-platelet agent and the second agent.

In another aspect, provided herein is a composition comprising freeze-dried platelet derivatives (FDPDs) for treating a coagulopathy in a subject, wherein the treating comprises: administering to the subject in need thereof, an effective amount of the composition comprising FDPDs such that the bleeding potential, or risk of bleeding of the subject is reduced, wherein the subject was administered an antiplatelet agent and a second agent that decreases platelet function, and wherein the subject is in need thereof because of an increased potential for, or risk of bleeding due to, or as a result of being administered the antiplatelet agent and the second agent, thereby treating the coagulopathy.

In another aspect, provided herein is a composition comprising freeze-dried platelet derivatives (FDPDs) for treating a coagulopathy in a subject having an increased potential for, or risk of bleeding as a result of being administered or having been administered an anticoagulant, wherein the treating comprises: administering to the subject having the increased potential for, or risk of bleeding, an effective amount of the composition comprising FDPDs such that the bleeding potential or risk of bleeding of the subject is reduced, wherein the composition comprising FDPDs comprises a population of FDPDs having a reduced propensity to aggregate such that no more than 10% of the FDPDs in the population aggregate under aggregation conditions comprising an agonist but no platelets and no divalent cations, thereby treating the coagulopathy.

In some embodiments of any of the method or use embodiments herein, a dose, and in illustrative embodiments an effective amount of a composition comprising platelets or platelet derivatives (e.g., FDPDs) administered to a subject or patient, can be a range of between about or exactly 1.0×10⁸, 5.0×10⁸, 1.0×10⁹, 3.0×10⁹, 4.0×10⁹, 5.0×10⁹, 1.0×10¹⁰, or 5.0×10¹⁰ to 1.0×10¹² particles (e.g. FDPDs)/kg of a subject. In some embodiments, and in illustrative embodiments wherein a subject has blood comprising two anti-platelet agents and/or has been administered dual anti-platelet therapy, a dose of a composition comprising platelets or platelet derivatives (e.g., FDPDs) can be a range of between about or exactly 3.0×10⁹, 4.0×10⁹, 5.0×10⁹, 1.0×10¹⁰, 2.5×10¹⁰, or 5.0×10¹⁰ to 5.0×10¹¹ particles (e.g. FDPDs)/kg of a subject. In some embodiments, and in illustrative embodiments wherein a subject has blood comprising two anti-platelet agents and/or has been administered dual anti-platelet therapy, a dose of a composition comprising platelets or platelet derivatives (e.g., FDPDs) can be a range of between about or exactly 3.0×10⁹, 4.0×10⁹, 5.0×10⁹, 1.0×10¹⁰, 2.5×10¹⁰, or 5.0×10¹⁰ to 1.0×10¹¹ particles (e.g. FDPDs)/kg of a subject. In some embodiments, and in illustrative embodiments wherein a subject has blood comprising two anti-platelet agents and/or has been administered dual anti-platelet therapy, a dose of a composition comprising platelets or platelet derivatives (e.g., FDPDs) can be a range of between about or exactly 3.0×10⁹, 4.0×10⁹, or 5.0×10⁹ to 1.0×10¹⁰ particles (e.g. FDPDs)/kg of a subject. In one illustrative embodiment, and in illustrative embodiments wherein a subject has blood comprising i) an anti-platelet agent and a second agent that decreases platelet function; ii) two anti-platelet agents; and/or iii) has been administered dual anti-platelet therapy, a dose or an effective amount of a composition comprising FDPDs is between 5.0×10¹⁰ to 1.0×10¹²/kg of the subject, 5.0×10¹⁰ to 5.0×10¹¹/kg of the subject, 5.0×10¹⁰ to 1.0×10¹¹/kg of the subject, 5.0×10⁹ to 1.0×10¹¹/kg of the subject, 5.0×10⁹ to 5.0×10¹⁰/kg of the subject, or 5.0×10⁹ to 1.0×10¹⁰/kg of the subject. In some embodiments, and in illustrative embodiments wherein a subject has blood comprising two anti-platelet agents and/or has been administered dual anti-platelet therapy, a dose of a composition comprising platelets or platelet derivatives (e.g., FDPDs) can be in a range of greater than 1.5×10⁹ FDPDs/kg of the subject on the low end of the range and 1.5×10¹⁰, 1.4×10¹⁰, 1.3×10¹⁰, 1.2×10¹⁰, or 1.1×10¹⁰ FDPDs/kg of the subject on the high end; or greater than 1.0×10¹⁰ FDPDs/kg of the subject on the low end of the range and 1.5×10¹⁰, 1.4×10¹⁰, 1.3×10¹⁰, 1.2×10¹⁰, or 1.1×10¹⁰ FDPDs/kg of the subject on the high end; or 1.1×10¹⁰ FDPDs/kg of the subject on the low end of the range and 1.5×10¹⁰, 1.4×10¹⁰, 1.3×10¹⁰, 1.2×10¹⁰, or 1.1×10¹⁰ FDPDs/kg of the subject on the high end; or 1.1×10¹⁰ FDPDs/kg of the subject on the low end and less than 1.5×10¹⁰, 1.4×10¹⁰, 1.3×10¹⁰, or 1.2×10¹⁰ FDPDs/kg: of the subject on the high end.

In some embodiments, for example of aspects wherein a subject was administered the antiplatelet agent and the second agent that decreases platelet function, such a method further comprises before the administering the composition comprising FDPDs, determining that the subject was administered the antiplatelet agent and the second agent that decreases platelet function. In some embodiments, the antiplatelet agent is a first antiplatelet agent and the second agent is a second antiplatelet agent. In some embodiments, the first antiplatelet agent and the second anti-platelet agent are each different antiplatelet agents selected from aspirin, cangrelor, ticagrelor, clopidogrel, prasugrel, eptifibatide, tirofiban, abciximab, terutroban, picotamide, elinogrel, ticlopidine, ibuprofen, vorapaxar, atopaxar, cilostazol, prostaglandin E1, epoprostenol, dipyridamole, treprostinil sodium, and sarpogrelate. In some embodiments, the first antiplatelet agent and the second anti-platelet agent have different mechanisms of action. In some embodiments, the first antiplatelet agent and the second anti-platelet agent are each different antiplatelet agents selected from aspirin, cangrelor, ticagrelor, clopidogrel, prasugrel, eptifibatide, tirofiban, abciximab, terutroban, picotamide, elinogrel, ticlopidine, ibuprofen, vorapaxar, atopaxar, cilostazol, prostaglandin E1, epoprostenol, dipyridamole, treprostinil sodium, and sarpogrelate.

In some embodiments of any of the aspects herein, before, immediately before, at the moment before, at the moment of, and/or at an initial time of, the administering of the composition comprising platelet derivatives, for example FDPDs, the subject was or is at an increased risk of bleeding due to being administered or having been administered the anti-platelet agent. Furthermore, the subject can be at an increased risk of bleeding at 7, 6, 5, 4, 3, 2, or 1 day, 12 hours, 8 hours, 4 hours, 2 hours, 1 hour or 45, 30, 15, 10, 5, 4, 3, 2, or 1 minute before the administering of the composition comprising the platelet derivatives. In some optional embodiments, this is confirmed by laboratory testing. However, in some embodiments no laboratory testing of bleeding risk or any clotting parameter is performed 7, 6, 5, 4, 3, 2, or 1 day or sooner before and/or after the administering of the composition comprising the platelet derivatives. Bleeding risk is typically decreased after administration of an effective dose of the composition comprising platelet derivatives, in illustrative embodiments FDPDs. Furthermore, the subject may remain at an increased risk of bleeding even after the administering of the composition comprising platelet derivatives (e.g. FDPDs), for example for 1, 2, 3, 4, 5, 10, 15, 20, 30, or 45 minutes, or 1, 2, 3, 4, 5, or 8 hours, or longer after the administering, depending on how long it takes for the FDPDs to decrease the risk in the subject after they are administered. Furthermore, in some embodiments, the administration of the composition comprising the platelet derivatives (e.g. FDPDs) decreases but does not completely resolve the increased risk of bleeding in the subject.

In some embodiments, for example of aspects wherein a subject was administered the antiplatelet agent and the second agent that decreases platelet function, administration of the second agent is stopped, for example before administrating the composition comprising the platelet derivatives. In other embodiments of such aspects, administration of the second agent is continued, for example after administering the composition comprising the platelet derivatives.

In certain embodiments of any of the aspects provided herein, the method further comprises before administering the composition comprising platelet derivatives, determining in a pre-administering evaluation, that the subject has an abnormal value for one or more clotting parameters. The pre-administration evaluation, in illustrative embodiments, is an in vitro laboratory test.

In certain embodiments of any of the aspects provided herein, the antiplatelet agent is selected from aspirin, cangrelor, ticagrelor, clopidogrel, prasugrel, eptifibatide, tirofiban, abciximab, terutroban, picotamide, elinogrel, ticlopidine, ibuprofen, vorapaxar, atopaxar, cilostazol, prostaglandin E1, epoprostenol, dipyridamole, treprostinil sodium, and sarpogrelate. In other embodiments, the antiplatelet agent is selected from cangrelor, ticagrelor, abciximab, terutroban, picotamide, elinogrel, ibuprofen, vorapaxar, atopaxar, cilostazol, prostaglandin E1, epoprostenol, dipyridamole, treprostinil sodium, and sarpogrelate.

### Plurality of Containers Containing Platelet Derivative Composition

The platelet derivative composition as described herein can be contained in containers/vials, which further can be packed into a plurality of containers for shipping to a customer, which can be part of a commercialization process to fulfill an order for such platelet derivative composition. The containers/vials can be included with packaging that contains instructions for performing methods herein. The containers, in certain embodiments, are 5 ml vials, 10 ml vials, 20 ml vials, 25 ml vs, 30 ml vials, 40 ml vials, 50 ml vials, 60 ml vials, 75 ml vials, 100 ml vials, 125 ml vials, 150 ml vials, 200 ml vials, or 250 ml vials. The vial(s) can be a cryovial, or a cryotube especially in illustrative embodiments where the TFF-treated composition that includes platelets is lyophilized to obtain the platelet derivative composition in the form of a powder, which further can be baked or not baked after it is lyophilized. In some embodiments, the volume of the containers in a plurality of containers (e.g. vials or tubes), which for example can be all from one lot, or from more than one lot (e.g. 2, 3, 4, 5, 6, 7, 8, 9 or 10 lots), can vary from one or more than one size between 10-100 ml. Typically, the volume of the vial/container in embodiments where the platelet derivative is a freeze-dried solid/powder, is 1× the volume of, or 1.10, 1.25, 1.5, 2, 2.5, 3, 4 or 5 times the volume of a composition that was filled in the vial before lyophilization, and/or the volume in which the powder in the vials will be rehydrated, which is an illustrative embodiment. Thus, the maximum volume of such vials can be the same or more than the volume of the composition that was filled inside prior to lyophilization or the volume in which the platelet derivative composition in the form of a powder can be rehydrated. For example, in one non-limiting embodiment, a vial with a maximum capacity of 100 ml, can be used to fill 10 ml of a TFF-treated composition that includes platelets for lyophilization. In certain embodiments, the capacity of a vial in which a TFF-treated composition that includes platelets is lyophilized, is 1-2.5 times and in other embodiments, 1-2 times, 1-3 times, 1-4 times, 1-5 times, and in certain illustrative embodiments, 1.1 to 2 times or 1.25 to 2 times the volume of a TFF-treated composition that is lyophilized therein.

The TFF-treated platelet composition before lyophilization, or in some embodiments, the platelet derivative composition obtained after the lyophilization step, with or without post-lyophilization heat treatment (baking), can be filled into a plurality of vessels or other powder and liquid-holding containers, such as vials, in a sterile manner. In some embodiments, the containers can vary in volume from 5-100 ml, 10-90 ml, 25-75 ml, or 5-40 ml. In some embodiments, the volume of containers can be 5 ml, 10 ml, 15 ml, 20 ml, 25 ml, 30 ml, 35 ml, 40 ml, 45 ml, 50 ml, 55 ml, 60 ml, 65 ml, 70 ml, 75 ml, 80 ml, 85 ml, 90 ml, 95 ml, or 100 ml. In some embodiments, the volume of containers can be above 100 ml, for example, 125 ml, 150 ml, 175 ml, or 200 ml. The platelet derivative composition as described herein can be filled in vials of different volumes as per the commercialization requirements. A plurality (or collection) of containers having the platelet derivative composition as per any of the embodiments herein, obtained by lyophilizing the composition that includes platelets during one process (e.g. TFF or other process) for preparing a platelet derivative, can be referred to as a “batch” or a “lot”. In some embodiments, a batch/lot can have 10-500 vials, 25-450 vials, 50-350 vials, 100-300 vials, or 150-250 vials. In some embodiments, a batch/lot can have 10, 20, 30, 40, 50, 60, 70, 80, 90, 100, 110, 120, 130, 140, 150, 160, 170, 180, 190, 200, 250, 300, 350, 400, 450, or 500 vials. In some embodiments, the number of vials per batch/lot can be increased to more than 500 as per the requirements, for example, 600, 700, 800, 900, or 1000 vials. In some embodiments, the number of vials can be 10-1000, 50-1000, 100-900, 200-800, 100-500, 100-400, 150-700, or 150-500 vials. The containers in a batch/lot can have a volume in the range of 5-100 ml, for example, such that a lot has several containers with the same volume or containers with different volumes. For example, 200 vials/containers in a batch/lot can have a volume of 10 ml each, 100 vials/containers in the same or a separate batch/lot can have a volume of 20 ml each, 100 vials/containers in the same or another batch/lot can have a volume of 30 ml each, or 300 vials/containers in the same or a different batch/lot can have a volume of 10 ml each. The number of containers (e.g. vials) in which a platelet derivative composition as per one of the embodiments or aspects described herein can be packed in a batch/lot can vary with manufacturing requirements, the requirements of downstream processes, for example clinical processes, and the amount of starting material comprising platelet composition.

The quantity of platelet derivatives that is present in a batch/lot can vary based on the units of starting material comprising platelets that is used to produce the platelet derivatives. Certain methods provided herein, such as the TFF methods provided herein, allow more platelet units to be used to make platelet derivatives with the characteristics provided herein than prior methods. This is the result, for example, of the ability to reduce the level of certain components in a platelet composition starting material, such as HLA antibodies, HNA antibodies, and/or microparticles, to very low levels, as provided herein. Accordingly, the starting material comprising platelets, the corresponding composition (e.g. TFF-treated composition) that is lyophilized in illustrative embodiments, and the resulting platelet derivative composition powder, can vary, such that for example, in some embodiments, the starting material, the TFF-treated composition, and/or the resulting platelet derivative composition powder can include 10-500 units of platelets or platelet derivatives (e.g. 0.5 to 2.5 μm in diameter), with one unit being 3×10¹¹ platelets or platelet derivatives. In some embodiments, the starting platelet material, the composition to be lyophilized, and/or the platelet derivative (e.g. 0.5 to 2.5 μm in diameter) composition can include, for example, 20-500 units, 30-400 units, 40-350 units, or 50-200 units of platelets or platelet derivatives, respectively. In some embodiments, the platelet units in the starting platelet composition can be a pooled platelet product from multiple donors as described herein, or multiple batches of processed platelet compositions, such as TFF-treated compositions comprising platelets, can be pooled before lyophilization. In some embodiments, there can be 1×10⁹ to 1×10¹⁶ platelets in a starting platelet composition for processing, in a platelet composition that is lyophilized, and/or of platelet derivatives in a platelet derivative composition that is produced after lyophilization, per batch/lot. In some embodiments, the platelet-containing starting composition, the platelet composition that is lyophilized, and/or the platelet derivatives that are produced, typically after lyophilization per batch/lot can vary from 1×10¹⁰ to 1×10¹⁵, 1×10¹¹ to 1×10¹⁵, 1×10¹² to 1×10¹⁶, 1×10¹³ to 1×10¹⁵ or 1×10¹³ to 1×10¹⁴.

In certain illustrative embodiments, platelet derivative compositions that are present in a liquid, or in illustrative embodiments, a solid form such as a dried powder in the plurality of containers (e.g. vials), in illustrative embodiments of a 1 or more lots, are compositions that include platelet derivatives, wherein at least 50% of the platelet derivatives are CD 41-positive platelet derivatives, wherein less than 15%, 10%, or in further, non-limiting illustrative embodiments less than 5% of the CD 41-positive platelet derivatives are microparticles having a diameter of less than 0.5 μm, and wherein the platelet derivatives have a potency of at least 0.5, 1.0 and in further, non-limiting illustrative embodiments 1.5 thrombin generation potency units (TGPU) per 10⁶ platelet derivatives. In certain illustrative embodiments, including non-limiting examples of the illustrative embodiment in the preceding sentence, the platelet derivatives are 0.5 to 2.5 um in diameter. Such platelet derivatives and platelet derivative compositions comprising the same, can have additional characteristics disclosed herein for such derivatives and compositions.

Processes provided herein for producing platelet derivative compositions, provide better lot to lot consistency than prior processes. For example, TFF methods provided herein provide improved lot to lot variability with respect to the components of compositions that include platelet derivatives prepared therein, in illustrative embodiments, compositions that include freeze-dried platelet derivates. Such freeze-dried platelet derivatives can be one of, or the main active ingredient(s). In some embodiments, a plurality of containers provided herein comprise the platelet derivative composition from at least 2 different lots in separate containers. In some embodiments, the amount of plasma protein in the powder of any two containers chosen from different lots, differs by less than 50%, 40%, 30%, 25%, or 20%, and in illustrative embodiments less than 10%, 5%, 2%, 1%, or 0.5%. The TFF process is highly controllable and can be stopped at a certain A280 for example, from 2.0 AU to 0.01 AU, or when it reaches 15% to 0.01% protein concentration in the composition that is to be lyophilized (e.g. TFF-treated composition), therefore, the plasma protein content can be very consistent not only within the containers/vials of a lot, but even between lots as well. Since different lots of platelet derivative compositions provided herein are typically prepared from platelets from different subjects or different combinations of subjects (e.g. pooled platelets from 2, 3, 4, 5, 6, 7, 8, 9, 10, 15, 20, 25, 30, 40, 50, 60, 70, 75, 80, or 100 subjects), different lots in illustrative embodiments differ in amino acid sequence of at least one (e.g., 1, 2, 3, 4, 5, 6, 7, 8, 9, 10, 1-100, or 1-10) of the proteins in, on, and/or associated with platelet derivatives of the compositions therein between the lots. In illustrative embodiments, these one or more amino acid differences occur at one or more (e.g., 1, 2, 3, 4, 5, 6, 7, 8, 9, 10, 1-100, or 1-10) of the site(s) of SNPs, in illustrative embodiments non-synonymous SNPs. In certain embodiments, such SNPs or non-synonymous SNPs have a minor allele frequency of less than or equal to 5%. In some embodiments, such pooled platelets are provided by processes provided herein, for example because HLA or HNA antibody levels can be reduced to very low or non-existent levels. Thus, not only can platelets be pooled from more subjects, before processing to form platelet derivatives, but those subjects can be males or females. As a result, platelet derivatives, in illustrative embodiments, FDPDs, of compositions herein, for example liquid or dried compositions, in some embodiments have different amino acid sequences for at least 1 or a plurality of FDPD proteins. Furthermore, as a result, in certain embodiments, within a lot or between lots, greater than 10%, 20%, 25%, 30%, or 40%, and in illustrative embodiments greater than 50%, 60%, 70%, 75%, 80%, 90%, or 95% of amino acids encoded by SNPs, in illustrative embodiments encoded by non-synonymous SNPs in one or more proteins that are bound to or otherwise associated with or part of a platelet derivative, are present for SNPs, for example with a minor allele frequency of greater than 5%, in certain embodiments including in proteins that result from expression of coding sequences comprising SNPs, in illustrative embodiments non-synonymous SNPs on a mammalian X and Y chromosome.

In some embodiments, the amount of microparticles that are less than 0.5 μm in the powder of any two containers chosen from different lots, differs in amount by less than 10%, 5%, 2%, or 1%. Since, for example, a TFF process disclosed herein is very controllable, the concentration of microparticles to be obtained in the platelet derivative composition can be optimized, for example, by performing scattering intensity studies at different time points. Once the desired level is achieved, the TFF-treated composition can be lyophilized and packed in the vials with or without the baking step.

In some embodiments, the percentage by weight of platelet derivative in the powder of any two containers chosen from different lots, differs by less than 10%, 5%, 2%, or 1%. The TFF process can be optimized to achieve a pre-determined level of platelet derivatives in the TFF-treated composition. Such a TFF-treated composition when lyophilized gives a platelet composition in the form of a powder having a certain weight percentage of platelet derivatives. Since, the TFF process is controllable, in some embodiments, there can be a minimum or a negligible variation in the weight percentages of the platelet derivatives in any two containers chosen from different lots.

In some embodiments, at least one container comprises a first lot of platelet derivatives and the one or more other containers comprise a second lot of platelet derivatives. In some embodiments, plurality of containers comprises the platelet derivative composition from at least 2, 3, 4, 5, 6, 7, 8, 9, or 10 different lots, wherein the platelet derivative composition in at least 2 of the lots have a different amino acid sequences for at least one protein of a collection of protein gene products from a corresponding collection of encoding genes. In illustrative embodiments all, of the lots have a different amino acid sequences for at least one protein of a collection of protein gene products from a corresponding collection of encoding genes. In some embodiments, the amino acid difference(s) is at one or more residues corresponding to amino acid residues encoded by a non-synonymous single nucleotide polymorphism (SNP).

As per one of the embodiments, a platelet derivative composition as described herein can be prepared from multiple donors of a single species, for example, mammals, such as for example canine, equine, porcine and in illustrative embodiments humans that are genetically different, in order to obtain a platelet derivative composition to prepare allogenic platelet derivatives, an allogenic platelet derivative product, and/or a composition comprising allogenic platelet derivatives. Such a platelet derivative composition can be filled in vials and a plurality of such vials can be packaged in containers, for example boxes for commercialization as described herein, to obtain a commercial product that is a composition comprising allogeneic platelet derivatives, in illustrative embodiments allogeneic freeze-dried platelet derivatives. The allogenic platelet derivatives as described herein, in some embodiments, can be a U.S. FDA-approved product comprising an allogenic platelet derivative composition. In some embodiments, a platelet derivative composition as described herein can be a European EMA-approved product comprising an allogenic platelet derivative composition. In some other embodiments, a platelet derivative composition as described herein can be a China FDA-approved product comprising an allogenic platelet derivative composition.

In some embodiments, platelets are pooled from a plurality of donors before they are used as starting material for a process for producing a platelet derivative as provided herein. Such platelets pooled from a plurality of donors can be also referred herein to as pooled platelets. In some embodiments, the donors are more than 5, such as more than 10, such as more than 20, such as more than 50, such as up to about 100 donors. In some embodiments, the donors are from 5 to 100, such as from 10 to 50, such as from 20 to 40, such as from 25 to 35. Pooled platelets can be used to make any of the platelet derivative compositions as described herein. The platelets can be pooled wherein the platelets are donated by mammalian (e.g. bovine, feline, porcine, canine, and in illustrative embodiments, human) subjects. In some embodiments, the gender of the subjects can be male or female. In some embodiments, the donor can vary from any number of male to any number of female subjects, for example, from a total of 100 donors, any number can be female donors, ranging from 0-100, 5-95, 10-90, 20-80, 30-70, or 40-60, and the rest can be male donors. In some other embodiments, the donor can be a non-human animal. In some embodiments, the donor can be a canine, equine, porcine, bovine, or feline subject.

In some embodiments of any of the aspects and embodiments herein that include a composition, or in some compositions used in or formed by a process that includes a plurality of containers each filled with a platelet derivative composition in the form of a powder, each of the plurality of containers are purged with at least one inert gas. In some embodiments, the inert gas can be argon, or nitrogen. In some embodiments, platelet derivatives in the plurality of containers herein can be used in any of the methods disclosed herein. For example, platelet derivatives in the plurality of containers herein can be used for administering platelet derivatives to a subject, in illustrative embodiments, a subject having a low platelet count.

### Preparation Agent and Additional Components

In some embodiments, a composition provided herein can include one or more additional components. In some embodiments, a composition provided herein can include a preparation agent (e.g., any of the preparation agents described herein). In some embodiments, the composition can include a buffering agent, a base, a loading agent, optionally a salt, and optionally at least one organic solvent. A buffering agent can be any appropriate buffering agent. In some embodiments, a buffering agent can be HEPES (4-(2-hydroxyethyl)-1-piperazineethanesulfonic acid). A base can be any appropriate base. In some embodiments, a base can be sodium bicarbonate. A loading agent can be any appropriate loading agent. In some embodiments, a loading agent can be a monosaccharide, a polysaccharide, or a combination thereof. In some embodiments, a loading agent can be selected from the group consisting of sucrose, maltose, trehalose, glucose, mannose, and xylose. In some embodiments, a loading agent can be trehalose. In some embodiments, a polysaccharide can be polysucrose. A salt can be any appropriate salt. In some embodiments, a salt can be sodium chloride, potassium chloride, or a combination thereof. An organic solvent can be any appropriate organic solvent. In some embodiments, an organic solvent can be selected from the group consisting of ethanol, acetic acid, acetone, acetonitrile, dimethylformamide, dimethyl sulfoxide, dioxane, methanol, n-propanol, isopropanol, tetrahydrofuran (THF), N-methyl pyrrolidone, dimethylacetamide (DMAC), and combinations thereof.

A preparation agent can include any appropriate components. In some embodiments, the preparation agent may comprise a liquid medium. In some embodiments the preparation agent may comprise one or more salts selected from phosphate salts, sodium salts, potassium salts, calcium salts, magnesium salts, and any other salt that can be found in blood or blood products, or that is known to be useful in drying platelets, or any combination of two or more of these.

In some embodiments, the preparation agent comprises one or more salts, such as phosphate salts, sodium salts, potassium salts, calcium salts, magnesium salts, and any other salt that can be found in blood or blood products. Exemplary salts include sodium chloride (NaCl), potassium chloride (KCl), and combinations thereof. In some embodiments, the preparation agent includes from about 0.5 mM to about 100 mM of the one or more salts. In some embodiments, the preparation agent includes from about 0.5 mM to about 100 mM (e.g., about 0.5 to about 2 mM, about 2 mM to about 90 mM, about 2 mM to about 6 mM, about 50 mM to about 100 mM, about 60 mM to about 90 mM, about 70 to about 85 mM) of the one or more salts. In some embodiments, the preparation agent includes about 5 mM, about 75 mM, or about 80 mM of the one or more salts. In some embodiments, the preparation agent comprises one or more salts selected from calcium salts, magnesium salts, and a combination of the two, in a concentration of about 0.5 mM to about 2 mM.

Preferably, these salts are present in the composition comprising platelets or platelet derivatives, such as freeze-dried platelets, at an amount that is about the same as is found in whole blood.

In some embodiments, the preparation agent further comprises a carrier protein. In some embodiments, the carrier protein comprises human serum albumin, bovine serum albumin, or a combination thereof. In some embodiments, the carrier protein is present in an amount of about 0.05% to about 1.0% (w/v).

The preparation agent may be any buffer that is non-toxic to the platelets and provides adequate buffering capacity to the solution at the temperatures at which the solution will be exposed during the process provided herein. Thus, the buffer may comprise any of the known biologically compatible buffers available commercially, such as phosphate buffers, such as phosphate buffered saline (PBS), bicarbonate/carbonic acid, such as sodium-bicarbonate buffer, N-2-hydroxyethylpiperazine-N-2-ethanesulfonic acid (HEPES), and tris-based buffers, such as tris-buffered saline (TBS). Likewise, it may comprise one or more of the following buffers: propane-1,2,3-tricarboxylic (tricarballylic); benzenepentacarboxylic; maleic; 2,2-dimethylsuccinic; EDTA; 3,3-dimethylglutaric; bis(2-hydroxyethyl)imino-tris(hydroxymethyl)-methane (BIS-TRIS); benzenehexacarboxylic (mellitic); N-(2-acetamido)imino-diacetic acid (ADA); butane-1,2,3,4-tetracarboxylic; pyrophosphoric; 1,1-cyclopentanediacetic (3,3 tetramethylene-glutaric acid); piperazine-1,4-bis-(2-ethanesulfonic acid) (PIPES); N-(2-acetamido)-2-amnoethanesulfonic acid (ACES); 1,1-cyclohexanediacetic; 3,6-endomethylene-1,2,3,6-tetrahydrophthalic acid (EMTA; ENDCA); imidazole; 2-(aminoethyl)trimethylammonium chloride (CHOLAMINE); N,N-bis(2-hydroxyethyl)-2-aminoethanesulfonic acid (BES); 2-methylpropane-1,2,3-triscarboxylic (beta-methyltricarballylic); 2-(N-morpholino)propane-sulfonic acid (MOPS); phosphoric; and N-tris(hydroxymethyl)methyl-2-amminoethane sulfonic acid (TES). In some embodiments, the preparation agent includes one or more buffers, e.g., N-2-hydroxyethylpiperazine-N′-2-ethanesulfonic acid (HEPES), or sodium-bicarbonate (NaHCO₃). In some embodiments, the preparation agent includes from about 5 to about 100 mM of the one or more buffers. In some embodiments, the preparation agent includes from about 5 to about 50 mM (e.g., from about 5 mM to about 40 mM, from about 8 mM to about 30 mM, about 10 mM to about 25 mM) about of the one or more buffers. In some embodiments, the preparation agent includes about 10 mM, about 20 mM, about 25 mM, or about 30 mM of the one or more buffers.

In some embodiments, the preparation agent includes one or more saccharides, such as monosaccharides and disaccharides, including sucrose, maltose, trehalose, glucose, mannose, dextrose, and xylose. In some embodiments, the saccharide is a monosaccharide. In some embodiments, the saccharide is a disaccharide. In some embodiments, the saccharide is a monosaccharide, a disaccharide, or a combination thereof. In some embodiments, the saccharide is a non-reducing disaccharide. In some embodiments, the saccharide is sucrose, maltose, trehalose, glucose (e.g., dextrose), mannose, or xylose. In some embodiments, the saccharide comprises trehalose. In some embodiments, the preparation agent comprises a starch. In some embodiments, the preparation agent includes polysucrose, a polymer of sucrose and epichlorohydrin. In some embodiments, the preparation agent includes from about 10 mM to about 1,000 mM of the one or more saccharides. In some embodiments, the preparation agent includes from about 50 to about 500 mM of the one or more saccharides. In some embodiments, one or more saccharides is present in an amount of from 10 mM 10 to 500 mM. In some embodiments, one or more saccharides is present in an amount of from 50 mM to 200 mM. In some embodiments, one or more saccharides is present in an amount from 100 mM to 150 mM. In some embodiments, the one or more saccharides are the lyophilizing agent; for example, in some embodiments, the lyophilizing agent comprises trehalose, polysucrose, or a combination thereof. In some embodiments, the preparation agent comprises trehalose in the range of 0.4-35%, or 1-35%, or 2-30%, or 1-10%, or 1-5%, or 0.5-5%. In an exemplary embodiment, the composition comprises 3.5% trehalose. In some embodiments, the preparation agent comprises polysucrose in the range of 2-8%, or 2.25-7.75%, or 2.5-7.5%, or 2.5-6.5%, wherein the composition is in a rehydrated form. In an exemplary embodiment, the composition comprises 3% polysucrose. In another exemplary embodiment, the composition comprises 6% polysucrose. Different ionic forms of polysucrose can be used in the preparation agent that would be used to lyophilize the platelet derivatives. The ionic forms of polysucrose can be exploited to increase the efficiency of the lyophilization process. The ionic forms can be optimized to accommodate higher concentrations of platelet concentrations in the solution for performing lyophilization process. In some embodiments of the composition, wherein the composition comprises polysucrose, the polysucrose is a cationic form of polysucrose. In some embodiments, the cationic form of polysucrose is diethylaminoethyl (DEAE)-polysucrose. In some embodiments, the polysucrose is an anionic form of polysucrose. In some embodiments, the anionic form of polysucrose is carboxymethyl-polysucrose. Polysucrose of different molecular weight can be used to increase the efficiency of the lyophilization process. In some embodiments of the composition, polysucrose has a molecular weight in the range of 70,000 MW to 400,000 MW. In some embodiments, polysucrose has a molecular weight in the range of 80,000 MW to 350,000 MW, or 100,000 MW to 300,00 MW. In some exemplary embodiments, polysucrose has a molecular weight in the range of 120,000 MW to 200,000 MW. In some exemplary embodiments, polysucrose has a molecular weight of 150,000 MW, or 160,000 MW, or 170,000 MW, or 180,000 MW, 190,000 MW, or 200,000 MW.

In some embodiments the composition comprising platelets or platelet derivatives, (e.g., thrombosomes), may comprise one or more of water or a saline solution. In some embodiments the composition comprising platelets or platelet derivatives, such as freeze-dried platelets, may comprise DMSO.

In some embodiments, the preparation agent comprises an organic solvent, such as an alcohol (e.g., ethanol). In such a preparation agent, the amount of solvent can range from 0.1% to 5.0% (v/v). In some embodiments, the organic solvent can range from about 0.1% (v/v) to about 5.0% (v/v), such as from about 0.3% (v/v) to about 3.0% (v/v), or from about 0.5% (v/v) to about 2% (v/v).

In some embodiments, suitable organic solvents include, but are not limited to alcohols, esters, ketones, ethers, halogenated solvents, hydrocarbons, nitriles, glycols, alkyl nitrates, water or mixtures thereof. In some embodiments, suitable organic solvents includes, but are not limited to methanol, ethanol, n-propanol, isopropanol, acetic acid, acetone, methyl ethyl ketone, methyl isobutyl ketone, methyl acetate, ethyl acetate, isopropyl acetate, tetrahydrofuran, isopropyl ether (IPE), tert-butyl methyl ether, dioxane (e.g., 1,4-dioxane), acetonitrile, propionitrile, methylene chloride, chloroform, toluene, anisole, cyclohexane, hexane, heptane, ethylene glycol, nitromethane, dimethylformamide, dimethyl sulfoxide, N-methyl pyrrolidone, dimethylacetamide, and combinations thereof. In some embodiments the organic solvent is selected from the group consisting of ethanol, acetic acid, acetone, acetonitrile, dimethylformamide, dimethyl sulfoxide (DMSO), dioxane, methanol, n-propanol, isopropanol, tetrahydrofuran (THF), N-methyl pyrrolidone, dimethylacetamide (DMAC), or combinations thereof. In some embodiments, the organic solvent comprises ethanol, DMSO, or a combination thereof. The presence of organic solvents, such as ethanol, can be beneficial in the processing of platelets, platelet derivatives, or thrombosomes (e.g., freeze-dried platelet derivatives).

In some embodiments the preparation agent does not include an organic solvent. In some embodiments, the preparation agent comprises an organic solvent. In some embodiments the preparation agent comprises DMSO.

A preparation agent can have any appropriate pH. For example, in some embodiments, a preparation agent can have a pH of about 5.5 to about 8.0 (e.g., about 6.5 to about 6.9, or about 6.6 to about 6.8). In some embodiments, the preparation agent has a pH in the range of 5.5 to 8.0, or 6.0 to 8.0, or 6.0 to 7.5. In an exemplary embodiment, the preparation agent has a pH of 6.5. In another exemplary embodiment, the preparation agent has a pH of 7.4.

In some embodiments, one or more other components may be combined with in the platelets (e.g., as part of a preparation agent). Exemplary components may include Prostaglandin E1 or Prostacyclin and or EDTA/EGTA to prevent platelet aggregation and activation.

In some embodiments, a preparation agent can be Buffer A, as shown in Example 1. In some embodiments, a preparation agent can comprise Buffer A, as shown in Example 1, wherein one or more components (e.g., ethanol) is present in an amount up to three times the amount shown in Example 1. Non-limiting examples of preparation agent compositions that may be used are shown in Tables P1-P6.

TABLE P1
Table P1. A preparation agent that can be used
Buffer

| | Concentration (mM unless |
|---|---|
| Component | otherwise specified) |
| NaCl | 75.0 |
| KCl | 4.8 |
| HEPES | 9.5 |
| NaHCO₃ | 12.0 |
| Dextrose | 3 |
| Trehalose | 100 |
| Ethanol | 1% (v/v) |

TABLE P2
Table P2. A preparation agent that can be used
Buffer

| | Concentration (mM unless |
|---|---|
| Component | otherwise specified) |
| NaCl | 75.0 |
| KCl | 4.8 |
| HEPES | 9.5 |
| NaHCO₃ | 12.0 |
| Dextrose | 3 |
| Trehalose | 100 |

TABLE P3
Table P3. A preparation agent that can be used.
Buffer A1 (pH 6.5)

| | Concentration (mM unless |
|---|---|
| Component | specified otherwise) |
| CaCl₂ | 1.8 |
| MgCl₂ | 1.1 |
| KCl | 2.7 |
| NaCl | 137 |
| NaH₂PO₄ | 0.4 |
| HEPES | 10 |
| D-glucose | 5.6 |

TABLE P4
Table P4. Buffer B can used when incubating platelets, e.g., for
flow cytometry. Such an incubation can be done
at room temperature in the dark. Albumin
is an optional component of Buffer B.
Buffer B

| | Concentration (mM unless |
|---|---|
| Component | otherwise specified) |
| Buffer and Salts | Table P5 (below) |
| BSA | 0.35% |
| Dextrose | 5 |
| pH | 7.4 |

TABLE P5
Concentration of HEPES and of Salts in Buffer B

| | Concentration (mM unless |
|---|---|
| Component | otherwise specified) |
| HEPES | 25 |
| NaCl | 119 |
| KCl | 5 |
| CaCl₂ | 2 |
| MgCl₂ | 2 |
| glucose | 6 g/l |

Table P5 shows the concentrations of HEPES and salts in Buffer B. The pH can be adjusted to 7.4 with NaOH. Albumin is an optional component of Buffer B.

TABLE P6
Tyrode’s HEPES Buffer (plus PGE1)

| Component | Concentration (mM) |
|---|---|
| CaCl₂ | 1.8 |
| MgCl₂ | 1.1 |
| KCl | 2.7 |
| NaCl | 137 |
| NaH₂PO₄ | 0.4 |
| HEPES | 10 |
| D-glucose | 5.6 |
| pH | 6.5 |
| Prostagalandin E1 (PGE1) | 1 μg/ml |

Table P6 is another exemplary preparation agent.

### Rehydration of the Composition Comprising Platelet Derivatives

In some aspects, the platelet derivative composition of the present disclosure is in the form of a powder. In some aspects, the process for preparing the platelet derivative composition results in the final product which is in a dry powdered form. The platelet derivative composition in its dry form comprises platelet derivatives. The platelet derivative composition in its dry form comprises platelet derivatives, and/or freeze-dried platelets. It is well-known to a skilled artisan that the platelet derivatives in the dried form shall preserve the characteristics which it is intended to observe once the platelet derivatives are rehydrated for clinical application and/or studying the characteristics such as, the presence of platelet activation markers.

In some embodiments, rehydrating the composition comprising platelets or platelet derivatives comprises adding to the platelets an aqueous liquid. In some embodiments, the aqueous liquid is water. In some embodiments, the aqueous liquid is an aqueous solution (e.g., a buffer). In some embodiments, the aqueous liquid is a saline solution. In some embodiments, the aqueous liquid is a suspension.

Methods for preparing platelet derivatives herein, in some embodiments, do not include contacting platelets or platelet derivatives with an agent that can lead to cross-linking of platelet membranes. The agent or agents that can lead to cross-linking of platelet membranes can be referred to as cross-linking agents. Cross-linking of platelet membranes can include crosslinking via proteins and/or lipids present on the membranes. It can be understood by a skilled artisan that even in certain processes that do not include contacting platelets or platelet derivatives with cross-linking agents, there can be a type of crosslinking observed on the platelet membranes, referred to as endogenous cross-linking that can be different from the cross-linking observed when platelets are exposed to cross-linking agents, referred to as exogenous cross-linking. Accordingly, the endogenous cross-linking in the platelet derivatives, in some embodiments can be less than 5%, 4%, 3%, 2%, 1%, 0.5%, 0.1%, or 0.01%. For example, the endogenous cross-linking can be in the range of 0.0001-5%, 0.0001-4%, 0.0001-3%, 0.0001-2%, 0.0001-1%, 0.0001-0.5%, or 0.0001-0.1%. The exogenous cross-linking of platelet membranes observed on platelet derivatives herein because of the treatment with cross-linking agents can be less than 5%, 4%, 3%, 2%, 1%, 0.5%, 0.1%, or 0.01%. For example, the exogenous cross-linking can be in the range of 0.0001-5%, 0.0001-4%, 0.0001-3%, 0.0001-2%, 0.0001-1%, 0.0001-0.5%, or 0.0001-0.1%. Typically, methods herein do not include contacting platelets or platelet derivatives with a cross-linking agent, therefore, the platelet derivatives herein do not have any exogenous cross-linking, i.e., 0% exogenous cross-linking. Chemicals used for fixing platelets can be considered as cross-linking agent(s), for example, formaldehyde. Since the process herein do not include contacting platelets or platelet membranes with any aldehydes, such as, formaldehyde, the platelet derivatives herein do not have any exogenous cross-linking. In some embodiments, the platelets or platelet derivatives (e.g., thrombosomes) have less than about 10%, such as less than about 8%, such as less than about 6%, such as less than about 4%, such as less than about 2%, such as less than about 0.5% crosslinking of platelet membranes via proteins and/or lipids present on the membranes. In some embodiments, the rehydrated platelets or platelet derivatives (e.g., thrombosomes), have less than about 10%, such as less than about 8%, such as less than about 6%, such as less than about 4%, such as less than about 2%, such as less than about 0.5% crosslinking of platelet membranes via proteins and/or lipids present on the membranes.

In some embodiments, rehydrating the composition comprising platelets or platelet derivatives comprises adding to the platelets or platelet derivatives sterile water (e.g., sterile water for injection) over about 10 minutes at about room temperature. In general, the rehydration volume is about equal to the volume used to fill each vial of platelet derivative composition prior to drying, for example, freeze-drying. In some embodiments, the rehydrating includes rehydrating the platelet derivatives in a volume of a liquid, for example, water. In some embodiments, the rehydrating includes rehydrating the platelet derivatives in a volume of 10-100 ml, 20-80 ml, 20-60 ml, 30-60 ml, or 30-50 ml.

### Process for Preparing a Platelet Derivative Composition

In some embodiments, the platelets or pooled platelets can be initially diluted, further diluted (e.g. if initially diluted in an acidified buffer) or suspended in a preparation agent as described herein before being loaded onto a TFF unit to exchange the solution, buffer or diluted preparation agent with a preparation agent in the TFF unit. A skilled artisan would understand that before being loaded onto a TFF unit, the input composition can be initially diluted to a desirable dilution in order to carry out the TFF process in an effective manner. In some embodiments, the platelets or pooled platelets comprised in a composition can be diluted with an acidified washing buffer for example, and/or with a preparation agent as described herein before loading onto a TFF unit. In some embodiments, the platelets or pooled platelets are diluted 1:0.5, 1:1, 1:2, 1:4, 1:5, or 1:10, in a preparation agent, which in illustrative embodiments is the preparation in which the platelets will be freeze dried. In illustrative embodiments, the platelets or the pooled platelets can be diluted or suspended in a preparation agent comprising trehalose and in illustrative embodiments polysucrose before being loaded onto a TFF unit, followed by performing TFF with the preparation agent in the TFF unit. In illustrative embodiments, the platelets or the pooled platelets can be diluted or suspended in a preparation agent comprising 0.4 to 35% trehalose and 2% to 8% polysucrose, before being loaded onto a TFF unit, followed by performing TFF with the preparation agent in the TFF unit. A skilled artisan would understand that performing TFF is a continuous process of fluid exchange between the preparation agent and the platelets or the pooled platelets. The preparation agent that is used to dilute or suspend the platelets or the pooled platelets before being loaded onto a TFF unit can be the same preparation agent that is used for performing the TFF or it can be a different solution (e.g., acidified washing buffer) typically that is compatible with processing viable platelets. In some embodiments, the preparation agent used to dilute or suspend the platelets or the pooled platelets before being loaded onto a TFF unit can have the same ingredients but differ in the concentration of the ingredients than the preparation agent used for performing the TFF. A skilled artisan would understand the extent of the difference, if at all needed, based upon the dilution required to perform the TFF.

In some embodiments, the platelets or pooled platelets may be acidified to a pH of about 5.5 to about 8.0 prior to TFF or being diluted with the preparation agent. In some embodiments, the method comprises acidifying the platelets to a pH of about 6.5 to about 6.9. In some embodiments, the method comprises acidifying the platelets to a pH of about 6.6 to about 6.8. In some embodiments, the method comprises acidifying the platelets to a pH of about 6.6 to 7.5. In some embodiments, the acidifying comprises adding to the pooled platelets a solution comprising Acid Citrate Dextrose (ACD).

In some embodiments, the platelets are isolated prior to the step comprising tangential flow filtration (TFF) or being diluted with the preparation agent. In some embodiments, the method further comprises isolating platelets by using centrifugation. In some embodiments, the centrifugation occurs at a relative centrifugal force (RCF) of about 1000×g to about 2000×g. In some embodiments, the centrifugation occurs at relative centrifugal force (RCF) of about 1300×g to about 1800×g. In some embodiments, the centrifugation occurs at relative centrifugal force (RCF) of about 1500×g. In some embodiments, the centrifugation occurs for about 1 minute to about 60 minutes. In some embodiments, the centrifugation occurs for about 10 minutes to about 30 minutes. In some embodiments, the centrifugation occurs for about 30 minutes.

In some embodiments, platelets are isolated, for example in a liquid medium, prior to treating a subject.

In some embodiments, platelets are donor-derived platelets. In some embodiments, platelets are obtained by a process that comprises an apheresis step. In some embodiments, platelets are pooled platelets.

In some embodiments, platelets are pooled from a plurality of donors. Such platelets pooled from a plurality of donors may be also referred herein to as pooled platelets. In some embodiments, the donors are more than 5, such as more than 10, such as more than 20, such as more than 50, such as up to about 100 donors. In some embodiments, the donors are from about 5 to about 100, such as from about 10 to about 50, such as from about 20 to about 40, such as from about 25 to about 35. Pooled platelets can be used to make any of the compositions described herein. The platelets can be pooled wherein the platelets are donated by human subjects. In some other embodiments, the donor can be a non-human animal. In some embodiments, the donor can be a canine subject. In some embodiments, the donor can be an equine subject. In some embodiments, the donor can be a feline subject.

In some embodiments, platelets are derived in vitro. In some embodiments, platelets are derived or prepared in a culture. In some embodiments, preparing the platelets comprises deriving or growing the platelets from a culture of megakaryocytes. In some embodiments, preparing the platelets comprises deriving or growing the platelets (or megakaryocytes) from a culture of human pluripotent stem cells (PSCs), including embryonic stem cells (ESCs) and/or induced pluripotent stem cells (iPSCs).

Accordingly, in some embodiments, platelets or platelet derivatives (e.g., thrombosomes) are prepared prior to treating a subject as described herein. In some embodiments, the platelets or platelet derivatives (e.g., thrombosomes) are lyophilized. In some embodiments, the platelets or platelet derivatives (e.g., thrombosomes) are cryopreserved. For example, in some embodiments, the platelets or platelet derivatives can be cryopreserved in plasma and DMSO (e.g., 3-9% DMSO (e.g., 6% DMSO)). In some embodiments, the platelets or platelet derivatives are cryopreserved as described in U.S. Patent Application Publication No. 2020/0046771 A1, published on Feb. 13, 2020, incorporated herein by reference in its entirety.

In some embodiments, platelets (e.g., apheresis platelet, platelets isolated from whole blood, pooled platelets, or a combination thereof) form a suspension in a preparation agent comprising a liquid medium at a concentration from 10,000 platelets/μL to 10,000,000 platelets/μL, such as 50,000 platelets/μL to 2,000,000 platelets/μL, such as 100,000 platelets/μL to 500,000 platelets/μL, such as 150,000 platelets/μL to 300,000 platelets/μL, such as 200,000 platelets/μL.

In some embodiments, the method further comprises drying the platelets or platelet derivatives (e.g., thrombosomes). In some embodiments, the drying step comprises lyophilizing the platelets or platelet derivatives (e.g., thrombosomes). In some embodiments, the drying step comprises freeze-drying the platelets or platelet derivatives (e.g., thrombosomes). In some embodiments, the method further comprises rehydrating the platelets or platelet derivatives (e.g., thrombosomes) obtained from the drying step.

In some embodiments, the platelets or platelet derivatives (e.g., thrombosomes) are cold stored, cryopreserved, or lyophilized (e.g., to produce thrombosomes) prior to use in therapy or in functional assays.

Any known technique for drying platelets can be used in accordance with the present disclosure, as long as the technique can achieve a final residual moisture content of less than 5%. Preferably, the technique achieves a final residual moisture content of less than 2%, such as 1%, 0.5%, or 0.1%. Non-limiting examples of suitable techniques are freeze-drying (lyophilization) and spray-drying. A suitable lyophilization method is presented in Table LA. Additional exemplary lyophilization methods can be found in U.S. Pat. Nos. 7,811,558, 8,486,617, and 8,097,403. An exemplary spray-drying method includes: combining nitrogen, as a drying gas, with a preparation agent according to the present disclosure, then introducing the mixture into GEA Mobile Minor spray dryer from GEA Processing Engineering, Inc. (Columbia MD, USA), which has a Two-Fluid Nozzle configuration, spray drying the mixture at an inlet temperature in the range of 150° C. to 190° C., an outlet temperature in the range of 65° C. to 100° C., an atomic rate in the range of 0.5 to 2.0 bars, an atomic rate in the range of 5 to 13 kg/hr, a nitrogen use in the range of 60 to 100 kg/hr, and a run time of 10 to 35 minutes. The final step in spray drying is preferentially collecting the dried mixture. The dried composition in some embodiments is stable for at least six months at temperatures that range from −20° C. or lower to 90° C. or higher.

TABLE LA
Exemplary Lyophilization Protocol

| | Step | Temp. Set | Type | Duration | Pressure Set |
|---|---|---|---|---|---|
| Freezing Step | F1 | −50° C. | Ramp | Var | N/A |
| | F2 | −50° C. | Hold | 3 Hrs | N/A |
| Vacuum Pulldown | F3 | −50° | Hold | Var | N/A |
| Primary Dry | P1 | −40° | Hold | 1.5 Hrs | 0 mT |
| | P | | | | 0 |
| | P2 | −35° | Ramp | 2 Hrs | 0 mT |
| | P3 | −25° | Ramp | 2 Hrs | 0 mT |
| | P4 | −17° C. | Ramp | 2 Hrs | 0 mT |
| | P5 | 0° C. | Ramp | 1.5 Hrs | 0 mT |
| | P6 | 27° C. | Ramp | 1.5 Hrs | 0 mT |
| | P7 | 27° C. | Hold | 16 Hrs | 0 mT |
| Secondary Dry | S1 | 27° C. | Hold | >8 Hrs | 0 mT |

In some embodiments, the step of drying the platelets or platelet derivatives (e.g., thrombosomes) that are obtained as disclosed herein, such as the step of freeze-drying the platelets and/or platelet derivatives that are obtained as disclosed herein, comprises incubating the platelet and/or platelet derivatives with a lyophilizing agent (e.g., a non-reducing disaccharide). Accordingly, in some embodiments, the methods for preparing platelets and/or platelet derivatives further comprises incubating the platelets with a lyophilizing agent. In some embodiments the lyophilizing agent is a saccharide. In some embodiments the saccharide is a disaccharide, such as a non-reducing disaccharide.

In some embodiments, the platelets and/or platelet derivatives are incubated with a lyophilizing agent for a sufficient amount of time and at a suitable temperature to incubate the platelets with the lyophilizing agent. Non-limiting examples of suitable lyophilizing agents are saccharides, such as monosaccharides and disaccharides, including sucrose, maltose, trehalose, glucose (e.g., dextrose), mannose, and xylose. In some embodiments, non-limiting examples of lyophilizing agent include serum albumin, dextran, polyvinyl pyrolidone (PVP), starch, and hydroxyethyl starch (HES). In some embodiments, exemplary lyophilizing agents can include a high molecular weight polymer. By “high molecular weight” it is meant a polymer having an average molecular weight of about or above 70 kDa and up to 1,000,000 kDa. Non-limiting examples are polymers of sucrose and epichlorohydrin (e.g., polysucrose). In some embodiments, the lyophilizing agent is polysucrose. Although any amount of high molecular weight polymer can be used as a lyophilizing agent, it is preferred that an amount be used that achieves a final concentration of about 3% to 10% (w/v), such as 3% to 7%, for example 6%. In some embodiments, polysucrose is used in the range of 2% to 8%%, or 2.25-7.75%, or 2.5-7.5%, or 2.5-6.5%. In an exemplary embodiment, the composition comprises 3% polysucrose. In another exemplary embodiment, the composition comprises 6% polysucrose. In some embodiments of the composition, wherein the composition comprises polysucrose, the polysucrose is a cationic form of polysucrose. In some embodiments, the cationic form of polysucrose is diethylaminoethyl (DEAE)-polysucrose. In some embodiments, the polysucrose is an anionic form of polysucrose. In some embodiments, the anionic form of polysucrose is carboxymethyl-polysucrose. In some embodiments of the composition, polysucrose has a molecular weight in the range of 70,000 Da to 400,000 Da. In some embodiments, polysucrose has a molecular weight in the range of 80,000 Da to 350,000 Da, or 100,000 Da to 300.00 Da. In some exemplary embodiments, polysucrose has a molecular weight in the range of 120,000 Da to 200,000 Da. In some exemplary embodiments, polysucrose has a molecular weight of 150,000 Da, or 160,000 Da, or 170,000 Da, or 180,000 Da, 190,000 Da, or 200,000 Da.

An exemplary saccharide for use in the compositions disclosed herein is trehalose. Regardless of the identity of the saccharide, it can be present in the composition in any suitable amount. For example, it can be present in an amount of 1 mM to 1 M. In embodiments, it is present in an amount of from 10 mM 10 to 500 mM. In some embodiments, it is present in an amount of from 20 mM to 200 mM. In embodiments, it is present in an amount from 40 mM to 100 mM. In some embodiments, the composition comprises trehalose in the range of 0.4-35%, or 1-35%, or 2-30%, or 1-10%, or 1-5%, or 0.5-5%. In an exemplary embodiment, the composition comprises 3.5% trehalose.

In various embodiments, the saccharide is present in different specific concentrations within the ranges recited above, and one of skill in the art can immediately understand the various concentrations without the need to specifically recite each herein. Where more than one saccharide is present in the composition, each saccharide can be present in an amount according to the ranges and particular concentrations recited above.

In some cases, preparation of thrombosomes further comprises one or more of the procedures described in U.S. Pat. No. 8,486,617 (such as, e.g., Examples 1-5) and U.S. Pat. No. 8,097,403 (such as, e.g., Examples 1-3), incorporated herein by reference in their entirety. In some cases, a starting material (e.g., one or more donor platelet units) are initially pooled into a common vessel. In some embodiments, a starting material can comprise one or more donor platelet units. In some embodiments, a starting material can comprise donor plasma. The starting material may or may not be acidified with an anti-coagulation buffer (i.e. ACD-A) before centrifugation. Plasma can be aspirated off of the platelet pellet after centrifugation. Cell compatible buffer containing cryoprotectants (e.g., a loading buffer, which can be similar to or the same as a preparation agent) can be added to the platelet pellet before resuspending the cells into suspension. Platelets may or may not be diluted to a pre-determined concentration (e.g., 2200 k/ul to 2800 k/ul) with buffer if desired. Platelets in buffer may be incubated between 0 minutes and 240 minutes at an incubation temperature from 18° C. to 37° C. A lyoprotectant bulking agent (e.g., polysucrose) can be added to the platelets in buffer to achieve a final bulking agent concentration from 1% to 10% w/v (with preference at 6% w/v). The centrifuged processed platelets can then be filled into vials, lyophilized and thermally treated.

### Platelet Derivatives and Microparticles

Platelet derivatives herein have been observed to have numerous surprising properties, as disclosed in further detail herein. It will be understood, as illustrated in the Examples provided herein, that although platelet derivatives in some aspects and embodiments are in a solid, such as a powder form, the properties of such platelet derivatives can be identified, confirmed, and/or measured when a composition comprising such platelet derivatives is in liquid form.

A skilled artisan would be well-versed with different techniques that are available for measuring particle sizes of platelets, platelet derivatives or FDPDs, and microparticles. One such technique, in a non-limiting manner, that can be used for measuring particle sizes is flow cytometry. Flow Cytometry is a technique for quantifying characteristics of cells such as cell number, size and complexity, fluorescence, phenotype, and viability. In general, the forward scatter in a flow cytometry is located in line with the laser intercept and is typically considered a measure of the relative cell size. The side scatter is typically located perpendicular to the laser beam intercept and is used to measure the relative complexity of the cell. Commercially available sizing beads can be used to obtain the forward scatter values to calibrate the instrument in order to measure the sizes of the particles.

Liquid and dried compositions provided herein, in illustrative embodiments those prepared using freeze drying, and more specifically in some embodiments, prepared using methods provided herein, include particles that can be categorized broadly into populations based on at least one physical property, for example, but not limiting to, the size of the particles obtained. In some embodiments, the particles can be categorized into two populations based on size, typically in embodiments where exosomes are not present in detectable quantities, are not resolvable by the instrument analyzing particle size, and/or are not considered particles: For example, a first population comprising larger particles similar, or much more similar in size to in-dated stored platelets, which can be referred to herein as platelet derivatives, FDPDs, platelet-sized particles, a population of platelet derivatives with a size distribution centered around ˜1,000 nm radius, or ˜1,000 nm radius particles, and a second population comprising relatively smaller particles, which can be referred to herein as microparticles, a population of microparticles with a size distribution centered around ˜50 nm radius, or ˜50 nm radius particles where the main population of human in-dated stored platelets is centered at around 1,100 or 1,500 nm radius respectively, with a smaller (i.e. microparticle) peak at around 100 nm radius; and the particles in the FDPD composition (i.e. thrombosomes), which have a ˜platelet-sized major peak with a radius of approximately 1,100 nm or 1,000 nm (i.e. platelet derivatives) and a microparticle radius peak at approximately 50-75 nm).

A skilled artisan would further understand that the sizes determined for such populations of particles may not always be accurate enough to provide an exact cut-off value/range between these two particle size peaks. However, the difference in the sizes of the two populations can be resolved reproducibly using known methods, for example, using flow cytometry, or by using a particle/cell counter. And approximate size values or size range values can be obtained using such techniques optionally with sizing standards. In some embodiments, a composition comprising platelet derivatives or FDPDs as described herein or prepared according to methods described herein. can have a population comprising platelet derivatives or FDPDs that includes between 95.1% to 99.9% of total particles in the composition, and the rest of the measurable particles, for example above 1 nm radius, can be microparticles. In some embodiments, platelet derivatives or FDPDs in such a composition can have a diameter of at least 0.4 μm (i.e., radius of at least 200 nm), and the microparticles in such a composition can have a diameter less than 0.4 μm (i.e., radius less than 200 nm). In other embodiments, platelet derivatives or FDPDs in such a composition can have a diameter of at least 0.5 μm (i.e., radius of at least 250 nm), and the microparticles in such a composition can have a diameter less than 0.5 μm (i.e., radius of less than 250 nm). In some embodiments, the platelet derivatives, or FDPDs, or aggregates thereof, can have a diameter of at least 0.4 μm, for example in the range of 0.5 μm to 22 μm (i.e., radius in the range of 200 nm or 250 nm to 11,000 nm), and the microparticles can have a diameter less than 0.5 μm (i.e., less than 250 nm radius), for example in the range of 0.04 μm to 0.350 μm (i.e., radius in the range of 20 nm to 175 nm). In some embodiments, the platelet derivatives, or FDPDs can have a diameter in the range of 1 μm to 18 μm (i.e., radius in the range of 500 nm to 9,000 nm), and the microparticles can have a diameter in the range of 0.06 μm to 0.2 μm (i.e., radius in the range of 30 nm to 100 nm). In some embodiments, the composition comprises platelet derivatives or FDPDs, and microparticles as the only or essentially the only particles present in the composition, optionally or typically other than exosomes, in embodiments where exosomes are not present in detectable quantities, are not resolvable by the instrument analyzing particle size, and/or are not considered particles. Of course, a composition as described herein may comprise any specific percentage number, or fraction thereof, of platelet derivatives, FDPDs or microparticles within the ranges discussed herein.

In some embodiments, the platelets or platelet derivatives (e.g., thrombosomes) have a particle size, for example a diameter, max dimension, or radius of at least about 0.5 μm (e.g., at least about at least about 0.6 μm, at least about 0.7 μm, at least about 0.8 μm, at least about 0.9 μm, at least about 1.0 μm, at least about 1.2 μm, at least about 1.5 μm, at least about 2.0 μm, at least about 2.5 μm, or at least about 5.0 μm). In some embodiments, the particle size, for example the diameter, max dimension, or radius, is less than about 5.0 μm (e.g., less than about 2.5 μm, less than about 2.0 μm, less than about 1.5 μm, less than about 1.0 μm, less than about 0.9 μm, less than about 0.8 μm, less than about 0.7 μm, less than about 0.6 μm, less than about 0.5 μm, less than about 0.4 μm, or less than about 0.3 μm). From this disclosure, it will be apparent that microparticles typically have a size of less than 250 nm radius (i.e. less than 500 nm diameter). In some embodiments, the particle size is from about 0.5 μm to about 5.0 μm (e.g., from about 0.5 μm to about 4.0 μm, from about 0.5 μm to about 2.5 μm, from about 0.6 μm to about 2.0 μm, from about 0.7 μm to about 1.0 μm, from about 0.5 μm to about 0.9 μm, or from about 0.6 μm to about 0.8 μm).

In some embodiments, at least 50% (e.g., at least about 55%, at least about 60%, at least about 65%, at least about 70%, at least about 75%, at least about 80%, at least about 85%, at least about 90%, at least about 95%, or at least about 99%) of platelets or platelet derivatives (e.g., thrombosomes), have a particle size of at least 0.5 μm, for example in the range of about 0.5 μm to about 25.0 μm, 20.0 μm, 15.0 μm, 12.5 μm, 10.0 μm, or 5.0 μm (e.g., from about 0.5 μm to about 4.0 μm, from about 0.5 μm to about 2.5 μm, from about 0.6 μm to about 2.0 μm, from about 0.7 μm to about 1.0 μm, from about 0.5 μm to about 0.9 μm, or from about 0.6 μm to about 0.8 μm). In some embodiments, at most 99% (e.g., at most about 95%, at most about 80%, at most about 75%, at most about 70%, at most about 65%, at most about 60%, at most about 55%, or at most about 50%) of the platelets or platelet derivatives (e.g., thrombosomes), are in the range of about 0.5 μm to about 5.0 μm (e.g., from about 0.5 μm to about 4.0 μm, from about 0.5 μm to about 2.5 μm, from about 0.6 μm to about 2.0 μm, from about 0.7 μm to about 1.0 μm, from about 0.5 μm to about 0.9 μm, or from about 0.6 μm to about 0.8 μm). In some embodiments, about 50% to about 99% (e.g., about 55% to about 95%, about 60% to about 90%, about 65% to about 85, about 70% to about 80%) of the platelets or platelet derivatives (e.g., thrombosomes) are in the range of about 0.5 μm to about 5.0 μm (e.g., from about 0.5 μm to about 4.0 μm, from about 0.5 μm to about 2.5 μm, from about 0.6 μm to about 2.0 μm, from about 0.7 μm to about 1.0 μm, from about 0.5 μm to about 0.9 μm, or from about 0.6 μm to about 0.8 μm).

In some illustrative embodiments, a microparticle can be a particle having a particle size (e.g., diameter, max dimension) of less than about 0.5 μm (less than about 0.45 μm or 0.4 μm) In some cases, a microparticle can be a particle having a particle size of about 0.01 μm to about 0.5 μm (e.g., about 0.02 μm to about 0.5 μm).

Compositions comprising platelets or platelet derivatives (e.g., thrombosomes), such as those prepared according to methods described herein, can have a microparticle content that contributes to less than about 5.0% (e.g., less than about 4.5%, 4.0%, 3.5%, 3.0%, 2.5%, 2.0%, 1.5%, 1.0%, 0.5%, or 0.1%) of the total scattering intensity of all particles from about 1 nm to about 60,000 nm in radius in the composition. In some embodiments, the platelet derivative composition comprises a population of platelet derivatives comprising CD41-positive platelet derivatives, wherein less than 15%, 10%, 7.5%, 5%, 4.5%, 4%, 3.5%, 3%, 2.5%, 2%, 1.5%, 1%, 0.5%, or 0.1% of the CD41-positive platelet derivatives are microparticles having a diameter of less than 1 μm, 0.9 μm, 0.8 μm, 0.7 μm, 0.6 μm, 0.5 μm, 0.4 μm, 0.3 μm, 0.2 μm, or 0.1 μm, which in certain illustrative embodiments are less than 0.5 μm. In some embodiments, the platelet derivative composition comprises a population of platelet derivatives comprising CD42-positive platelet derivatives, wherein less than 15%, 10%, 7.5%, 5%, 4.5%, 4%, 3.5%, 3%, 2.5%, 2%, 1.5%, 1%, 0.5%, or 0.1% of the CD42-positive platelet derivatives are microparticles having a diameter of less than 1 μm, 0.9 μm, 0.8 μm, 0.7 μm, 0.6 μm, 0.5 μm, 0.4 μm, 0.3 μm, 0.2 μm, or 0.1 μm, which in certain illustrative embodiments are less than 0.5 μm. In some embodiments, the platelet derivative composition comprises a population of platelet derivatives comprising CD61-positive platelet derivatives, wherein less than 15%, 10%, 7.5, 5%, 4.5%, 4%, 3.5%, 3%, 2.5%, 2%, 1.5%, 1%, 0.5%, or 0.1% of the CD61-positive platelet derivatives are microparticles having a diameter of less than 1 μm, 0.9 μm, 0.8 μm, 0.7 μm, 0.6 μm, 0.5 μm, 0.4 μm, 0.3 μm, 0.2 μm, or 0.1 μm, which in certain illustrative embodiments are less than 0.5 μm. In some illustrative embodiments, the microparticles have a diameter of less than 0.5 μm. In some embodiments of any of the aspects and embodiments herein that include a platelet derivative composition in a powdered form, the diameter of the microparticles is determined after rehydrating the platelet derivative composition with an appropriate solution. In some embodiments, the amount of solution for rehydrating the platelet derivative composition is equal to the amount of buffer or preparation agent present at the step of freeze-drying. As used herein, a content of microparticles “by scattering intensity” refers to the microparticle content based on the scattering intensity of all particles from about 1 nm to about 60,000 nm in radius in the composition. The microparticle content can be measured by any appropriate method, for example, by dynamic light scattering (DLS). In some cases, the viscosity of a sample used for DLS can be at about 1.060 cP (or adjusted to be so), as this is the approximate viscosity of plasma. In some embodiments, the platelet derivative composition as per any aspects, or embodiments comprises a population of platelet derivatives, and microparticles, wherein the numerical ratio of platelet derivatives to the microparticles is at least 90:1, 91:1, 92:1, 93:1, 94:1, 95:1, 96:1, 97:1, 98:1, or 99:1. In some embodiments, the platelet derivatives have a diameter in the range of 0.5-2.5 μm, and the microparticles have a diameter less than 0.5 μm.

Platelets or platelet derivatives (e.g., thrombosomes) as described herein typically have cell surface markers. Such platelet derivatives in illustrative embodiments have properties of activated platelets. Accordingly, in illustrative embodiments they have an activated collagen and thrombin-activated (COAT) phenotype. In such non-limiting embodiments, they express major adhesion receptors, adhere to collagen, aggregate with themselves and endogenous platelets at a site of injury, and express phosphatidyl serine resulting in thrombin generation.

The presence of cell surface markers can be determined using any appropriate method. In some embodiments, the presence of cell surface markers can be determined using binding proteins (e.g., antibodies) specific for one or more cell surface markers and flow cytometry (e.g., as a percent positivity, e.g., using approximately 2.7×10⁵ thrombosomes/μL; and about 4.8 μL of an anti-CD41 antibody, about 3.3 μL of an anti-CD42 antibody, about 1.3 μL of annexin V, or about 2.4 μL of an anti-CD62 antibody). Non-limiting examples of cell-surface markers include CD41 (also called glycoprotein IIb or GPIIb, which can be assayed using e.g., an anti-CD41 antibody), CD42 (which can be assayed using, e.g., an anti-CD42 antibody), CD62 (also called CD62P or P-selectin, which can be assayed using, e.g., an anti-CD62 antibody), phosphatidylserine (which can be assayed using, e.g., annexin V (AV)), and CD47 (which is used in self-recognition; absence of this marker, in some cases, can lead to phagocytosis). The percent positivity of any cell surface marker can be any appropriate percent positivity. For example, platelets or platelet derivatives (e.g., thrombosomes), such as those prepared by methods described herein, can have an average CD41 percent positivity of at least 55% (e.g., at least 60%, at least 65%, at least 67%, at least 70%, at least 75%, at least 80%, at least 85%, at least 90%, or at least 95%). In some embodiments, at least 90%, 91%, 92%, 93%, 94%, 95%, 96%, 97%, 98%, 99% platelet derivatives that are positive for CD 41 have a size in the range of 0.5-2.5 μm. In some embodiments, at least 90%, 91%, 92%, 93%, 94%, 95%, 96%, 97%, 98%, or 99% platelet derivatives that are positive for CD 41 have a size in the range of 0.4-2.8 μm. In some embodiments, at least 90%, 91%, 92%, 93%, 94%, 95%, 96%, 97%, 98%, or 99% platelet derivatives that are positive for CD 41 have a size in the range of 0.3-3 μm.

As another example, platelets or platelet derivatives (e.g., thrombosomes), such as those described herein, can have an average CD42 percent positivity of at least 65% (e.g., at least 67%, at least 70%, at least 75%, at least 80%, at least 85%, at least 90%, or at least 95%). In some embodiments, platelets or platelet derivatives can have an average CD42 percent positivity of at least 76%, 77%, 78%, or 79%. In some embodiments, platelets or platelet derivatives can have an average CD42 percent positivity in the range of 76-95%, 76-94%, 77-93%, or 78-90%. In some embodiments, at least 90%, 91%, 92%, 93%, 94%, 95%, 96%, 97%, 98%, or 99% platelet derivatives that are positive for CD 42 have a size in the range of 0.5-2.5 μm. In some embodiments, at least 90%, 91%, 92%, 93%, 94%, 95%, 96%, 97%, 98%, or 99% platelet derivatives that are positive for CD 42 have a size in the range of 0.4-2.8 μm. In some embodiments, at least 90%, 91%, 92%, 93%, 94%, 95%, 96%, 97%, 98%, or 99% platelet derivatives that are positive for CD 42 have a size in the range of 0.3-3 μm. In some embodiments, the platelet derivatives herein have less amount of CD 42 as compared to the amount of CD 42 present in fresh platelets or apheresis platelets. In some embodiments, the platelet derivatives have at least 10%, 15%, 20%, 25%, 30%, 35%, 40%, 45%, 50%, or 55% amount of CD 42 as compared to the amount of CD 42 in fresh platelets or apheresis platelets. For example, the platelet derivatives have 12% to 64%, 20% to 64%, 20% to 60%, 20% to 50%, 25% to 50%, 25% to 40%, 20% to 40%, or 25% to 35% amount of CD42 as compared to the amount of CD42 in fresh platelets or apheresis platelets.

As another example, platelets or platelet derivatives (e.g., thrombosomes), such as those prepared by methods described herein, can have an average CD62 percent positivity of at least 10% (e.g., at least 15%, at least 20%, at least 25%, at least 30%, at least 35%, at least 40%, at least 45%, at least 50%, at least 55%, at least 60%, at least 65%, at least 70%, at least 75%, at least 80%, at least 82%, at least 83%, at least 84%, at least 85%, at least 90%, or at least 95%). In some embodiments, at least 70%, 75%, 80%, 85%, 90%, 91%, 92%, 93%, 94%, 95%, 96%, 97%, 98%, or 99% platelet derivatives that are positive for CD 62 have a size in the range of 0.5-2.5 μm. In some embodiments, at least 70%, 75%, 80%, 85%, 90%, 91%, 92%, 93%, 94%, 95%, 96%, 97%, 98%, or 99% platelet derivatives that are positive for CD 62 have a size in the range of 0.4-2.8 μm. In some embodiments, at least 70%, 75%, 80%, 85%, 90%, 91%, 92%, 93%, 94%, 95%, 96%, 97%, 98%, or 99% platelet derivatives that are positive for CD 62 have a size in the range of 0.3-3 μm.

As yet another example, platelets or platelet derivatives (e.g., thrombosomes), such as those prepared by methods described herein, can have an average annexin V positivity of at least 25% (e.g., at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, or at least 99%). In some embodiments, at least 90%, 91%, 92%, 93%, 94%, 95%, 96%, 97%, 98%, 99% platelet derivatives that are positive for annexin V have a size in the range of 0.5-2.5 μm. In some embodiments, at least 90%, 91%, 92%, 93%, 94%, 95%, 96%, 97%, 98%, or 99% platelet derivatives that are positive for annexin V have a size in the range of 0.4-2.8 μm. In some embodiments, at least 90%, 91%, 92%, 93%, 94%, 95%, 96%, 97%, 98%, or 99% platelet derivatives that are positive for annexin V have a size in the range of 0.3-3 μm.

In some embodiments, the platelet derivatives as described herein are activated to a maximum extent such that in the presence of an agonist, the platelet derivatives are not able to show an increase in the platelet activation markers on them as compared to the level of the platelet activation markers which were present prior to the exposure with the agonist. In some embodiments, the platelet derivatives as described herein show an inability to increase expression of a platelet activation marker in the presence of an agonist as compared to the expression of the platelet activation marker in the absence of an agonist. In some embodiments, the agonist is selected from the group consisting of collagen, epinephrine, ristocetin, arachidonic acid, adenosine di-phosphate, and thrombin receptor associated protein (TRAP). In some embodiments, the platelet activation marker is selected from the group consisting of Annexin V, and CD 62. In some embodiments, the platelet derivatives as described herein show an inability to increase expression of Annexin V in the presence of TRAP. An increased amount of the platelet activation markers on the platelets indicates the state of activeness of the platelets. However, in some embodiments, the platelet derivatives as described herein are not able to increase the amount of the platelet activation markers on them even in the presence of an agonist. This property indicates that the platelet derivatives as described herein are activated to a maximum extent. In some embodiments, the property can be beneficial where maximum activation of platelets is required, because the platelet derivatives as described herein is able to show a state of maximum activation in the absence of an agonist.

As another example, platelets or platelet derivatives (e.g., thrombosomes), such as those prepared by methods described herein, can have an average CD47 percent positivity of at least about 8% (e.g., at least about 10%, 15%, 20%, 25%, 30%, 35%, 40%, 45%, 50%, or 55%).

Glycoprotein VI (GPVI) is a platelet receptor for collagen, and the binding of collagen to GVPI activates the platelet. Receptor binding can be noticeably reduced in thrombosomes compared to fresh platelets. Without being bound by any particular theory, it is believed that the manufacturing process is blocking or destroying some copies of this receptor in thrombosomes, possibly to a reduction in collagen binding in thrombosomes relative to fresh platelets.

Platelets or platelet derivatives (e.g., thrombosomes) as described herein can have fibrinogen associated with the cell membrane. Aggregation of activated platelets is mediated by the formation of the GPIIb/IIIa complex, which can bind to fibrinogen (also called Factor 1) and form a clot. GPIIb/IIIa is a platelet fibrinogen receptor also known as CD41/CD61 complex. The GPIIb/IIIa clone PAC-1 binds to the active form of the GPIIb/IIIa. Without being bound by any particular theory, it is believed that the presence of fibrinogen on the cell membrane may be indicative of platelets or platelet derivatives (e.g., thrombosomes) capable of forming clots. Similarly, without being bound by any particular theory, it is believed that a lack of binding by anti-PAC1 antibodies to the platelets or platelet derivatives (e.g., thrombosomes), such as those prepared by methods described herein, can be indicative of fibrinogen bound to the active form of GPIIb/GPIIIa, as PAC-1 binds to the same complex. In some cases, platelets or platelets derivatives (e.g., thrombosomes), such as those prepared by methods described herein, can have a greater amount of bound fibrinogen when they retain a higher amount of residual plasma. In some embodiments of a platelet derivative composition as described herein, the platelet derivatives can have an amount of fibrinogen on their surface that is greater than that present on the surface of resting platelets, activated platelets, or lyophilized fixed platelets. In some embodiments, the platelet derivatives have at least 25%, 30%, 35%, 40%, 45%, 50%, 55%, 60%, 65%, 70%, 75%, 80%, 85%, 90%, 95%, or 100% higher fibrinogen on their surface as compared to resting platelets, or activated platelets, or fixed platelets. In some embodiments, the platelet derivatives when analyzed for the binding of anti-fibrinogen antibody to the platelet derivatives using flow cytometry exhibit at least 2 folds higher mean fluorescent intensity (MFI) in the absence of an agonist as compared to the MFI of binding of anti-fibrinogen antibody to the lyophilized fixed platelets. In some embodiments, the platelet derivatives when analyzed for the binding of anti-fibrinogen antibody to the platelet derivatives using flow cytometry exhibit at least 10, 15, 20, 25, 30, 35, or 40 folds higher mean fluorescent intensity (MFI) in the absence of an agonist as compared to the MFI of binding of anti-fibrinogen antibody to the fixed platelets. In some embodiments, the greater amount of fibrinogen present on the surface of the platelet derivatives as described herein as compared to that of lyophilized fixed platelets is beneficial. Without being bound by any particular theory, it is believed that the higher amount of fibrinogen on the cell membrane of the platelet derivatives (e.g., thrombosomes) as compared to that of lyophilized fixed platelets can make the platelet derivatives superior in terms of its ability to form clots as compared to lyophilized fixed platelets.

Von Willebrand factor (vWF) is a multimeric glycoprotein that plays a major role in blood coagulation. vWF serves as a bridging molecule that promotes platelet binding to sub-endothelium and other platelets, thereby promoting platelet adherence and aggregation. vWF also binds to collagens to facilitate clot formation at sites of injury. In some embodiments, the platelet derivatives as described herein have the presence of von Willebrand factor (vWF) on their surface at a level that is greater than that on the surface of resting platelets, activated platelets, or lyophilized fixed platelets. In some embodiments, the platelet derivatives have the presence of von Willebrand factor (vWF) on their surface at a level that is at least 10%, 20%, 25%, 30%, 50%, 60%, 70%, 80%, 90%, or 100% higher than on the surface of resting platelets, or lyophilized fixed platelets. In some embodiments, the platelet derivatives when analyzed for the binding of anti-von Willebrand factor (vWF) antibody to the platelet derivatives using flow cytometry exhibits at least 1.5 folds, 2 folds, or 3 folds, or 4 folds higher mean fluorescent intensity (MFI) in the absence of an agonist as compared to the MFI of binding of anti-vWF antibody to the resting platelets, or lyophilized fixed platelets. In some embodiments, the platelet derivatives when analyzed for the binding of anti-von Willebrand factor (vWF) antibody to the platelet derivatives using flow cytometry exhibits 2-4 folds, or 2.5-3.5 higher mean fluorescent intensity (MFI) in the absence of an agonist as compared to the MFI of binding of anti-vWF antibody to the resting platelets, or lyophilized fixed platelets.

Thrombospondin is a glycoprotein secreted from the α-granules of platelets upon activation. In the presence of divalent cations, the secreted protein binds to the surface of the activated platelets and is responsible for the endogenous lectin-like activity associated with activated platelets. In some embodiments, the platelet derivatives have the presence of thrombospondin (TSP-1) on their surface at a level that is greater than that presence on the surface of resting platelets, activated platelets, or lyophilized fixed platelets. In some embodiments, the platelet derivatives have the presence of thrombospondin (TSP-1) on their surface at a level that is at least 10%, 20%, 25%, 30%, 50%, 60%, 70%, 80%, 90%, or 100% higher than on the surface of resting platelets, or lyophilized fixed platelets. In some embodiments, the platelet derivatives have the presence of thrombospondin (TSP-1) on their surface at a level that is more than 100% higher than on the surface of resting platelets, or lyophilized fixed platelets. In some embodiments, the platelet derivatives when analyzed for the binding of anti-thrombospondin (TSP) antibody to the platelet derivatives using flow cytometry exhibit at least 2 folds, 5 folds, 7 folds, 10 folds, 20 folds, 30 folds, 40 folds, 50 folds, 60 folds, 70 folds, 80 folds, 90 folds, or 100 folds higher mean fluorescent intensity (MFI) in the absence of an agonist as compared to the MFI of binding of anti-TSP antibody to the resting platelets. In some embodiments, the platelet derivatives when analyzed for the binding of anti-thrombospondin (TSP) antibody to the platelet derivatives using flow cytometry exhibit at least 2 folds, 5 folds, 7 folds, 10 folds, 20 folds, 30 folds, or 40 folds higher mean fluorescent intensity (MFI) in the absence of an agonist as compared to the MFI of binding of anti-TSP antibody to the lyophilized fixed platelets. In some embodiments, the platelet derivatives when analyzed for the binding of anti-thrombospondin (TSP) antibody to the platelet derivatives using flow cytometry exhibit 10-800 folds, 20-800 folds, 100-700 folds, 150-700 folds, 200-700 folds, or 250-500 folds higher mean fluorescent intensity (MFI) in the absence of an agonist as compared to the MFI of binding of anti-TSP antibody to the resting platelets. In some embodiments, the platelet derivatives when analyzed for the binding of anti-thrombospondin (TSP) antibody to the platelet derivatives using flow cytometry exhibit at least 2 folds, 5 folds, 7 folds, 10 folds, 20 folds, 30 folds, or 40 folds higher mean fluorescent intensity (MFI) in the absence of an agonist as compared to the MFI of binding of anti-TSP antibody to the active platelets. In some embodiments, the platelet derivatives when analyzed for the binding of anti-thrombospondin (TSP) antibody to the platelet derivatives using flow cytometry exhibit 2-40 folds, 5-40 folds, 5-35 folds, 10-35 folds, or 10-30 folds higher mean fluorescent intensity (MFI) in the absence of an agonist as compared to the MFI of binding of anti-TSP antibody to the active platelets.

Platelets or platelet derivatives (e.g., thrombosomes) as described herein can be capable of generating thrombin, for example, when in the presence of a reagent containing tissue factor and phospholipids. For example, in some cases, platelets or platelet derivatives (e.g., thrombosomes) (e.g., at a concentration of about 4.8×10³ particles/μL) as described herein can generate a thrombin peak height (TPH) of at least 25 nM (e.g., at least 30 nM, 35 nM, 40 nM, 45 nM, 50 nM, 52 nM, 54 nM, 55 nM, 56 nM, 58 nM, 60 nM, 65 nM, 70 nM, 75 nM, or 80 nM) when in the presence of a reagent containing tissue factor (e.g., at 0.25 pM, 0.5 pM, 1 pM, 2 pM, 5 pM or 10 pM) and optionally phospholipids. For example, in some cases, platelets or platelet derivatives (e.g., thrombosomes) (e.g., at a concentration of about 4.8×10³ particles/μL) as described herein can generate a TPH of about 25 nM to about 100 nM (e.g., about 25 nM to about 50 nM, about 25 to about 75 nM, about 50 to about 100 nM, about 75 to about 100 nM, about 35 nM to about 95 nM, about 45 to about 85 nM, about 55 to about 75 nM, or about 60 to about 70 nM) when in the presence of a reagent containing tissue factor and (e.g., at 0.25 pM, 0.5 pM, 1 pM, 2 pM, 5 pM or 10 pM) and optionally phospholipids. In some cases, platelets or platelet derivatives (e.g., thrombosomes) (e.g., at a concentration of about 4.8×10³ particles/μL) as described herein can generate a TPH of at least 25 nM (e.g., at least 30 nM, 35 nM, 40 nM, 45 nM, 50 nM, 52 nM, 54 nM, 55 nM, 56 nM, 58 nM, 60 nM, 65 nM, 70 nM, 75 nM, or 80 nM) when in the presence of PRP Reagent (cat #TS30.00 from Thrombinoscope), for example, using conditions comprising 20 μL of PRP Reagent and 80 μL of a composition comprising about 4.8×10³ partilces/μL of platelets or platelet derivatives (e.g., thrombosomes). In some cases, platelets or platelet derivatives (e.g., thrombosomes) (e.g., at a concentration of about 4.8×10³ particles/μL) as described herein can generate a TPH of about 25 nM to about 100 nM (e.g., about 25 nM to about 50 nM, about 25 to about 75 nM, about 50 to about 100 nM, about 75 to about 100 nM, about 35 nM to about 95 nM, about 45 to about 85 nM, about 55 to about 75 nM, or about 60 to about 70 nM) when in the presence of PRP Reagent (cat #TS30.00 from Thrombinoscope), for example, using conditions comprising 20 μL of PRP Reagent and 80 μL of a composition comprising about 4.8×10³ partilces/μL of platelets or platelet derivatives (e.g., thrombosomes). In some embodiments, platelet derivatives herein can generate more peak thrombin as compared to apheresis platelet unit (APU) per particle. For example, under the same experimental conditions, typically, in vitro experiments used to assess peak thrombin, or thrombin generation, platelet derivatives herein can generate more peak thrombin as compared to APU per particle. In some embodiments, platelet derivatives can generate at least 1.15, 1.25, 1.50, 1.75, 2, 2.25, 2.5, 2.75, 3, 3.25, 3.5, 3.75, or 4 fold peak thrombin as compared to APU per particle. For example, platelet derivatives can generate 1.15-5, 1.15-4, 1.15-3, or 1.15-2 fold peak thrombin as compared to APU per particle. In some embodiments, platelet derivatives herein can generate maximum thrombin (time to maximum thrombin generation) in less time as compared to apheresis platelet unit (APU). For example, under the same experimental conditions, typically, in vitro experiments used to assess peak thrombin, or thrombin generation, platelet derivatives herein can generate maximum thrombin in at least 1.15, 1.25, 1.50, 1.75, 2, 2.25, 2.5, 2.75, 3, 3.25, 3.5, 3.75, or 4 fold less time as compared to APU. For example, platelet derivatives herein can generate maximum thrombin in 1.15-5, 1.15-4, 1.15-3, or 1.15-2 fold less time as compared to APU.

Platelets or Platelet derivatives (e.g., thrombosomes) as described herein can be capable of generating thrombin, for example, when in the presence of a reagent containing tissue factor and phospholipids. For example, in some cases, platelets or platelet derivatives (e.g., thrombosomes) can have a potency of at least 1.2 (e.g., at least 1.3, 1.4, 1.5, 1.6, 1.7, 1.8, 1.9, 2.0, 2.1, 2.2, 2.3, 2.4, or 2.5) thrombin generation potency units (TGPU) per 10⁶ particles. For example, in some cases, platelets or platelet derivatives (e.g., thrombosomes) can have a potency of between 1.2 and 2.5 TPGU per 10⁶ particles (e.g., between 1.2 and 2.0, between 1.3 and 1.5, between 1.5 and 2.25, between 1.5 and 2.0, between 1.5 and 1.75, between 1.75 and 2.5, between 2.0 and 2.5, or between 2.25 and 2.5 TPGU per 10⁶ particles). TPGU can be calculated as follows: TGPU/million particles=[TPH in nM]*[Potency Coefficient in IU/(nM)]/[0.576 million particles in the well]. Similarly, the Potency Coefficient for a sample of thrombin can be calculated as follows: Potency Coefficient=Calculated Calibrator Activity (IU)/Effective Calibrator Activity (nM). In some cases, the calibrator activity can be based on a WHO international thrombin standard.

Platelets or platelet derivatives (e.g., thrombosomes) as described herein can be capable of clotting, as determined, for example, by using a total thrombus-formation analysis system (T-TAS®). In some cases, platelets or platelet derivatives as described herein, when at a concentration of at least 70×10³ particles/μL (e.g., at least 73×10³, 100×10³, 150×10³, 173×10³, 200×10³, 250×10³, or 255×10³ particles/μL) can result in a T-TAS occlusion time (e.g., time to reach kPa of 80) of less than 30, 25, 20, 18, 15, or 14 minutes (e.g., less than 13.5, 13, 12.5, 12, 11.5, or 11 minutes), for example, in platelet-reduced citrated whole blood. For example, platelet derivatives herein, when at a concentration of at least 70×10³ particles/μL can result in the occlusion time in the range of 30 to 5 minutes, 28 to 5 minutes, 25 to 5 minutes, 22 to 5 minutes, 20 to 5 minutes, 18 to 5 minutes, 15 to 5 minutes, 14 to 5 minutes, 13 to 5 minutes, or 14 to 7 minutes. In some cases, platelets or platelet derivatives as described herein, when at a concentration of at least 70×10³ particles/μL (e.g., at least 73×10³, 100×10³, 150×10³, 173×10³, 200×10³, 250×10³, or 255×10³ particles/μL) can result in an area under the curve (AUC) of at least 1300 (e.g., at least 1380, 1400, 1500, 1600, or 1700), for example, in platelet-reduced citrated whole blood.

Platelets or platelet derivatives (e.g., thrombosomes) as described herein can be capable of thrombin-induced trapping in the presence of thrombin. In some cases, platelets or platelet derivatives (e.g., thrombosomes) as described herein can have a percent thrombin-induced trapping of at least 5% (e.g., at least 10%, 15%, 20%, 25%, 30%, 35%, 40%, 45%, 50%, 55%, 60%, 65%, 67%, 70%, 75%, 85%, 90%, or 99%) in the presence of thrombin. In some cases, platelets or platelet derivatives (e.g., thrombosomes) as described herein can have a percent thrombin-induced trapping of about 25% to about 100% (e.g., about 25% to about 50%, about 25% to about 75%, about 50% to about 100%, about 75% to about 100%, about 40% to about 95%, about 55% to about 80%, or about 65% to about 75%) in the presence of thrombin. Thrombin-induced trapping can be determined by any appropriate method, for example, light transmission aggregometry. Without being bound by any particular theory, it is believed that the thrombin-induced trapping is a result of the interaction of fibrinogen present on the surface of the platelet derivatives with thrombin.

Platelets or platelet derivatives (e.g., thrombosomes) as described herein can be capable of co-aggregating, for example, in the presence of an aggregation agonist, and fresh platelets. Non-limiting examples of aggregation agonists include, collagen, epinephrine, ristocetin, arachidonic acid, adenosine di-phosphate, and thrombin receptor associated protein (TRAP). In some cases, platelets or platelet derivatives (e.g., thrombosomes) as described herein can have a percent co-aggregation of at least 5% (e.g., at least 10%, 15%, 20%, 25%, 30%, 35%, 40%, 45%, 50%, 55%, 60%, 65%, 67%, 70%, 75%, 85%, 90%, or 99%) in the presence of an aggregation agonist, and fresh platelets. In some cases, platelets or platelet derivatives (e.g., thrombosomes) as described herein can have a percent co-aggregation of about 25% to about 100% (e.g., about 25% to about 50%, about 25% to about 75%, about 50% to about 100%, about 75% to about 100%, about 40% to about 95%, about 55% to about 80%, or about 65% to about 75%) in the presence of an aggregation agonist. Percent co-aggregation can be determined by any appropriate method, for example, light transmission aggregometry.

Platelet derivative compositions in certain illustrative embodiments herein, comprise a population of platelet derivatives having a reduced propensity to aggregate under aggregation conditions comprising an agonist but no fresh platelets, compared to the propensity of fresh platelets and/or activated to aggregate under these conditions. Platelets or platelet derivatives (e.g., thrombosomes) as described herein in illustrative embodiments, display a reduced propensity to aggregate under aggregation conditions comprising an agonist but no fresh platelets, compared to the propensity of fresh platelets and/or activated to aggregate under these conditions. It is noteworthy that aggregation of platelet derivatives is different from co-aggregation in that aggregation conditions typically do not include fresh platelets, whereas co-aggregation conditions include fresh platelets. Exemplary aggregation and co-aggregation conditions are provided in the Examples herein. Thus, in some embodiments, the platelet derivatives as described herein have a higher propensity to co-aggregate in the presence of fresh platelets and presence of an agonist, while having a reduced propensity to aggregate in the absence of fresh platelets and presence of an agonist, compared to the propensity of fresh platelets to aggregate under these conditions. In some embodiments, a platelet derivative composition comprises a population of platelet derivatives having a reduced propensity to aggregate, wherein no more than 1%, 2%, 3%, 4%, 5%, 7.5%, 10%, 12.5%, 15%, 17.5%, 20%, 22.5%, or 25% of the platelet derivatives in the population aggregate under aggregation conditions comprising an agonist but no platelets, in illustrative embodiments no fresh platelets, and in other illustrative embodiments in the absence of a divalent cation. In some embodiments, the population of platelet derivatives aggregate in the range of 0-1%, 0-2%, 0-3%, 0-4%, 0-5%, 0-7.5%, 0-10%, 2-30%, 5-25%, 10-30%, 10-25%, or 12.5-25%, and in illustrative embodiments 0-1% or 0 to about 1%, of the platelet derivatives under aggregation conditions comprising an agonist but no platelets, in illustrative embodiments no fresh platelets, and in other illustrative embodiments in the absence of a divalent cation. In these and other illustrative embodiments the agonist is other than arachidonic acid. It will be understood that if an aggregation reaction control sample produces a background aggregation above 0% then aggregation values/ranges for FDPDs disclosed herein would be increased by the background value obtained with the control sample. Thus, if a background aggregation produced using a control sample was 1%, then the above ranges would be increased by 1% (e.g., 1-2%, 1-3%, 1-4%, 1-5%, 1-6%, 1-8.5%, and 1-11% etc.). Accordingly, in some embodiments, the values and ranges provided herein for aggregation are values above background values, for example obtained using a control sample or no sample, and thus can be referred to control-corrected, or control-adjusted aggregation values. In some embodiments, a platelet derivative composition comprises a population of platelet derivatives having a reduced propensity to aggregate, such that less than ⅕, 1/10, or 1/20 of the platelet derivatives in the population aggregate under aggregation conditions comprising an agonist but no platelets, compared to platelet rich plasma, in illustrative embodiments prepared from fresh platelets. In some embodiments, a population of platelet derivatives can aggregate in the absence of an agonist and in illustrative embodiments in the absence of thrombin, but in the presence of a divalent cation. For example, the population of platelet derivatives can aggregate in the presence of a divalent cation in the range of 5-80%, 5-75%, 5-70%, 5-65%, 5-60%, 5-50%, 5-40%, 10-80%, 15-80%, 20-80%, 25-80%, 30-80%, 35-80%, 40-80%, or 45-80%. Non-limiting examples of divalent cations can include magnesium (Mg²⁺), barium (Ba²⁺), copper (Cu²⁺), calcium (Ca²⁺), manganese (Mn²⁺), zinc (Zn²⁺), iron (Fe²⁺), nickel (Ni²⁺), and cobalt (Co²⁺). A skilled artisan can understand that any suitable salt of the divalent cations can be used for the aggregation assay, for example, a chloride salt of magnesium, barium, copper, calcium, manganese, zinc, iron, nickel, or cobalt. In some embodiments, a population of platelet derivatives can show a higher percentage of aggregation in the absence of an agonist, but in the presence of a divalent cation as compared to platelets, in illustrative embodiments, fresh platelets. In illustrative embodiments, aggregation conditions comprise an agonist but no platelets, and no divalent cations. In some embodiments, platelet derivatives herein can aggregate more in the absence of an agonist and in illustrative embodiments in the absence of thrombin, but in the presence of a divalent cation as compared to apheresis platelet units (APU). For example, platelet derivatives herein can aggregate at least 1.15, 1.25, 1.50, 1.75, 2, 2.25, 2.5, 2.75, 3, 3.25, 3.5, 3.75, or 4 folds more as compared to APU. For example, platelet derivatives herein can aggregate 1.15-5, 1.15-4, 1.15-3, or 1.15-2 folds more as compared to APU.

Compositions comprising platelets or platelet derivatives (e.g., thrombosomes) as described herein can have appropriate conditions and amounts of cellular substrates and/or metabolites, such as pH, pCO₂, pO₂, HCO₃ concentration, total carbon dioxide (TCO₂), sO₂, and lactate concentration. Lactate can be the products of glycolysis. Without being bound by any particular theory, a starting material can have high lactate concentration because it has been stored ex vivo, respirating and performing glycolysis, for a period of time (e.g., about 3 days) by the time of manufacturing. For example, in some cases, the pH can be about 5.5 to about 8.0 (e.g., about 6.0 to about 7.4, about 6.9 to about 7.5, or about 7.0 to about 7.3). As another example, the pCO₂ can be about 10 to about 20 mmHg (e.g., about 10 to about 15 mmHg, about 15 to about 20 mmHg, or about 17 to about 19 mmHg). The pO₂ can be about 140 to about 165 mmHg (e.g., about 140 to about 150 mmHg, about 150 to about 160 mmgH, or about 160 to about 165 mmHg). The HCO₃ concentration can be about 4.5 to about 6.5 mmol/L (e.g., about 5.0 to about 6.0 mmol/L). The total carbon dioxide can be about 4 to about 8 mmol/L (e.g., about 5 to about 7 mmol/L). The sO₂ can be at least about 98% (e.g., at least about 99%). The lactate concentration can be less than about 2.0 mmol/L (e.g., less than 1.5 mmol/L or 1.0 mmol/L). The lactate concentration can be about 0.4 to about 1.3 mmol/L (e.g., about 0.5 to about 0.6 mmol/L, about 0.5 to about 1.0 mmol/L, or about 0.8 to about 1.3 mmol/L).

### Membrane Integrity of the Platelet Derivatives

Platelet derivatives in certain illustrative aspects and embodiments herein are surrounded by a compromised plasma membrane. In these illustrative aspects and embodiments, the platelet derivatives lack an integrated membrane around them. Instead, the membrane has pores on them that are larger than pores observed on living cells. Not to be limited by theory, it is believed that in embodiments where platelet derivatives have a compromised membrane, such platelet derivatives have a reduced ability to, or are unable to transduce signals from the external environment into a response inside the particle that are typically transduced in living platelets. A compromised membrane can be identified through a platelet derivative's inability to retain more than 50% of lactate dehydrogenase (LDH) as compared to fresh platelets, or cold stored platelets, or cryopreserved platelets. In some embodiments, the platelet derivatives are incapable of retaining more than 35%, 40%, 45%, 50%, 55%, 60%, 65%, 70%, or 75% of lactate dehydrogenase as compared to lactate dehydrogenase retained in fresh platelets, or cold stored platelets, or cryopreserved platelets. In some embodiments, the platelet derivatives exhibit an increased permeability to antibodies. In some embodiments, the antibodies can be IgG antibodies. The increased permeability can be identified by targeting IgG antibodies against a stable intracellular antigen. One non-limiting type of stable intracellular antigen is β tubulin. The compromised membrane of the platelet derivatives can also be determined by flow cytometry studies.

Platelet or platelet derivatives (e.g., thrombosomes) as described herein can retain some metabolic activity, for example, as evidenced by lactate dehydrogenase (LDH) activity. In some cases, platelets or platelet derivatives (e.g., thrombosomes) as described herein can retain at least about 10% (e.g., at least about 12%, 15%, 20%, 25%, 30%, 35%, 40%, or 45%) of the LDH activity of donor apheresis platelets. Without being bound by any particular theory, it is believed that the addition of increasing amounts of polysucrose increases the amount of LDH activity remained (e.g., products of a preparation agent with 8% polysucrose have more retained LDH activity than products of a preparation agent with 4% polysucrose). Similarly unbound by any particular theory, it is believed that thermal treatment of a lyophilized composition comprising platelets or platelet derivatives (e.g., thrombosomes) increases the amount of LDH activity retained. As another example, metabolic activity can be evidenced by retained esterase activity, such as the ability of the cells to cleave the acetate groups on carboxyfluorescein diacetate succinimidyl ester (CFDASE) to unmask a fluorophore.

### Pathogen Reduction

The reduction of pathogens is generally desirable in blood products. Without being bound by any particular theory, it is believed that some methods of pathogen reduction can cause the formation of microparticles in the treated blood product. One method of pathogen reduction involves the use of a photosensitive nucleic acid-intercalating compound to alter the nucleic acids of pathogens upon illumination with an appropriate wavelength. The INTERCEPT® system (made by Cerus) uses amotosalen, a nucleic acid intercalating compound that forms cross-links in nucleic acid upon illumination with UVA.

### Starting Material Comprising Platelets or a Platelet Composition

A final blood product (e.g., platelets, cryopreserved platelets, freeze-dried platelets (e.g., thrombosomes)) as described herein can be prepared by any appropriate method. A final blood product (e.g., platelets, cryopreserved platelets, freeze-dried platelets (e.g., thrombosomes)) as described herein can be prepared by a method as disclosed herein. In some embodiments described herein, a final blood product can be a composition that includes platelets and an aqueous medium. In some embodiments, a final blood product can be the result of freeze-drying a composition that includes platelets and an aqueous medium, as described herein. In some embodiments, a final blood product can be prepared using tangential flow filtration (TFF) of a starting material (e.g., an unprocessed blood product (e.g., donor apheresis material (e.g., pooled donor apheresis material)), or a partially processed blood product (e.g., a blood product that has undergone filtration)). In some embodiments, a final blood product can be prepared using centrifugation of a starting material (e.g., an unprocessed blood product (e.g., donor apheresis material (e.g., pooled donor apheresis material)), or a partially processed blood product (e.g., a blood product that has undergone filtration)). It will be appreciated that while the methods described herein are generally described in the context of a starting material being apheresis material, other materials, such as platelets cultured in vitro, or whole blood, may be used. In some cases, platelets may be isolated from whole blood (e.g. pooled whole blood).

A starting material can be any appropriate starting material. In some embodiments, a starting material can have a protein concentration of about 60 to about 80 mg/mL. In some embodiments, a protein concentration can be based on the protein concentration in the plasma of whole blood. In some embodiments, a protein concentration can be based on the protein concentration of donor apheresis plasma. In some embodiments, a starting material can be donor blood product (e.g., whole blood or fractionated blood). In some embodiments, the starting material can be pooled donor blood product (e.g., pooled whole blood or pooled fractionated blood). In some embodiments, a starting material can include donor apheresis plasma. In some embodiments, a starting material can be derived from donor apheresis plasma. As used herein, “donor apheresis plasma” can refer to the plasma component of apheresis material, whether or not the material contains platelets or other blood cells.

In some embodiments, a starting material can be donor apheresis material (e.g., donor platelets or a pool of donor platelets). In some embodiments, a starting material is positive for one or more of: HLA Class I antibodies, HLA Class II antibodies, and HNA antibodies based on a regulatory agency-approved assay (e.g., an FDA cleared assay). In some embodiments, starting material can test positive for HLA Class I antibodies in a regulatory agency approved assay (e.g., an FDA cleared assay). In some embodiments, a starting material can test positive for HLA Class II antibodies in a regulatory agency approved assay (e.g., an FDA cleared assay). In some embodiments, starting material can test positive for HNA antibodies in a regulatory agency approved assay (e.g., an FDA cleared assay). A regulatory agency approved assay can be any appropriate regulatory agency approved assay. In some embodiments, a regulatory agency approved test can be the LABSCREEN™ Mixed by One Lambda. In some implementations, a regulatory agency approved test can be carried out using a LUMINEX® 100/200 or a LUMINEX® XY and the HLA FUSION™ software.

In some embodiments, a starting material can undergo a pathogen reduction step, for example, a nucleic acid intercalating compound that forms cross-links in nucleic acid upon illumination with UVA.

In some embodiments, a starting material (e.g., one or more units of donor platelets) can be initially pooled into a common vessel. The starting material may or may not be initially diluted with an acidified washing buffer (e.g., a control buffer). Without being bound by any particular theory, it is believed that washing with an acidified washing buffer can reduce platelet activation during processing. In some cases, a starting material can undergo two general processing pathways; either washed with control buffer (e.g. using TFF) until a desired residual component is reached (e.g., a percentage of residual donor plasma) before being concentrated to a final concentration; or the starting material can be concentrated to a final concentration before being washed with control buffer (e.g., using TFF) until a desired residual component is reached (e.g., a percentage of residual donor plasma). TFF processed material can then be filled into vials, lyophilized and thermally treated.

### Different Steps in Processing of a Starting Material, and TFF

In some embodiments, the method can include an initial dilution step, for example, a starting material (e.g., an unprocessed blood product (e.g., donor apheresis material (e.g., pooled donor apheresis material)) can be diluted with a preparation agent (e.g., any of the preparation agents described herein) to form a diluted starting material. In some cases, the initial dilution step can include dilution with a preparation agent with a mass of preparation agent equal to at least about 10% of the mass of the starting material (e.g., at least about 15%, 25%, 50%, 75%, 100%, 150%, or 200% of the mass of the starting material. In some embodiments, an initial dilution step can be carried out using the TFF apparatus.

In some embodiments, the method can include concentrating (e.g., concentrating platelets) (e.g., concentrating a starting material or a diluted starting material) to form a concentrated platelet composition. For example, concentrated can include concentrating to a about 1000×10³ to about 4000×10¹ platelets/μL (e.g., about 1000×10³ to about 2000×10³, about 2000×10³ to about 3000×10³, or about 4000×10³ platelets/μL). In some embodiments, a concentration step can be carried out using the TFF apparatus.

The concentration of platelets or platelet derivatives (e.g., thrombosomes) can be determined by any appropriate method. For example, a counter can be used to quantitate concentration of blood cells in suspension using impedance (e.g., a Beckman Coulter AcT 10 or an AcT diff 2).

In some embodiments, TFF can include diafiltering (sometimes called “washing”) of a starting material, a diluted starting material, a concentrated platelet composition, or a combination thereof. In some embodiments, diafiltering can include washing with at least 2 (e.g., at least 3, 4, 5, 6, 7, 8, 9, 10, or more) diavolumes. In some embodiments, TFF can include buffer exchange. In some embodiments, a buffer can be used in TFF. A buffer can be any appropriate buffer. In some embodiments, the buffer can be a preparation agent (e.g., any of the preparation agents described herein). In some embodiments, the buffer can be the same preparation agent as was used for dilution. In some embodiments, the buffer can be a different preparation than was used for dilution. In some embodiments, a buffer can include a lyophilizing agent, including a buffering agent, a base, a loading agent, optionally a salt, and optionally at least one organic solvent such as an organic solvent selected from the group consisting of ethanol, acetic acid, acetone, acetonitrile, dimethylformamide, dimethyl sulfoxide, dioxane, methanol, n-propanol, isopropanol, tetrahydrofuran (THF), N-methyl pyrrolidone, dimethylacetamide (DMAC), or combinations thereof. A buffering agent can be any appropriate buffering agent. In some embodiments, a buffering agent can be HEPES ((4-(2-hydroxyethyl)-1-piperazineethanesulfonic acid). A base can be any appropriate base. In some embodiments, a base can be sodium bicarbonate. In some embodiments, a saccharide can be a monosaccharide. In some embodiments, a loading agent can be a saccharide. In some embodiments, a saccharide can include sucrose, maltose, trehalose, glucose (e.g., dextrose), mannose, or xylose. In some embodiments, a monosaccharide can be trehalose. In some embodiments, the loading agent can include polysucrose. A salt can be any appropriate salt. In some embodiments, a salt can be selected from the group consisting of sodium chloride (NaCl), potassium chloride (KCl), or a combination thereof.

In some embodiments, a membrane with a pore size of about 0.1 μm to about 1 μm (e.g., about 0.1 μm to about 1 μm, about 0.1 μm to about 0.5 μm, about 0.2 to about 0.45 μm, about 0.45 to about 1 μm, about 0.1 μm, about 0.2 μm, about 0.45 μm, about 0.65 μm, or about 1 μm) can be used in TFF. A membrane can be made from any appropriate material. In some cases, a membrane can be a hydrophilic membrane. In some embodiments, a membrane can be a hydrophobic membrane. In some embodiments, a membrane with a nominal molecular weight cutoff (NMWCO) of at least about 100 kDa (e.g., at least about 200 kDa, 300 kDa, 500 kDa, or 1000 kDa) can be used in TFF. The TFF can be performed with any appropriate pore size within the range of 0.1 μm to 1.0 μm with the aim of reducing the microparticles content in the composition and increasing the content of platelet derivatives in the composition. A skilled artisan can appreciate the required optimization of the pore size in order to retain the platelet derivatives and allow the microparticles to pass through the membrane. The pore size in illustrative embodiments, is such that the microparticles pass through the membrane allowing the TFF-treated composition to have less than 5% microparticles. The pore size in illustrative embodiments is such that a maximum of platelet derivatives gets retained in the process allowing the TFF-treated composition to have a concentration of the platelet derivatives in the range of 100×10³ to 20,000×10³. The pore size during the TFF process can be exploited to obtain a higher concentration of platelet derivatives in the platelet derivative composition such that a person administering the platelet derivatives to a subject in need has to rehydrate/reconstitute fewer vials, therefore, being efficient with respect to time and effort during the process of preparing such platelet derivatives for a downstream procedure, for example a method of treating provided herein. TFF can be performed at any appropriate temperature. In some embodiments, TFF can be performed at a temperature of about 20° C. to about 37° C. (e.g., about 20° C. to about 25° C., about 20° C. to about 30° C., about 25° C. to about 30° C., about 30° C. to about 35° C., about 30° C. to about 37° C., about 25° C. to about 35° C., or about 25° C. to about 37° C.). In some embodiments, TFF can be carried out at a flow rate (e.g., a circulating flow rate) of about 100 ml/min to about 800 ml/min (e.g., about 100 to about 200 ml/min, about 100 to about 400 ml/min, about 100 to about 600 ml/min, about 200 to about 400 ml/min, about 200 to about 600 ml/min, about 200 to about 800 ml/min, about 400 to about 600 ml/min, about 400 to about 800 ml/min, about 600 to about 800 ml/min, about 100 ml/min, about 200 ml/min, about 300 ml/min, about 400 ml/min, about 500 ml/min, about 600 ml/min, about 700 ml/min, or about 800 ml/min).

In some embodiments, TFF can be performed until a particular endpoint is reached, forming a TFF-treated composition. An endpoint can be any appropriate endpoint. In some embodiments, an endpoint can be a percentage of residual plasma (e.g., less than or equal to about 50%, 40%, 30%, 20%, 150%, 10%, 9%, 8%, 7%, 6%, 5%, 4%, 3%, 2%, 10%, 0.9%, 0.8%, 0.7%, 0.6%, 0.50%, 0.4%, 0.30%, 0.2%, or 0.10% of residual plasma). In some embodiments, an endpoint can be a relative absorbance at 280 nm (A280). For example, an endpoint can be an A280 (e.g., using a path length of 0.5 cm) that is less than or equal to about 50% (e.g., less than or equal to about 40%, 30%, 20%, 15%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, or 0.1%) of the A280 (e.g., using a path length of 0.5 cm) prior to TFF (e.g., of a starting material or of a diluted starting material). In some embodiments, an A280 can be relative to a system that measures 7.5% plasma=1.66 AU. In some embodiments, an instrument to measure A280 can be configured as follows: a 0.5 cm gap flow cell can be attached to the filtrate line of the TFF system. The flow cell can be connected to a photometer with fiber optics cables attached to each side of the flow cell (light source cable and light detector cable). The flow cell can be made with a silica glass lens on each side of the fiber optic cables. Apart from the relative protein concentration of proteins in the aqueous medium, the protein concentration in the aqueous medium can also be measured in absolute terms. In some embodiments, the protein concentration in the aqueous medium is less than or equal to 15%, or 14%, or 13%, or 12%, or 11%, or 10%, or 9%, or 8%, or 7%, or 6%, or 5%, or 4%, or 3%, or 2%, or 1%, or 0.1%, or 0.01%. In some exemplary embodiments, the protein concentration is less than 3% or 4%. In some embodiments, the protein concentration is in the range of 0.01-15%, or 0.1-15%, or 1-15%, or 1-10%, or 0.01-10%, or 3-12%, or 5-10% in the TFF-treated composition. In some embodiments, an endpoint can be an absolute A280 (e.g., using a path length of 0.5 cm). For example, an endpoint can be an A280 that is less than or equal to 2.50 AU, 2.40 AU, 2.30 AU, 2.20 AU, 2.10 AU, 2.0 AU, 1.90 AU, 1.80 AU, or 1.70 AU (e.g., less than or equal to 1.66, 1.6, 1.5, 1.4, 1.3, 1.2, 1.1, 1.0, 0.9, 0.8, 0.7, 0.6, 0.5, 0.4, 0.3, 0.2, or 0.1 AU) (e.g., using a path length of 0.5 cm). In some embodiments, a percentage of residual plasma, a relative A280, or an A280 can be determined based on the aqueous medium of a composition comprising platelets and an aqueous medium. In some embodiments, a percentage of residual plasma can be determined based on a known correlation to an A280. In some embodiments, an endpoint can be a platelet concentration, as TFF can include concentration or dilution of a sample (e.g., using a preparation agent). For example, an endpoint can be a platelet concentration of at least about 2000×10³ platelets/μL (e.g., at least about 2050×10³, 2100×10³, 2150×10³, 2200×10³, 2250×10³, 2300×10³, 2350×10³, 2400×10³, 2450×10³, or 2500×10³ platelets/μL). As another example, an endpoint can be a platelet concentration of about 1000×10³ to about 2500 platelets/μL (e.g., about 1000×10³ to about 2000×10³, about 1500×10³ to about 2300×10³, or about 1700×10³ to about 2300×10³ platelets/μL). In some embodiments, an endpoint can be a concentration of platelets in the TFF-treated composition are at least 100×10³ platelets/μL, 200×10³ platelets/μL, 400×10³ platelets/μL, 1000×10³ platelets/μL, 1250×10³ platelets/μL, 1500×10³ platelets/μL, 1750×10³ platelets/μL, 2000×10³ platelets/μL, 2250×10³ platelets/μL, 2500×10³ platelets/μL, 2750×10³ platelets/μL, 3000×10³ platelets/μL, 3250×10³ platelets/μL, 3500×10³ platelets/μL, 3750×10³ platelets/μL, 4000×10³ platelets/μL, 4250×10³ platelets/μL, 4500×10³ platelets/μL, 4750×10³ platelets/μL, 5000×10³ platelets/μL, 5250×10³ platelets/μL, 5500×10³ platelets/μL, 5750×10³ platelets/μL, 6000×10³ platelets/μL, 7000×10³ platelets/μL, 8000×10³ platelets/μL, 9000×10³ platelets/μL, 10,000×10³ platelets/μL, 11,000×10³ platelets/μL, 12,000×10³ platelets/μL, 13,000×10³ platelets/μL, 14,000×10³ platelets/μL, 15,000×10³ platelets/μL, 16,000×10³ platelets/μL, 17,000×10³ platelets/μL, 18,000×10³ platelets/μL, 19,000×10³ platelets/μL, 20,000×10³ platelets/μL. In some embodiments, the platelets or platelet derivatives in the TFF-treated composition is in the range of 100×10³-20,000×10³ platelets/μL, or 1000×10³-20,000×10³ platelets/μL, or 1000×10³-10,000×10³ platelets/μL, or 500×10³-5,000×10³ platelets/μL, or 1000×10³-5,000×10³ platelets/μL, or 2000×10³-8,000×10³ platelets/μL, or 10,000×10³-20,000×10³ platelets/μL, or 15,000×10³-20,000×10³ platelets/μL.

In some embodiments, an endpoint can include more than one criterion (e.g., a percentage of residual plasma and a platelet concentration, a relative A280 and a platelet concentration, or an absolute A280 and a platelet concentration).

Typically, a TFF-treated composition is subsequently lyophilized, optionally with a thermal treatment step, to form a final blood product (e.g., platelets, cryopreserved platelets, freeze-dried platelets (e.g., thrombosomes)). However, in some cases, a TFF-treated composition can be considered to be a final blood product.

In some embodiments, a blood product can be prepared using centrifugation of a blood product (e.g., an unprocessed blood product (e.g., donor apheresis material (e.g., pooled donor apheresis material)), or a partially processed blood product (e.g., a blood product that has undergone TFF)). In some embodiments, a blood product can be prepared without centrifugation of a blood product (e.g., an unprocessed blood product (e.g., donor apheresis material), or a partially processed blood product (e.g., a blood product that has undergone TFF)). Centrifugation can include any appropriate steps. In some embodiments, centrifugation can include a slow acceleration, a slow deceleration, or a combination thereof. In some embodiments, centrifugation can include centrifugation at about 1400×g to about 1550×g (e.g., about 1400 to about 1450×g, about 1450 to about 1500×g, or 1500 to about 1550×g, about 1400×g, about 1410×g, about 1430×g, about 1450×g, about 1470×g, about 1490×g, about 1500×g, about 1510×g, about 1530×g, or about 1550×g). In some embodiments, the duration of centrifugation can be about 10 min to about 30 min (e.g., about 10 to about 20 min, about 20 to about 30 min, about 10 min, about 20 min, or about 30 min).

In some embodiments, a final blood product can be prepared using both TFF and centrifugation (e.g., TFF followed by centrifugation or centrifugation followed by TFF).

Also provided herein are compositions prepared by any of the methods described herein.

In some embodiments, a composition as described herein can be analyzed at multiple points during processing. In some embodiments, a starting material (e.g., donor apheresis material (e.g., pooled donor apheresis material)) can be analyzed for antibody content (e.g., HLA or HNA antibody content). In some embodiments, a starting material (e.g., donor apheresis material (e.g., pooled donor apheresis material)) can be analyzed for protein concentration (e.g., by absorbance at 280 nm (e.g., using a path length of 0.5 cm)). In some embodiments, a composition in an intermediate step of processing (e.g., when protein concentration reduced to less than or equal to 75% (e.g., less than or equal to 70%, 65%, 60%, 55%, 50%, 45%, 40%, 35%, 30%, 25%, 20%, 15%, 10%, 9%, 8%, 7%, 6%, 5%, 4%, 3%, 2%, 1%, or less) of the protein concentration of an unprocessed blood product) can be analyzed for antibody content (e.g., HLA or HNA antibody content). In some embodiments, the antibody content (e.g., HLA or HNA antibody content) of a blood product in an intermediate step of processing can be at least 5% reduced (e.g., 10%, 20%, 30%, 40%, 50%, 60%, 70%, 75%, 80%, 85%, 90%, 95%, 96%, 97%, 98%, 99%, or more, reduced) compared to the antibody content of the starting material. In some embodiments, a final blood product (e.g., (e.g., platelets, cryopreserved platelets, freeze-dried platelets (e.g., thrombosomes)) can be analyzed for antibody content (e.g., HLA or HNA antibody content). In some embodiments described herein, a final blood product can be a composition that includes platelets and an aqueous medium. In some embodiments, the antibody content (e.g., HLA or HNA antibody content) of a final blood product (e.g., (e.g., platelets, cryopreserved platelets, freeze-dried platelets (e.g., thrombosomes)) can be at least 5% reduced (e.g., 10%, 20%, 30%, 40%, 50%, 60%, 70%, 75%, 80%, 85%, 90%, 95%, 96%, 97%, 98%, 99%, or more, reduced) compared to the antibody content of the starting material. In some embodiments, a final blood product can have no detectable level of an antibody selected from the group consisting of HLA Class I antibodies, HLA Class II antibodies, and HNA antibodies. In some embodiments, the aqueous medium of a composition as described herein can be analyzed as described herein.

In some embodiments, a composition as described herein can be analyzed at multiple points during processing. In some embodiments, donor apheresis plasma can be analyzed for antibody content (e.g., HLA or HNA antibody content). In some embodiments, donor apheresis plasma can be analyzed for protein concentration (e.g., by absorbance at 280 nm). In some embodiments, a composition in an intermediate step of processing (e.g., when protein concentration reduced to less than or equal to 75% (e.g., less than or equal to 70%, 65%, 60%, 55%, 50%, 45%, 40%, 35%, 30%, 25%, 20%, 15%, 10%, 9%, 8%, 7%, 6%, 5%, 4%, 3%, 2%, 1%, or less) of the protein concentration of an unprocessed blood product) can be analyzed for antibody content (e.g., HLA or HNA antibody content). In some embodiments, the antibody content (e.g., HLA or HNA antibody content) of a blood product in an intermediate step of processing can be at least 5% reduced (e.g., 10%, 20%, 30%, 40%, 50%, 60%, 70%, 75%, 80%, 85%, 90%, 95%, 96%, 97%, 98%, 99%, or more, reduced) compared to the antibody content of donor apheresis plasma. In some embodiments, a final blood product (e.g., (e.g., platelets, cryopreserved platelets, freeze-dried platelets (e.g., thrombosomes)) can be analyzed for antibody content (e.g., HLA or HNA antibody content). In some embodiments described herein, a final blood product can be a composition that includes platelets and an aqueous medium. In some embodiments, the antibody content (e.g., HLA or HNA antibody content) of a final blood product (e.g., (e.g., platelets, cryopreserved platelets, freeze-dried platelets (e.g., thrombosomes)) can be at least 5% reduced (e.g., 10%, 20%, 30%, 40%, 50%, 60%, 70%, 75%, 80%, 85%, 90%, 95%, 96%, 97%, 98%, 99%, or more, reduced) compared to the antibody content of donor apheresis plasma. In some embodiments, a final blood product can have no detectable level of an antibody selected from the group consisting of HLA Class I antibodies, HLA Class II antibodies, and HNA antibodies. In some embodiments, the aqueous medium of a composition as described herein can be analyzed as described herein.

The protein concentration of a blood product can be measured by any appropriate method. In some embodiments, the protein concentration of a blood product can be measured using absorbance at 280 nm.

The antibody content (e.g., HLA or HNA antibody content) of a blood product can be measured by any appropriate method.

In some embodiments, a FLOWPRA™ Screening or a LABScreen Multi test kits from One Lambda, Thermo Fisher Scientific can be used as a method of HLA detection. Raw materials can be tested prior to the TFF or centrifugation processes to determine a baseline level of class I and II antibodies for Human Leukocyte Antigen (HLA) and Human Neutrophil Antigens (HNA). Testing can be repeated after processing by centrifugation or TFF to measure the removal of HLA and HNA. Additional testing points can be performed throughout the TFF procedure to maintain in-process control. Post-lyophilization and annealing, random samples can be selected from a batch and qualitative HLA/HNA antibody testing can be performed to ensure reduction and compliance with current FDA testing and acceptance requirements.

In some embodiments, the antibody content (e.g., HLA or HNA antibody content) of two blood products can be compared by determining the percentage of beads positive for a marker (e.g., HLA or HNA coated beads bound to HLA or HNA antibodies, respectively). Any appropriate comparative method can be used. In some embodiments, the antibody content of two blood products can be compared using a method as described herein. In some embodiments, such a method can be carried out as follows. An aliquot of plasma (e.g., about 1 mL) platelet-poor plasma can be obtained. In some embodiments, an aliquot of filtered (e.g., using a 0.2 μm filter) platelet-poor plasma (PPP) (e.g., about 1 mL) can be obtained. Beads coated with Class I HLA and/or beads coated with Class II HLA can be added to the plasma (e.g., about 5 μL of each type of bead to about 20 μL of PPP) to form a mixture of PPP and beads. The mixture of PPP and beads can be vortexed. The mixture of PPP and beads can be incubated to form an incubated mixture. Any appropriate incubation conditions can be used. For example, in some embodiments, incubation can occur for a time (e.g., for about 30 minutes) at a temperature (e.g., at room temperature) with other conditions (e.g., in the dark) to form an incubated mixture. In some embodiments, incubation can include agitation (e.g., gentle rocking). The beads in the incubated mixture can be washed using any appropriate conditions. In some embodiments, the beads in the incubated mixture can be washed with a wash buffer. Washed beads can be separated from the incubated mixture by any appropriate method. In some embodiments, the washed beads can be separated by centrifugation (e.g., at 9,000×g for 2 minutes) to obtain pelleted beads. In some embodiments, the washing step can be repeated. The beads can be resuspended to form a bead solution. An antibody (e.g., an antibody that will bind to the assayed antibody content (e.g., HLA or HNA antibody content)) conjugated to a detectable moiety can be added to the bead solution (e.g., an αIgG conjugated to a fluorescent reporter, such as FITC). The antibody can be incubated with the bead solution under any appropriate conditions. In some embodiments, the antibody can be incubated for a time (e.g., for about 30 minutes) at a temperature (e.g., at room temperature) with other conditions (e.g., in the dark) to form labeled beads. Labeled beads can be washed to remove unbound antibody conjugated to a detectable moiety. The labeled beads can be washed using any appropriate conditions. In some embodiments, the labeled beads can be washed with a wash buffer. Washed labeled beads can be separated by any appropriate method. In some embodiments, the washed labeled beads can be separated by centrifugation (e.g., at 9,000 g for 2 minutes) to obtain pelleted labeled beads. In some embodiments, the washing step can be repeated. Labeled beads can be detected by any appropriate method. In some embodiments, labeled beads can be detected by flow cytometry. In some embodiments, detection can include measurement of the percentage of beads that are positive for the detectable moiety as compared to a negative control. In some embodiments, a negative control can be prepared as above, using a PPP sample that is known to be negative for antibodies (e.g. HLA Class I, HLA Class II, or HNA antibodies).

In some embodiments, a blood product (e.g., platelets, cryopreserved platelets, freeze-dried platelets (e.g., thrombosomes)) can be analyzed at multiple points during processing. In some embodiments, a starting material (e.g., donor apheresis material) can be analyzed to determine the percent of positive beads (e.g., HLA or HNA coated beads). In some embodiments, a starting material (e.g., donor apheresis material) can be analyzed for protein concentration (e.g., by absorbance at 280 nm). In some embodiments, a blood product in an intermediate step of processing (e.g., when protein concentration reduced to less than or equal to 75% (e.g., less than or equal to 70%, 65%, 60%, 55%, 50%, 45%, 40%, 35%, 30%, 25%, 20%, 15%, 10%, 9%, 8%, 7%, 6%, 5%, 4%, 3%, 2%, 1%, or less) of the protein concentration of a starting material) can be analyzed to determine the percent of positive beads (e.g., HLA or HNA coated beads). In some embodiments, a blood product in an intermediate step of processing (e.g., when protein concentration reduced to less than or equal to 75% (e.g., less than or equal to 70%, 65%, 60%, 55%, 50%, 45%, 40%, 35%, 30%, 25%, 20%, 15%, 10%, 9%, 8%, 7%, 6%, 5%, 4%, 3%, 2%, 1%, or less) of the protein concentration of a starting material) can be analyzed to determine the percent of positive beads (e.g., HLA or HNA coated beads). In some embodiments, the percent of positive beads (e.g., HLA or HNA coated beads) from a blood product in an intermediate step of processing can be at least 5% reduced (e.g., 10%, 20%, 30%, 40%, 50%, 60%, 70%, 75%, 80%, 85%, 90%, 95%, 96%, 97%, 98%, 99%, or more, reduced) compared to the percent of positive beads from a starting material. In some embodiments, the percent of positive beads (e.g., HLA or HNA coated beads) from a blood product in an intermediate step of processing can be less than or equal to 75% (e.g., less than or equal to 70%, 65%, 60%, 55%, 50%, 45%, 40%, 35%, 30%, 25%, 20%, 15%, 10%, 9%, 8%, 7%, 6%, 5%, 4%, 3%, 2%, 1%, or less) of the total amount of beads. In some embodiments, a final blood product (e.g., (e.g., platelets, cryopreserved platelets, freeze-dried platelets (e.g., thrombosomes)) can be analyzed to determine the percent of positive beads (e.g., HLA or HNA coated beads). In some embodiments, the percent of positive beads (e.g., HLA or HNA coated beads) from a final blood product (e.g., (e.g., platelets, cryopreserved platelets, freeze-dried platelets (e.g., thrombosomes)) can be at least 5% reduced (e.g., 10%, 20%, 30%, 40%, 50%, 60%, 70%, 75%, 80%, 85%, 90%, 95%, 96%, 97%, 98%, 99%, or more, reduced) compared to the percent of positive beads from a starting material. In some embodiments, the percent of positive beads (e.g., HLA or HNA coated beads) from a final blood product can be less than or equal to 75% (e.g., less than or equal to 70%, 65%, 60%, 55%, 50%, 45%, 40%, 35%, 30%, 25%, 20%, 15%, 10%, 9%, 8%, 7%, 6%, 5%, 4%, 3%, 2%, 1%, or less) of the total amount of beads. In some embodiments, the aqueous medium of a composition as described herein can be analyzed as described herein.

In some embodiments, a blood product (e.g., platelets, cryopreserved platelets, freeze-dried platelets (e.g., thrombosomes)) can be analyzed at multiple points during processing. In some embodiments, donor apheresis plasma can be analyzed to determine the percent of positive beads (e.g., HLA or HNA coated beads). In some embodiments, donor apheresis plasma can be analyzed for protein concentration (e.g., by absorbance at 280 nm). In some embodiments, a blood product in an intermediate step of processing (e.g., when protein concentration reduced to less than or equal to 75% (e.g., less than or equal to 70%, 65%, 60%, 55%, 50%, 45%, 40%, 35%, 30%, 25%, 20%, 15%, 10%, 9%, 8%, 7%, 6%, 5%, 4%, 3%, 2%, 1%, or less) of the protein concentration of a starting material) can be analyzed to determine the percent of positive beads (e.g., HLA or HNA coated beads). In some embodiments, a blood product in an intermediate step of processing (e.g., when protein concentration reduced to less than or equal to 75% (e.g., less than or equal to 70%, 65%, 60%, 55%, 50%, 45%, 40%, 35%, 30%, 25%, 20%, 15%, 10%, 9%, 8%, 7%, 6%, 5%, 4%, 3%, 2%, 1%, or less) of the protein concentration of a starting material) can be analyzed to determine the percent of positive beads (e.g., HLA or HNA coated beads). In some embodiments, the percent of positive beads (e.g., HLA or HNA coated beads) from a blood product in an intermediate step of processing can be at least 5% reduced (e.g., 10%, 20%, 30%, 40%, 50%, 60%, 70%, 75%, 80%, 85%, 90%, 95%, 96%, 97%, 98%, 99%, or more, reduced) compared to the percent of positive beads from donor apheresis plasma. In some embodiments, the percent of positive beads (e.g., HLA or HNA coated beads) from a blood product in an intermediate step of processing can be less than or equal to 75% (e.g., less than or equal to 70%, 65%, 60%, 55%, 50%, 45%, 40%, 35%, 30%, 25%, 20%, 15%, 10%, 9%, 8%, 7%, 6%, 5%, 4%, 3%, 2%, 1%, or less) of the total amount of beads. In some embodiments, a final blood product (e.g., (e.g., platelets, cryopreserved platelets, freeze-dried platelets (e.g., thrombosomes)) can be analyzed to determine the percent of positive beads (e.g., HLA or HNA coated beads). In some embodiments, the percent of positive beads (e.g., HLA or HNA coated beads) from a final blood product (e.g., (e.g., platelets, cryopreserved platelets, freeze-dried platelets (e.g., thrombosomes)) can be at least 5% reduced (e.g., 10%, 20%, 30%, 40%, 50%, 60%, 70%, 75%, 80%, 85%, 90%, 95%, 96%, 97%, 98%, 99%, or more, reduced) compared to the percent of positive beads from donor apheresis material. In some embodiments, the percent of positive beads (e.g., HLA or HNA coated beads) from a final blood product can be less than or equal to 75% (e.g., less than or equal to 70%, 65%, 60%, 55%, 50%, 45%, 40%, 35%, 30%, 25%, 20%, 15%, 10%, 9%, 8%, 7%, 6%, 5%, 4%, 3%, 2%, 1%, or less) of the total amount of beads. In some embodiments, the aqueous medium of a composition as described herein can be analyzed as described herein.

A percentage of positive beads can be determined using any appropriate method. In some embodiments, positive beads can be determined compared to a negative control sample. A negative control sample can be any appropriate negative control sample. In some embodiments, a negative control sample can be used to determine positivity gating such that less than a certain percentage (e.g., between about 0.01% and about 1% (e.g., about 0.01% to about 0.05%, about 0.05% to about 0.1%, about 0.1% to about 0.5%, about 0.5% to about 1%, about 0.01%, about 0.05%, about 0.1%, about 0.5%, or about 1%)) of the negative control sample is present within the positivity gate. In some embodiments, a negative control sample can be a buffer (e.g., PBS). In some embodiments, a negative control sample can be a synthetic plasma composition. In some embodiments, a negative control sample can be a blood product known to be negative for the assayed antibodies (e.g., HLA or HNA antibodies).

Also provided herein is a method of reducing the percentage of an antibody (e.g., a HLA antibody (e.g., a HLA Class I antibody or a HLA Class II antibody) or a HNA antibody) in a composition (e.g., a blood product) comprising platelets, the method comprising filtering the composition by tangential flow filtration. Also provided herein is a method of reducing the amount of an antibody (e.g., a HLA antibody (e.g., a HLA Class I antibody or a HLA Class II antibody) or a HNA antibody) in a composition (e.g., a blood product) comprising platelets, the method comprising filtering the composition by tangential flow filtration. Also provided herein is a method of reducing the percentage of beads positive for an antibody (e.g., a HLA antibody (e.g., a HLA Class I antibody or a HLA Class II antibody) or a HNA antibody) in a composition (e.g., a blood product) comprising platelets, the method comprising filtering the composition by tangential flow filtration.

Also provided herein is a method of reducing the percentage of an antibody (e.g., a HLA antibody (e.g., a HLA Class I antibody or a HLA Class II antibody) or a HNA antibody) in a composition (e.g., a blood product) comprising platelets, the method comprising filtering the composition by centrifugation. Also provided herein is a method of reducing the amount of an antibody (e.g., a HLA antibody (e.g., a HLA Class I antibody or a HLA Class II antibody) or a HNA antibody) in a composition (e.g., a blood product) comprising platelets, the method comprising filtering the composition by centrifugation. Also provided herein is a method of reducing the percentage of beads positive for an antibody (e.g., a HLA antibody (e.g., a HLA Class I antibody or a HLA Class II antibody) or a HNA antibody) in a composition (e.g., a blood product) comprising platelets, the method comprising filtering the composition by centrifugation.

In some embodiments of any of the methods described herein, the amount of an antibody (e.g., a HLA antibody (e.g., a HLA Class I antibody or a HLA Class II antibody) or a HNA antibody) in a composition (e.g., a blood product) can be reduced to below a reference level. A reference level can be any appropriate reference level. In some embodiments of any of the methods described herein, the percentage of beads positive an antibody (e.g., a HLA antibody (e.g., a HLA Class I antibody or a HLA Class II antibody) or a HNA antibody) in a composition (e.g., a blood product) can be reduced as compared to the blood product before undergoing the methods described herein. A percentage of beads positive for an antibody can be reduced by any appropriate amount. In some embodiments, a percentage of beads positive for an antibody can be reduced by at least 5% (e.g., reduced by at least 10%, 20%, 30%, 40%, 50%, 60%, 70%, 75%, 80%, 85%, 90%, 95%, 96%, 97%, 98%, 99%, or more) compared to the blood product before undergoing any of the methods described herein.

In some embodiments, a composition as described herein can undergo any appropriate additional processing steps. In some embodiments, a composition as described herein can be freeze-dried. In some embodiments, freeze-dried platelets can be thermally treated (e.g., at about 80° C. for about 24 hours).

For example, in some embodiments, a composition can be cryopreserved or freeze-dried. In some embodiments, a first composition (e.g., a composition comprising platelets and an aqueous medium as described herein) can be treated with a mixture. In some embodiments, a mixture can include a lyophilizing agent, including a base, a loading agent, and optionally at least one organic solvent such as an organic solvent selected from the group consisting of ethanol, acetic acid, acetone, acetonitrile, dimethylformamide, dimethyl sulfoxide, dioxane, methanol, n-propanol, isopropanol, tetrahydrofuran (THF), N-methyl pyrrolidone, dimethylacetamide (DMAC), or combinations thereof, to form a second composition comprising platelets. In some embodiments, a loading agent can be a saccharide. In some embodiments, a saccharide can be a monosaccharide. In some embodiments, a saccharide can be sucrose, maltose, trehalose, glucose (e.g., dextrose), mannose, or xylose. In some embodiments, the loading agent can be polysucrose.

In some embodiments, a first composition or a second composition can be dried. In some embodiments, a first composition or a second composition can be dried with a cryoprotectant. In some embodiments, a cryoprotectant can include a saccharide, optionally a base, and optionally at least one organic solvent such as an organic solvent selected from the group consisting of ethanol, acetic acid, acetone, acetonitrile, dimethylformamide, dimethyl sulfoxide, dioxane, methanol, n-propanol, isopropanol, tetrahydrofuran (THF), N-methyl pyrrolidone, dimethylacetamide (DMAC), or combinations thereof to form a third composition. In some embodiments, a cryoprotectant can be polysucrose.

In some embodiments, a first composition or a second composition can be freeze-dried. In some embodiments, a first composition or a second composition can be freeze-dried with a cryoprotectant. In some embodiments, a cryoprotectant can include a saccharide, optionally a base, and optionally at least one organic solvent such as an organic solvent selected from the group consisting of ethanol, acetic acid, acetone, acetonitrile, dimethylformamide, dimethyl sulfoxide, dioxane, methanol, n-propanol, isopropanol, tetrahydrofuran (THF), N-methyl pyrrolidone, dimethylacetamide (DMAC), or combinations thereof to form a fourth composition. In some embodiments freeze-drying can occur at a temperature of about −40° C. to about 5° C. In some embodiments, freeze-drying can occur over a gradient (e.g., about −40° C. to about 5° C.). In some embodiments, a secondary drying step can be carried out (e.g., at about 20° C. to about 40° C.).

Also provided herein are blood products (e.g., platelets, cryopreserved platelets, freeze-dried platelets (e.g., thrombosomes)) produced by any of the methods described herein.

In some embodiments, the percentage of beads positive for an antibody selected from the group consisting of HLA Class I antibodies, HLA Class II antibodies, and HNA antibodies, as determined for a composition as described herein by flow cytometry using beads coated with Class I HLAs, Class II HLAs, or HNAs, respectively, is reduced by at least 10% (e.g., at least 20%, 30%, 40%, 50%, 60%, 70%, 75%, 80%, 85%, 90%, 95%, or 99%) as compared to a similar composition not prepared by a process comprising tangential flow filtration of a composition comprising platelets, centrifugation of a composition comprising platelets, or a combination thereof.

In some embodiments, the percentage of beads positive for HLA Class I antibodies, as determined for a composition as described herein by flow cytometry using beads coated with Class I HLAs, is reduced by at least 10% (e.g., at least 20%, 30%, 40%, 50%, 60%, 70%, 75%, 80%, 85%, 90%, 95%, or 99%) as compared to a similar composition not prepared by a process comprising tangential flow filtration of a composition comprising platelets, centrifugation of a composition comprising platelets, or a combination thereof.

In some embodiments, the percentage of beads positive for HLA Class II antibodies, as determined for a composition as described herein by flow cytometry using beads coated with Class II HLAs, is reduced by at least 10% (e.g., at least 20%, 30%, 40%, 50%, 60%, 70%, 75%, 80%, 85%, 90%, 95%, or 99%) as compared to a similar composition not prepared by a process comprising tangential flow filtration of a composition comprising platelets, centrifugation of a composition comprising platelets, or a combination thereof.

In some embodiments, the percentage of beads positive for HNA antibodies, as determined for a composition as described herein by flow cytometry using beads coated with HNAs, is reduced by at least 10% (e.g., at least 20%, 30%, 40%, 50%, 60%, 70%, 75%, 80%, 85%, 90%, 95%, or 99%) as compared to a similar composition not prepared by a process comprising tangential flow filtration of a composition comprising platelets, centrifugation of a composition comprising platelets, or a combination thereof.

Within the process provided herein for making the compositions provided herein, optional addition of a lyophilizing agent can be the last step prior to drying. However, in some embodiments, the lyophilizing agent can be added at the same time or before other components of the composition, such as a salt, a buffer, optionally a cryoprotectant, or other components. In some embodiments, the lyophilizing agent is added to a preparation agent, thoroughly mixed to form a drying solution, dispensed into a drying vessel (e.g., a glass or plastic serum vial, a lyophilization bag), and subjected to conditions that allow for drying of a TFF-treated composition to form a dried composition.

In various embodiments, the lyophilization bag is a gas-permeable bag configured to allow gases to pass through at least a portion or all portions of the bag during the processing. The gas-permeable bag can allow for the exchange of gas within the interior of the bag with atmospheric gas present in the surrounding environment. The gas-permeable bag can be permeable to gases, such as oxygen, nitrogen, water, air, hydrogen, and carbon dioxide, allowing gas exchange to occur in the compositions provided herein. In some embodiments, the gas-permeable bag allows for the removal of some of the carbon dioxide present within an interior of the bag by allowing the carbon dioxide to permeate through its wall. In some embodiments, the release of carbon dioxide from the bag can be advantageous to maintaining a desired pH level of the composition contained within the bag.

In some embodiments, the container of the process herein is a gas-permeable container that is closed or sealed. In some embodiments, the container is a container that is closed or sealed and a portion of which is gas-permeable. In some embodiments, the surface area of a gas-permeable portion of a closed or sealed container (e.g., bag) relative to the volume of the product being contained in the container (hereinafter referred to as the “SA/V ratio”) can be adjusted to improve pH maintenance of the compositions provided herein. For example, in some embodiments, the SAV ratio of the container can be at least about 2.0 cm²/mL (e.g., at least about 2.1 cm²/mL, at least about 2.2 cm²/mL, at least about 2.3 cm²/mL, at least about 2.4 cm²/mL, at least about 2.5 cm²/mL, at least about 2.6 cm²/mL, at least about 2.7 cm²/mL, at least about 2.8 cm²/mL, at least about 2.9 cm²/mL, at least about 3.0 cm²/mL, at least about 3.1 cm²/mL, at least about 3.2 cm²/mL, at least about 3.3 cm²/mL, at least about 3.4 cm²/mL, at least about 3.5 cm²/mL, at least about 3.6 cm²/mL, at least about 3.7 cm²/mL, at least about 3.8 cm²/mL, at least about 3.9 cm²/mL, at least about 4.0 cm²/mL, at least about 4.1 cm²/mL, at least about 4.2 cm²/mL, at least about 4.3 cm²/mL, at least about 4.4 cm²/mL, at least about 4.5 cm²/mL, at least about 4.6 cm²/mL, at least about 4.7 cm²/mL, at least about 4.8 cm²/mL, at least about 4.9 cm²/mL, or at least about 5.0 cm²/mL. In some embodiments, the SAV ratio of the container can be at most about 10.0 cm²/mL (e.g., at most about 9.9 cm²/mL, at most about 9.8 cm²/mL, at most about 9.7 cm²/mL, at most about 9.6 cm²/mL, at most about 9.5 cm²/mL, at most about 9.4 cm²/mL, at most about 9.3 cm²/mL, at most about 9.2 cm²/mL, at most about 9.1 cm²/mL, at most about 9.0 cm²/mL, at most about 8.9 cm²/mL, at most about 8.8 cm²/mL, at most about 8.7 cm²/mL, at most about 8.6, cm²/mL at most about 8.5 cm²/mL, at most about 8.4 cm²/mL, at most about 8.3 cm²/mL, at most about 8.2 cm²/mL, at most about 8.1 cm²/mL, at most about 8.0 cm²/mL, at most about 7.9 cm²/mL, at most about 7.8 cm²/mL, at most about 7.7 cm²/mL, at most about 7.6 cm²/mL, at most about 7.5 cm²/mL, at most about 7.4 cm²/mL, at most about 7.3 cm²/mL, at most about 7.2 cm²/mL, at most about 7.1 cm²/mL, at most about 6.9 cm²/mL, at most about 6.8 cm²/mL, at most about 6.7 cm²/mL, at most about 6.6 cm²/mL, at most about 6.5 cm²/mL, at most about 6.4 cm²/mL, at most about 6.3 cm²/mL, at most about 6.2 cm²/mL, at most about 6.1 cm²/mL, at most about 6.0 cm²/mL, at most about 5.9 cm²/mL, at most about 5.8 cm²/mL, at most about 5.7 cm²/mL, at most about 5.6 cm²/mL, at most about 5.5 cm²/mL, at most about 5.4 cm²/mL, at most about 5.3 cm²/mL, at most about 5.2 cm²/mL, at most about 5.1 cm²/mL, at most about 5.0 cm²/mL, at most about 4.9 cm²/mL, at most about 4.8 cm²/mL, at most about 4.7 cm²/mL, at most about 4.6 cm²/mL, at most about 4.5 cm²/mL, at most about 4.4 cm²/mL, at most about 4.3 cm²/mL, at most about 4.2 cm²/mL, at most about 4.1 cm²/mL, or at most about 4.0 cm²/mL. In some embodiments, the SAV ratio of the container can range from about 2.0 to about 10.0 cm²/mL (e.g., from about 2.1 cm²/mL to about 9.9 cm²/mL, from about 2.2 cm²/mL to about 9.8 cm²/mL, from about 2.3 cm²/mL to about 9.7 cm²/mL, from about 2.4 cm²/mL to about 9.6 cm²/mL, from about 2.5 cm²/mL to about 9.5 cm²/mL, from about 2.6 cm²/mL to about 9.4 cm²/mL, from about 2.7 cm²/mL to about 9.3 cm²/mL, from about 2.8 cm²/mL to about 9.2 cm²/mL, from about 2.9 cm²/mL to about 9.1 cm²/mL, from about 3.0 cm²/mL to about 9.0 cm²/mL, from about 3.1 cm²/mL to about 8.9 cm²/mL, from about 3.2 cm²/mL to about 8.8 cm²/mL, from about 3.3 cm²/mL to about 8.7 cm²/mL, from about 3.4 cm²/mL to about 8.6 cm²/mL, from about 3.5 cm²/mL to about 8.5 cm²/mL, from about 3.6 cm²/mL to about 8.4 cm²/mL, from about 3.7 cm²/mL to about 8.3 cm²/mL, from about 3.8 cm²/mL to about 8.2 cm²/mL, from about 3.9 cm²/mL to about 8.1 cm²/mL, from about 4.0 cm²/mL to about 8.0 cm²/mL, from about 4.1 cm²/mL to about 7.9 cm²/mL, from about 4.2 cm²/mL to about 7.8 cm²/mL, from about 4.3 cm²/mL to about 7.7 cm²/mL, from about 4.4 cm²/mL to about 7.6 cm²/mL, from about 4.5 cm²/mL to about 7.5 cm²/mL, from about 4.6 cm²/mL to about 7.4 cm²/mL, from about 4.7 cm²/mL to about 7.3 cm²/mL, from about 4.8 cm²/mL to about 7.2 cm²/mL, from about 4.9 cm²/mL to about 7.1 cm²/mL, from about 5.0 cm²/mL to about 6.9 cm²/mL, from about 5.1 cm²/mL to about 6.8 cm²/mL, from about 5.2 cm²/mL to about 6.7 cm²/mL, from about 5.3 cm²/mL to about 6.6 cm²/mL, from about 5.4 cm²/mL to about 6.5 cm²/mL, from about 5.5 cm²/mL to about 6.4 cm²/mL, from about 5.6 cm²/mL to about 6.3 cm²/mL, from about 5.7 cm²/mL to about 6.2 cm²/mL, or from about 5.8 cm²/mL to about 6.1 cm²/mL.

Gas-permeable closed containers (e.g., bags) or portions thereof can be made of one or more various gas-permeable materials. In some embodiments, the gas-permeable bag can be made of one or more polymers including fluoropolymers (such as polytetrafluoroethylene (PTFE) and perfluoroalkoxy (PFA) polymers), polyolefins (such as low-density polyethylene (LDPE), high-density polyethylene (HDPE)), fluorinated ethylene propylene (FEP), polystyrene, polyvinylchloride (PVC), silicone, and any combinations thereof.

In some embodiments, dried platelets or platelet derivatives (e.g., thrombosomes) can undergo heat treatment. Heating can be performed at a temperature above about 25° C. (e.g., greater than about 40° C., 50° C., 60° C., 70° C., 80° C. or higher). In some embodiments, heating is conducted between about 70° C. and about 85° C. (e.g., between about 75° C. and about 85° C., or at about 75° C. or 80° C.). The temperature for heating can be selected in conjunction with the length of time that heating is to be performed. Although any suitable time can be used, typically, the lyophilized platelets are heated for at least 1 hour, but not more than 36 hours. Thus, in embodiments, heating is performed for at least 2 hours, at least 6 hours, at least 12 hours, at least 18 hours, at least 20 hours, at least 24 hours, or at least 30 hours. For example, the lyophilized platelets can be heated for 18 hours, 19 hours, 20 hours, 21 hours, 22 hours, 23 hours, 24 hours, 25 hours, 26 hours, 27 hours, 28 hours, 29 hours, or 30 hours. Non-limiting exemplary combinations include: heating the dried platelets or platelet derivatives (e.g., thrombosomes) for at least 30 minutes at a temperature higher than 30° C.; heating the dried platelets or platelet derivatives (e.g., thrombosomes) for at least 10 hours at a temperature higher than 50° C.; heating the dried platelets or platelet derivatives (e.g., thrombosomes) for at least 18 hours at a temperature higher than 75° C.; and heating the dried platelets or platelet derivatives (e.g., thrombosomes) for 24 hours at 80° C. In some embodiments, heating can be performed in sealed container, such as a capped vial. In some embodiments, a sealed container be subjected to a vacuum prior to heating. The heat treatment step, particularly in the presence of a cryoprotectant such as albumin or polysucrose, has been found to improve the stability and shelf-life of the freeze-dried platelets. Indeed, advantageous results have been obtained with the particular combination of serum albumin or polysucrose and a post-lyophilization heat treatment step, as compared to those cryoprotectants without a heat treatment step. A cryoprotectant (e.g., sucrose) can be present in any appropriate amount (e.g. about 3% to about 10% by mass or by volume of the platelets or platelet derivatives (e.g., thrombosomes).

In some cases, compositions comprising platelets or platelet derivatives (e.g., thrombosomes) can be rehydrated with water (e.g., sterile water for injection) over about 10 minutes at about room temperature. In general, the rehydration volume is about equal to the volume used to fill each vial of thrombosomes prior to drying.

In some embodiments, the platelets or platelet derivatives (e.g., thrombosomes) prepared as disclosed herein have a storage stability that is at least about equal to that of the platelets prior to the preparation.

In some embodiments, the method further comprises cryopreserving the platelets or platelet derivatives prior to administering the platelets or platelet derivatives (e.g., with a preparation agent, e.g., a preparation agent described herein).

In some embodiments, the method further comprises drying a composition comprising platelets or platelet derivatives, (e.g., with a preparation agent e.g., a preparation agent described herein) prior to administering the platelets or platelet derivatives (e.g., thrombosomes). In some embodiments, the method may further comprise heating the composition following the drying step. In some embodiments, the method may further comprise rehydrating the composition following the freeze-drying step or the heating step.

In some embodiments, the method further comprises freeze-drying a composition comprising platelets or platelet derivatives (e.g., with a preparation agent e.g., a preparation agent described herein) prior to administering the platelets or platelet derivatives (e.g., thrombosomes) In some embodiments, the method may further comprise heating the composition following the freeze-drying step. In some embodiments, the method may further comprise rehydrating the composition following the freeze-drying step or the heating step.

In some embodiments, the method further comprises cold storing the platelets, platelet derivatives, or the thrombosomes prior to administering the platelets, platelet derivatives, or thrombosomes (e.g., with a preparation agent, e.g., a preparation agent described herein).

Storing conditions include, for example, standard room temperature storing (e.g., storing at a temperature ranging from about 20 to about 30° C.) or cold storing (e.g., storing at a temperature ranging from about 1 to about 10° C.). In some embodiments, the method further comprises cryopreserving, freeze-drying, thawing, rehydrating, and combinations thereof, a composition comprising platelets or platelet derivatives (e.g., thrombosomes) (e.g., with a preparation agent e.g., a preparation agent described herein) prior to administering the platelets or platelet derivatives (e.g., thrombosomes). For example, in some embodiments, the method further comprises drying (e.g., freeze-drying) a composition comprising platelets or platelet derivatives (e.g., with a preparation agent e.g., a preparation agent described herein) (e.g., to form thrombosomes) prior to administering the platelets or platelet derivatives (e.g., thrombosomes). In some embodiments, the method may further comprise rehydrating the composition obtained from the drying step.

In some embodiments, provided herein is a method for preparing a composition comprising platelets or platelet derivatives (e.g., thrombosomes). The method can include diluting a starting material comprising platelets with an approximately equal weight (±10%) of a preparation agent (e.g., Buffer A, as provided in Example 1), concentrating the platelets to about 2250×10³ cells/μL (±250×10³) and then washed with 2-4 diavolumes (DV) (e.g., about 2 diavolumes) of the preparation agent to form a TFF-treated composition. The residual plasma percentage can be less than about 15% relative plasma (as determined by plasma protein content). Following washing, if the concentration of the cells in the TFF-treated composition is not about 2000×10³ cells/μL (±300×10³), the cells can be diluted with the preparation agent or can be concentrated to fall within this range. The method can further include lyophilizing the TFF-treated composition and subsequently treating the lyophilized composition comprising platelets or platelet derivatives (e.g., thrombosomes) at about 80° C. for about 24 hours. In some embodiments, the method can further include a pathogen reduction step, for example, before diluting the starting material.

### Exemplary Embodiments

Provided in this Exemplary Embodiments section are non-limiting exemplary aspects and embodiments provided herein and further discussed throughout this specification. For the sake of brevity and convenience, all of the aspects and embodiments disclosed herein, and all of the possible combinations of the disclosed aspects and embodiments are not listed in this section. Additional embodiments and aspects are provided in other sections herein. Furthermore, it will be understood that embodiments are provided that are specific embodiments for many aspects and that can be combined with any other embodiment, for example as discussed in this entire disclosure. It is intended in view of the full disclosure herein, that any individual embodiment recited below or in this full disclosure can be combined with any aspect recited below or in this full disclosure where it is an additional element that can be added to an aspect or because it is a narrower element for an element already present in an aspect. Such combinations are sometimes provided as non-limiting exemplary combinations and/or are discussed more specifically in other sections of this detailed description.

Also provided herein are compositions produced by any of the methods described herein. In some embodiments, any of the compositions provided herein can be made by the methods described herein. Specific embodiments disclosed herein may be further limited in the claims using “consisting of” or “consisting essentially of” language.

In one aspect, provided herein is a method of treating a subject, for example, reducing or decreasing bleeding in the subject, comprising:
administering a dose of platelet derivatives in a platelet derivative composition to the subject,
wherein the subject is refractory to platelet transfusion,
wherein the dose of the platelet derivatives is an amount of between 5.1×10⁸ to 9.9×10⁸/kg, or 1.0×10⁹ to 1.0×10¹⁰/kg of the subject, and
wherein after the administering, the method leads to the subject having decreased bleeding without a bleeding score of WHO Grade 2A or a greater bleeding score during the first 10 days after the administering a first dose of the platelet derivatives. In some embodiments, the method further comprises rehydrating a platelet derivative composition in the form of a powder to form a rehydrated platelet derivative composition comprising a dose of platelet derivatives, and administering the rehydrated platelet derivative composition to the subject. Typically, the platelet derivatives have one or more of the following: at least 70% of the platelet derivatives in the rehydrated platelet derivative composition are CD41 positive platelet derivatives when measured using flow cytometry, and less than 5% of the CD41 positive platelet derivatives are microparticles, wherein the platelet derivatives in the rehydrated platelet derivative composition have a compromised membrane, wherein at least 70% of the platelet derivatives in the rehydrated platelet derivative composition are CD42 positive platelet derivatives, when measured using flow cytometry, wherein at least 70% of the platelet derivatives in the rehydrated platelet derivative composition are CD62 positive platelet derivatives, when measured using flow cytometry, wherein the platelet derivatives are capable of generating thrombin in an in vitro thrombin formation assay, and wherein the platelet derivatives show an inability to increase expression of a platelet activation marker in the presence of an agonist as compared to the expression of the platelet activation marker in the absence of an agonist, and wherein the platelet activation marker is Annexin V, CD62, or a combination thereof.

In one aspect, provided herein is a method of treating a subject, for example, reducing or decreasing bleeding in the subject, comprising:
administering a dose of platelet derivatives in a platelet derivative composition to the subject,
wherein the subject is refractory to platelet transfusion,
wherein the dose of the platelet derivatives is an amount of between 5×10⁸ particles/kg to 1.0×10¹² particles/kg of the subject, and
wherein after the administering, the bleeding potential of the subject is reduced, and the method leads to the subject having decreased bleeding without a bleeding score of WHO Grade 2A or a greater bleeding score during the first 10 days after the administering a first dose of the platelet derivatives. In some embodiments, the method further comprises rehydrating a platelet derivative composition in the form of a powder to form a rehydrated platelet derivative composition comprising a dose of platelet derivatives, and administering the rehydrated platelet derivative composition to the subject.

In one aspect, provided herein is a method of treating a subject, for example, reducing or decreasing bleeding in the subject, comprising:
rehydrating a platelet derivative composition in the form of a powder to form a rehydrated platelet derivative composition comprising rehydrated platelet derivatives, and
administering a dose of the rehydrated platelet derivative composition to the subject, wherein the dose comprises a first dose of the rehydrated platelet derivatives, wherein the subject is refractory to platelet transfusion, wherein the first dose of the rehydrated platelet derivatives is an amount of between 5.1×10⁸ and 9.9×10⁸/kg, or 1.0×10⁹ and 1.0×10¹⁰/kg of the subject, wherein the administering leads to the subject having decreased bleeding without a bleeding score of WHO Grade 2A or greater at 15 minutes, 30 minutes, 45 minutes, 1 hour, 2, 4, 6, 8, 10, or 12 hours after the administering the first dose of the platelet derivatives.

In one aspect, provided herein is a method of treating a subject, for example, reducing or decreasing bleeding in the subject, comprising:
rehydrating a platelet derivative composition in the form of a powder to form a rehydrated platelet derivative composition comprising rehydrated platelet derivatives, and
administering a dose of the rehydrated platelet derivative composition to the subject, wherein the dose comprises a first dose of the rehydrated platelet derivatives,
wherein the subject is refractory to platelet transfusion,
wherein the first dose of the rehydrated platelet derivatives is an amount of between 5×10⁸ particles/kg and 1.0×10¹² particles/kg of the subject,
wherein the administering leads to the subject having decreased bleeding without a bleeding score of WHO Grade 2A or greater at 15 minutes, 30 minutes, 45 minutes, 1 hour, 2, 4, 6, 8, 10, or 12 hours after the administering the first dose of the platelet derivatives.

In one aspect, provided herein is a method of controlling bleeding or reducing bleeding, and/or treating a subject having refractory thrombocytopenia, comprising:
administering at least 1 dose of platelet derivatives in a platelet derivative composition to the subject,
wherein a dose of the platelet derivatives is an amount of at least 4×10⁸ particles/kg, or 5×10⁸ particles/kg of the subject, and
wherein after the administering, the method leads to the subject having decreased bleeding as compared to a bleeding before the administering. In illustrative embodiments, the subject is bleeding. In some embodiments, the administering includes administering multiple doses, such as 1 to 10, 2 to 10, 3 to 10, 4 to 10, 1 to 8, 2 to 8, 3 to 8, 3 to 6, 4 to 8, 4 to 6, or 5 to 8 doses. In some embodiments, the dose of the platelet derivatives is an amount in the range of 4×10⁸ particles/kg to 1.0×10¹² particles/kg, or 5×10⁸ particles/kg to 1.0×10¹² particles/kg. In some embodiments, the administering occurs during a timeframe within 1, 2, 3, 4, 5, 6, or 7 days from a first dose. In illustrative embodiment, the method leads to the subject having decreased bleeding to a bleeding score of less than or equal to a WHO Grade 2A. In illustrative embodiments, the method leads to the subject having decreased bleeding to a bleeding score of less than a WHO Grade 2A.

In one aspect, provided herein is a method for controlling and/or reducing bleeding in a subject having a platelet count of between 10,000 and 70,000 platelets/μl of blood, comprising:
administering at least one dose of platelet derivatives in a platelet derivative composition to the subject in an amount of at least 1.6×10⁸, 1.7×10⁸, 1.8×10⁸, or 1.9×10⁸ particles/kg; or
wherein at the time of the administering the subject is bleeding,
wherein the administering occurs within 1, 2, 3, 4, 5, 6, or 7 days from a first dose, and
wherein after the administering, the method leads to the subject having decreased bleeding to a bleeding score of less than a WHO Grade 2A. In some embodiments, the administering includes administering 2 to 10, 3 to 10, 4 to 10, 5 to 10, 1 to 8, 2 to 8, 3 to 8, 3 to 6, 4 to 8, 4 to 6, or 5 to 8 doses. In some embodiments, the administering includes administering between 5×10⁸ particles/kg to 1.0×10¹² particles/kg. In some embodiments, the administering includes administering 3 to 10 doses of the platelet derivatives between 1.6×10⁸ particles/kg to 1.0×10¹² particles/kg.

In one aspect, provided herein is a method for controlling and/or reducing bleeding in a subject having a platelet count of between 10,000 and 70,000 platelets/μl of blood, comprising:
administering at least one dose of platelet derivatives in a platelet derivative composition to the subject in an amount of at least 1.6×10⁹ particles/kg; or
wherein at the time of the administering the subject is bleeding,
wherein the administering occurs within 1, 2, 3, 4, 5, 6, or 7 days from a first dose, and
wherein after the administering, the method leads to the subject having decreased bleeding to a bleeding score of less than a WHO Grade 2A. In some embodiments, the administering includes administering 2 to 10, 3 to 10, 4 to 10, 5 to 10, 1 to 8, 2 to 8, 3 to 8, 3 to 6, 4 to 8, 4 to 6, or 5 to 8 doses.

In one aspect, provided herein is a method for administering a platelet derivative composition to a subject, comprising: administering a chemotherapeutic agent to a subject in need thereof, according to a pre-platelet derivative chemotherapeutic dosing regimen, determining that the subject has a low platelet count, wherein the low platelet count is a blood platelet count of less than 150,000 platelets/μl of circulating blood, administering an effective dose of platelet derivatives in the platelet derivative composition to the subject, and administering the chemotherapeutic agent to the subject according to a post-platelet derivative chemotherapeutic dosing regimen, wherein the first dose administered in the post-platelet derivative chemotherapeutic dosing regimen is not delayed and/or the first dose is not reduced due to the low platelet count.

In one aspect, provided herein is a method for administering a platelet derivative composition to a subject, comprising: administering a cancer therapeutic agent to a subject in need thereof, according to a pre-platelet derivative cancer therapeutic dosing regimen, determining that the subject has a low platelet count, wherein the low platelet count is a blood platelet count of less than 150,000 platelets/μl of circulating blood, administering an effective dose of platelet derivatives in the platelet derivative composition to the subject, and administering the cancer therapeutic agent to the subject according to a post-platelet derivative cancer therapeutic dosing regimen, wherein the first dose administered in the post-platelet derivative cancer therapeutic dosing regimen is not delayed and/or the first dose is not reduced due to the low platelet count.

In one aspect, provided herein is a method for administering a platelet derivative composition to a subject, comprising: administering a chemotherapeutic agent to a subject in need thereof, according to a pre-platelet derivative chemotherapeutic dosing regimen, administering an effective dose of platelet derivatives in the platelet derivative composition to the subject, and administering the chemotherapeutic agent to the subject according to a post-platelet derivative chemotherapeutic dosing regimen, wherein the first dose administered in the post-platelet derivative chemotherapeutic dosing regimen is not delayed and/or the first dose is not reduced due to a low platelet count in the subject.

In one aspect, provided herein is a method for administering a platelet derivative composition to a subject, comprising: administering a cancer therapeutic agent to a subject in need thereof, according to a pre-platelet derivative cancer therapeutic dosing regimen, administering an effective dose of platelet derivatives in the platelet derivative composition to the subject, and administering the cancer therapeutic agent to the subject according to a post-platelet derivative cancer therapeutic dosing regimen, wherein the first dose administered in the post-platelet derivative cancer therapeutic dosing regimen is not delayed and/or the first dose is not reduced due to a low platelet count in the subject.

In one aspect, provided herein is a method for administering a platelet derivative composition to a subject, comprising: administering an effective dose of platelet derivatives in the platelet derivative composition to the subject before and/or after an n^{th} round of administering a chemotherapeutic agent according to a chemotherapeutic dosing regimen, and continuing the chemotherapeutic dosing regimen by administering the chemotherapeutic agent in an additional round to the subject, wherein the first dose of the chemotherapeutic agent in the additional round is not reduced and/or the first dose is not delayed due to a low platelet count in the subject.

In one aspect, provided herein is a method for administering a platelet derivative composition to a subject, comprising: administering an effective dose of platelet derivatives in the platelet derivative composition to the subject before and/or after an n^{th} round of administering a cancer therapeutic agent according to a cancer therapeutic dosing regimen, and continuing the cancer therapeutic dosing regimen by administering the cancer therapeutic agent in an additional round to the subject, wherein the first dose of the cancer therapeutic agent in the additional round is not reduced and/or the first dose is not delayed due to a low platelet count in the subject.

In one aspect, provided herein in one aspect is a method for administering a platelet derivative composition to a subject, comprising: administering a chemotherapeutic agent to a subject in need thereof, according to a pre-platelet derivative chemotherapeutic dosing regimen, determining that the subject has a low platelet count, wherein the low platelet count is a blood platelet count of less than 150,000 platelets/μl of circulating blood, administering an effective dose of platelet derivatives in the platelet derivative composition to the subject, and administering the chemotherapeutic agent to the subject according to a post-platelet derivative chemotherapeutic dosing regimen.

In one aspect, provided herein in one aspect is a method for administering a platelet derivative composition to a subject, comprising: administering a cancer therapeutic agent to a subject in need thereof, according to a pre-platelet derivative cancer therapeutic dosing regimen, determining that the subject has a low platelet count, wherein the low platelet count is a blood platelet count of less than 150,000 platelets/μl of circulating blood, administering an effective dose of platelet derivatives in the platelet derivative composition to the subject, and administering the cancer therapeutic agent to the subject according to a post-platelet derivative cancer therapeutic dosing regimen.

In one aspect, provided herein in is a method for administering a platelet derivative composition to a subject, comprising: administering a chemotherapeutic agent to a subject in need thereof, according to a pre-platelet derivative chemotherapeutic dosing regimen, administering an effective dose of platelet derivatives in the platelet derivative composition to the subject, and administering the chemotherapeutic agent to the subject according to a post-platelet derivative chemotherapeutic dosing regimen.

In one aspect, provided herein in is a method for administering a platelet derivative composition to a subject, comprising: administering a cancer therapeutic agent to a subject in need thereof, according to a pre-platelet derivative cancer therapeutic dosing regimen, administering an effective dose of platelet derivatives in the platelet derivative composition to the subject, and administering the cancer therapeutic agent to the subject according to a post-platelet derivative cancer therapeutic dosing regimen.

In another aspect, provided herein in is a method for administering a platelet derivative composition to a subject, comprising: administering an effective dose of platelet derivatives in the platelet derivative composition to the subject before and/or after an n^{th} round of administering a chemotherapeutic agent according to a chemotherapeutic dosing regimen, and continuing the chemotherapeutic dosing regimen by administering the chemotherapeutic agent in an additional round to the subject.

In another aspect, provided herein in is a method for administering a platelet derivative composition to a subject, comprising: administering an effective dose of platelet derivatives in the platelet derivative composition to the subject before and/or after an n^{th} round of administering a cancer therapeutic agent according to a cancer therapeutic dosing regimen, and continuing the cancer therapeutic dosing regimen by administering the cancer therapeutic agent in an additional round to the subject.

In one aspect, provided herein is a method for administering a platelet derivative composition to a subject having thrombocytopenia, comprising: administering a first round of platelets at an initial timepoint to the subject having thrombocytopenia; after the administering, determining that a platelet count in the subject did not increase by an expected amount; administering an effective dose of the platelet derivatives in a platelet derivative composition to the subject; optionally, administering a subsequent round(s) of platelets to the subject at a subsequent timepoint(s); and after the administering of the platelet derivatives, or the administering of the subsequent round(s) of the platelets, determining that the platelet count in the subject is increased to an amount closer to the expected amount, than after the first round of administering, or fall within the expected amount. In some embodiments, wherein after the administering the subsequent round(s) of the platelets, determining that the platelet count in the subject is increased by an amount to fall within the expected amount.

In another aspect, provided herein is a method for administering a platelet derivative composition to a subject having thrombocytopenia, comprising: administering platelets at an initial timepoint to the subject having thrombocytopenia, in an amount expected to increase a platelet count by an expected amount within a target platelet increase timeframe after the initial timepoint; after the administering and during the target platelet increase timeframe, determining that the platelet count did not increase by the expected amount; and within 24, 12, 8, 6, 4, 2, or 1 hour after the initial timepoint, administering an effective dose of the platelet derivatives in the platelet derivative composition to the subject. In some embodiments, wherein after the administering the effective dose of the platelet derivatives, the platelet count in the subject is increased closer to the expected amount or is increased by the expected amount without or before adding any additional platelets after the initial timepoint. In some embodiments, the platelet count in the subject is increased closer to the expected amount without or before adding any additional platelets after the initial timepoint. In other embodiments, the platelet count in the subject is increased by the expected amount without or before adding any additional platelets after the initial timepoint. In some embodiments, wherein after the administering the effective dose of the platelet derivatives, administering platelets at a subsequent timepoint to the subject, and wherein after the administering the platelets, the platelet counts in the subject is increased closer to the expected amount or is increased by the expected amount. In some embodiments, after the administering the platelets at a subsequent timepoint to the subject, the platelet counts in the subject is increased closer to the expected amount. In some embodiments, after the administering the platelets at a subsequent timepoint to the subject, the platelet counts in the subject is increased by the expected amount.

In another aspect, provided herein is a method for increasing platelet counts in a subject having thrombocytopenia, comprising: administering platelets at an initial timepoint to the subject having thrombocytopenia; after the administering and during a target platelet increase timeframe after the initial timepoint, determining that the platelet count in the subject did not increase by an expected amount based on the number of platelets that were administered; within 24, 12, 8, 6, 4, 2, 1 hour, 45 minutes, 30 minutes, 15 minutes, or 10 minutes, after the initial timepoint, administering an effective dose of the platelet derivatives in the platelet derivative composition to the subject; and after the administering the effective dose of the platelet derivatives, administering platelets at a subsequent timepoint to the subject, wherein the platelet count in the subject is increased closer to the expected amount, than the administering platelets at the initial timepoint or is increased by the expected amount. In some embodiments, in such aspects, the method can further include comprising counting platelets in the subject after the subsequent timepoint, and determining that the platelet count in the subject is increased closer to the expected amount, than the administering platelets at the initial timepoint or is increased by the expected amount.

In another aspect, provided herein is a method of treating a subject having refractory thrombocytopenia, comprising: administering an effective dose of platelet derivatives in a platelet derivative composition to the subject having refractory thrombocytopenia. In some embodiments, wherein after the administering the effective dose of the platelet derivatives, a platelet count in the subject increases. In some embodiments, the platelet count in the subject increases by an expected amount. In some embodiments, wherein after the administering the effective dose of the platelet derivatives, administering platelets at a subsequent timepoint to the subject, and wherein after the administering the platelets, the platelet count in the subject increases, and in some embodiments, the platelet count in the subject increases by an expected amount.

In another aspect, provided herein is a method for administering a platelet derivative composition to a subject having an initial low platelet count, comprising: administering platelets to the subject having the initial low platelet count at an initial timepoint in an amount predicted to increase a platelet count by an expected amount; after the administering the platelets, determining that the platelet count in the subject remains below the expected amount; administering an effective dose of the platelet derivatives in the platelet derivative composition to the subject; and after the administering the effective dose of the platelet derivatives, administering platelets at a subsequent timepoint to the subject, wherein the administering the platelets increases the platelet count closer to or by the expected amount. In some embodiments, the initial low count can be less than 300,000, 280,000, 270,000, 260,000, 250,000, 225,000, 200,000, 175,000, or 160,000 platelets per microliter of the blood.

In another aspect, provided herein is a method of treating a subject having thrombocytopenia, for example, decreasing or reducing bleeding in the subject comprising: a) administering an effective dose of platelet derivatives in a platelet derivative composition at an initial platelet derivative timepoint to the subject having thrombocytopenia; and b) within less than 1 hour, 50 minutes, 45 minutes, 35 minutes, 30 minutes, 15 minutes, or 10 minutes after the administering the effective dose of the platelet derivatives, administering platelets to the subject, wherein after the administering platelets, the number of platelets in the subject increases.

In another aspect herein, is a is a method of treating a subject having thrombocytopenia, comprising: a) administering platelets at an initial timepoint to the subject having thrombocytopenia, b) administering an effective dose of platelet derivatives in a platelet derivative composition at an initial platelet derivative timepoint to the subject, wherein the initial timepoint is more than 6, 7, 8, 9, 10, 11, or 12 hours before the initial platelet derivative timepoint; and c) administering platelets to the subject at a subsequent timepoint, wherein after the administering platelets, the number of platelets in the subject increases. In some embodiments of such aspects, the subsequent time point is less than 1 hour, 50 minutes, 45 minutes, 35 minutes, 30 minutes, 20 minutes, 15 minutes, or 10 minutes after the administering the effective dose of the platelet derivative.

In any of the aspects and embodiment herein that includes administering platelet derivatives or rehydrated platelet derivatives to a recipient or a subject herein, a dose, an effective dose, dose for continuous infusion procedure (/min), therapeutically effective dose or amount of the platelet derivatives or the rehydrated platelet derivatives in a platelet derivative composition or rehydrated platelet derivative composition or for administering is in the range of 1.0×10⁷ to 1.0×10¹²/kg of the subject. In some embodiments, the administering can be as a continuous infusion procedure and the dose can be in the range of 1.0×10⁷ to 1.0×10¹²/kg/min of the subject. In some embodiments, the administering can be the effective amount or the dose is in the range of 1.0×10⁹ to 1.0×10¹², 1.2×10⁹ to 1.0×10¹², 1.4×10⁹ to 1.0×10¹², 1.6×10⁹ to 1.0×10¹², 1.8×10⁹ to 1.0×10¹², 2.0×10⁹ to 1.0×10¹², 3.0×10⁹ to 1.0×10¹², 3.5×10⁹ to 1.0×10¹², 4.0×10⁹ to 1.0×10¹², 5.0×10⁹ to 1.0×10¹², 5.5×10⁹ to 1.0×10¹², 6.0×10⁹ to 1.0×10¹², 6.2×10⁹ to 1.0×10¹², 6.4×10⁹ to 1.0×10¹², or 6.5×10⁹ to 1.0×10¹²/kg of the subject.

In any of the aspects and embodiment herein that includes administering platelet derivatives, a dose, an effective dose, dose for continuous infusion procedure (/min), or a therapeutically effective dose can be in the range of 1.0×10⁵ to 1.0×10¹²/kg of the subject. In some embodiments, administering can be done as a continuous infusion procedure, and the dose can be in the range of 1.0×10⁵ to 1.0×10¹²/kg/min. In some embodiments, a continuous infusion procedure using any dose herein, for example, a dose from 1.0×10¹ to 1.0×10¹²/kg/min can be done for at least 15 minutes, 30 minutes, 1 hour, 2 hours, 3 hours, 4 hours, 5 hours, 6 hours, 8 hours, 10 hours, 12 hours, 18 hours, 24 hours. In some embodiments, continuous infusion using any dose herein, for example, a dose from 1.0×10⁵ to 1.0×10¹²/kg/min can be done for a time period in the range of 30 minutes to 72 hours, 30 minutes to 48 hours, 30 minutes to 24 hours, 30 minutes to 18 hours, 30 minutes to 12 hours, or 30 minutes to 6 hours. In some embodiments continuous infusion procedure can be done with platelet derivatives at a dose in the range of 1.0×10⁵ to 1.0×10⁷/kg/min, 1.0×10⁵ to 1.0×10⁸/kg/min, 1.0×10⁵ to 1.0×10⁹/kg/min, 1.0×10⁵ to 1.0×10¹⁰/kg/min, 1.0×10⁵ to 1.0×10¹¹/kg/min, 1.0×10⁶ to 1.0×10¹²/kg/min, 1.0×10⁸ to 1.0×10¹²/kg/min, or 1.0×10¹⁰ to 1.0×10¹²/kg/min. In some embodiments, administering can be done multiple times by administering a single, double, triple, or more doses, in illustrative embodiments between 1.0×10⁵ to 1.0×10¹²/kg at a frequency, for example, at every 2 minutes, 3 minutes, 4 minutes, 5 minutes, 6 minutes, 7 minutes, 8 minutes, 9 minutes, 10 minutes, 15 minutes, 20 minutes, 30 minutes, 1 hour, 2 hours, 3 hours, 4 hours, 5 hours, 6 hours, 7 hours, 8 hours, 9 hours, 10 hours, 11 hours, 12 hours, or 24 hours. In certain illustrative embodiments, dosing is performed at a time interval between every 5 minutes and every 60 minutes. In some embodiments, a single, double, triple, or more doses can be administered at a frequency of a time within the range of 2 minutes to 24 hours, 2 minutes to 12 hours, 2 minutes to 6 hours, 2 minutes to 3 hours, 2 minutes to 2 hours, 2 minutes to 1 hour, 2 minutes to 45 minutes, 2 minutes to 30 minutes, 2 minutes to 20 minutes, 2 minutes to 15 minutes, 2 minutes to 10 minutes, or 2 minutes to 5 minutes. In some embodiments, the dosage in a single, double, triple or more doses can vary as per the time from the administration of the first dose. In some embodiments, the number of doses administered over a frequency of time herein can vary from 1 dose to 10 doses, 1 dose to 8 doses, 1 dose to 6 doses, 1 dose to 4 doses, or 1 dose to 3 doses. In some embodiments, the number of doses administered over a frequency of time herein can vary from 2 doses to 10 doses, 2 doses to 8 doses, 2 doses to 6 doses, 2 dose to 4 doses, 3 doses to 10 doses, 3 doses to 8 doses, 3 doses to 6 doses, 4 doses to 10 doses, or 4 doses to 8 doses. In some embodiments, administering can be done in multiple times, and the dose can vary in the range of 1.0×10⁵ to 1.0×10¹⁰/kg, 1.0×10⁵ to 1.0×10⁸/kg, 1.0×10⁵ to 1.0×10⁷/kg, 1.0×10⁸ to 1.0×10¹²/kg, 1.0×10⁹ to 1.0×10¹²/kg, 1.0×10¹⁰ to 1.0×10¹²/kg, or 1.0×10¹¹ to 1.0×10¹²/kg of the subject. In some embodiments, the continuous infusion procedure or providing doses at a time frequency herein can be done depending on whether bleeding is reduced to a satisfactory level, for example such that it is no longer considered life-threatening, or no longer considered severe or serious, or continued until the bleeding is mild, or stops. For example, administering can be performed at a frequency of at least one dose every 2 to 5 minutes or more frequently starting from the first dose until the bleeding potential of the subject is reduced as compared to the bleeding potential before the administering. In some embodiments, administering of platelet derivatives, FDPDs, or FPH herein can be performed as a mixed procedure in which the continuous infusion can be interrupted with a specific dose of platelet derivatives followed by a specific interval as per the requirement, for example, status of bleeding of the subject or the recipient. In some embodiments, administering platelet derivatives, FDPDs, or FPH as a continuous infusion procedure, or providing single or multiple doses as per a time frequency as disclosed herein can be done as a part of a surgical procedure. In other words, a continuous infusion procedure or single or multiple dose administration can be done at a time frequency disclosed herein for a subject undergoing surgery. For example, administering platelet derivatives, FDPDs, or FPH can be done as a continuous infusion procedure during a surgery, such that any dose as disclosed herein can be infused 10, 20, 30, 40, 50, or 60 minutes, within 1 hour before, during, or within one hour after a surgery, or before a surgery is scheduled to end. In other embodiments, any dose can be provided as a continuous infusion procedure starting 60, 50, 40, 30, 20, or 10 minutes within 1 hour before, during or within 1 hour after a surgery, or before a surgery is scheduled to end. In some embodiments, the continuous infusion procedure can be done during the entire duration of the surgery. In some embodiments, the continuous infusion procedure can be done whenever there might be a risk of increased bleeding during the surgery. In some embodiments, platelet derivatives, FDPDs, or FPH can be administered every 2, 3, 4, 5, 6, 7, 8, or 9 minutes within 1 hour before, during, or within 1 hour after the surgery. In some embodiments, platelet derivatives, FDPDs, or FPH can be administered every 10, 20, 30, 40, 50, or 60 minutes within 1 hour before, during, or within 1 hour after the surgery.

In some embodiments of any aspects or embodiments herein that include a method of treating a subject having thrombocytopenia, the thrombocytopenia can be drug-induced thrombocytopenia, chemotherapy-induced thrombocytopenia, or radiation-induced thrombocytopenia.

In any aspects, or embodiments herein that include a method for administering a platelet derivative composition to a subject, the first dose of the cancer therapeutic agent administered in the post-platelet derivative cancer therapeutic dosing regimen is not delayed due to the low platelet count in the subject. In some embodiments, the first dose of the cancer therapeutic agent administered in the additional round is not delayed due to the low platelet count in the subject. In some embodiments, the first dose of the cancer therapeutic agent administered in the post-platelet derivative cancer therapeutic dosing regimen is not reduced due to the low platelet count in the subject. In some embodiments, the first dose of the cancer therapeutic agent administered in the additional round is not reduced due to the low platelet count in the subject. In some embodiments, the first dose of the cancer therapeutic agent administered in the post-platelet derivative cancer therapeutic dosing regimen is not reduced and is not delayed due to the low platelet count in the subject. In some embodiments, the first dose of the cancer therapeutic agent administered in the additional round is not reduced and is not delayed due to the low platelet count in the subject.

In any aspects, or embodiments herein that include a method for administering a platelet derivative composition to a subject, the cancer therapeutic agent is selected from the group consisting of a chemotherapeutic agent, radiation, a targeted cancer therapeutic agent, and a combination thereof. In some embodiments, the cancer therapeutic agent is the chemotherapeutic agent, radiation, or a combination thereof. In some embodiments, the cancer therapeutic agent is a chemotherapeutic agent. In some embodiments, the cancer therapeutic agent is radiation. In some embodiments, the targeted cancer therapeutic agent comprises a small-molecule cancer therapeutic agent or a biologic cancer therapeutic agent. In some embodiments, the targeted cancer therapeutic agent is selected from the group consisting of poly ADP ribose polymerase (PARP) inhibitors, immune checkpoint inhibitors (ICIs), kinase inhibitors, receptor tyrosine kinase inhibitors, non-receptor tyrosine kinase inhibitors, serine/threonine kinase inhibitors, epigenetic inhibitors, BCL-2 inhibitors, hedgehog pathway inhibitors, proteasome inhibitors, and a combination thereof. In some embodiments, the subject is further administered stem cells, T cells, NK cells, a gene therapy vector, a cancer vaccine, and/or an anti-cancer peptide. In some embodiments, the subject is bleeding at the time of the administering the platelet derivatives, and the administering leads to a decrease in the bleeding at a primary site. In such embodiments, the administering leads to a decrease or cessation in the bleeding at a primary site. In some embodiments, the administering leads to a cessation or a decrease in bleeding at a primary bleeding site at or within 15 minutes, 30 minutes, 45 minutes, 60 minutes, 2 hours, 4 hours, 6 hours, 8 hours, 10 hours, 12 hours, 24 hours, 48 hours, 72 hours, 4 days, 5 days, 6 days, or 7 days after administering the platelet derivatives. In some embodiments, administering platelet derivatives to the subject prevents bleeding in the subject. In some embodiments, the subject is diagnosed with cancer. In some embodiments, the subject is not diagnosed with cancer. In some embodiments, the subject is set to undergo or is undergoing hematopoietic stem cell transplantation or bone marrow transplantation. In some embodiments, the subject is set to undergo or is undergoing cell therapy or gene therapy.

In any aspects, or embodiments herein that include a method for administering platelet derivatives or a platelet derivative composition to a subject with thrombocytopenia or refractive thrombocytopenia the subject has taken and/or is taking a therapeutic agent for treating thrombocytopenia. In some embodiments, the subject has taken a therapeutic agent for treating thrombocytopenia. In some embodiments, the subject is taking a therapeutic agent for treating thrombocytopenia. In some embodiments, the subject has a prescribed, active, and/or detectable amount of a therapeutic agent for treating thrombocytopenia in their blood. In some embodiments, the therapeutic agent is capable of binding the thrombopoietin (TPO) receptor. In some embodiments, the therapeutic agent is TPO, romiplostim, recombinant human TPO, eltrombopag, avatrombopag, lusutrombopag, or hetrombopag.

In any aspects, or embodiments herein that include a method for administering platelet derivatives or a platelet derivative composition to a subject, after the administering the platelets at the initial timepoint, the number of platelets in the subject increases to a level that can be less than 10%, 20%, 25%, 40%, 50%, 60%, 70%, 75%, 80%, 90%, or 95% of the expected amount based on the number of platelets administered. In some embodiments, the level can be in the range of 1-95%, 1-90%, 1-80%, 1-75%, 1-65%, 1-60%, 1-50%, 1-40%, 1-30%, 1-25%, 1-20%, 1-10%, or 1-5%. In some embodiments, wherein after the administering the platelets at the subsequent timepoint, the number of platelets in the subject increases to a level that can be at least 10%, 20%, 25%, 40%, 50%, 60%, 70%, 75%, 80%, 90%, or 95% of the expected amount based on the number of platelets administered. In some embodiments, after the administering the platelets at the subsequent timepoint, the number of platelets in the subject increases around 10-99%, 10-95%, 10-90%, 10-80%, 10-75%, 10-65%, 10-50%, or 10-40%. In some embodiments, the platelet count does not increase or increases by less than 10% of the platelet count before the administering, and wherein after the administering the platelets at the subsequent timepoint, the platelet count is equal to or greater than 10% of the platelet count before the administering the platelets at the initial timepoint. In some embodiments, the target platelet increase timeframe is between 5 minutes on the low end and 36, 24, 12, 8, 6, 4, 2, or 1 hour, or 30, 15, or 10 minutes on the high end of the range, or between 10 minutes on the low end and 36, 24, 12, 8, 6, 4, 2, or 1 hour, or 30, or 15 minutes on the high end of the range, or between 10 minutes and 1 hour. In illustrative embodiments, the target platelet increase timeframe is both between 10 minutes and 1 hour in a first timeframe, and between 18 and 36 hours in a subsequent timeframe. In some embodiments, the increase in the platelet count after the administering the platelets at the subsequent timepoint is determined in a subsequent target platelet increase timeframe between 5 minutes on the low end and 36, 24, 12, 8, 6, 4, 2, or 1 hour, or 30, 15, or 10 minutes on the high end of the range, or between 10 minutes on the low end and 36, 24, 12, 8, 6, 4, 2, or 1 hour, or 30, or 15 minutes on the high end of the range, or between 10 minutes and 1 hour. In illustrative embodiments, the target platelet increase timeframe is both between 10 minutes and 1 hour in a first timeframe, and between 18 and 36 hours in a second timeframe.

In any aspects or embodiments herein, the platelet count in the subject after the administering the platelets at the initial timepoint is below a normal range of platelets, and the platelet count after the administering the platelets at the subsequent timepoint is closer to or within the normal range of platelets. In some embodiments, the method can further include wherein the administering the platelets at the initial timepoint or the subsequent timepoint comprises single or multiple (for example, two, three, four, five, or more) platelet transfusion, or platelet administration.

In any aspects or embodiments herein, the expected amount can be calculated in terms of corrected count increment (CCI), a 1-hour CCI of more than 5,000 can be the expected amount after a single platelet transfusion. In some embodiments, a CCI of more than 5000/μl can be the expected amount after two platelet transfusions of blood-compatible platelet transfusion, in illustrative embodiments, ABO-compatible platelet transfusion. In some embodiments, a 1-hour corrected count increment (CCI) of more than 7,500 can be the expected amount after a single platelet transfusion. In some embodiments, a 1-hour corrected count increment (CCI) of more than 10,000 can be the expected amount after a single platelet transfusion.

In any aspects or embodiments herein, the expected amount can be calculated in terms of percent platelet recovery (PPR), a percent platelet recovery (PPR) of more than 30%, 35%, or 40% can be the expected amount after a single platelet transfusion of blood-compatible platelets, in illustrative embodiments, ABO-compatible platelets.

In any aspects or embodiments herein, the expected amount can be calculated in terms of the platelet count increments, platelet count increments above 5×10⁹ platelets/L at a standard platelet increase timeframe can be the expected amount after the transfusion of blood-compatible platelets, for example, ABO-compatible platelets. In illustrative embodiments, the platelet count increments can be assessed at a timeframe of 18-24 hours.

In any aspects or embodiments herein, the administering the platelets at an initial timepoint or a subsequent timepoint comprises a platelet transfusion. Platelet transfusion herein can comprise of 1 or more than 1 number of transfusions at an initial timepoint or at a subsequent timepoint. In some embodiments, administering platelets can comprise 1, 2, 3, 4, 5, 6, 7, 8, 9, or 10 platelet transfusions at an initial timepoint. In some embodiments, administering platelets can comprise 2-10, 2-8, 2-6, or 2-4 platelet transfusions at an initial timepoint. In some embodiments, administering platelets can comprise 1, 2, 3, 4, 5, 6, 7, 8, 9, or 10 platelet transfusions at an subsequent timepoint. In some embodiments, administering platelets can comprise 2-10, 2-8, 2-6, or 2-4 platelet transfusions at an subsequent timepoint.

In any aspects or embodiments herein, after a platelet transfusion at an initial timepoint, a CCI at 1 hour of target platelet increase timeframe (or 1-hour CCI) can be less than 10,000, 7,500, 5,000, 4,000, or 3,000. In some embodiments, after a platelet transfusion at an initial timepoint, a CCI at 2 hours of target platelet increase timeframe (or 2-hour CCI) can be less than 10,000, 7,500, 5,000, 4,000, or 3,000. In some embodiments, after a single platelet transfusion at an initial timepoint at 1-hour or 2-hour CCI can be less than 10,000, 7,500, 5,000, 4,000, or 3,000. In some embodiments, after 2 platelet transfusions at an initial timepoint at 1-hour or 2-hour CCI can be less than 10,000, 7,500, 5,000, 4,000, or 3,000.

In any aspects or embodiments herein, after a platelet transfusion at a subsequent timepoint, a CCI at 1 hour of target platelet increase timeframe (or 1-hour CCI) can be more than 10,000, 7,500, 5,000, 4,000, or 3,000. In some embodiments, after a platelet transfusion at a subsequent timepoint, a CCI at 2 hours of target platelet increase timeframe (or 2-hour CCI) can be more than 10,000, 7,500, 5,000, 4,000, or 3,000. In some embodiments, after a single platelet transfusion at a subsequent timepoint at 1-hour or 2-hour CCI can be more than 10,000, 7,500, 5,000, 4,000, or 3,000. In some embodiments, after 2 platelet transfusions at a subsequent timepoint at 1-hour or 2-hour CCI can be more than 10,000, 7,500, 5,000, 4,000, or 3,000.

In any aspects or embodiments herein, the method further comprising within 24, 12, 8, 6, 4, 2, or 1 hour before the administering the effective dose of the platelet derivatives, determining that the subject has refractory thrombocytopenia. In some embodiments, comprising in a time range of 1-24 hours, 1-18 hours, 1-12 hours, 1-8 hours, 1-6 hours, or 1-4 hours before the administering the effective dose of the platelet derivatives, determining that the subject has refractory thrombocytopenia. In some embodiments, at the time of the administering platelets at the initial timepoint, the subject has refractory thrombocytopenia. In some embodiments, the subject has been diagnosed with refractory thrombocytopenia within 7, 6, 5, 4, 3, 2, or 1 day, or within 12, 8, 6, 4, 3, 2, or 1 hour, or within 45, 30, 15, 10, or 5 minutes, or at the time of, the administering platelet at the initial timepoint. In some embodiments, the subject has been diagnosed with refractory thrombocytopenia within 1-10 days, 1-7 days, 1-5 days, or 2-6 days, or at the time of, the administering platelet at the initial timepoint. In some embodiments, the subject has been diagnosed with refractory thrombocytopenia within 5 minutes-12 hours, 5 minutes-10 hours, 5 minutes-6 hours, 5 minutes-3 hours, or 5 minutes-1 hour, or at the time of, the administering platelet at the initial timepoint. In some embodiments, after the administering the platelets at the initial timepoint, the platelet count is predicted to increase to the expected amount within a target platelet increase timeframe. In some embodiments, the target platelet increase timeframe is between 5 minutes on the low end and 36, 24, 12, 8, 6, 4, 2, or 1 hour, or 30, 15, or 10 minutes on the high end of the range, or between 10 minutes on the low end and 36, 24, 12, 8, 6, 4, 2, or 1 hour, or 30, or 15 minutes on the high end of the range, or between 10 minutes and 1 hour. In some embodiments, after the administering the platelets at the subsequent timepoint, the platelet count is closer to the expected amount than the platelet count after the administering at the initial timepoint.

In any aspects or embodiments herein, the subsequent timepoint can be after at least 90, 95, or 99% of the platelet derivatives have been cleared from the blood of the subject. In such embodiments, the platelet count can be closer to the expected amount than the platelet count after the administering at the first timepoint. In some embodiments, at least 50, 60, 70, 75, 80, 90, 95, or 99% of the platelet derivatives are cleared from the subject's blood within 2 hours, 1 hour, 45 minutes, 30 minutes, 15 minutes, 10 minutes, or 5 minutes after the administering the platelet derivatives. In some embodiments, at least 90, 95, or 99% of the platelet derivatives are cleared from the subject's blood within 2 hours, 1 hour, 45 minutes, 30 minutes, 15 minutes, 10 minutes, or 5 minutes after the administering the platelet derivatives, and in illustrative embodiments, within 30 minutes, 15 minutes, 10 minutes, or 5 minutes after the administering the platelet derivatives. In some embodiments, at least 99% of the platelet derivatives are cleared from the subject's blood within 30 minutes, 15 minutes, 10 minutes, or 5 minutes after the administering the Platelet derivatives, and in illustrative embodiments, wherein 99.9% of detectable platelet derivatives are cleared from the subject's blood within 30 minutes, 15 minutes, 10 minutes, or 5 minutes after the administering the platelet derivatives.

In another aspect, provided herein is a platelet derivative composition for use in administering to a subject having Hermansky Pudlak Syndrome (HPS), wherein the administering comprises administering an effective dose of platelet derivatives in the platelet derivative composition to the subject, wherein the platelet derivatives a) have the ability to generate thrombin in vitro in the presence of tissue factor and phospholipids; b) have the ability to occlude a collagen-coated microchannel in vitro; or c) both a) and b).

In another aspect, provided herein is a platelet derivative composition for use in administering to a subject having Hermansky Pudlak Syndrome (HPS) or Bernard Soulier Syndrome (BSS), wherein the administering comprises administering an effective dose of platelet derivatives in the platelet derivative composition to the subject.

In another aspect, provided herein, is a method for administering a platelet derivative composition to a subject having Hermansky Pudlak Syndrome (HPS) or Bernard Soulier Syndrome (BSS), comprising: administering an effective dose of the platelet derivatives in a platelet derivative composition to the subject, wherein the platelet derivative composition comprises a population of platelet derivatives.

In any aspects, or embodiments herein that include a platelet derivative composition for use, or a method of administrating a platelet derivative composition for use, the subject can be bleeding at the start of the administering. In some embodiments, the administering can leads to cessation of bleeding. In some embodiments, the administering can lead to a decrease in bleeding within 6, 12, 18, 24, 30, or 36 hours after the start of the administering. In some embodiments, the administering can be performed until the bleeding stops. In some embodiments, the administering can be performed for no more than 36, 30, 24, 18, 12, 6 hours or less. In some embodiments, the administering can be performed until there is cessation of bleeding at a primary bleeding site. In some embodiments, the administering can lead to cessation of bleeding within 6, 12, 18, 24, 30, or 36 hours after the administering.

In some embodiments of any aspects or embodiments herein that include administering, the platelet derivatives can have a compromised plasma membrane, wherein at least 30%, 40%, 50%, 60%, 70%, or 80% of the platelet derivatives are CD 41-positive platelet derivatives, and wherein the platelet derivatives a) have the ability to generate thrombin in vitro in the presence of tissue factor and phospholipids; b) have the ability to occlude a collagen-coated microchannel in vitro; or c) both a) and b). In some embodiments, the administering can increase the levels of at least one, two, and/or all platelet biomarker selected from CD62P, PAC-1, and CD63 for endogenous platelets of the subject as compared to before the administering. In some embodiments, the levels of platelet biomarkers CD62P and PAC-1 can be increased.

In some embodiments of any aspects or embodiments herein, the subject can have HPS, and the administering can be performed to treat the subject, and wherein either a) at least one HPS-related hemostatic abnormality and/or HPS-related biomarker abnormality observed in the subject is improved in the subject after the administering compared to before the administering; or b) normal levels of hemostasis and/or the HPS-related biomarker abnormalities are maintained in the subject. In some embodiments of any aspects or embodiments herein, subject has BSS, and the administering can be performed to treat the subject, and the ability to occlude a collagen-coated microchannel in vitro can be restored or improved in the subject after the administering compared to before the administering.

In any aspects, or embodiments herein that include administering, the administering can lead to an improvement in thrombin generation in the subject as compared to the subject before the administering and/or an improvement in clot formation in the subject as compared to the subject before the administering. In some embodiments, the administering can lead to an improvement in clot formation in the subject as compared to the subject after being administered apheresis platelets, but before the administering of the platelet derivatives. In some embodiments, the administering is performed to treat the subject, and normal levels of hemostasis and/or HPS-related biomarker abnormalities can be maintained in the subject. In some embodiments, the subject is taking a) one or more anti-coagulants, b) one or more anti-platelet agents, or both a) and b). In some embodiments, no more than 2%, 3%, 4%, 5%, 6%, 7%, 8%, 9%, 10%, or 25% of the platelet derivatives in the population aggregate under aggregation conditions comprising an agonist but no platelets, and in the absence of a divalent cation. In some embodiments, the agonist is selected from the group consisting of collagen, epinephrine, ristocetin, arachidonic acid, adenosine di-phosphate, and thrombin receptor associated protein (TRAP).

In any aspects, or embodiments herein that provide a platelet derivative composition for use, or a method of administrating a platelet derivative composition for use, at least 50% of the platelet derivatives have a diameter in the range of 0.5 to 25.0 μm, 20.0 μm, 15.0 μm, 12.5 μm, 10.0 μm, or 2.5 μm by flow cytometry, and wherein at least at least 40%, 45%, 50%, 55%, 60%, 65%, 70%, or 75% of the platelet derivatives are CD42 positive. In some embodiments, the composition when rehydrated comprises less than or equal to 5%, 10%, or 15% plasma protein, and wherein the platelet derivatives have less than 5% microparticles having a diameter less than 0.1 μm, 0.2 μm, 0.25 μm, or 0.5 μm by scattering intensity. In some embodiments, less than 5% of CD 41-positive platelet derivatives are microparticles having a diameter of less than 0.5 μm, at least 40%, 45%, 50%, 55%, 60%, 65%, 70%, or 75% of the platelet derivates are CD 42 positive, the composition comprises a population of platelet derivatives having a reduced propensity to aggregate such that no more than 4%, 6%, 8%, 10%, 12%, 14%, or 16% of the platelet derivatives in the population aggregate under aggregation conditions comprising an agonist but no platelets, and no divalent cation, at least 50% of the platelet derivatives in the composition are at least 0.25 μm, 0.5 μm, 1.0, 1.5 μm, 2.0 μm, 3.0 μm, 4.0 μm, 5.0 μm, 10 μm, 15 μm, or 25 μm in diameter by scattering intensity, and/or and at least 50% of the platelet derivatives in the composition are between the range of 0.5 to 25.0 μm, 20.0 μm, 15.0 μm, 12.5 μm, 10.0 μm, or 2.5 μm in diameter by scattering intensity.

In any aspects, or embodiments herein that provide a platelet derivative composition for use, or a method of administrating a platelet derivative composition for use, the effective dose of the platelet derivatives is in the range of 5.0×10¹⁰ to 1.0×10¹²/kg of the subject, 5.0×10¹⁰ to 5.0×10¹¹/kg of the subject, 5.0×10¹⁰ to 1.0×10¹¹/kg of the subject, 5.0×10⁹ to 1.0×10¹¹/kg of the subject, 5.0×10⁹ to 5.0×10¹⁰/kg of the subject, or 5.0×10⁹ to 1.0×10¹⁰/kg of the subject. In illustrative embodiments, the effective dose of the platelet derivatives is in the range of 1.0×10⁷ to 1.0×10¹¹/kg of the subject, 1.0×10⁷ to 1.0×10¹⁰/kg of the subject, or 1.0×10⁹ to 1.0×10¹²/kg of the subject, or 5×10⁸ particles/kg to 1.0×10¹² particles/kg of the subject, or 1×10⁹ particles/kg to 1.0×10¹⁰ particles/kg of the subject.

In any aspect or embodiment herein, the platelets derivatives can be obtained from one or more of plurality of containers each containing a platelet derivative composition in the form of a powder, wherein the platelet derivative composition comprises a population of platelet derivatives comprising CD 41-positive platelet derivatives, wherein less than 10%, 8%, in illustrative embodiments, less than 5% of the CD 41-positive platelet derivatives are microparticles having a diameter of less than 0.3 μm, 0.4 μm, 0.5 μm, 0.6 μm, 0.7 μm, 0.8 μm, 0.9 μm, or 1 μm having a reduced propensity to aggregate such that no more than 2%, 3%, 4%, 5%, 6%, 7%, 8%, 9%, 10%, or 25% of the platelet derivatives in the population aggregate under aggregation conditions comprising an agonist but no platelets, and having a potency of at least 0.5, 1.0, and in illustrative embodiments, 1.5 thrombin generation potency units (TGPU) per 10⁶ platelet derivatives.

In any aspect or embodiment herein, the platelets derivatives can be prepared using a process comprising performing tangential flow filtration (TFF) of a platelet composition in a solution to at least partially exchange the solution with a preparation agent having a pH in the range of 5.5 to 8.0 and comprising 0.4 to 35% trehalose and 2% to 8% polysucrose, wherein said TFF is performed using a 0.3 to 1 micron filter, thereby preparing a TFF-treated composition comprising 100×10³-20,000×10³ platelets/μL, 1000×10³-20,000×10³ platelets/μL, 1000×10³-10,000×10³ platelets/μL, 500×10³-5,000×10³ platelets/μL, 1000×10³-5,000×10³ platelets/μL, 2000×10³-8,000×10³ platelets/μL, or 15,000×10³-20,000×10³ platelets/μL, in illustrative embodiments, 10,000×10³ to 20,000×10³ platelets/μl in an aqueous medium having less than or equal to 5%, 10%, or 15% plasma protein, and having less than 10%, 8%, in illustrative embodiments, less than 5.0% microparticles having a radius less than 0.1 μm, 0.2 μm, 0.25 μm, or 0.5 μm, by scattering intensity; and freeze drying the TFF-treated composition comprising platelets in the aqueous medium to form a freeze-dried composition comprising platelet derivatives to form the platelet derivative composition, wherein the platelet derivatives in the platelet derivative composition display a potency of at least 0.5, 1.0, and in illustrative embodiments, 1.5 thrombin generation potency units (TGPU) per 10⁶ platelet derivatives, and a reduced propensity to aggregate such that no more than 2%, 3%, 4%, 5%, 6%, 7%, 8%, 9%, 10%, or 25% of the platelet derivatives in the population aggregate under aggregation conditions comprising an agonist but no platelets.

In any aspect or embodiment herein, the platelet derivative composition can initially be, and/or is typically a rehydrated form of a platelet derivative composition that before rehydration was in the form of a powder, comprising a population of platelet derivatives having a compromised plasma membrane and a reduced propensity to aggregate such that no more than 2%, 3%, 4%, 5%, 6%, 7%, 8%, 9%, 10%, or 25% of the platelet derivatives in the population aggregate under aggregation conditions comprising an agonist but no platelets, wherein at least 30%, 40%, 50%, 60%, 70%, or 80% of the platelet derivatives are CD 41-positive platelet derivatives, wherein less than 1%, 2%, 3%, 4%, or 5% of the CD 41-positive platelet derivatives are microparticles having a diameter or radius of less than 0.3 μm, 0.4 μm, 0.5 μm, 0.7 μm, or 1 μm and wherein the platelet derivatives have a potency of at least 0.5, 1.0, and in illustrative embodiments, 1.5 thrombin generation potency units (TGPU) per 10⁶ platelet derivatives.

To reiterate, any embodiments herein in this section and in this specification and associated claims, can be combined and/or used in any of the aspects herein and in combination with any of the other embodiments herein. Furthermore, a “powder” recited in any aspect or embodiment can alternatively be a solid, or a composition comprising less than 1% water content in such aspect or embodiment.

In certain illustrative embodiments of a composition, or in some compositions used in a method herein, or formed by a process herein, the platelet derivatives in a composition, as a non-limited example a powder, and/or formed by a process disclosed herein, are surrounded by a compromised plasma membrane, are positive for CD 41, and/or are 0.5 to 25.0 μm, 20.0 μm, 15.0 μm, 12.5 μm, 10.0 μm, or 2.5 μm in radius or diameter. In some embodiments, the composition comprises platelet derivatives such that at least 95% platelet derivatives positive for CD 41 have a radius or diameter in the range of 0.5 to 25.0 μm, 20.0 μm, 15.0 μm, 12.5 μm, 10.0 μm, or 2.5 μm. Such radius or diameter can be measured, for example by flow cytometry technique as known to a skilled artisan in the art.

In some embodiments of any of the aspects and embodiments herein that include platelet derivatives in a hydrated or rehydrated form, the protein concentration, or plasma protein concentration, is in the range of 0.01%-50%, 5%-50%, 5%-30%, 5-15%, 8%-10%, 7%-10%, or 3-7% of the protein concentration of donor apheresis plasma. In some embodiments of a composition or in some compositions used in or formed by a process herein, the protein concentration, or plasma protein concentration is less than or equal to 30%, 25%, 20%, 15%, 10%, 5%, 4%, 3%, 2%, or 1% of the protein concentration of donor apheresis plasma. In some embodiments of a composition or a process herein, the protein concentration, or plasma protein concentration is less than or equal to 15%, 14%, 13%, 12%, 11%, 10%, 9%, 8%, 7%, 6%, 5%, 4%, 3%, 2%, 1%, 0.1%, or 0.01%. In some exemplary embodiments, the protein concentration, or plasma protein concentration is less than 3% or 4%. In some embodiments, the protein concentration, or plasma protein concentration is between 0.01% and 20%, 0.01% and 15%, 0.01% and 10%, 0.010% and 5%, 0.10% and 20%, 0.10% and 15%, 0.10% and 10%, 0.10% and 5%, 1% and 20%, 1% and 15%, 1% and 10%, 1% and 5%, 2% and 10%, 2% and 5%, 2.5% and 5%, 2.5% and 7.5%, or between 3% and 5%. In some embodiments of a composition or a process herein, the protein concentration is in the range of 0.01-15%, 0.1-15%, 1-15%, 1-10%, 0.01-10%, 3-12%, or 5-10%. In some embodiments, the absorbance at 280 nm is less than or equal to 2.0 AU, or 1.90 AU, or 1.80 AU, or 1.7 AU, or 1.66 AU, or 1.6 AU when measured using a path length of 0.5 cm.

In some embodiments of any of the aspects and embodiments herein that include platelet derivatives in a powdered form, the protein concentration is in the range of 0.01-15%, 0.1-15%, 1-15%, 1-10%, 0.01-10%, 3-12%, or 5-10%. In some embodiments, the protein concentration is less than or equal to 25%, 20%, 15%, 10%, 7.5% 5%, 2.5%, 1%, or 0.1%.

In some embodiments of any of the aspects and embodiments herein that include a process for preparing a platelet derivative composition, the process comprises performing TFF of a platelet composition in a solution to at least partially exchange the solution with a preparation agent having a pH in the range of 5.5 to 8.0 and comprising trehalose and polysucrose, wherein said TFF is performed using a 0.3 to 1 micron filter, thereby preparing a TFF-treated composition.

In some embodiments of any of the aspects and embodiments herein that include a composition or in some compositions used in or formed by a process that includes a population of platelet derivatives in a hydrated or rehydrated form, the composition comprises less than 10%, 7.5%, 7%, 6%, 5%, 4.5%, 4%, 3.5%, 3%, 2.5%, 2%, 1.5%, 1.0%, 0.75%, 0.5%, 0.25%, or 0.1% (by scattering intensity) microparticles. In some embodiments, the composition comprises microparticles (by scattering intensity) in the range of 0.01%-10%, 0.01%-7.5%, 0.01%-5%, 0.1%-10%, 0.1%-5%, 0.1%-4.9%, 0.5%-4.5%, 1%-10%, 1%-5%, 0.01%-4%, −0.1%-4%, 1%-4%, 1.5%-3%, 0.1%-3%, or 1%-3%. In some embodiments, the microparticles have a diameter less than 1 μm. In illustrative embodiments, the microparticles have a radius or diameter less than 0.5 μm. In some embodiments, the microparticles have a radius or diameter in the range of 0.01-0.5 μm, 0.1-0.5 μm, or 0.1-0.49 μm, 0.1-0.47 μm, or 0.1-0.45 μm, or 0.1-0.4 μm, or 0.2-0.49 μm, or 0.25-0.49 μm, or 0.3-0.47 μm. In some embodiments, the radius or diameter of the microparticles is measured using flow cytometry.

In some embodiments of any of the aspects and embodiments herein that include a platelet derivative composition in a powdered form, the platelet derivative composition comprises a population of platelet derivatives comprising CD41-positive platelet derivatives, wherein less than 5%, 4.5%, 4%, 3.5%, 3%, 2.5%, 2%, 1.5%, 1%, 0.5%, or 0.1% of the CD41-positive platelet derivatives are microparticles having a diameter of less than 1 μm, 0.9 μm, 0.8 μm, 0.7 μm, 0.6 μm, 0.5 μm 0.4 μm, 0.3 μm, 0.2 μm, or 0.1 μm. In some embodiments, 0.01%-4.9%, 0.1%-4.9%, 0.5%-4.5%, 0.01%-4%, 0.1%-4%, 1%-4%, 1.5%-3%, 0.1%-3%, or 1%-3% of the CD-41-positive platelet derivatives are microparticles.

In some embodiments, the platelet derivative composition comprises a population of platelet derivatives comprising CD42-positive platelet derivatives, wherein less than 5%, 4.5%, 4%, 3.5%, 3%, 2.5%, 2%, 1.5%, 1%, 0.5%, or 0.1% of the CD42-positive platelet derivatives are microparticles having a diameter of less than 1 μm, 0.9 μm, 0.8 μm, 0.7 μm, 0.6 μm, 0.5 μm, 0.4 μm, 0.3 μm, 0.2 μm, or 0.1 μm. In some embodiments, 0.01%-4.9%, 0.1%-4.9%, 0.5%-4.5%, 0.01%-4%, 0.1%-4%, 1%-4%, 1.5%-3%, 0.1%-3%, or 1%-3% of the CD-42-positive platelet derivatives are microparticles. In some embodiments, the platelet derivative composition comprises a population of platelet derivatives comprising CD61-positive platelet derivatives, wherein less than 5%, 4.5%, 4%, 3.5%, 3%, 2.5%, 2%, 1.5%, 1%, 0.5%, or 0.1% of the CD61-positive platelet derivatives are microparticles having a diameter of less than 1 μm, 0.9 μm, 0.8 μm, 0.7 μm, 0.6 μm, 0.5 μm, 0.4 μm, 0.3 μm, 0.2 μm, or 0.1 μm. In some embodiments, 0.01%-4.9%, 0.1%-4.9%, 0.5%-4.5%, 0.01%-4%, 0.1%-4%, 1%-4%, 1.5%-3%, 0.1%-3%, or 1%-3% of the CD-62-positive platelet derivatives are microparticles. In some illustrative embodiments, the microparticles are having a diameter of less than 0.5 μm. In some embodiments of any of the aspects and embodiments herein that include a platelet derivative composition in a powdered form, the diameter of the microparticles is determined after rehydrating the platelet derivative composition with an appropriate solution. In some embodiments, the amount of solution for rehydrating the platelet derivative composition is equal to the amount of buffer or preparation agent present at the step of freeze-drying. In some embodiments, the diameter of the microparticles is determined by flow cytometry.

In some embodiments of any of the aspects and embodiments herein that include a composition or in some compositions used in or formed by a process, the platelet derivatives have a radius or diameter greater than 0.25 μm, greater than 0.3 μm, greater than 0.4 μm, or in illustrative embodiments, greater than 0.5 μm. In some embodiments, the platelet derivatives have a radius or diameter greater than 0.75 μm. In some embodiments, the platelet derivatives have a radius or diameter in the range of 0.25-4 μm, 0.27-3.5 μm, 0.3-3.25 μm, 0.35-3.50 μm, or 0.4-3 μm. In illustrative embodiments, the platelet derivatives have a radius or diameter of at least 0.5 μm, for example in the range of 0.5 μm on the low end of the range to 25.0 μm, 20.0 μm, 15.0 μm, 12.5 μm, 10.0 μm, 5.0 μm or 2.5 μm on the high end of the range. In some embodiments, the diameter of the platelet derivatives is measured using flow cytometry.

In some embodiments of any of the aspects and embodiments herein that include a composition or in some compositions used in or formed by a process, the platelet derivatives are CD-41 positive. In some embodiments, at least 40%, 45%, 50%, 55%, 60%, 65%, 70%, or 75% of the platelet derivatives are CD-41 positive. In some embodiments, the platelet derivatives in the range of 35-97%, 40-97%, 50-97%, 60-97%, 40-95%, 45-90%, 50-95%, 60-90%, or 75-95% are positive for CD-41. In some embodiments, at least 50% of the platelet derivatives are CD 41-positive platelet derivatives, wherein less than 5% of the CD 41-positive platelet derivatives are microparticles having a diameter of less than 0.5 μm, and wherein the platelet derivatives have a potency of at least 1.5 thrombin generation potency units (TGPU) per 10⁶ platelet derivatives.

In some embodiments of any of the aspects and embodiments herein that include a platelet derivative composition in a powdered form, the platelet derivatives in the platelet derivative composition have a weight percentage of at least 0.5%, 0.75%, 1%, 1.5%, 2%, 2.5% 3%, 3.5%, 4%, 4.5%, 5%, 5.5%, 6%, 6.5%, 7%, 7.5%, 8%, 8.5%, 9%, 9.5%, or 10%. In some embodiments, the platelet derivatives in the platelet derivative composition have a weight percentage in the range of 0.5 to 25%, 0.5% to 20%, 1% to 20%, 2.5% to 20%, 5% to 20%, 5% to 10%, 2.5% to 20%, 2.5% to 15%, 2.5% to 10%, or 2.5% to 7.5%.

In some embodiments of the aspects and embodiments herein that include a platelet derivative composition in a powdered form, a platelet derivative composition is devoid of plasma protein. In some embodiments, the plasma protein is in the range of 0.01-15%, 0.1-15%, 1-10%, 2-15%, 3-9%, 1-5%, 1-3%, 0.1-3%, 0.5-2%, or 0.25-2%.

In some embodiments of the aspects and embodiments herein that include a platelet derivative composition in a powdered form, a platelet derivative composition comprises a buffering agent in the range of 0.5-3%, 0.75-2.75%, 1-2.5%, or 1.5-2.5%. In some embodiments, the buffering agent is HEPES.

In some embodiments of any of the aspects and embodiments herein that include a composition or in some compositions used in or formed by a process, the platelet derivatives have an inability to increase expression of a platelet activation marker in the presence of an agonist as compared to the expression of the platelet activation marker in the absence of the agonist. In some embodiments, the platelet activation marker is selected from the group consisting of Annexin V, and CD 62. In some embodiments, the platelet activation marker is Annexin V. In some embodiments, the platelet activation marker is CD 62. In some embodiments, the agonist is selected from the group consisting of collagen, epinephrine, ristocetin, arachidonic acid, adenosine di-phosphate, and thrombin receptor associated protein (TRAP).

In some embodiments of any of the aspects and embodiments herein that include a composition, or in some compositions used in or formed by a process, the aqueous medium has a concentration of human leukocyte antigen (HLA) Class I antibodies that is less than 30%, 10%, 5%, 3%, or 1% of the human leukocyte antigen (HLA) Class I antibody concentration in donor apheresis plasma. In some embodiments, the aqueous medium has a concentration of human leukocyte antigen (HLA) Class II antibodies that is less than 30%, 10%, 5%, 3%, or 1% of the human leukocyte antigen (HLA) Class II antibody concentration in donor apheresis plasma. In some embodiments, the composition is negative for HLA Class I antibodies based on a regulatory agency approved test. In some embodiments, the composition is negative for HLA Class II antibodies based on a regulatory agency approved test. In some embodiments of the composition, a percentage of beads positive for HLA Class I antibodies, as determined for the composition by flow cytometry using beads coated with Class I HLAs, is less than 5%, 3%, or 1%. In some embodiments of the composition, a percentage of beads positive for HLA Class II antibodies, as determined for the composition by flow cytometry using beads coated with Class II HLAs is less than 5%, 3%, or 1%.

In some embodiments of any of the aspects and embodiments herein that include a composition, or in some compositions used in or formed by a process, the aqueous medium has a concentration of human neutrophil antigen (HNA) antibodies that is less than 30%, 10%, 5%, 3%, or 1% of the HNA antibody concentration in donor apheresis plasma. In some embodiments, the composition is negative for HNA antibodies based on a regulatory agency approved test. In some embodiments of the composition, a percentage of beads positive for HNA antibodies, as determined for the composition by flow cytometry using beads coated with HNAs is less than 5%, 3%, or 1%.

In some embodiments of any of the aspects and embodiments herein that include a composition, or in some compositions used in or formed by a process, a percentage of beads positive for an antibody selected from the group consisting of HLA Class I antibodies, HLA Class II antibodies, and HNA antibodies, as determined for the composition by flow cytometry using beads coated with Class I HLAs, Class II HLAs, or HNAs, respectively, is less than 5%, 3%, or 1%. In some embodiments of the composition, a percentage of beads positive for HLA Class I antibodies, HLA Class II antibodies, and HNA antibodies, as determined for the composition by flow cytometry using beads coated with Class I HLAs, Class II HLAs, or HNAs, respectively, is less than 5%, 3%, or 1%. In some embodiments, the composition is negative for the antibodies selected from the group consisting of HLA Class I antibodies, HLA Class II antibodies, and HNA antibodies based on a regulatory agency approved test for the respective antibodies. In some embodiments, the composition is negative for HLA Class I antibodies, HLA Class II antibodies, and HNA antibodies based on a regulatory agency approved test for the respective antibodies.

In some embodiments of any of the aspects and embodiments herein that include a composition, or in some compositions used in or formed by a process that includes a population of platelet derivatives in a hydrated or rehydrated form, the platelets or platelet derivatives in a composition are at least 100×10³ platelets/μL, or 200×10³ platelets/μL, or 400×10³ platelets/μL, or 1000×10³ platelets/μL, or 1250×10³ platelets/μL, or 1500×10³ platelets/μL, or 1750×10³ platelets/μL, 2000×10³ platelets/μL, or 2250×10³ platelets/μL, or 2500×10³ platelets/μL, or 2750×10³ platelets/μL, or 3000×10³ platelets/μL, 3250×10³ platelets/μL, 3500×10³ platelets/μL, 3750×10³ platelets/μL, 4000×10³ platelets/μL, or 4250×10³ platelets/μL, or 4500×10³ platelets/μL, or 4750×10³ platelets/μL, or 5000×10³ platelets/μL, or 5250×10³ platelets/μL, or 5500×10³ platelets/μL, or 5750×10³ platelets/μL, or 6000×10³ platelets/μL, or 7000×10³ platelets/μL, or 8000×10³ platelets/μL, or 9000×10³ platelets/μL, or 10,000×10³ platelets/μL, or 11,000×10³ platelets/μL, or 12,000×10³ platelets/μL, or 13,000×10³ platelets/μL, or 14,000×10³ platelets/μL, or 15,000×10³ platelets/μL, or 16,000×10³ platelets/μL, or 17,000×10³ platelets/μL, or 18,000×10³ platelets/μL, or 19,000×10³ platelets/μL, or 20,000×10³ platelets/μL. In some embodiments of the composition, the platelets or platelet derivatives in the composition is in the range of 100×10³-20,000×10³ platelets/μL, 1000×10³-20,000×10³ platelets/μL, 1000×10³-10,000×10³ platelets/μL, 500×10³-5,000×10³ platelets/μL, 1000×10³-5,000×10³ platelets/μL, 2000×10³-8,000×10³ platelets/μL, 10,000×10³-20,000×10³ platelets/μL, 15,000×10³-20,000×10³ platelets/μL, 5000×10³ to 20,000×10³ platelets/μl, 6000×10³ to 18,000×10³ platelets/μl or 6000×10³ to 15,000×10³ platelets/μl.

In some embodiments, the above concentrations are at any point in a process herein, such as in the volume that is freeze dried. In some embodiments, the above concentrations are for platelet-derivatives herein. It is contemplated that the platelet derivative composition in the form of a powder has to be rehydrated with a solution to determine the platelet-derivative concentration, typically in the intended volume for rehydration of a powder, e.g. freeze-dried, composition, which in illustrative embodiments is a recommended volume of a container containing the powder and/or a same volume as the composition was in before it was dried to form the powder. In some embodiments, the solution for rehydrating can be water. In some embodiments, the solution for rehydrating can be a well-known buffer. In some embodiments, the amount of solution for rehydrating the platelet derivative composition is equal to the amount of buffer or preparation agent present at the step of freeze-drying. In some embodiments, the platelet concentration is in

In some embodiments of any of the aspects and embodiments herein that include a composition, or in some compositions used in or formed by a process, the composition(s) comprises a population of platelet derivatives having a reduced propensity to aggregate. In certain embodiments, no more than 25%, 22.5%, 20%, 17.5%, 12.5%, 10%, 7.5%, 5%, 4%, 3%, 2%, or 1% of the platelet derivatives in the population aggregate under aggregation conditions. In an illustrative embodiment no more than 10% of the platelet derivatives in the population aggregate under aggregation conditions. Illustrative embodiments of exemplary aggregation conditions are provided herein. For example, in illustrative embodiments such aggregation conditions comprise an agonist but no platelets are present in the aggregation conditions. In some embodiments, the agonist is selected from the group consisting of collagen, epinephrine, ristocetin, arachidonic acid, adenosine di-phosphate, and thrombin receptor associated protein (TRAP). In some embodiments, the population of platelet derivatives aggregate in the range of 2-30%, 5-25%, 10-30%, 10-25%, 12.5-25%, 2-10%, 2-8%, 2-7.5%, 2-5%, 2-4%, 0-1%, 0-2%, 0-3%, 0-4%, 0-5%, 0-7.5%, or 0-10%, or in illustrative embodiments 0 to about 1% of the platelet derivatives under aggregation conditions comprising an agonist but no platelets. It can be contemplated that aggregation conditions involve rehydrating the platelet derivative composition in an appropriate amount of water or an appropriate buffer.

In some embodiments of any of the aspects and embodiments herein that include a composition, or in some compositions used in or formed by a process, comprises erythrocytes in an amount lesser than 0.2×10⁶ erythrocytes/μL, or 0.1×10⁶ erythrocytes/μL, or 0.5×10⁵ erythrocytes/μL, or 0.1×10⁵ erythrocytes/μL. In some embodiments, the erythrocytes in the composition is in the range of 0.1×10⁵ erythrocytes/μL to 0.2×10⁶ erythrocytes/μL, or 0.5×10⁵ erythrocytes/μL to 0.1×10⁶ erythrocytes/μL.

In some embodiments of any of the aspects and embodiments herein that include a composition, or in some compositions used in or formed by a process that includes a population of platelet derivatives in a hydrated or rehydrated form, comprises trehalose in the range of 0.4-35%, or 1-35%, or 2-30%, 1-20%, or 1-10%, or 1-5%, or 0.5-5%. In an exemplary embodiment, the composition comprises 3.5% trehalose.

In some embodiments of any of the aspects and embodiments herein that include a composition, or in some compositions used in or formed by a process that includes a platelet composition in a powdered form, comprises trehalose having a weight percentage in the range of 10-60%, 15-55%, 20-60%, 20-50%, 25-60%, 25-50%, 10-50%, 20-40%, 20-35%, or 1-20%. In some embodiments, the weight percentage of trehalose can vary on the weight percentage of other components in the composition like, polysucrose, platelet derivatives, plasma protein, and buffering agents.

In some embodiments of any of the aspects and embodiments herein that include a composition, or in some compositions used in or formed by a process that includes a population of platelet derivatives in a hydrated or rehydrated form, comprises polysucrose in the range of 2-8%, 2.25-7.75%, 2.5-7.5%, or 2.5-6.5%. In an exemplary embodiment, the composition comprises 3% polysucrose. In another exemplary embodiment, the composition comprises 6% polysucrose.

In some embodiments of any of the aspects and embodiments herein that include a composition, or in some compositions used in or formed by a process that includes a platelet composition in a powdered form, comprises polysucrose having a weight percentage in the range of 20-80%, 25-75%, 30-70%, 35-65%, 30-80%, or 45-60%. In some embodiments, the weight percentage of trehalose can vary on the weight percentage of other components in the composition like, trehalose, platelet derivatives, plasma protein, and buffering agents.

In some embodiments of any of the aspects and embodiments herein that include a composition, or in some compositions used in or formed by a process that includes a platelet composition in a powdered form, comprises trehalose and polysucrose having a combined weight percentage in the range of 30-95%, 35-95%, 40-90%, 40-90%, 45-90%, or 60-95%.

In some embodiments of any of the aspects and embodiments herein that include a composition or in some compositions used in or formed by a process herein, comprises platelet derivatives that are positive for at least one platelet activation marker selected from the group consisting of Annexin V, and CD 62. In some embodiments, the platelet derivatives are positive for at least one platelet marker selected from the group consisting of CD 41, CD 42, and CD 61. In some embodiments, the platelet derivatives are positive for CD 47. In some embodiments, the platelet derivatives are positive for Annexin V. In some embodiments, the platelet derivatives are positive for Annexin V. In some embodiments, at least 25%, 50%, or 75% of the platelet derivatives in the platelet derivative composition are Annexin V positive. In some embodiments, the platelet derivatives are positive for CD 41. In some embodiments, at least 30%, 35%, 40%, 45%, 50%, 55%, 60%, or 65% of the platelet derivatives in the platelet derivative composition are CD41 positive. In some embodiments, at least 90%, 91%, 92%, 93%, 94%, 95% 96%, 97%, 98%, or 99% platelet derivatives that are positive for CD 41 have a size in the range of 0.5-2.5 μm. In some exemplary embodiments, at least 95% platelet derivatives that are positive for CD 41 have a size in the range of 0.5-2.5 μm. In some embodiments, the platelet derivatives are positive for CD 42. In some embodiments, at least 65%, 80%, or 90% of the platelet derivatives in the platelet derivative composition are CD42 positive. In some embodiments, the platelet derivatives are positive for CD 47. In some embodiments, at least 8%, 10%, 15%, or 20% of the platelet derivatives in the platelet derivative composition are CD47 positive. In some embodiments, the platelet derivatives are positive for CD 62. In some embodiments, at least 10%, 50%, 65%, 80%, or 90% of the platelet derivatives in the platelet derivative composition are CD62 positive. In some embodiments, the platelet derivatives in the platelet derivative composition are positive for CD41, CD62, and Annexin V. In some embodiments, the platelet derivatives in the platelet derivative composition are at least 50% platelet derivatives are positive for CD41, at least 70% platelet derivatives are positive for CD62, and at least 70% platelet derivatives are positive for Annexin V.

In some embodiments of any of the aspects and embodiments herein that include a composition or in some compositions used in or formed by a process herein that includes a population of platelet derivatives in a hydrated or rehydrated form, the platelet derivatives in the platelet derivative composition retain at least 10%, or 15%, or 20% of the lactate dehydrogenase activity of donor apheresis platelets. In some embodiments, the aqueous medium has a lactate concentration of less than 2.0 mmol/L, or 1.5 mmol/L. In some embodiments, the lactate concentration is in the range of 0.4 to 1.3 mmol/L, or 0.5 to 1.0 mmol/L.

In some embodiments of any of the aspects and embodiments herein that include a composition or in some compositions used in or formed by a process, the platelet derivatives, when at a concentration of at least about 70×10³ particles/μL, produce an occlusion time of less than 30 minutes, less than 25 minutes, less than 20 minutes, 14 minutes, or less than 12 minutes in a total thrombus-formation analysis system (T-TAS) assay. In some embodiments, the occlusion time is in the range of 1 to 13 minutes, 1 to 11 minutes, 1 to 10 minutes, or 1 to 7 minutes. In any of the aspects or embodiments, the platelet derivatives when at a concentration of about 255×10³ platelet derivatives/μL are capable of producing occlusion by attaining a pressure of 80 kPa in less than 14 minutes in platelet-reduced citrated whole blood in an in vitro total thrombus-formation analysis system (T-TAS). In any of the aspects of embodiments, the platelet derivatives are capable of yielding+/−25% of one or more occlusion start time, occlusion time, or area under the curve (AUC) in an in vitro T-TAS assay in the presence versus absence of anti-HLA and/or anti-HPA antibodies. In any of the aspects or embodiments, the platelet derivatives are capable of yielding+/−25% of one or more occlusion start time, occlusion time, or area under the curve (AUC) in an in vitro T-TAS assay in the presence versus absence of anti-HPA antibodies.

In some embodiments of any of the aspects and embodiments herein that include a composition or in some compositions used in or formed by a process, the platelet derivatives in the platelet derivative composition comprise thrombosomes. In some embodiments, the platelet derivatives comprise freeze-dried platelets. In some embodiments, the platelet derivatives comprise thermally-treated freeze-dried platelets.

In some embodiments of any of the aspects and embodiments herein that include a process, comprises tangential flow filtration (TFF), centrifugation of a starting material comprising platelet composition, or a combination thereof. In some embodiments of the process, the starting material comprising platelet composition is: a) positive for HLA Class I antibodies based on a regulatory agency approved test; or b) positive for HLA Class II antibodies based on a regulatory agency approved test; or c) positive for HNA antibodies based on a regulatory agency approved test; or two or more of a), b), and c). In some embodiments, the starting material comprising platelet composition has a protein concentration in the range of 60 to 80 mg/mL, or 65 to 75 mg/mL. In some embodiments, the starting material comprising platelet composition comprises donor blood product. In some embodiments, the donor blood product is pooled donor blood product. In some embodiments, the starting material comprising platelet composition comprises donor apheresis material.

In some embodiments of any of the aspects and embodiments herein that include a process, that does not comprise centrifugation of the starting material comprising platelets or platelet composition, the diluted starting material comprising platelets or platelet composition, the concentrated platelet composition, the TFF-treated composition, or the combination thereof. In some embodiments, the process does not comprise centrifugation of a composition comprising platelets or platelet derivatives.

In some embodiments of any of the aspects and embodiments herein that include a process, the TFF comprises concentrating. In some embodiments, the TFF comprises diafiltering. In some embodiments, the diafiltering comprises diafiltering with at least two diavolumes. In some embodiments, the diafiltering is done with at least three diavolumes, or four diavolumes, or five diavolumes, or six diavolumes. In some embodiments, the diafiltering is done with diavolumes in the range of two to ten. In some embodiments, the TFF comprises buffer exchange.

In some embodiments of any of the aspects and embodiments herein that include a process, diluting comprises diluting with an approximately equal weight (±10%) of the preparation agent.

In some embodiments of the process of any of the aspects and embodiments herein that include a process, further comprises a pathogen reduction step. In some embodiments, the pathogen reduction step occurs before the diluting of the starting material. In some embodiments, the pathogen reduction step precedes TFF.

In some embodiments of any of the aspects and embodiments herein that include a process, wherein following washing if the concentration of the platelets or cells in the TFF-treated composition is not 2000×10³ cells/μL (±300×10³), 3000×10³ cells/μL (±300×10³), 4000×10³ cells/μL (±300×10³ 5000×10³ cells/μL (±300×10³), 6000×10³ cells/μL (±300×10³), 7000×10³ cells/μL (±300×10³), 8000×10³ cells/μL (±300×10³), 10000×10³ cells/μL (±300×10³), 12000×10³ cells/μL (±300×10³), 14000×10³ cells/μL (±300×10³), 16000×10³ cells/μL (±300×10³), 18000×10³ cells/μL (±300×10³ or 20000×10³ cells/μL (±300×10³), diluting the preparation agent or concentrating the platelets or the cells to fall within this range.

In some embodiments of any of the aspects and embodiments herein that include a process, further comprises lyophilizing or freeze-drying the TFF-treated composition to form a lyophilized composition. In some embodiments, a process further comprises treating the lyophilized composition at a temperature in the range of 60-90° C., or 65-85° C., or 70-90° C. for a time period in the range of 1-36 hours, or 5-30 hours, or 10-25 hours.

In some embodiments of any of the aspects and embodiments herein that include a process, the TFF is carried out using a membrane with a pore size in the range of 0.2 μm to 1 μm. In some embodiments the TFF is carried out using a membrane with pore size in the range of 0.3 μm to 1 μm, or 0.4 μm to 1 μm, or 0.4 μm to 0.8 μm, or 0.4 μm to 0.7 μm. In illustrative embodiments, the TFF is carried out using a membrane with a pore size of 0.45 μm, or 0.65 μm.

In some embodiments of any of the aspects and embodiments herein that include a process, the TFF is carried out until the absorbance at 280 nm of the aqueous medium is less than or equal to 50%, or 30%, or 10%, or 5%, or 3%, or 1% of the absorbance at 280 nm of the starting material comprising platelet composition, using a path length of 0.5 cm. In some embodiments, the TFF is carried out until the protein concentration or plasma protein concentration in the aqueous medium is less than or equal to 20%, 15%, 12.5%, 10%, 7.5%, 5%, 2.5%, or 1%. In some embodiments, the TFF is carried out until the protein concentration or plasma protein concentration is in between 0.01% and 20%, 0.01% and 15%, 0.01% and 10%, 0.01% and 5%, 0.1% and 20%, 0.1% and 15%, 0.1% and 10%, 0.1% and 5%, 1% and 20%, 1% and 15%, 1% and 10%, 1% and 5%, 2% and 10%, 2% and 5%, 2.5% and 5%, 2.5% and 7.5%, or between 3% and 5%. In some embodiments, the TFF is carried out until the absorbance at 280 nm of the aqueous medium is less than or equal to 2.0 AU, or 1.90 AU, or 1.80 AU, or 1.70 AU, or 1.66 AU, or 1.60 AU, using a path length of 0.5 cm. In some embodiments, the TFF is carried out until the platelet concentration is at least 2000×10³ platelets/μL, 2250×10³ platelets/μL, 3000×10³ platelets/μL, 3250×10³ platelets/μL, 3500×10³ platelets/μL, 4000×10³ platelets/μL, 4250×10³ platelets/μL, 4500×10³ platelets/μL, 4750×10³ platelets/μL, 5000×10³ platelets/μL, 5250×10³ platelets/μL, 5500×10³ platelets/μL, 5750×10³ platelets/μL, 6000×10³ platelets/μL, 7000×10³ platelets/μL, 8000×10³ platelets/μL, 9000×10³ platelets/μL, 10,000×10³ platelets/μL, 11,000×10³ platelets/μL, 12,000×10³ platelets/μL, 13,000×10³ platelets/μL, 14,000×10³ platelets/μL, 15,000×10³ platelets/μL, 16,000×10³ platelets/μL, 17,000×10³ platelets/μL, 18,000×10³ platelets/μL, 19,000×10³ platelets/μL, or 20,000×10³ platelets/μL.

In some embodiments of any of the aspects and embodiments herein that include a process, the TFF-treated composition comprises at least 1000×10³ platelets/μL, 2000×10³ platelets/μL, 2250×10³ platelets/μL, 3000×10³ platelets/μL, 3250×10³ platelets/μL, 3500×10³ platelets/μL, 4000×10³ platelets/μL, 4250×10³ platelets/μL, 4500×10³ platelets/μL, 4750×10³ platelets/μL, 5000×10³ platelets/μL, 5250×10³ platelets/μL, 5500×10³ platelets/μL, 5750×10³ platelets/μL, 6000×10³ platelets/μL, 7000×10³ platelets/μL, 8000×10³ platelets/μL, 9000×10³ platelets/μL, 10,000×10³ platelets/μL, 11,000×10³ platelets/μL, 12,000×10³ platelets/μL, 13,000×10³ platelets/μL, 14,000×10³ platelets/μL, 15,000×10³ platelets/μL, 16,000×10³ platelets/μL, 17,000×10³ platelets/μL, 18,000×10³ platelets/μL, 19,000×10³ platelets/μL, or 20,000×10³ platelets/μL. In some embodiments, the TFF-treated composition comprises 1000×10³ platelets/μL to 20,000×10³ platelets/μL, 10,000×10³ platelets/μL to 20,000×10³ platelets/μL, 5000×10³ platelets/μL to 20,000×10³ platelets/μL, or 5000×10³ platelets/μL to 10,000×10³ platelets/μL.

In some embodiments of any of the aspects and embodiments herein that include a process, the TFF comprises diafiltering with a preparation agent comprising a buffering agent, a base, a loading agent, optionally a salt, and optionally at least one organic solvent. In some embodiments of the process, the TFF comprises buffer exchange into a preparation agent comprising a buffering agent, a base, a loading agent, optionally a salt, and optionally at least one organic solvent. In some embodiments, the buffering agent is HEPES (4-(2-hydroxyethyl)-1-piperazineethanesulfonic acid). In some embodiments, the base is sodium bicarbonate. In some embodiments, the loading agent is a monosaccharide, a polysaccharide, or a combination thereof. In some embodiments, the monosaccharide is selected from the group consisting of sucrose, maltose, trehalose, glucose, mannose, xylose, and combinations thereof. In some embodiments, the monosaccharide is trehalose. In some embodiments, the polysaccharide is polysucrose. In some embodiments, the salt is sodium chloride, potassium chloride, or a combination thereof. In some embodiments, the organic solvent is selected from the group consisting of ethanol, acetic acid, acetone, acetonitrile, dimethylformamide, dimethyl sulfoxide, dioxane, methanol, n-propanol, isopropanol, tetrahydrofuran (THF), N-methyl pyrrolidone, dimethylacetamide (DMAC), and combinations thereof.

In some embodiments of any of the aspects and embodiments herein that include a process, the preparation agent has a pH in the range of 5.5 to 8.0, or 6.0 to 8.0, or 6.0 to 7.5. In an illustrative embodiment, the preparation agent has a pH of 6.5. In another illustrative embodiment, the preparation agent has a pH of 7.4.

In some embodiments of any of the aspects and embodiments herein that include a process, that does not comprise a step for fixing the platelets, or platelet derivatives. In some embodiments, the process does not comprise fixing the platelets, or platelet derivatives using a fixative agent known in the art for fixing the platelets or platelet derivatives. In some embodiments, the process does not comprise contacting the platelets, or platelet derivatives with at least one fixative agent. In some embodiments, the fixative agent is an aldehyde. In some embodiments, the fixative agent is an alcohol. In illustrative embodiments, the fixative agent is selected from the group consisting of formaldehyde, paraformaldehyde, glutaraldehyde, and isopropanol.

In some embodiments of any of the aspects and embodiments herein that include a process, further comprises lyophilizing the composition comprising platelets or platelet derivatives.

In some embodiments of any of the aspects and embodiments herein that include a process, further comprises cryopreserving the composition comprising platelets or platelet derivatives.

In some embodiments of any of the aspects and embodiments herein that include a process, further comprises thermally treating the composition comprising platelets or platelet derivatives.

In some of the embodiments of any of the aspects and embodiments herein that include a process, the TFF is performed at a temperature in the range of 20° C. to 37° C., or 25° C. to 37° C., or 20° C. to 35° C., or 25° C. to 35° C.

In some embodiments of any of the aspects and embodiments herein that include a process, a percentage of beads positive for an antibody selected from the group consisting of HLA Class I antibodies, HLA Class II antibodies, and HNA antibodies, as determined for the composition by flow cytometry using beads coated with Class I HLAs, Class II HLAs, or HNAs, respectively, is reduced by at least 50%, or by at least 75%, or by at least 90%, or by at least 95%, as compared to a similar composition not prepared by a process comprising tangential flow filtration of a blood product composition, centrifugation of a blood product composition, or a combination thereof.

In some embodiments of any of the aspects and embodiments herein that include a composition, or in some compositions used in or formed by a process, the platelet derivatives are derived from human platelets and are positive for at least one marker selected from the group consisting of CD 41, CD 42, and CD 61. In some embodiments, the platelet derivatives are derived from human platelets that are positive for CD 41. In some embodiments, embodiments, the platelet derivatives are derived from human platelets that are positive for CD 42. In some embodiments, embodiments, the platelet derivatives are derived from human platelets that are positive for CD 61. In some illustrative embodiments, the platelet derivatives are derived from human platelets that are positive for CD 41, CD 42, and CD 61.

In some embodiments of any of the aspects and embodiments herein that include a composition, or in some compositions used in or formed by a process, the platelet derivatives are derived from a non-human animal. In some embodiments, the non-human animal is selected from the group consisting of canines, equines, and felines. In some exemplary embodiments, the platelet derivatives are derived from canines.

In some embodiments of any of the aspects and embodiments herein that include a composition, or in some compositions used in or formed by a process that includes a population of platelet derivatives a powdered form, the platelet derivative composition comprises no more than 0.1%, 0.2%, 0.3%, 0.4%, 0.5%, 0.6%, 0.7%, 0.8% r 0.9%, 1%, 1.5%, 2%, 2.5%, 3%, 3.5%, 4.0%, 4.5%, or 4.9% residual moisture. In some embodiments, wherein the platelet derivative composition is in a powdered form, the platelet derivative composition comprises residual moisture in the range of 0.1-2%, 0.2-1.5%, 0.5-1.5%, 0.75-1.25%, 2-3%, 2.5-4.9%, 3-4.5%, 1.5-3%, or 1-2% residual moisture. In some illustrative embodiments, the platelet derivative composition comprises no more than 0.5% residual moisture.

In some embodiments of any of the aspects and embodiments herein that include a composition, or in some compositions used in or formed by a process that includes a plurality of containers each filled with a platelet derivative composition in the form of a powder, the platelet derivative composition in at least one of the plurality of containers comprises or is associated with a first protein from a first gene that has a different amino acid sequence than found in all the versions of the first protein from the first gene in the platelet derivative composition in one or more other containers of the plurality.

In some embodiments of any of the aspects and embodiments herein that include a composition, or in some compositions used in or formed by a process that includes a plurality of containers each filled with a platelet derivative composition in the form of a powder, the amount of plasma protein in the powder of any two containers chosen from different lots, differs by less than 50%, 40%, 30%, 20%, 1%, 50%, 2%, 1%, or 0.5%.

In some embodiments of any of the aspects and embodiments herein that include a composition, or in some compositions used in or formed by a process that includes a plurality of containers each filled with a platelet derivative composition in the form of a powder, the amount of microparticles that are less than 0.5 μm in the powder of any two containers chosen from different lots, differs by less than 50%, 40%, 30%, 20%, 10%, 5%, 2%, 1%, or 0.5%.

In some embodiments of any of the aspects and embodiments herein that include a composition, or in some compositions used in or formed by a process that includes a plurality of containers each filled with a platelet derivative composition in the form of a powder, the platelet derivative composition from the at least 2 lots have different amino acid sequences for at least one, two, three, four, or five protein of a collection of protein gene products from a corresponding collection of encoding genes. In some embodiments, the different amino acid sequences differ at one or more residues corresponding to amino acid residues encoded by a non-synonymous single nucleotide polymorphism (SNP).

In some embodiments of any of the aspects and embodiments herein that includes a plurality of containers each filled with a platelet derivative composition in the form of a powder, the containers can vary in volume from 5-100 ml, 10-90 ml, 25-75 ml, or 5-40 ml. In some embodiments, the volume of containers can be 5 ml, 10 ml, 15 ml, 20 ml, 25 ml, 30 ml, 35 ml, 40 ml, 45 ml, 50 ml, 55 ml, 60 ml, 65 ml, 70 ml, 75 ml, 80 ml, 85 ml, 90 ml, 95 ml, or 100 ml, or any range of volumes between these volumes. In some embodiments, the volume of containers can be above 100 ml, for example, 125 ml, 150 ml, 175 ml, or 200 ml. In some illustrative embodiments, the volume of vials is 30 ml. In some other illustrative embodiments, the volume of vials is 10 ml. In some embodiments, the plurality of containers can have 10-500 vials, 25-450 vials, 50-350 vials, 100-300 vials, or 150-250 vials. In some embodiments, the plurality of containers can have 10, 20, 30, 40, 50, 60, 70, 80, 90, 100, 110, 120, 130, 140, 150, 160, 170, 180, 190, 200, 250, 300, 350, 400, 450, or 500 vials. In some embodiments, the plurality of containers can be increased to more than 500 as per the requirements, for example, 600, 700, 800, 900, or 1000 vials. In some embodiments, the number of vials can be 10-1000, 50-1000, 100-900, 200-800, or 150-700, or 150-500 vials. The number of vials in which a platelet derivative composition as per one of the embodiments, or aspects described herein can be filled and/or present can vary with the manufacturing requirements and the amount of starting material comprising platelets.

In some embodiments of any of the aspects and embodiments herein that includes a plurality of containers each filled with a platelet derivative composition in the form of a powder, the amount of platelet derivatives when the plurality of containers is taken as a whole can be 1×10⁹ to 1×10¹⁶, 1×10¹⁰ to 1×10¹¹, 1×10 to 1×10¹, 1×10¹² to 1×10¹⁶, or 1×10¹³ to 1×10¹⁵.

In some embodiments of any of the aspects and embodiments herein that include a composition, or in some compositions used in or formed by a process, the platelet derivatives are allogenic platelet derivatives. In some embodiments, the platelet derivatives are allogenic platelet derivative product. In some embodiments, a platelet derivative composition as per any of the embodiments or aspects herein, is a composition comprising allogenic platelet derivatives. In some embodiments, a platelet derivative composition as described herein is a U.S. FDA-approved product comprising allogenic platelet derivative composition. In some embodiments, a platelet derivative composition as described herein is a European EMA-approved product comprising allogenic platelet derivative composition. In some embodiments, a platelet derivative composition as described herein is a China FDA-approved product comprising an allogenic platelet derivative composition.

In some embodiments of the aspects and embodiments herein that include a composition, or in some compositions used in or formed by a process, or a process for preparing a platelet derivative composition, the platelets in the starting material can be donated from a human subject. In some embodiments, the human subject can be a male, or a female. In some embodiments, the platelets can be a pooled product from a number of male and female donors. In some embodiments, from a total of 100 donors, any number can be female donors, ranging from 0-100, 5-95, 10-90, 20-80, 30-70, or 40-60, and the rest can be male donors.

In some embodiments of the aspects and embodiments herein that include a composition, or in some compositions used in or formed by a process, or a process for preparing a platelet derivative composition, a starting material can comprise 10-500 units of platelets. In some embodiments, the starting material can comprise 20-500 units, 30-400 units, 40-350 units, or 50-200 units. In some embodiments, the units can be a pooled platelet product from multiple donors as described herein.

In some embodiments of any of the aspects and embodiments herein that include a method for treating a clotting-related disorder in a subject, said method comprising administering to the subject a therapeutically effective amount of the platelet derivative composition of any of the aspects or embodiments herein, or the platelet derivative composition prepared by any of the process described in the aspects or embodiments herein. In some embodiments, the clotting-related disorder is selected from the group consisting of Von Willebrand Disease, hemophilia, thrombasthenia, thrombocytopenia, thrombocytopenic purpura, trauma, or a combination thereof. In some embodiments, the composition is passed through a filter of 18 μm before administering to the subject.

In some embodiments, the platelet derivative composition of any of the aspects or embodiments herein is provided for use in the treatment of a disorder selected from the group consisting of alopecia areata, Von Willebrand Disease, hemophilia, thrombasthenia, thrombocytopenia, thrombocytopenic purpura, trauma, or a combination thereof.

In some embodiments, the platelet derivatives as described herein can be used for healing wounds in a subject. In some embodiments, there is provided a method for healing a wound in a subject, comprising administering a therapeutically effective amount of a platelet derivative composition of any of the aspects or embodiments herein, or the platelet derivative composition prepared by any of the process described in the aspects or embodiments herein, to the subject. In some embodiments, the platelet derivative composition of any of the aspects or embodiments herein is provided for use in wound healing in a subject.

In some embodiments, there is provided a composition comprising platelets or platelet derivatives prepared by any of the process described in any of the aspects or embodiments herein. In some embodiments, there is provided a composition comprising platelets or platelet derivatives and an aqueous medium prepared by any of the process described in any of the aspects or embodiments herein. In some of the embodiments, there is provided a composition comprising freeze-dried platelets prepared by any of the process described in any of the aspects or embodiments herein. In some of the embodiments, there is provided a composition comprising thrombosomes prepared by any of the aspects or embodiments herein. In some of the embodiments of any of the aspects and embodiments herein that include a composition, or in some compositions used in or formed by a process, a composition prepared by a process comprising tangential flow filtration (TFF) of a starting material comprising platelets, centrifugation of a starting material comprising platelets, or a combination thereof. In some embodiments, the centrifugation comprises centrifugation at 1400×g to 1550×g, or 1450×g to 1500×g. In some embodiments, the composition is prepared by a process that does not comprise centrifugation.

In some embodiments of the aspects and embodiments herein that include a platelet derivative composition, or in some compositions used in or formed by a process, or a process for preparing a platelet derivative composition, or a method for treating a subject, or a platelet derivative composition comprising platelet derivatives for use as a medicament in treating a subject, a therapeutically effective dose of platelet derivatives is based on units of thrombin generation activity administered per kilogram of body weight of the subject. In further embodiments of these embodiments the effective dose is not based on the number of platelet derivatives delivered to the subject. In some embodiments of any aspect or embodiment herein the effective dose is based on both A) units of thrombin generation activity administered per kilogram of body weight of the subject; and B) the number of platelet derivatives administered to the subject. In some embodiments of any aspect or embodiment herein the effective dose is based on the weight of the subject.

In some embodiments of any aspect or embodiment herein the subject is suffering from a condition, or a disease selected from the group including only thrombocytopenia, hematologic malignancy, bone marrow aplasia, myeloproliferative disorders, myelodysplastic syndromes, and platelet refractoriness. In some embodiments, the subject is suffering from thrombocytopenia. In some embodiments, the subject is suffering from hematologic malignancy. In some embodiments, the subject is suffering from bone marrow aplasia. In some embodiments, the subject is suffering from myeloproliferative disorders. In some embodiments, the subject is suffering from myelodysplastic syndromes. In some embodiments, the subject is suffering from platelet refractoriness. In some embodiments, the subject is suffering from two or more of the disease or condition selected from the group consisting of thrombocytopenia, hematologic malignancy, bone marrow aplasia, myeloproliferative disorders, myelodysplastic syndromes, and platelet refractoriness.

In some embodiments of any aspect or embodiment herein a therapeutically effective dose or amount of the platelet derivatives in a platelet derivative composition is in the range of 1.0×10⁷ to 1.0×10¹²/kg of the subject.

In some embodiments of any aspect or embodiment herein a therapeutically effective dose or amount of the platelet derivatives is an amount that has a potency in the range of 250 to 5000 TGPU per kg of the subject.

In some embodiments of any aspect or embodiment herein, a method of treatment or a composition for use as a medicament as described herein, a method or a medicament leads to cessation or decrease in bleeding at a primary bleeding site at 12 hours, 24 hours, 48 hours, 72 hours, 4 days, 5 days, 6 days, and/or 7 days after administering the platelet derivative composition. In some embodiments, the primary bleeding site is based upon the most severe bleeding location of the subject within 12 hours prior to administering the platelet derivative composition. In some embodiments, the administering involves infusing a platelet derivative composition. In some embodiments, a platelet derivative composition is administered on Day 1 of the treatment. In some embodiments, the cessation or decrease is evidenced by an ordinal change in WHO bleeding score of the subject evaluated at 24 hours after administering the platelet derivative composition to the subject. In some embodiments, a method or a medicament as described herein leads to cessation or decrease in bleeding at bleeding sites other than primary bleeding site at 12 hours, 24 hours, 48 hours, 72 hours, 4 days, 5 days, 6 days, and 7 days after administering the platelet derivative composition.

In some embodiments of any aspect or embodiment herein, a method of treatment or a composition for use as a medicament as described herein, a method or a medicament leads to an increase in platelet count in the subject at 12 hours, 24 hours, 48 hours, 72 hours, 4 days, 5 days, 6 days, and 7 days after administering the platelet derivative composition. In some embodiments, the increase is at least 500 platelets/μl, 1000 platelets/μl, 2000 platelets/μl, 3000 platelets/μl, 4000 platelets/μl, 5000 platelets/μl, 6000 platelets/μl, 7000 platelets/μl, 8000 platelets/μl, 9000 platelets/μl, or 10000 platelets/μl in the subject. In some embodiments, the increase is in the range of 500 to 10000 platelets/μl, 1000 to 10000 platelets/μl, 2000 to 8000 platelets/μl, or 3000 to 7000 platelets/μl in the subject.

In some embodiments of any aspect or embodiment herein, a method of treatment or a composition for use as a medicament as described herein, a method or a medicament leads to changes, or in some embodiments, does not lead to changes, in one or more markers of endothelial cell injury in the subject from a pre-administration time through 12 hours to 35 days, 24 hours to 32 days, 24 hours to 30 days, or 48 hours to 28 days after administering the platelet derivative composition. In some embodiments, the method or the medicament leads to changes, or in some embodiments, does not lead to changes, in one or more markers of endothelial cell injury in the subject at 72 hours after administering the platelet derivative composition. In some embodiments, the one or more markers of endothelial cell injury is selected from the group consisting of Syndecan-1, hyaluronan, thrombomodulin, vascular endothelial growth factor (VEGF), interleukin 6, and sVE cadherin. In some embodiments, the method or the medicament leads to changes in two or more markers, three or markers, four or more markers, five or more markers, or all of the markers selected from the group consisting of Syndecan-1, hyaluronan, thrombomodulin, vascular endothelial growth factor (VEGF), interleukin 6, and sVE cadherin. In some embodiments, the changes can be an increase or a decrease in the markers of endothelial cell injury in the subject as compared to a control.

In some embodiments of any aspect or embodiment herein, a method of treatment or a composition for use as a medicament as described herein, a method or a medicament leads to acceptable measures of coagulation in the subject at 12 hours to 35, 24 hours to 32 days, 24 hours to 30 days, or 48 hours to 28 days after administering the platelet derivative composition. In some embodiments, a method or a medicament leads to acceptable measures of coagulation in the subject at 72 hours after administering the platelet derivative composition. In some embodiments, the acceptable measure of coagulation includes one or more, two or more, three or more, four or more, five or more, or all of prothrombin time (PT), international normalized ratio (INR), fibrinogen, D-dimer, activated partial thromboplastin time (aPTT), and thromboelastography (TEG) or rotational thromboelastometry (ROTEM). In some embodiments, a method or a medicament leads to an increase or a decrease in the acceptable measure of coagulation in the subject as compared to a control.

In some embodiments of any aspect or embodiment herein, a method of treatment or a composition for use as a medicament as described herein, a method or a medicament leads to acceptable measures of hematology in the subject from a pre-administration time through 12 hours to 35 days, 24 hours to 32 days, 24 hours to 30 days, or 48 hours to 28 days after administering the platelet derivative composition. In some embodiments, the acceptable measures of hematology are one or more, two or more, three or more, four or more, five or more, or all selected from the group consisting of Prothrombin Fragment 1+2, thrombin generation assay (TGA), Thrombopoietin, activated Protein C, tissue plasminogen activator (TPA), and/or plasminogen activator inhibitor (PAI). In some embodiments, the acceptable measures of hematology can be an increase or a decrease in the subject as compared to a control.

In some embodiments of any aspect or embodiment herein, a method of treatment or a composition for use as a medicament as described herein, a method or a medicament leads to survival of the subject without WHO Grade 2A or greater bleeding during the first 3, 4, 5, 6, 7, 8, 9, or 10 days after administering of a platelet derivative composition.

In some embodiments of any aspect or embodiment herein, a method of treatment or a composition for use as a medicament as described herein, administering is performed in a maximum of 2, 3, 4, 5, 6, 7, 8, 9, or 10 doses in a 72-hour period of treatment. In some embodiments, the subject has a count of total circulating platelets (TCP) between 5,000 to 100,000 platelets/μl, 10,000 to 90,000 platelets/μl, 10,000 to 80,000 platelets/μl, or 10,000 to 70,000 platelets/μl of blood at the time of administering. In some embodiments, the subject is undergoing one or more, two or more, three or more, or all of chemotherapy, immunotherapy, radiation therapy or hematopoietic stem cell transplantation at the time of administering. In some embodiments, the subject is refractory to platelet transfusion, wherein refractory is a two 1-hour CCI [corrected count increment] of <5000 on consecutive transfusions of liquid stored platelets. In some embodiments, the subject, for example, at the time of the administering a first dose of the platelet derivatives or the rehydrated platelet derivatives has a WHO bleeding score of 2 excluding cutaneous bleeding.

In some embodiments of any aspect or embodiment herein the subject at the time of administering has two or more, or all of: confirmed diagnosis of hematologic malignancy, myeloproliferative disorder, myelodysplastic syndrome, or aplasia; undergoing chemotherapy, immunotherapy, radiation therapy or hematopoietic stem cell transplantation; or refractory to platelet transfusion wherein refractory is a two 1-hour CCI of <5000 on consecutive transfusions of liquid stored platelets.

In some embodiments of any aspect or embodiment herein the administering confers an improved survival at 10, 15, 20, 25, 30, 35, 40, 45, or 50 days after administering the platelet derivatives. In some embodiments of any aspect or embodiment herein the administering leads to a decrease in administration of secondary blood products, platelets, or platelet derivatives to the subject for the first 5, 6, 7, 8, 9, or 10 days after the administering of the platelet derivatives.

In some embodiments of any aspect or embodiment herein that include delivering platelet derivatives to a subject, or administering platelet derivatives to a subject, or treating a subject, or use of platelet derivative composition or platelet derivatives as described in any of the aspects or embodiments, platelet derivatives can have an effective dose or a therapeutically effective dose in the range of 1.0×10⁷ to 1.0×10¹¹ particles/kg of the subject. In some embodiments, platelet derivatives can have an effective dose or a therapeutically effective dose in the range 250 and 5000 TGPU per kg of the subject.

In some aspects and embodiments, the pre-platelet derivative cancer therapeutic dosing regimen and/or the post-platelet derivative cancer therapeutic dosing regimen is selected from the group consisting of: a dosing regimen provided in a regulatory agency approved dosing amount and schedule; a dosing regimen provided in a dosing and/or clinical studies section of the regulatory agency approved full prescribing information for the cancer therapeutic agent; a dosing regimen used in a clinical trial for the cancer therapeutic agent to establish efficacy based on tumor burden response rates, progression free survival and/or overall survival; and a dosing regimen recommended by a national or international oncology society or network.

In some aspects and embodiments, the pre-platelet derivative cancer therapeutic dosing regimen and the post-platelet derivative cancer therapeutic dosing regimen are the same. In some embodiments, the administering a dose or an effective dose of the platelet derivatives to the subject is repeated. In some embodiments, the administering of a dose or an effective dose of the platelet derivatives is repeated between the pre-platelet derivative cancer therapeutic dosing regimen and the post-platelet derivative cancer therapeutic regimen. In some embodiments, the administering of a dose or an effective dose of the platelet derivatives is repeated after the administering of the cancer therapeutic agent to the subject according to the post-platelet derivative cancer therapeutic dosing regimen, such that the subject does not become thrombocytopenic during the administering with the cancer therapeutic agent. In some embodiments, after the administering of a dose or an effective dose of the platelet derivatives and before the administering the cancer therapeutic agent to the subject according to the post-platelet derivative cancer therapeutic dosing regimen, determining that the platelet count in the subject is increased.

In some aspects and embodiments, the method herein includes determining that the subject has a low platelet count after administering a cancer therapeutic agent to the subject, according to a pre-platelet derivative cancer therapeutic dosing regimen, and after determining that the subject has a low platelet count, the method further includes,
administering platelets to the subject in an amount expected to increase a platelet count by an expected amount within a target platelet increase timeframe; and
after the administering and during the target platelet increase timeframe, determining that the platelet count did not increase by the expected amount.

In some aspects and embodiments, a pre-platelet derivative cancer therapeutic dosing regimen comprises administering more than 1 dose of the cancer therapeutic agent, for example, 2, 3, 4, 5, or more doses, and wherein a post-platelet derivative cancer therapeutic dosing regimen comprises administering 1 dose of the cancer therapeutic agent.

In some aspects and embodiments, a pre-platelet derivative cancer therapeutic dosing regimen comprises administering 1 dose of the cancer therapeutic agent, and wherein a post-platelet derivative cancer therapeutic dosing regimen comprises administering more than 1 dose of the cancer therapeutic agent, for example, 2, 3, 4, 5, or more doses.

In some aspects and embodiments, a pre-platelet derivative cancer therapeutic dosing regimen and a post-platelet derivative cancer therapeutic dosing regimen are the same. In some embodiments, the pre-platelet derivative or the post-platelet derivative cancer therapeutic dosing regimen is selected from the group consisting of: a dosing regimen provided in a regulatory agency approved dosing amount and schedule; a dosing regimen provided in a dosing and/or clinical studies section of the regulatory agency approved full prescribing information for the cancer therapeutic agent; a dosing regimen used in a clinical trial for the cancer therapeutic agent to establish efficacy based on tumor burden response rates, progression free survival and/or overall survival; and a dosing regimen recommended by a national or international oncology society or network. In some embodiments, the administering of the effective dose of the platelet derivatives is repeated. In some embodiments, the administering of the effective dose of the platelet derivatives is repeated after the administering of the cancer therapeutic agent to the subject according to the post-platelet derivative cancer therapeutic dosing regimen, such that the subject does not become thrombocytopenic during the administering with the cancer therapeutic agent. In some embodiments, before the administering of the effective dose of the platelet derivatives to the subject according to the post-platelet derivative cancer therapeutic dosing regimen, the method further includes determining that the platelet counts in the subject is above a threshold level.

In some aspects and embodiments, the cancer therapeutic dosing regimen is selected from the group consisting of: a dosing regimen provided in a regulatory agency approved dosing amount and schedule; a dosing regimen provided in a dosing and/or clinical studies section of the regulatory agency approved full prescribing information for the cancer therapeutic agent; a dosing regimen used in a clinical trial for the cancer therapeutic agent to establish efficacy based on tumor burden response rates, progression free survival and/or overall survival; and a dosing regimen recommended by a national or international oncology society or network. In some embodiments, “n” is a round of administering the cancer therapeutic agent according to the cancer therapeutic dosing regimen after which the subject is diagnosed to have low platelet counts. In some embodiments, “n” is a round of administering the cancer therapeutic agent according to the cancer therapeutic dosing regimen after which the subject is suspected to have low platelet counts. In some embodiments, “n” is a 1^{st}, 2^{nd}, 3^{rd}, 4^{th}, 5^{th}, 6^{th}, 7^{th}, 8^{th}, 9^{th}, 10^{th}, 11^{th}, 12^{th}, 13^{th}, 14^{th}, or 15^{th}, round of administering the cancer therapeutic agent according to the cancer therapeutic dosing regimen. In some embodiments, the administering the platelet derivatives is repeated such that “n” is two or more of a 1^{st}, 2^{nd}, 3^{rd}, 4^{th}, 5^{th}, 6^{th}, 7^{th}, 8^{th}, 9^{th}, 10^{th}, 11^{th}, 12^{th}, 13^{th}, 14^{th}, or 15^{th}, round of administering the cancer therapeutic agent according to the cancer therapeutic dosing regimen. In some embodiments, the administering of the effective dose of the platelet derivatives to the subject is before the n^{th} round of administering the cancer therapeutic according to the cancer therapeutic dosing regimen, and wherein “n” is each round after the 1^{st} round of administering the cancer therapeutic agent according to the cancer therapeutic dosing regimen. In some embodiments, the administering of the effective dose of the platelet derivatives to the subject is after the n^{th} round of administering the cancer therapeutic agent according to the cancer therapeutic dosing regimen, and wherein “n” is each of the round of administering the cancer therapeutic agent according to the cancer therapeutic dosing regimen. In some embodiments, the method provides an un-interrupted cancer therapeutic dosing regimen. In some embodiments, the administering the effective dose of the platelet derivatives to the subject during the duration of the cancer therapeutic dosing regimen maintains the platelet levels in the subject above a threshold level. In some embodiments, the methods herein provide no interruption in the cancer therapeutic dosing regimen of the cancer therapeutic agent to the subject due to low platelet count in the subject. In some embodiments, the threshold level is a blood platelet count of 150,000 platelets/μl of circulating blood. In some embodiments, the low platelet count is below 150×10⁹/L, 100×10⁹/L, 75×10⁹/L, 50×10⁹/L, or 25×10⁹/L.

In some aspects and embodiments, the national or international oncology society or network comprises National Institutes of Health (NIH), National Cancer Institute (NCI), National Comprehensive Cancer Network (NCCN), American Society of Clinical Oncology (ASCO), American Society of Hematology (ASH), American Cancer Society (ACS), The American Association for Cancer Research (AACR), American Society for Radiation Oncology (ASTRO), Society for Neuro-Oncology (SNO), Society for Immunotherapy of Cancer (SITC), American Society for Transplantation and Cellular Therapy (ASTCT), Society of Surgical Oncology (SSO), American Urological Association (AUA), Children's Oncology Group (COG), Oncology Nursing Society (ONS), Leukemia and Lymphoma Society (LLS), UPMC Hillman Cancer Center, The National Surgical Adjuvant Breast and Bowel Project (NSABP), The Pancreatic Cancer Action Network (PanCAN), The Breast Cancer Foundation (BCRF), Susan G Koman Foundation, The Multiple Myeloma Research Foundation (MMRF), European Cancer Organisation, European Organisation for Research and Treatment of Cancer (EORTC), European Society for Medical Oncology (ESMO), European Hematology Association (EHA), European association for Cancer Research (EACR), European Association of Neuro-Oncology (EANO), European Society for Blood and Marrow Transplantation (EBMT), and European Association of Urology (EAU).

In some aspects and embodiments, the methods herein can include administering 1 to 10, 1 to 9, 1 to 8, 1 to 6, 1 to 5, 1 to 4, 1 to 3, 2 to 10, 2 to 6, 2 to 5, 3 to 10, 3 to 8, 3 to 6, 4 to 10, or 4 to 8 doses of platelet derivatives in a platelet derivative composition to the subject, and in some embodiments, one dose of the platelet derivatives can be in an amount of between 5×10⁸ particles/kg to 1.0×10¹² particles/kg. In some embodiments, the subject has no sites that are actively bleeding with a bleeding score WHO Grade 2A or greater. In some embodiments, the administering leads to cessation of bleeding at the primary bleeding site. In some embodiments, the administering leads to cessation of bleeding at all primary and bleeding sites other than the primary bleeding site. In some embodiments, the administering leads to cessation of bleeding at all bleeding sites.

In some aspects and embodiments, the subject experiences no adverse events attributable to the administering the platelet derivatives when the decreased bleeding is detected. In some embodiments, the subject experiences no grade 3 or grade 4 adverse events attributable to the administering the platelet derivatives, when the decreased bleeding is detected. In some embodiments, the subject experiences no adverse events or no grade 2 or higher adverse events, and in illustrative embodiments no grade 3 or grade 4 adverse events, attributable to the administering the platelet derivatives, within 2, 4, 5, 7, 8, 10, 12, 14, 15, 20, 25, or 30 days of the first dose. In some embodiments, the subject experiences no adverse events, or no grade 2 or higher adverse events, and in illustrative embodiments no grade 3 or grade 4 adverse events, attributable to the administering the platelet derivatives, within 15 minutes, 30 minutes, 45 minutes, 60 minutes, 1 hour, 2, 4, 6, 8, 10, 12, 14, 16, 18, 20, 22, or 24 hours of the first dose. In some embodiments, the subject experiences no adverse events, or no grade 2 or higher adverse events, and in illustrative embodiments no grade 3 or grade 4 adverse events, attributable to the administering the platelet derivatives when the decreased bleeding is detected. In some embodiments, the subject experiences no adverse events, or no grade 2 or higher adverse events, and in illustrative embodiments no grade 3 or grade 4 adverse events, attributable to the administering the platelet derivatives, within 15 minutes, 30 minutes, 45 minutes, 60 minutes, 1 hour, 2, 4, 6, 8, 10, 12, 14, 16, 18, 20, 22, 24, 28, 30, 32, 34, 36, 48, or 72 hours of the first dose.

In some aspects, and embodiments, the subject, at the time of the administering, has a WHO bleeding score of Grade 2, Grade 3, or a higher bleeding score. In some embodiments, within 15 minutes, 30 minutes, 45 minutes, 60 minutes, 2, 4, 6, 8, 10, 12, 14, 16, 18, 20, 22, or 24 hours, 2 days, or 3 days after the administering to the subject, the bleeding of the subject is decreased to a WHO bleeding score of less than Grade 2 or 2A, less than Grade 1, or decreased to Grade 0, at a primary site, at a bleeding site other than the primary site, or at all the bleeding sites. In illustrative embodiments, after the administering, the bleeding of the subject is decreased to a WHO bleeding score of Grade 1, less than Grade 1, or decreased to Grade 0, at all the bleeding sites within 15 minutes, 30 minutes, 45 minutes, 1, 2, 4, 6, 8, 10, 12, 14, 16, 18, 20, 22, or 24 hours after the administering of a first dose of the platelet derivatives. In some embodiments, the platelet derivatives restore thrombin levels identical 4,800 particles/microliter. In some embodiments, the subject has anti-human platelet antigen (HPA) antibodies. In some embodiments, the subject has cross-reactive antibodies against the platelet derivatives. In some embodiments, the subject has Glanzmann thrombasthenia. In some embodiments, the subject has been treated or is being treated with an antiplatelet agent. In some embodiments, the antiplatelet agent is selected from the group consisting of abciximab, aspirin, cangrelor, ticagrelor, clopidogrel, prasugrel, eptifibatide, tirofiban, and a combination thereof. In some embodiments, the platelet derivatives are capable of yielding comparable in vitro thrombin generation activity in the presence versus absence of cross-reactive antibodies against the platelet derivatives. In some embodiments, the platelet derivatives are capable of yielding comparable hemostatic activity in an in vitro T-TAS assay in the presence versus absence of cross-reactive antibodies against the platelet derivatives. In some embodiments, the platelet derivatives are capable of yielding comparable in vitro thrombin generation activity in the presence versus absence of anti-HLA and/or anti-HPA antibodies. In some embodiments, the platelet derivatives are capable of yielding comparable hemostatic activity in an in vitro T-TAS assay in the presence versus absence of anti-HLA and/or anti-HPA antibodies. In some embodiments, the subject is not undergoing another treatment for thrombocytopenia at the time between the administering and a determination that the bleeding is reduced. In some embodiments, the method includes a treatment for thrombocytopenia that does not comprise platelet derivatives, in illustrative embodiment a standard of care treatment for thrombocytopenia, is administered to the subject within 3 days, 1 day, 12 hours, 8 hours, 4 hours, 2 hours, 1 hour or 5 minutes after the determination that the bleeding is reduced. In some embodiments, platelets are administered to the subject within 3 days, 1 day, 12 hours, 8 hours, 4 hours, 2 hours, 1 hour or 5 minutes after the determination that the bleeding is reduced.

In some aspects and embodiments, the subject, at the time of administering the first dose to the subject, does not have another thrombotic or Ischemic Condition. In some embodiments, the subject, at the time of administering the first dose to the subject, is not taking any pro or anti-thrombotic medications. In some embodiments, the subject, at the time of administering the first dose to the subject, does not have a liver enzyme or blood creatinine levels>3×ULN. In some embodiments, methods herein include rehydrating the platelet derivatives in the platelet derivative composition, for example in between 10 to 250 ml, 25 to 100 ml, 25 to 75, or 30 to 50 ml of water or a buffered solution, before the administering.

The disclosed embodiments, examples and experiments are not intended to limit the scope of the disclosure or to represent that the experiments below are all or the only experiments performed. Efforts have been made to ensure accuracy with respect to numbers used (e.g., amounts, temperature, etc.) but some experimental errors and deviations should be accounted for. It should be understood that variations in the methods as described may be made without changing the fundamental aspects that the experiments are meant to illustrate.

Those skilled in the art can devise many modifications and other embodiments within the scope and spirit of the present disclosure. Indeed, variations in the materials, methods, drawings, experiments, examples, and embodiments described may be made by skilled artisans without changing the fundamental aspects of the present disclosure. Any of the disclosed embodiments can be used in combination with any other disclosed embodiment.

In some instances, some concepts have been described with reference to specific embodiments. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of invention.

### EXAMPLES

### Example 1. Freeze-Dried Platelet Derivate Clinical Trial, a Dose and Impact on Bleeding Study

Freeze-dried platelet derivatives at low, medium and high doses, are compared to liquid stored platelets (LSP) in their effectiveness to cease or decrease bleeding. Freeze-dried platelet derivatives have a short circulation time and are not expected to increase the platelet count immediately post infusion. As such, clinical presentation of the patient is used to evaluate bleeding during the treatment phase and guide the treatment scheme rather than only platelet count.

### The Study

A prospective, multicenter, randomized, open-label, Phase 2, parallel, dose ranging, multidose trial enrolled patients into three freeze-dried platelet derivatives dose groups and one control liquid stored platelets (LSP) group in order to evaluate, in a dose-escalation manner, the impact on bleeding, and the preliminary effect on coagulation measures of increasing doses of allogeneic freeze-dried platelet derivatives. Freeze-dried platelet derivatives were prepared according to the method as described in Example 1 of U.S. Pat. No. 11,529,587 B2, incorporated herein by reference in its entirety. These platelet derivatives in dried form (platelet derivative composition in the form of a powder), were resuspended in water and delivered in one of three doses indicated herein in Table 1.

The patients, 21 in total selected, were: 18 years and older, female or male, that met the following inclusion criteria:
1. Adults (≥18 years) that met the following criteria (a) and (b):
   a. a platelet count of less than or equal to 70,000 platelets/μL blood,
   b. any one or more of (i-iii):
      i. confirmed diagnosis of hematologic malignancy, myeloproliferative disorder, myelodysplastic syndrome, or aplasia;
      ii. undergoing chemotherapy, immunotherapy, radiation therapy or hematopoietic stem cell transplantation; and
      iii. refractory to platelet transfusion defined as two 1-hour corrected count increment (CCI) of <5000 on consecutive transfusions of LSP (Sacher, 2003) or as defined by local site policy,
2. WHO Bleeding Score of 2, or 3.
3. All patients provide informed consent directly or through a legally authorized representative, comply with treatment and monitoring, and all women of childbearing potential test negative for pregnancy.

A maximum of 6 doses of the freeze-dried platelet derivatives were allowed to be administered in a 72-hour period at the investigator's discretion to treat enrolled bleeding patients. The study evaluates the safety and potential efficacy of three different doses of freeze-dried platelet derivatives v/s control. Freeze-dried platelet derivatives Low Dose (1.6×10⁸ particles/kg to 1.9×10⁸ particles/kg), Medium Dose (8×10⁸ particles/kg), High Dose (1.6×10⁹ particles/kg), or control (LSP) are listed in Table 1.

TABLE 1

| Trial-Arm | Platelet derivative |
|---|---|
| Description | particles/kg (TGPU) |
| Low Dose | 1.6-1.9 × 10⁸/kg |
| Medium Dose | 8 × 10⁸/kg |
| High Dose | 1.6 × 10⁹/kg |
| Control | One unit of room temperature irradiated, leukocyte |
| | reduced apheresis platelets or whole blood derived |
| | pooled platelet concentrate equivalent (4-6 units) |

N/A = not applicable; NOD = non-obese diabetic; SCID = severe combined immunodeficiency; TGPU = thrombin generating potency unit.

The specific outcomes analyzed are tabulated below in Table 2.

TABLE 2

| | |
|---|---|
| Efficacy Endpoint | Time Frame (Evaluated at) |

Primary Outcome Measures

| | |
|---|---|
| Cessation or decrease in bleeding at | 24 hours post initial infusion. |
| primary bleeding site, based upon the | |
| most severe bleeding location at Day | |
| 1 baseline taken within 12 hours prior | |
| to infusion, as evidenced by ordinal | |
| change in WHO (World Health | |
| Organization) Bleeding Score. | |

Secondary Outcome Measures

| | |
|---|---|
| Number of days alive and without | 7 days after first freeze-dried |
| WHO (World Health Organization) | platelet derivatives or LSP |
| Grade 2a or greater bleeding through | infusion. |
| initial 7 days after first freeze-dried | |
| platelet derivative composition or LSP | |
| Infusion | |
| 30-day mortality post first infusion of | 30 days post first infusion |
| freeze-dried platelet derivative | (+/−2 days). |
| composition or post first infusion of | |
| LSP as control | |
| Cessation or decrease in each | 12, 24, 48, 72 hours and Day |
| additional bleeding site (other than | 7 post first infusion of freeze- |
| primary bleeding site), as evidenced | dried platelet derivatives or |
| by ordinal change in WHO (World | LSP infusion as control. Also |
| Health Organization) Bleeding Score | evaluate at Days 4-6 if patient |
| after first infusion of freeze-dried | is hospitalized at that time. |
| platelet derivative composition or LSP | |
| infusion as control. | |
| Number, timing, type and reason for | 7 days after first freeze-dried |
| administration of all blood products | platelet derivative |
| including platelets and freeze-dried | composition or LSP infusion |
| platelet derivative composition during | |
| the initial 7 days after first freeze- | |
| dried platelet derivative composition | |
| or LSP infusion. | |
| Platelet count | measured at 12, 24, 48, 72 |
| | hours and Day 7 of first |
| | infusion of freeze-dried |
| | platelet derivative |
| | composition or LSP. Also |
| | evaluate at Day 4-6 if patient |
| | is hospitalized at that time. |
| Measures of hematology including: | From baseline through last |
| Prothrombin Fragment 1 + 2; | study visit (up to 30 days |
| thrombin generation assay (TGA); | (+/−2 days). |
| Thrombopoietin; activated Protein C, | |
| tissue plasminogen activator (TPA), | |
| and plasminogen activator inhibitor | |
| (PAI) per schedule of assessments | |
| Measures of coagulation including: | From baseline through last |
| prothrombin time (PT); international | study visit (up to 30 days |
| normalized ratio (INR); fibrinogen; | (+/−2 days). |
| D-dimer; activated partial | |
| thromboplastin time (aPTT); and | |
| thromboelastography (TEG) or | |
| rotational thromboelastometry | |
| (ROTEM) per schedule of | |
| assessments. | |
| Changes in markers of endothelial cell | From baseline through last |
| injury/repair from preinfusion | study visit (up to 30 days |
| baseline through 72 hours after first | (+/−2 days). |
| infusion, including: Syndecan-1, | |
| hyaluronan, thrombomodulin, | |
| vascular endothelial growth factor | |
| (VEGF), interleukin 6, sVE cadherin | |
| per schedule of assessments. | |

Other Outcome Measures

| | |
|---|---|
| Safety: Serious Adverse Events | From baseline through last |
| (SAEs) | study visit (up to 30 days |
| | (+/−2 days)) |
| Safety: Adverse Events (SAEs) | From baseline through last |
| | study visit (up to 30 days |
| | (+/−2 days)) |
| Safety: Unanticipated problems | From baseline through last |
| involving risk to human subjects | study visit (up to 30 days |
| | (+/−2 days)) |

Patients with the Following Criteria are Excluded from the Study:

### Patient Exclusion Criteria:

Any disorder or condition related to thrombosis, embolism, vascular occlusion, or ischemia (except when a prior history of central line thrombosis has resolved), including but not limited to: past history or current diagnosis of arterial or venous thromboembolic disease including acute coronary syndrome, peripheral vascular disease, and retinal arterial or venous thrombosis, MI, stent placement, valve replacement and/or repair, sinusoidal obstruction syndrome (veno-occlusive disease) or cytokine storm syndrome associated with CAR-T cell therapy.
Refusal to accept blood products
Liver enzyme blood levels greater than 3× the upper limit of normal (ULN)
Blood creatinine level greater than 3×ULN
Received platelet inhibitor drugs, cyclooxygenase-2 (COX-2) inhibitors, or nonsteroidal anti-inflammatory drugs within 5 days prior to infusion
Currently receiving anticoagulant therapy or antiplatelet therapy
Receipt of any pro-coagulant agents other than tranexamic acid (TXA) [e.g., desmopressin (DDAVP), recombinant Factor VIIa or prothrombin complex concentrates (PCC)] within 48 hours of first infusion, or with known hypercoagulable state
WHO Bleeding Score of 2 solely due to cutaneous bleeding such as petechia, ecchymosis, or bruising
Receiving L-asparaginase as part of a current cycle of treatment
Known inherited or acquired bleeding disorder including, but not limited to: acquired storage pool deficiency or paraproteinemia with platelet inhibition

Known inherited or acquired prothrombotic disorders, including antiphospholipid syndrome (Those with lupus anticoagulant or positive antiphospholipid serology without thrombosis are NOT excluded.)
Anuria
On dialysis
Receipt of an investigational drug within 1 month before first infusion, other than for treatment of their underlying disease
Females pregnant or nursing or unwilling to use contraception during and for 30 days after taking the study product (females). Evidence of effective birth control may be used, at the discretion of the physician
Acute or chronic medical disorder that, in the opinion of the Investigator, would impair the ability of the patient to receive or respond to study treatment
Prior participation in this study with successful infusion of the investigational or control product
Currently enrolled in other trials not related to their primary disease process or involving platelet transfusions, platelet growth factors, or other pro-coagulant agents

Patients who signed the informed consent form and met the inclusion criteria but not the exclusion criteria are treated by administration of freeze-dried platelet derivative compositions according to one of the three active arms of the study or the control arm as provided in Table 1. The outcomes listed immediately above are analyzed at the timepoints indicated above for each outcome to assess the safety and efficacy of the freeze-dried platelet derivative compositions at the three different doses for the listed outcomes/indications. A total of 22 patients were screened, and 20 were enrolled and randomized. A total of 3 subjects received conventional liquid stored platelets (LSP) and were part of the “control” group. A total of 7 were randomized to the high dosage and 5 were randomized to the medium dosage, and a total of 5 subjects were randomized to the low dosage. The subjects were screened for the presence of anti-HLA and anti-HPA antibodies, and 5 of the 20 subjects had refractory HLA Class 1 antibodies. Further, 2 of the 5 subjects had antibodies that were cross-reactive with the platelet derivatives that were administered.

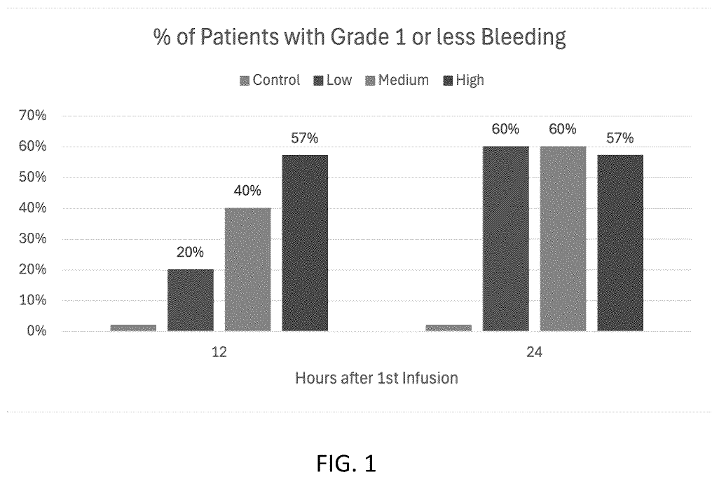

FIG. 1 illustrates the primary endpoint data at 12 hours (group of four bars on the left side labelled “12”) and 24 hours (group of four bars on the right side labelled “24”) from the administering of a first dose of the rehydrated platelet derivatives. At 12 hours and 24 hours after the administering, it can be observed that all the doses of the platelet derivatives were able to decrease the bleeding as compared to liquid stored platelets (LSP) depicted by the “control” bar (first bar from left side in both the groups labelled “12” and “24”). At 12 hours after the administering, it can be observed that the high dose (last bar in both the groups) performed better than the medium dose, and the medium dose performed better than the low dose of the platelet derivatives depicted by the percentage of subjects with Grade 1 or less bleeding. The percentage of subjects with Grade 1 or less bleeding at all the bleeding sites at 12 hours after the administering is the lowest (20%) in the low dose of the platelet derivatives, and is around 40% in the medium dose, and the highest (57%) in the high dose of the platelet derivatives. Referring to FIG. 1, however all the doses provide similar percentage of the subjects having a WHO bleeding score of Grade 1 or less at 24 hours timepoint, it can be observed that all the doses provide a superior result in controlling bleeding of the subjects at 12 hours timepoint as compared to the control, and medium dose and high dose provide a superior result in controlling bleeding as compared to the control and the low dose at 12 hours timepoint. Therefore, the data demonstrates the importance of the platelet derivatives herein in controlling bleeding during the initial hours of intervention that is critical to the outcome of the intervention.

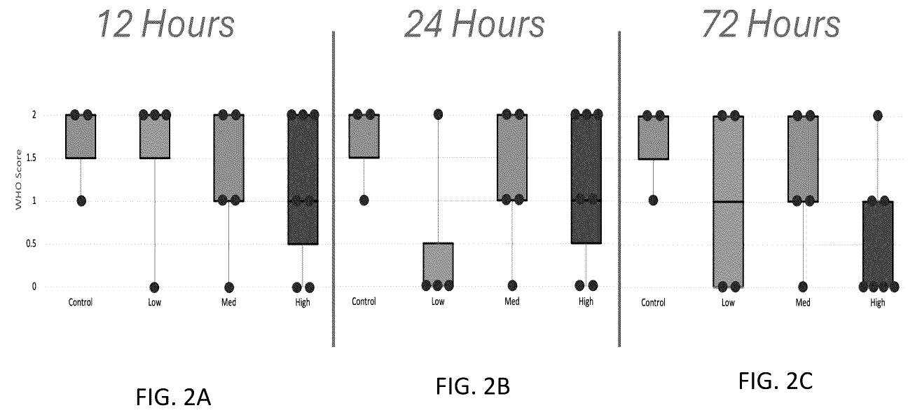

FIG. 2 illustrates the reduction in WHO bleeding score in the subjects at a primary site at 12 hours (FIG. 2A), 24 hours (FIG. 2B), and 72 hours (FIG. 2C) timepoint after the administering of a first dose of the platelet derivatives, as compared to the control. It can be observed that the medium and high dose of the platelet derivatives performed better in reducing the WHO bleeding score at the primary site to below Grade 2 at all the timepoints as compared to the low dose and the control. At 12 hours timepoint (FIG. 2A) the high dose and medium dose performed better in reducing the WHO bleeding score to less than Grade 2 at the primary site with the high dose performing better than the low dose.

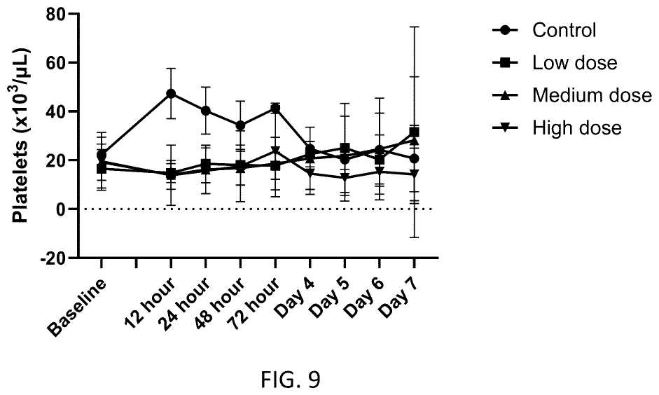

Referring to FIG. 9, the subjects of the study were monitored for platelet counts at different time intervals, for example, starting at baseline (pre-administration), the time of administration of a first dose, and 12 hours, 24 hours, 48 hours, 72 hours, day 4, day 5, day 6, and day 7 after the time of administering the first dose of the platelet derivatives. It can be observed that during the time points of 12 hours and 24 hours, the time points where decreased bleeding was detected in most of the subjects who received the platelet derivatives (see above), as well as later time points 48 hours (day 2), and 72 hours (day 3), from the time the first dose of platelet derivatives was administered, the vast majority of the subjects that were treated with the platelet derivatives exhibited platelet counts below 40×10³/μl of blood, and the majority of the subjects that were treated with the platelet derivatives exhibited platelet counts at or below 20×10³/μl of blood. Furthermore, it was surprising to note that although the platelet counts of the subjects administered with platelet derivatives remain lower than the subjects from the control group, a higher percentage of subjects administered with any dose of the platelet derivatives exhibited a decrease in bleeding to WHO bleeding score of 1 or less at the bleeding sites within the first 12 or 24 hours as compared to the decrease in bleeding of the subjects from the control group.

As part of the other outcomes, Table 3 depicts SAEs that were reported for the study.

TABLE 3
Grade 3* and 4 SAEs by System Organ Class and Treatment Group

| | Low | Medium | High | | |
|---|---|---|---|---|---|
| System Organ Class | Dose | Dose | Dose | Control | Total |
| General Disorders and | 1 | 2 | 2 | 0 | 5 |
| Administration Site | | | | | |
| Multiorgan Failure | 1 | 2 | 2 | 0 | 5 |
| Disease Progression | 0 | 1 | 1 | 0 | 2 |
| Infections and Infestations | 1 | 1 | 1 | 0 | 3 |
| Pneumonia | 1* | 2 | 1 | 0 | 4 |
| Sepsis | 0 | 1 | 0 | 0 | 1 |
| Neoplasms | 0 | 1 | 0 | 0 | 1 |
| AML | 0 | 1 | 0 | 0 | 1 |
| Nervous System Disorders | 1 | 0 | 0 | 0 | 1 |
| Brain Oedema | 1 | 0 | 0 | 0 | 1 |
| Respiratory, Thoracic, | 1 | 0 | 0 | 0 | 2** |
| Mediastinal Disorders | | | | | |
| Respiratory Failure | 1 | 0 | 0 | 0 | 2** |

**Two events reported in the same subject

A total of 29 SAEs were reported, and none of them were related to the administered platelet derivative treatment.

### Example 2: Crosshatching Assay to Detect Level of IgG Via Flow Cytometry

The presence of platelet antibodies (anti-HPA antibodies) in the plasma taken from thrombocytopenia (TCP) subjects or patients were detected by incubating the plasma and control samples with the platelet derivatives herein, for example, FPH in order to perform crossmatching assay.

The test was run with the samples of 20 TCP subjects, and compared with a control serum and a known reactive serum. Flow cytometry detection of platelet reactive antibodies is an effective method to detect alloantibodies bound to target platelets or lymphocytes.

Single donor apheresis platelet units (APU) were purchased from LifeShare Blood Center. The freeze-dried platelet derivatives were prepared according to the method as described in Example 1 of U.S. Pat. No. 11,529,587 B2. These freeze-dried platelet derivatives were rehydrated with sterile water equivalent to the fill volume prior to lyophilization.

The blood samples of TCP patients that have been tested for the presence of anti-HLA and anti-HPA antibodies by high resolution HLA Class 1 typing and HLA Class 1 antibody screening were received from the American Red Cross. Of the 20 blood samples of TCP patients, 5 samples (20%) were identified as positive for anti-HLA or anti-HPA antibodies. TCP citrated plasma was retrieved from TCP patient samples.

Each serum sample of a TCP patient was incubated with three different lots of the platelet derivatives (Lot A, Lot B, Lot C). The platelet derivatives were adjusted to a count of 30 k/μL in flow wash buffer (FWB) (1×PBS and 5% Heat-inactivated normal goat serum, Jackson Immuneresearch Laboratories, Inc, West Grove PA, jacksonimmuno.com). A volume of 200 μL of diluted suspension having platelet derivatives was added to a microcentrifuge tube, to which 200 μL of FWB was added for each individual test sample.

Samples were centrifuged at RCF of 1,100×g for 10 minutes at room temperature in a microcentrifuge to pellet the platelet derivatives. Supernatant was discarded and the pellet was resuspended in 60 μL of FWB plus 60 μL of TCP patient serum or control serum and incubated for 20 minutes at room temperature on a rocker. Controls consisted of a known positive sample “positive serum” (University of Michigan, serum 6) and normal human serum (negative control) (Innovative Research, Novi MI, innov-research.com). The platelet derivatives were incubated with FWB to create the “no serum” control. After incubation, cells were recovered by centrifugation at RCF of 1,100×g for 10 minutes, the supernatant was removed, and the pellet was resuspended with 500 μL FWB. The cells were centrifuged two more times, and finally resuspended with 30 μL of FWB. For staining, 10 μL of cells were incubated with 0.2 μg PE-labeled anti-Human IgG (Goat, F(ab′)₂ fragment) (Jackson ImmunoResearch Lab) and 62.5 ng/mL FITC-labeled CD61 (BD Biosciences 348093) and brought up to a final volume of 50 μL with HMTA. Cells were stained at room temperature for 20 minutes away from open light. After incubation, samples were diluted 1:20 with PBS and acquired on the NovoCyte Quanteon flow cytometer (Agilent, Santa Clara, CA, agilent.com). Mean fluorescence intensity (MFI) of IgG on the surface of the platelet derivatives within the CD61-positive population was analyzed using NovoExpress software.

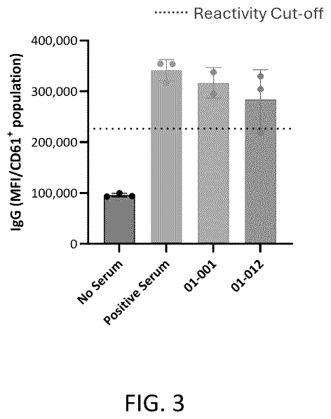

FIG. 3 shows the average MFI from the three lots of platelet derivatives for two TCP patient samples (01-001 and 01-012) that were positive for anti-HLA or anti-HPA antibodies, and had cross-reactivity with the platelet derivatives. The negative and positive controls behaved as expected. It can be observed that the two samples from TCP patients (01-001 and 01-012) showed an MFI similar to that of the positive serum, and the MFI is above the reactivity cut-off (dotted horizontal line) thereby demonstrating the cross-reactivity with the platelet derivatives. Not to be limited by theory, the cross-reactivity is believed to be caused by antibodies in the patient samples that bind the platelet derivates.

### Example 3: The Ability of Platelet Derivatives to Generate Thrombin is not Affected by the Cross-Reactivity of Plasma Samples to the Platelet Derivatives

The ability of the platelet derivatives to maintain thrombin generation ability (TGA) parameters compared to a normal plasma control was shown for two TCP plasma samples (01-001 and 01-012) that are positive for anti-HLA or anti-HPA antibodies and have cross reactivity to the platelet derivatives.

Single donor apheresis platelet units (APU) were purchased from LifeShare Blood Center. The freeze-dried platelet derivatives were prepared according to the method as described in Example 1 of U.S. Pat. No. 11,529,587 B2. These platelet derivatives were rehydrated with sterile water equivalent to the fill volume prior to lyophilization.

The blood samples from TCP patients that have been tested for the presence of anti-HLA and anti-HPA antibodies by high resolution HLA Class 1 typing and HLA Class 1 antibody screening were received from the American Red Cross. Of the 20 TCP patient blood samples, 5 samples (20%) were identified as positive for anti-HLA or anti-HPA antibodies. TCP citrated plasma was retrieved from TCP patient samples.

The platelet derivatives were adjusted to a count of 30,000 cells/μL in FWB (Jackson ImmuneResearch Laboratories, Inc). A volume of 200 μL of diluted suspension of platelet derivatives was added to a microcentrifuge tube, to which 200 μL of FWB for each individual test. Samples were centrifuged at RCF of 1,100×g for 10 minutes at room temperature in a microcentrifuge to pellet the platelet derivatives. Supernatant was discarded and the pellet was resuspended in 60 μL of FWB plus 60 μL of TCP patient serum or control serum and incubated for 20 minutes at room temperature on a rocker. After the incubation of the platelet derivatives with plasma, each sample was diluted to a final concentration of 7,200 FPH/μL in 30% Octoplas®/70% TGA dilution buffer. Each sample was assayed in triplicate by adding 80 μL of sample per well with either 20 μL of PRP reagent or calibrator (Stago, stago.com), as per the manufacture's protocol. Normal human plasma control was prepared by pooling plasma from 8 normal donors and incubating the pooled sample with the platelet derivatives as above. The run was started by injecting 20 μL of FluCa reagent (Stago, stago.com) on a Fluoroskan plate reader (Thermo Fisher Scientific, thermofisher.com) according to the manufacturer's protocols. The assay was set to run for 75 minutes, with measurements taken every 60 seconds. Peak thrombin was automatically calculated by the Thrombinoscope software. Data is plotted as the average from the three FPH lots, with error bars representing standard deviation.

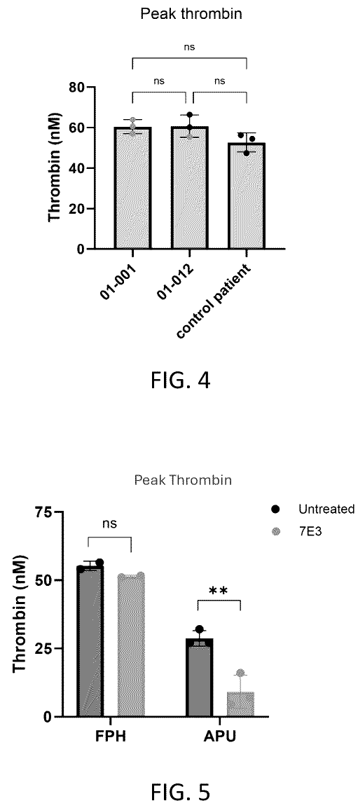

FIG. 4 shows the peak thrombin for the two TCP serum samples (01-001 and 01-012) that were both positive for anti-HLA or anti-HPA antibodies and have cross reactivity to the platelet derivatives. These results indicate that the ability of the platelet derivatives to generate thrombin was not significantly altered in the presence of TCP plasma with antibodies that cross-react to the platelet derivatives. The light-shaded circles on the top of the first two bars, corresponding to the samples 01-001 and 01-012, respectively denote that the samples are cross-reactive to the platelet derivatives. One-Way ANOVA with Dunnett's test was performed in GraphPad Prism and used to determine statistical difference compared to the normal control plasma sample. There is no statistical significance between the two TCP samples and the normal control (denoted as “control patient”).

### Example 4: Thrombin Generation of the Platelet Derivatives is Maintained Even in the Presence of Abciximab (7E3) Antiplatelet Agent in Comparison to Apheresis Platelets

Thrombin generation was determined for the platelet derivatives and apheresis platelet unit (APU) with and without the presence of abciximab (7E3).

Single donor apheresis platelet units (APU) were purchased from LifeShare Blood Center. The freeze-dried platelet derivatives were prepared according to the method as described in Example 1 of U.S. Pat. No. 11,529,587 B2. These platelet derivatives were rehydrated with sterile water equivalent to the fill volume prior to lyophilization.

For the platelet derivatives and APU samples treated with abciximab, 0.25 μg of abciximab to 1×10⁶ platelet derivatives or APU was incubated on the rocker for 20 minutes. After the incubation of the platelet derivatives or APU with abciximab, each sample was diluted to a final concentration of 7,200/μL in 30% Octoplas®/70% TGA dilution buffer. The untreated and abciximab-treated samples were tested for thrombin generation. Each sample was assayed in triplicate by adding 80 μL of sample per well with either 20 μL of PRP reagent or calibrator (Stago, stago.com), as per the manufacture's protocol. The run was started by injecting 20 μL of FluCa reagent (Stago, stago.com) on a Fluoroskan plate reader (Thermo Fisher Scientific, thermofisher.com) according to the manufacturer's protocols. The assay was set to run for 75 minutes, with measurements taken every 60 seconds. Peak thrombin was automatically calculated by the Thrombinoscope software. iOne-Way ANOVA with Dunnett's test was performed in GraphPad Prism and used to determine statistical difference between untreated and abciximab-treated samples for the platelet derivatives and APU. FIG. 5 shows the peak thrombin concentration for the platelet derivatives (two left bars labelled “FPH”) and APU (two right bars) with the presence of abciximab (light-shaded bars) and without the presence of abciximab (dark-shaded bars). It can be observed that there is no statistically significant difference for thrombin generation of the platelet derivatives between the untreated (dark-shaded bar labelled “FPH”) and abciximab-treated samples (light-shaded bar labelled “FPH”). The data demonstrates that the inhibition of GPIIb/IIIa receptor by the presence of abciximab does not affect the ability of the platelet derivatives to generate thrombin. In contrast, thrombin generation of APU in the presence of abciximab decreased (statistically significant** (p=0.0095)) as compared to the thrombin generation in the absence of abciximab.

### Example 5: Crossmatching Assay to Detect Level of IgG Via Flow Cytometry

Platelet antibodies in HPS patients' plasma and serum controls were detected by incubating samples with FPH to perform crossmatching assay. The test was run with 32 HPS patient samples and compared with a control serum and a known reactive serum. Flow cytometry detection of platelet reactive antibodies is an effective method to detect alloantibodies bound to target platelets or lymphocytes.

Single donor apheresis platelet units (APU) were purchased from LifeShare Blood Center. FPH was prepared according to the method as described in EXAMPLE 1 of U.S. Pat. No. 11,529,587 B2, incorporated herein by reference in its entirety. FPH were rehydrated with sterile water equivalent to the fill volume prior to lyophilization.

HPS patient blood samples that have been tested for the presence of anti-HLA and anti-HPA antibodies by high resolution HLA Class 1 typing and HLA Class 1 antibody screening were received from the American Red Cross. Of the 32 HPS patient blood samples (2004-001-HPS0001 to 2004-001-HPS0032), 10 samples were identified as positive for anti-HLA or anti-HPA antibodies, namely, 2004-001-HPS0001, 2004-001-HPS0004, 2004-001-HPS0007, 2004-001-HPS0008, 2004-001-HPS0014, 2004-001-HPS0015, 2004-001-HPS0016, 2004-001-HPS0020, 2004-001-HPS0027, and 2004-001-HPS0029. Out of the 10 samples, 4 of the samples were identified as positive for anti-HPA antibodies, namely, 2004-001-HPS0001, 2004-001-HPS0007, 2004-001-HPS0008, and 2004-001-HPS0029. HPS citrated plasma was retrieved from HPS patient samples.

Each HPS patient serum sample was incubated with three different lots of FPH (Lot A, Lot B, Lot C). FPH was adjusted to a count of 30 k/μL in flow wash buffer (FWB) (1×PBS and 5% Heat-inactivated normal goat serum, Jackson Immuneresearch Laboratories, Inc, West Grove PA, jacksonimmuno.com). 200 μL of diluted FPH suspension was added to a microcentrifuge tube plus 200 μL of FWB for each individual test.

Samples were centrifuged at RCF of 1,100×g for 10 minutes at room temperature in a microcentrifuge to pellet the FPH. Supernatant was discarded and the pellet was resuspended in 60 μL of FWB plus 60 μL of HPS patient serum or control serum and incubated for 20 minutes at room temperature on a rocker. Controls consisted of a known positive sample (University of Michigan, serum 6) and normal human serum (negative control) (Innovative Research, Novi MI, innov-research.com). FPH was incubated with FWB to create the no serum control. After incubation, cells were recovered by centrifugation at RCF of 1,100×g for 10 minutes, the supernatant was removed, and the pellet was resuspended with 500 μL FWB. The cells were centrifuged two more times, and finally resuspended with 30 μL of FWB. For staining, 10 μL of cells were incubated with 0.2 μg PE-labeled anti-Human IgG (Goat, F(ab′)2 fragment) (Jackson ImmunoResearch Lab) and 62.5 ng/mL FITC-labeled CD61 (BD Biosciences 348093) and brought up to a final volume of 50 μL with HMTA. Cells were stained at room temperature for 20 minutes away from open light. After incubation, samples were diluted 1:20 with PBS and acquired on the NovoCyte Quanteon flow cytometer (Agilent, Santa Clara, CA, agilent.com). Mean fluorescence intensity (MFI) of IgG on the surface of FPH within the CD61-positive population was analyzed using NovoExpress software.

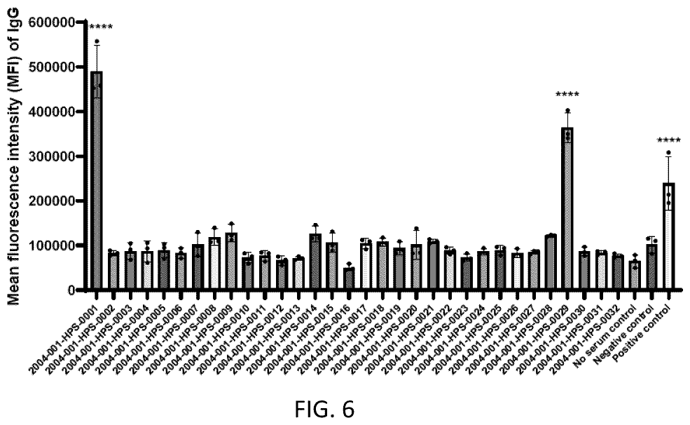

FIG. 6 shows the average MFI from the three FPH lots, with error bars representing standard deviation. One-Way ANOVA with Dunnett's test was performed in GraphPad Prism and used to determine statistical difference compared to the normal plasma sample. ****(p<0.0001). The negative and positive controls behaved as expected (average MFI of 101,465±17,928 vs 238,094±58,597, respectively). Two samples, HPS-0001 and HPS-0029, showed cross-reactivity to the three lots of FPH, both samples were previously identified as positive for anti-HLA and anti-HPA antibodies. A comparison of the compiled data for the three lots of FPH for HPS-0001 serum and the negative control shows a significant increase on IgG detected on the FPH surface (489,936±60,633 vs 101,465±17,928, p<0.0001). Increased levels of IgG were detected with HPS-0029 serum sample. Comparison of the compiled data for the three lots of FPH for HPS-0029 serum and the negative control shows a significant increase on IgG detected on the FPH surface (354,363±25,585 vs 101,465±17,928, p<0.0001).

### Example 6: FPH Ability to Generate Thrombin is not Affected by HPS Patient Plasma Samples

FPH ability to maintain thrombin generation ability (TGA) parameters compared to a normal plasma control was shown in HPS plasma samples overall, as well as HPS plasma samples positive for anti-HLA or anti-HPA antibodies and HPS plasma samples with cross reactivity to FPH.

Single donor apheresis platelet units (APU) were purchased from LifeShare Blood Center. FPH was prepared according to the method as described in EXAMPLE 1 of U.S. Pat. No. 11,529,587 B2, incorporated herein by reference in its entirety. FPH were rehydrated with sterile water equivalent to the fill volume prior to lyophilization.

HPS patient blood samples that have been tested for the presence of anti-HLA and anti-HPA antibodies by high resolution HLA Class 1 typing and HLA Class 1 antibody screening were received from the American Red Cross. Of the 32 HPS patient blood samples, 10 samples were identified as positive for anti-HLA or anti-HPA antibodies. HPS citrated plasma was retrieved from HPS patient samples.

FPH were adjusted to a count of 30,000 cells/μL in FWB (Jackson ImmuneResearch Laboratories, Inc). 200 μL of diluted FPH suspension was added to a microcentrifuge tube plus 200 μL of FWB for each individual test. Samples were centrifuged at RCF of 1,100×g for 10 minutes at room temperature in a microcentrifuge to pellet the FPH. Supernatant was discarded and the pellet was resuspended in 60 μL of FWB plus 60 μL of HPS patient serum or control serum and incubated for 20 minutes at room temperature on a rocker. After the incubation of FPH with plasma, each sample was diluted to a final concentration of 7,200 FPH/μL in 30% Octoplas®/70% TGA dilution buffer. Each sample was assayed in triplicate by adding 80 μL of sample per well with either 20 μL of PRP reagent or calibrator (Stago, stago.com), as per the manufacture's protocol. Cephalin control was prepared by diluting UPTT reagent (Bio/Data Corporation #105997, Horsham PA, biodatacorp.com) 1:50 in 30% Octaplas®/70% TGA dilution buffer. Lactadherin control was prepared by centrifuging FPH as above and resuspending the pellet in 120 uL FWB with 0.59 μg lactadherin (Haematologic Technologies #BLAC-1200, goprolytox.com). The sample was then incubated and diluted as above. The “no plasma control” was prepared by incubating with FWB in place of plasma. The normal human plasma control was prepared by pooling plasma from 8 normal donors and incubating the pooled sample with FPH as above. The run was started by injecting 20 μL of FluCa reagent (Stago, stago.com) on a Fluoroskan plate reader (Thermo Fisher Scientific, thermofisher.com) according to the manufacturer's protocols. The assay was set to run for 75 minutes, with measurements taken every 60 seconds. Peak thrombin, lag time, endogenous thrombin potential, time to peak, and velocity were automatically calculated by the Thrombinoscope software. Data is plotted as the average from the three FPH lots, with error bars representing standard deviation. One-Way ANOVA with Dunnett's test was performed in GraphPad Prism and used to determine statistical difference compared to the normal plasma sample. *(p≤0.05), **(p≤0.01), ***(p≤0.001), ****(p<0.0001).

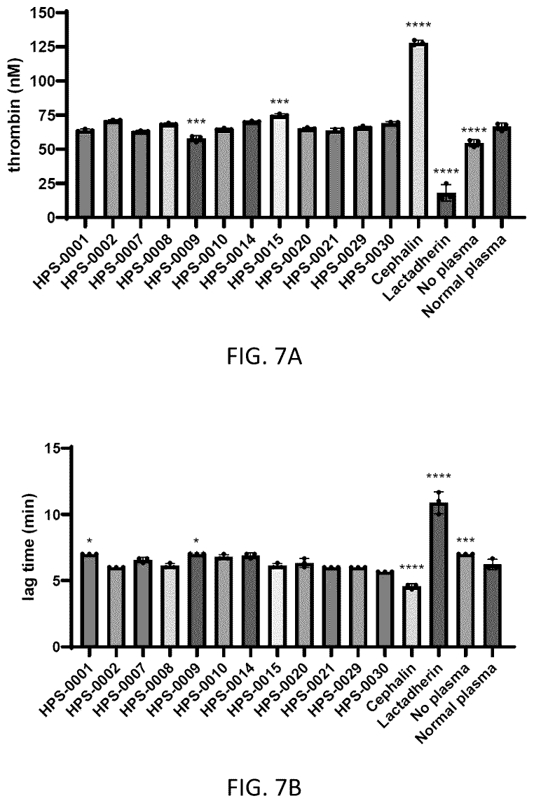

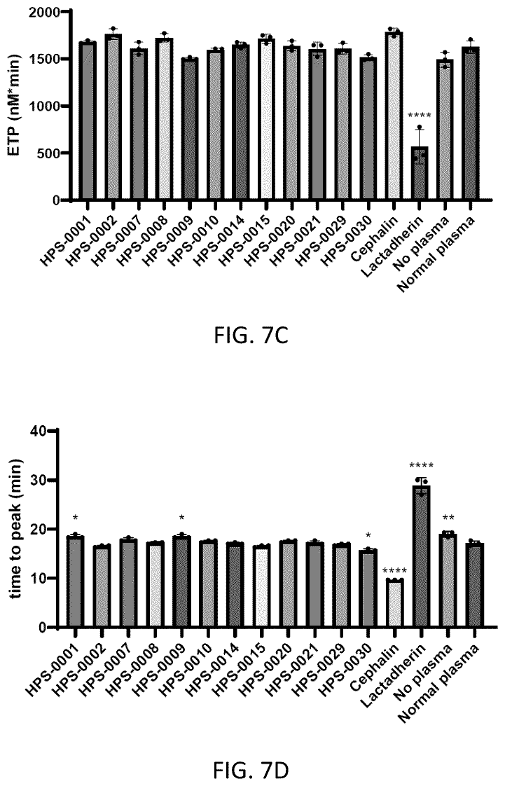

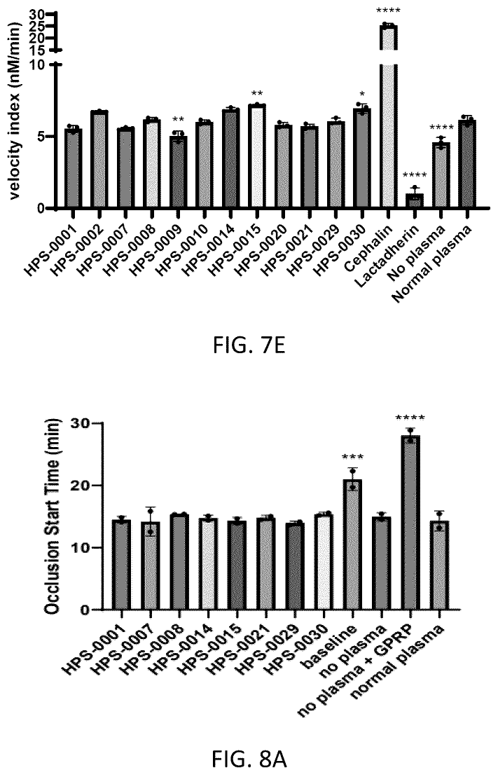

FIG. 7A shows the peak thrombin for the different HPS serum samples when incubated with FPH. FIG. 7B shows the lag time, time until thrombin first generated for the different HPS samples when incubated with FPH. FIG. 7C shows the endogenous thrombin potential or the net amount of thrombin that HPS plasma samples can generate when incubated with FPH. FIG. 7D shows the time to peak or the time for maximum concentration of thrombin generated. FIG. 7E shows the velocity index or the slope of the thrombin potential between the lag time and the time to peak.

Although some HPS plasma samples had statistically significant differences in TGA parameters compared to the normal plasma control, none of these differences are likely to be biologically meaningful rather they likely represent a variability within the human population. The only patient sample that demonstrated a consistent difference from normal plasma was HPS-0009, a sample negative for anti-HLA or anti-HPA antibodies. Patient sample HPS-0015, positive for anti-HLA antibodies, had a higher peak thrombin (74.8±1.06 nM vs. 66.5±2.76 nM; p=0.0004) and higher velocity index than normal (7.2±0.06 nM/min vs. 6.1±0.32 nM/min; p=0.0034).

Patient sample HPS-0001, positive for anti-HLA and anti-HPA antibodies (anti-GPIIbIIIa antibodies), was highly cross-reactive with FPH, had a longer lag time (7.0±0.0 min. vs. 6.2±0.4 min.; p=0.0157) and a longer time to peak (19±0.39 min. vs. 17±0.51 min; p=0.0188), but no change in peak thrombin, velocity, or ETP. Patient sample HPS-0029, having high FPH cross-reactivity, was not significantly different from the control for any other parameter.

These results indicate that the ability of FPH to generate thrombin was not significantly altered in the presence of HPS plasma, HPS plasma containing anti-HLA or anti-HPA antibodies, or HPS plasma with antibodies that cross-react to the FPH. The ability of FPH to generate thrombin is maintained regardless of the HPS plasma specification as shown in the TGA assay.

### Example 7: FPH Hemostatic Activity is Maintained on the T-TAS® when Incubated with Different HPS Plasma Samples

FPH pre-incubated with HPS plasma or normal plasma was tested on the T-TAS using a model of whole blood containing a low concentration of endogenous platelets. FPH hemostatic activity on the T-TAS is not altered due to incubation with HPS plasma, HPS plasma containing anti-HLA or anti-HPA antibodies, or FPH-cross-reactive HPS plasma.

Single donor apheresis platelet units (APU) were purchased from LifeShare Blood Center. FPH was prepared according to the method as described in EXAMPLE 1 of U.S. Pat. No. 11,529,587 B2, incorporated herein by reference in its entirety. FPH were rehydrated with sterile water equivalent to the fill volume prior to lyophilization.

HPS patient blood samples that have tested for the presence of anti-HLA and anti-HPA antibodies by high resolution HLA Class 1 typing and HLA Class 1 antibody screening were received from the American Red Cross. Of the 32 HPS patient blood samples, 10 samples were identified as positive for anti-HLA or anti-HPA antibodies. HPS citrated plasma was retrieved from HPS patient samples.

Normal donor blood was obtained on the day of the experiment and drawn into 3.2% sodium citrate vacutainers. Tubes were placed on a rocker for 30 minutes and then centrifuged at RCF of 180×g for 10 minutes and PRP was removed to a fresh tube. The buffy coat was removed and discarded. The tubes were centrifuged again at RCF of 1800×g for 10 minutes and the PPP was removed and discarded. The red blood cells (RBCs) were pipetted into a fresh tube. Octaplas® was combined with RBCs and PRP to achieve a platelet count between 30,000-50,000/μL, and a hematocrit of 40%+/−2%, as measured by the ActDiff2 hematology analyzer. The reassembled blood was tested using the T-TASO1® (Zacros, zacrosamerica.com) with HD Chip and CaCTI reagent for T-TASO1 (Zacros, zacrosamerica.com), following the manufacturer's recommendations. The platelet count was adjusted until the sample occluded at 20 minutes or longer, at which point a stock blood sample was prepared. The “No FPH” sample was prepared by adding 460 μL blood sample+20 μL HMTA+20 μL CaCTI reagent and running on the HD chip. To prepare the other samples, FPH were incubated with HPS patient plasma, normal plasma, or buffer (No plasma). FPH were diluted in HMTA±plasma to reach a total of 6.94×10\hat{ }6 cells at a ratio of 100,000 cells/μL plasma in 140 μL total volume. Samples were incubated for 20 minutes, then centrifuged at 1100×g, 10 minutes. The supernatant was discarded, and the pellet resuspended in 20 μL HMTA. 460 μL of the blood sample+20 μL CaCTI was added and the sample was run on the HD chip. For the GPRP control, an additional no plasma sample was created, and immediately before starting the run, 0.625 mM GPRP (BaChem) was added to the sample. Two replicates were performed for each sample on a single lot of FPH. Data is plotted as averages of the duplicate measurements for occlusion start time (OST), occlusion time (OT), and area under the curve (AUC), with error bars representing the standard deviation. One-Way ANOVA with Dunnett's test was performed in GraphPad Prism and used to determine statistical difference compared to the normal plasma sample. *(p≤0.05), **(p≤0.01), ***(p≤0.001), ****(p<0.0001).

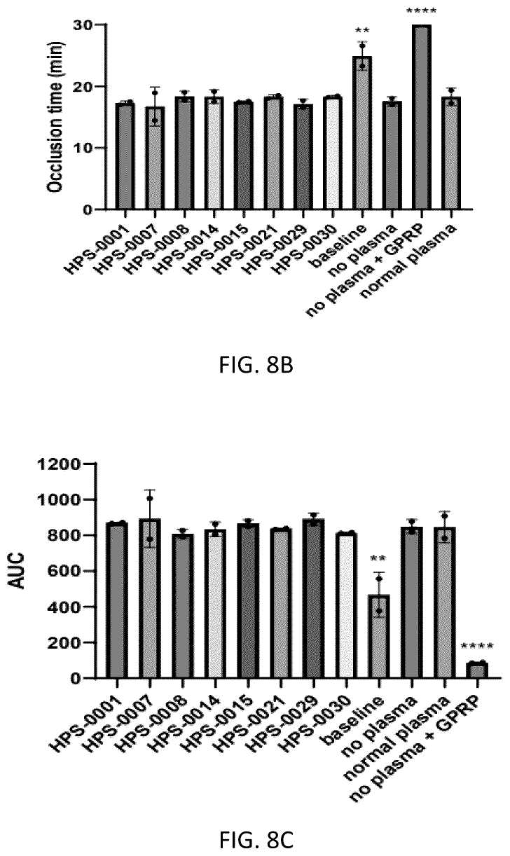

FIG. 8A, FIG. 8B, and FIG. 8C show the occlusion start time, the occlusion time, and the area under the curve (AUC), respectively, of the different samples in the HD chip. The T-TAS parameters for all HPS plasma samples were not statistically significant compared to normal plasma. In fact, two samples (HPS-001 and HPS-0029) that were determined to be cross reactive to FPH were not different from normal plasma control sample in occlusion start time (14+0.53 min, p>0.9999 and 14+0.35 min, p>0.9999, versus 14+1.6 min, respectively), occlusion time (17+0.36 min, p>0.9824 and 17+0.78 min, p>0.9598 versus 18+1.5 min, respectively), and AUC (869+4.0, p>0.9999 and 890+35, p>0.9950, versus 846+89, respectively). There was no statistical significance difference between any HPS sample and normal plasma sample regardless of the fact the sample was positive or negative for anti-HLA or anti-HPA antibodies or was cross reactive to FPH. Therefore, it can be concluded that the FPH hemostatic activity parameters in a T-TAS assay are maintained in all HPS patient plasma samples.

The disclosed embodiments, examples and experiments are not intended to limit the scope of the disclosure or to represent that the experiments below are all or the only experiments performed. Efforts have been made to ensure accuracy with respect to numbers used (e.g., amounts, temperature, etc.) but some experimental errors and deviations should be accounted for. It should be understood that variations in the methods as described may be made without changing the fundamental aspects that the experiments are meant to illustrate.

Those skilled in the art can devise many modifications and other embodiments within the scope and spirit of the present disclosure. Indeed, variations in the materials, methods, drawings, experiments, examples, and embodiments described may be made by skilled artisans without changing the fundamental aspects of the present disclosure. Any of the disclosed embodiments can be used in combination with any other disclosed embodiment.

In some instances, some concepts have been described with reference to specific embodiments. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of invention.

What is claimed is:

1. A method of decreasing bleeding of a subject, comprising:
rehydrating a platelet derivative composition in the form of a powder to form a rehydrated platelet derivative composition comprising rehydrated platelet derivatives, and
administering a dose of the rehydrated platelet derivative composition to the subject, wherein the dose comprises a first dose of the rehydrated platelet derivatives,
wherein at least 70% of the rehydrated platelet derivatives in the rehydrated platelet derivative composition are CD41 positive platelet derivatives when measured using flow cytometry, and less than 5% of the CD41 positive platelet derivatives are microparticles,
wherein the rehydrated platelet derivatives in the rehydrated platelet derivative composition have a compromised membrane,
wherein at least 70% of the rehydrated platelet derivatives in the rehydrated platelet derivative composition are CD42 positive platelet derivatives, when measured using flow cytometry,
wherein at least 70% of the rehydrated platelet derivatives in the rehydrated platelet derivative composition are CD62 positive platelet derivatives, when measured using flow cytometry,
wherein the rehydrated platelet derivatives are capable of generating thrombin in an in vitro thrombin formation assay,
wherein the rehydrated platelet derivatives show an inability to increase expression of a platelet activation marker in the presence of an agonist as compared to the expression of the platelet activation marker in the absence of an agonist, and wherein the platelet activation marker is Annexin V, CD62, or a combination thereof,
wherein the subject is refractory to platelet transfusion,
wherein the first dose of the rehydrated platelet derivatives is an amount of between 5.1×10⁸ and 9.9×10⁸/kg, or 1.0×10⁹ and 1.0×10¹⁰/kg of the subject, and
wherein the administering leads to the subject having decreased bleeding without a bleeding score of WHO Grade 2A or greater at 12 hours after the administering the first dose of the platelet derivatives.

2. The method of claim 1, wherein the subject has thrombocytopenia at the time of the administering the dose.

3. The method of claim 2, wherein the subject at the time of the administering the first dose of the rehydrated platelet derivatives has a bleeding score of WHO grade 2 but not solely the result of cutaneous bleeding.

4. The method of claim 1, wherein the first dose of the rehydrated platelet derivatives is an amount of between 1.0×10⁹ and 1.0×10¹⁰/kg of the subject.

5. The method of claim 1, wherein the administering the dose, comprises administering more than 1 dose to a maximum of 10 doses of the rehydrated platelet derivative composition, wherein each dose of the maximum of 10 doses is an amount of rehydrated platelet derivatives between 5.1×10⁸ and 9.9×10⁸/kg, or 1.0×10⁹ and 1.0×10¹⁰/kg of the subject.

6. The method of claim 3, wherein the subject has decreased bleeding without a bleeding score of WHO Grade 2A or greater at 24 hours after the administering the first dose of the platelet derivatives.

7. A method of decreasing bleeding of a subject, comprising:
rehydrating a platelet derivative composition in the form of a powder to form a rehydrated platelet derivative composition comprising rehydrated platelet derivatives, and
administering a dose of the rehydrated platelet derivative composition to the subject, wherein the dose comprises a first dose of the rehydrated platelet derivatives,
wherein at least 70% of the rehydrated platelet derivatives in the rehydrated platelet derivative composition are CD41 positive platelet derivatives when measured using flow cytometry, and less than 5% of the CD41 positive platelet derivatives are microparticles,
wherein the rehydrated platelet derivatives in the rehydrated platelet derivative composition have a compromised membrane,
wherein at least 70% of the rehydrated platelet derivatives in the rehydrated platelet derivative composition are CD42 positive platelet derivatives, when measured using flow cytometry,
wherein at least 70% of the rehydrated platelet derivatives in the rehydrated platelet derivative composition are CD62 positive platelet derivatives, when measured using flow cytometry,
wherein the rehydrated platelet derivatives are capable of generating thrombin in an in vitro thrombin formation assay,
wherein the rehydrated platelet derivatives show an inability to increase expression of a platelet activation marker in the presence of an agonist as compared to the expression of the platelet activation marker in the absence of an agonist, and wherein the platelet activation marker is Annexin V, CD62, or a combination thereof,
wherein the subject is refractory to platelet transfusion,
wherein the first dose of the rehydrated platelet derivatives is an amount of between 5×10⁸ particles/kg and 1.0×10¹² particles/kg of the subject, and
wherein the administering leads to the subject having decreased bleeding without a bleeding score of WHO Grade 2A or greater at 12 hours after the administering the first dose of the platelet derivatives.

8. The method of claim 7, wherein the subject has thrombocytopenia at the time of the administering the dose.

9. The method of claim 7, wherein the subject has severe thrombocytopenia at the time of the administering the dose.

10. The method of claim 7, wherein the first dose of the rehydrated platelet derivatives is an amount of between 5.1×10⁸ and 9.9×10⁸/kg of the subject.

11. The method of claim 7, wherein the first dose of the rehydrated platelet derivatives is an amount of between 1.0×10⁹ and 1.0×10¹²/kg of the subject.

12. The method of claim 7, wherein the first dose of the rehydrated platelet derivatives is an amount of between 1.0×10⁹ and 1.0×10¹⁰/kg of the subject.

13. The method of claim 7, wherein the administering the dose, comprises administering between 2 and 10 doses of the rehydrated platelet derivative composition, wherein each dose that is administered between 2 and 10 times is an amount of rehydrated platelet derivatives between 5×10⁸ particles/kg and 1.0×10¹² particles/kg of the subject.

14. The method of claim 7, wherein the subject at the time of the administering the first dose of the rehydrated platelet derivatives has a bleeding score of WHO grade 2 but not solely the result of cutaneous bleeding.

15. The method of claim 14, wherein the subject has decreased bleeding without a bleeding score of WHO Grade 2A or greater at 24 hours after the administering the first dose of the rehydrated platelet derivatives.

16. The method of claim 7, wherein the subject has anti-human leukocyte antigen (HLA) antibodies and/or anti-human platelet antigen (HPA) antibodies in their blood at the time of the administering the first dose of the rehydrated platelet derivatives.

17. The method of claim 7, wherein the subject has cross-reactive antibodies against the rehydrated platelet derivatives.

18. The method of claim 7, wherein the administering the dose, comprises administering between 2 and 8 times the dose of the rehydrated platelet derivative composition, wherein each dose that is administered between 2 and 8 times is an amount of rehydrated platelet derivatives between 5×10⁸ particles/kg and 1.0×10¹² particles/kg of the subject.

19. The method of claim 7, wherein the subject experiences no adverse events attributable to the administering the dose of the rehydrated platelet derivative composition when the decreased bleeding is detected.

20. The method of claim 14, wherein within 3 days after the administering the first dose of the rehydrated platelet derivatives, the bleeding of the subject is decreased to a WHO bleeding score of 0.

21. The method of claim 7, wherein the administering the dose, comprises administering between 2 and 6 times the dose of the rehydrated platelet derivative composition, wherein each dose that is administered between 2 and 6 times is an amount of rehydrated platelet derivatives between 5×10⁸ particles/kg and 1.0×10¹² particles/kg of the subject.

22. The method of claim 7, wherein at the time of the administering the first dose of the rehydrated platelet derivatives, the subject has a WHO bleeding score of Grade 2 or Grade 3.

23. The method of any one of the other claims herein, wherein the administering the dose of the rehydrated platelet derivative composition restores normal hemostasis in the subject.

24. The method of claim 7, wherein the subject has Glanzmann thrombasthenia.

25. The method of claim 7, wherein the subject is not undergoing another treatment for thrombocytopenia at the time between the administering the first dose of the rehydrated platelet derivatives and a determination that the bleeding is reduced.

26. The method of claim 7, wherein at the time of administering the first dose of the rehydrated platelet derivatives, the subject does not have another thrombotic or ischemic condition.

27. The method of claim 7, wherein at the time of administering the first dose of the rehydrated platelet derivatives, the subject is not taking any pro or anti-thrombotic medications.

28. The method of claim 14, wherein after the administering the dose of the rehydrated platelet derivative composition, the subject has no sites that are actively bleeding with a bleeding score WHO Grade 2A or greater.

29. The method of claim 7, wherein the administering the dose of the rehydrated platelet derivative composition leads to cessation of bleeding at the primary bleeding site.

30. The method of claim 29, wherein the administering the dose of the rehydrated platelet derivative composition leads to cessation of bleeding at all bleeding sites other than the primary bleeding site.

31. The method of claim 7, wherein the administering the dose of the rehydrated platelet derivative composition is performed until the bleeding stops at a primary bleeding site.

32. The method of claim 31, wherein the administering the dose of the rehydrated platelet derivative composition is performed until there is cessation of bleeding at a bleeding site other than the primary bleeding site.

33. The method of claim 31, wherein the administering the dose of the rehydrated platelet derivative composition is performed until there is cessation of bleeding at all bleeding sites in the subject.

34. The method of any one of the other claims herein, wherein the rehydrated platelet derivative composition comprises less than 3% microparticles.

35. The method of any one of the other claims herein, wherein at least 75% of the rehydrated platelet derivatives are CD41-positive, at least 75% of the rehydrated platelet derivatives are CD42-positive, at least 75% of the rehydrated platelet derivatives are CD62-positive.

36. The method of any one of the other claims herein, wherein the rehydrated platelet derivatives have one or both of:
thrombospondin (TSP) on their surface at a level that is at least 50% higher than on the surface of resting platelets; and
von Willebrand factor (vWF) on their surface at a level that is at least 50% higher than on the surface of resting platelets.

37. The method of any one of the other claims herein, wherein the rehydrated platelet derivative composition comprises microparticles having a diameter less than 0.5 μm and present in an amount in the range of 0.1 to 4.9%.

38. The method of any one of the other claims herein, wherein the administering the dose is performed by infusing the rehydrated platelet derivative composition to the subject.

39. The method of any one of the other claims herein, wherein the administering the dose leads to a cessation or a decrease in bleeding at a primary bleeding site at 12 hours, or 24 hours after the administering the first dose of the rehydrated platelet derivatives.

40. The method of any one of the other claims herein, wherein the administering the dose leads to a cessation or a decrease in bleeding at bleeding sites other than a primary bleeding site at 12 hours, or 24 hours after the administering the first dose of the rehydrated platelet derivatives.

41. The method of claim 7, wherein the administering the dose leads to cessation of bleeding at all bleeding sites within 12, or 24 hours after the administering the first dose of the rehydrated platelet derivatives.

42. The method of claim 7, wherein the subject experiences no adverse events, or no grade 2 or higher adverse events, attributable to the administering the dose of the rehydrated platelet derivative composition when the decreased bleeding is detected.

43. The method of claim 7, wherein the subject experiences no grade 3 or grade 4 adverse events attributable to the administering the dose of the rehydrated platelet derivative composition, when the decreased bleeding is detected.

44. The method of claim 7, wherein the subject experiences no adverse events or no grade 2 or higher adverse events, attributable to the administering the dose of the rehydrated platelet derivative composition, within 30 days of the first dose.

45. The method of claim 7, wherein the subject experiences no grade 2 or higher adverse events, attributable to the administering the dose of the rehydrated platelet derivative composition, within 24 hours of the first dose.

46. The method of claim 7, wherein the subject experiences no adverse events, or no grade 2 or higher adverse events, attributable to the administering the dose of the rehydrated platelet derivative composition, within 72 hours of the first dose.

47. The method of claim 7, wherein the subject experiences no adverse events, or no grade 2 or higher adverse events, attributable to the administering the dose of the rehydrated platelet derivative composition, within 12 hours of the first dose.

48. The method of any one of the other claims herein, wherein the rehydrated platelet derivatives when at a concentration of about 255×10³ platelet derivatives/μL are capable of producing occlusion by attaining a pressure of 80 kPa in less than 14 minutes in platelet-reduced citrated whole blood in an in vitro total thrombus-formation analysis system (T-TAS).

49. The method of claim 7, wherein the rehydrated platelet derivatives are capable of yielding+/−25% of one or more occlusion start time, occlusion time, or area under the curve (AUC) in an in vitro T-TAS assay in the presence versus absence of anti-HLA and/or anti-HPA antibodies.

50. The method of claim 7, wherein the rehydrated platelet derivatives are capable of yielding+/−25% of one or more occlusion start time, occlusion time, or area under the curve (AUC) in an in vitro T-TAS assay in the presence versus absence of anti-HPA antibodies.

51. The method of claim 7, wherein the rehydrated platelet derivatives restore thrombin levels identical 4,800 particles/microliter.

52. The method of claim 7, wherein the rehydrated platelet derivatives are capable of yielding comparable in vitro thrombin generation activity in the presence versus absence of cross-reactive antibodies against the platelet derivatives.

53. The method of claim 7, wherein the rehydrated platelet derivatives are capable of yielding comparable hemostatic activity in an in vitro T-TAS assay in the presence versus absence of cross-reactive antibodies against the platelet derivatives.

54. The method of claim 7, wherein the rehydrated platelet derivatives are capable of yielding comparable in vitro thrombin generation activity in the presence versus absence of anti-HLA and/or anti-HPA antibodies.

55. The method of claim 7, wherein the rehydrated platelet derivatives are capable of yielding comparable hemostatic activity in an in vitro T-TAS assay in the presence versus absence of anti-HLA and/or anti-HPA antibodies.

56. The method of claim 7, wherein the administering the dose of the rehydrated platelet derivative composition leads to an improvement in thrombin generation in the subject as compared to the subject before the administering and/or an improvement in clot formation in the subject as compared to the subject before the administering.

57. The method of claim 7, wherein the rehydrated platelet derivatives have 12% to 64%, 20% to 64%, 20% to 60%, 20% to 50%, or 25% to 50% amount of CD42 as compared to the amount of CD42 in fresh platelets or apheresis platelets.
